(19) United States
(12) Patent Application Publication
(10) Pub. No.: US 2024/0082378 A1
(43) Pub. Date: Mar. 14, 2024

(54) Staphylococcus aureus Antigen-Based Nucleic Acid Vaccines

(71) Applicant: University of Rochester, Rochester, NY (US)

(72) Inventors: Gowrishankar Muthukrishnan, Rochester, NY (US); Steven R. Gill, Webster, NY (US); Cal D. Palumbo, Rochester, NY (US); Edward M. Schwarz, Rochester, NY (US)

(21) Appl. No.: 18/261,568

(22) PCT Filed: Jan. 14, 2022
(86) PCT No.: PCT/US22/12440

§ 371 Date: Jul. 14, 2023

Related U.S. Application Data
(60) Provisional application No. 63/247,479, filed on Sep. 23, 2021.
(60) Provisional application No. 63/167,841, filed on Mar. 30, 2021.
(60) Provisional application No. 63/137,870, filed on Jan. 15, 2021.

(51) Int. Cl. A61K 39/085; A61P 37/04; C12N 9/24; C12N 9/36; C12N 9/80

(52) U.S. Cl. CPC A61K 39/085; A61P 37/04; C12N 9/2402; C12N 9/2462; C12N 9/80; C12Y 302/01017; C12Y 302/01096; C12Y 305/01028; A61K 2039/53

(57) ABSTRACT

The present disclosure generally relates to compositions and methods for inducing an immune response against S. aureus antigens in a subject. The invention also relates to nucleic acid vaccines and methods of use thereof to treat or prevent diseases or disorders associated with S. aureus infection.

### CROSS-REFERENCE TO RELATED APPLICATIONS

This application claims priority to U.S. Provisional Patent Application No. 63/137,870, filed on Jan. 15, 2021, U.S. Provisional Patent Application No. 63/167,841, filed on Mar. 30, 2021, and U.S. Provisional Patent Application No. 63/247,479, filed on Sep. 23, 2021, the contents of each of which are incorporated by reference herein in their entirety.

### STATEMENT REGARDING FEDERALLY SPONSORED RESEARCH OR DEVELOPMENT

This invention was made with government support under grant number AR072000 awarded by the National Institute of Health. The government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

The present application hereby incorporates by reference the entire contents of the text file named “204606-0136-00WO_Sequence_Listing_ST25.txt” in ASCII format. The text file containing the Sequence Listing of the present application was created on Jan. 13, 2022, and is 175,500 bytes in size.

### BACKGROUND

Staphylococcus aureus is a leading cause of mortality and morbidity worldwide, causing a diverse array of diseases by various pathogenic mechanisms and a variety of infections ranging from mild skin and soft-tissue infections to serious invasive diseases such as endocarditis, osteomyelitis, and necrotizing pneumonia (Lowy F., N. Engl. J. Med. 339(8):520-32, 1998; Klevens et al., JAMA 298(15):1763-71, 2007). The growing prevalence of antimicrobial-resistant strains in both hospital and community settings has inhibited the choice of therapy and therapeutic success considerably, resulting in increased rates of chronic and recurrent infections and rising healthcare costs.

Accordingly, alternative methods for the prevention and treatment of bacterial infection in general and S. aureus infections in particular are needed.

There is a great need for novel interventions of chronic osteomyelitis (OM) as approximately 112,000 orthopedic device-related infections occur per year in the US, at an approximate hospital cost of $15,000-70,000 per incident (Darouiche, “Treatment of Infections Associated With Surgical Implants,” N. Engl. J. Med. 350(14):1422-9, 2004). Although improvements in surgical technique and aggressive antibiotic prophylaxis have decreased the infection rate following orthopedic implant surgery to 1-5%, osteomyelitis (OM) remains a serious problem and appears to be on the rise from minimally invasive surgery (Mahomed et al., “Rates and Outcomes of Primary and Revision Total Hip Replacement in the United States Medicare Population,” J. Bone Joint Surg. Am. 85(A-1):27-32, 2003; WHO Global Strategy for Containment of Antimicrobial Resistance, 2001). The significance of this resurgence, 80% of which is due to Staphylococcus aureus, is amplified by the fact that ˜50% of clinical isolates are methicillin resistant S. aureus (MRSA). While the infection rates for joint prosthesis and fracture-fixation devices have been only 0.3-11% and 5-15% of cases, respectively, over the last decade (Lew and Waldvogel, “Osteomyelitis,” Lancet 364(9431):369-79 (2004); Toms et al., “The Management of Pen-Prosthetic Infection in Total Joint Arthroplasty,” J. Bone Joint Surg. Br. 88(2):149-55, 2006), this result may lead to amputation or death. Additionally, the popularization of “minimally invasive surgery” for elective total joint replacements (TJR) in which the very small incision often leads to complications from the prosthesis contacting skin during implantation, has markedly increased the incidence of OM (Mahomed et al., “Rates and Outcomes of Primary and Revision Total Hip Replacement in the United States Medicare Population,” J. Bone Joint Surg. Am. 85(A-1):27-32, 2003; WHO Global Strategy for Containment of Antimicrobial Resistance, 2001). These infections require a very expensive two-stage revision surgery, and recent reports suggest that success rates could be as low as 50% (Azzam et al., “Outcome of a Second Two-stage Reimplantation for Periprosthetic Knee Infection,” Clin. Orthop. Relat. Res. 467(7):1706-14, 2009). However, the greatest concern is the emergence of drug resistant strains, most notably MRSA, which has surpassed HIV as the most deadly pathogen in North America, and continues to make the management of chronic OM more difficult, placing a great demand for novel therapeutic interventions. There is a great need for alternative interventional strategies, particularly for immune compromised elderly who are the primary recipients of TJR.

Studies have documented that 80% of chronic OM is caused by S. aureus. These bacteria contain several factors that make them bone pathogens including several cell-surface adhesion molecules that facilitate their binding to bone matrix (Flock et al., “Cloning and Expression of the Gene for a Fibronectin-Binding Protein From Staphylococcus aureus,” Embo. J. 6(8):2351-7, 1987), toxins capable of stimulating bone resorption (Nair et al., “Surface-Associated Proteins From Staphylococcus aureus Demonstrate Potent Bone Resorbing Activity,” J. Bone Miner. Res. 10(5):726-34, 1995), through increased osteoclast activity (Marriott et al., “Osteoblasts Express the Inflammatory Cytokine Interleukin-6 in a Murine Model of Staphylococcus aureus Osteomyelitis and Infected Human Bone Tissue,” Am. J. Pathol. 164(4):1399-406, 2004). The rate-limiting step in the evolution and persistence of infection is the formation of biofilm around implanted devices (Costerton et al., “Bacterial Biofilms: A Common Cause of Persistent Infections,” Science 284(5418):1318-22, 1999). Shortly after implantation, a conditioning layer composed of host-derived adhesins (including fibrinogen, fibronectin, and collagen) forms on the surface of the implant and invites the adherence of free-floating bacteria derived from hematogenous seeding, including spread of infection from a contiguous area (the skin adjacent to a wound), surgical inoculation of bacteria into bone, or trauma coincident with significant disruption of the associated soft tissue bone envelope (Darouiche, “Treatment of Infections Associated With Surgical Implants,” N. Engl. J. Med. 350(14):1422-9, 2004). Over the next few days bacterial cell division, recruitment of additional planktonic organisms, and secretion of bacterial products (such as the glycocalyx) produces the biofilm. This biofilm serves as a dominant barrier to protect the bacteria from the action of antibiotics, phagocytic cells, antibodies and impairs lymphocyte functions (Gray et al., “Effect of Extracellular Slime Substance From Staphylococcus epidermidis on the Human Cellular Immune Response,” Lancet 1(8373):365-7, 1984; Johnson et al., “Interference With Granulocyte Function By Staphylococcus epidermidis Slime,” Infect. Immun. 54(1):13-20, 1986; Naylor et al., “Antibiotic Resistance of Biomaterial-Adherent Coagulase-Negative and Coagulase-Positive Staphylococci,” Clin. Orthop. Relat. Res. 261:126-33, 1990).

Another recent discovery is that S. aureus not only colonizes bone matrix, but is also internalized by osteoblasts in vitro (Ellington et al., “Involvement of Mitogen-Activated Protein Kinase Pathways in Staphylococcus aureus Invasion of Normal Osteoblasts,” Infect. Immun. 69(9):5235-42, 2001) and in vivo (Reilly et al., “In Vivo Internalization of Staphylococcus aureus by Embryonic Chick Osteoblasts,” Bone 26(1):63-70, 2000). This provides yet another layer of antibody and antibiotic resistance. This phase of infection occurs under conditions of markedly reduced metabolic activity and sometimes appears as so-called small-colony variants that likely accounts for its persistence (Proctor et al., “Persistent and Relapsing Infections Associated with Small-Colony Variants of Staphylococcus aureus,” Clin. Infect. Dis. 20(1):95-102, 1995). At this point the bacteria may also express phenotypic resistance to antimicrobial treatment, also explaining the high failure rate of short courses of therapy (Chuard et al., “Resistance of Staphylococcus aureus Recovered From Infected Foreign Body in Vivo to Killing by Antimicrobials,” J. Infect. Dis. 163(6):1369-73, 1991). Due to these extensive pathogenic mechanism, OM is notorious for its tendency to recur even after years of quiescence, and it is accepted that a complete cure is an unlikely outcome (Mader and Calhoun, “Long-Bone Osteomyelitis Diagnosis and Management,” Hosp. Pract. (Off Ed) 29(10):71-6, 9, 83 passim, 1994).

One of the key questions in the field of chronic OM is why current knowledge of factors that regulate chronic OM so limited. Supposedly, the experimental tools necessary to elucidate bacterial virulence gene have been available for over a century. There are three explanations for this anomaly. First, although the total number of osteomyelitis cases is high, its incidence of 1-5% is too low for rigorous prospective clinical studies, with the possible exception of revision arthropasty. Second, it is well known that in vitro cultures rapidly select for growth of organisms that do not elaborate an extracellular capsule, such that biofilm biology can only be studied with in vivo models (Costerton et al., “Bacterial Biofilms: A Common Cause of Persistent Infections,” Science 284(5418):1318-22, 1999). This leads to the “greatest obstacle” in this field, which is the absence of a quantitative animal model that can assess the initial planktonic growth phase of the bacteria prior to biofilm formation. To date, much of the knowledge of its pathogenesis comes from animal models (Norden, “Lessons Learned From Animal Models of Osteomyelitis,” Rev. Infect. Dis. 10(1):103-10, 1988), which have been developed for the chicken (Daum et al., “A Model of Staphylococcus aureus Bacteremia, Septic Arthritis, and Osteomyelitis in Chickens,” J. Orthop. Res. 8(6):804-13, 1990), rat (Rissing et al., “Model of Experimental Chronic Osteomyelitis in Rats,” Infect. Immun. 47(3):581-6, 1985), guinea pig (Passl et al., “A Model of Experimental Post-Traumatic Osteomyelitis in Guinea Pigs,” J. Trauma 24(4):323-6, 1984), rabbit (Worlock et al., “An Experimental Model of Post-Traumatic Osteomyelitis in Rabbits,” Br. J. Exp. Pathol. 69(2):235-44, 1988), dog (Varshney et al., “Experimental Model of Staphylococcal Osteomyelitis in Dogs,” Indian J. Exp. Biol. 27(9):816-9, 1989), sheep (Kaarsemaker et al., “New Model for Chronic Osteomyelitis With Staphylococcus aureus in Sheep,” Clin. Orthop. Relat. Res. 339:246-52, 1997) and most recently mouse (Marriott et al., “Osteoblasts Express the Inflammatory Cytokine Interleukin-6 in a Murine Model of Staphylococcus aureus Osteomyelitis and Infected Human Bone Tissue,” Am. J. Pathol. 164(4):1399-406 (2004)). While these models have been used to confirm the importance of bacterial adhesions identified from in vitro assays (Chuard et al., “Susceptibility of Staphylococcus aureus Growing on Fibronectin-Coated Surfaces to Bactericidal Antibiotics,” Antimicrob. Agents Chemother. 37(4):625-32, 1993; Buxton et al., “Binding of a Staphylococcus aureus Bone Pathogen to Type I Collagen,” Microb. Pathog. 8(6):441-8, 1990; Switalski et al., “A Collagen Receptor on Staphylococcus aureus Strains Isolated From Patients With Septic Arthritis Mediates Adhesion to Cartilage,” Mol. Microbiol. 7(1):99-107, 1993), they do not have an outcome measure of in vivo growth, bacterial load, or osteolysis. Thus, they cannot be efficiently used to assess drug effects, bacterial mutants, and the role of host factors with transgenic mice.

Based on over 150 years of research, a clear paradigm to explain microbial pathogenesis has emerged. This model also applies to OM. The initial step of infection occurs when a unicellular bacterium invades the body. At this point the microbe must respond to environmental changes and express virulence genes that will help it defeat innate immunity and provide it with adhesin receptors to attach to the host. The bacterium is also dependent on the stochastic availability of host adhesins from necrotic tissue or a foreign body such as an implant. Successful completion of these steps leads to an exponential growth phase, which ceases at the point of nutrient exhaustion and/or the development of adaptive immunity. Following the exponential growth phase the bacteria are forced to persist under dormant growth conditions within the biofilm. However, at this point the infection is now chronic and cannot be eradicated by drugs or host immunity. Thus, the focus in this field has been on cell surface adhesins that specifically interact with extracellular matrix components known as MSCRAMMs (microbial surface components recognizing adhesive matrix molecules) (Patti et al., “MSCRAMM-Mediated Adherence of Microorganisms to Host Tissues,” Annu. Rev. Microbiol. 48:585-617, 1994). In fact, essentially all anti-S. aureus vaccines that have been developed to date have been directed against MSCRAMMs that are important for host tissue colonization and invasion. The goal of these vaccines is to generate antibodies that bind to these surface antigens, thereby inhibiting their attachment to host tissue. By opsinizing the bacterial surface, these antibodies can also mediate S. aureus clearance by phagocytic cells. Unfortunately, S. aureus has many adhesins, such that inhibition of one or more may not be sufficient to prevent bacterial attachment. Furthermore, bacterial clearance by phagocytic cells may be limited in avascular tissue, such that mAb may need additional anti-microbial mechanism of action to significantly reduce the in vivo planktonic growth of S. aureus and prevent the establishment of chronic OM or reinfection during revision total joint replacement surgery.

In gram-positive and gram-negative bacteria, autolysins play an important role in cell separation and cell wall remodeling during normal binary fission. The 138-kDa S. aureus autolysin is proteolytically processed on the cell surface to produce two active enzymes, N-acetylmuramyl-L-alanine amidase (amidase, Amd; 62-kDa) and endo-β-N-acetylglucosaminidase (glucosaminidase, Gmd; 51-kDa), that remain non-covalently attached to the cell surface (Komatsuzawa H, et al. Microbiol Immunol, 1997, 41(6):469-479; Komatsuzawa H, et al. Antimicrob Agents Chemother, 1997, 41(11):2355-2361; Oshida T, et al. PNAS, 1995, 92(1):285-289; Yamada S, et al. J Bacteriol, 1996, 178(6):1565-1571). The use of Amd and Gmd as target antigens for immunization is supported by the discovery of the protection or augmentation of mouse immunity to S. aureus infections conferred by passive immunization with anti-Amd or anti-Gmd monoclonal antibodies (PCT/US2011/035033 and PCT/US2014/070337, incorporated herein by reference). These antigens have also been validated in murine models, (Varrone J J, et al. Bonekey Osteovision, 2011, 8:187-94; Varrone J J, et al. J Orthop Res, 2014, 32(10):1389-96; Yokogawa N, et al. J Orthop Res, 2018, 36(6):1590-98) and in non-intervention clinical research (Oh I, et al. Infect Immun, 2018, 86(12); Lee C C, et al. Eur Cell Mater, 2020, 39:96-107; Kates S L, et al. J Bone Joint Surg Am, 2020, 102(21):1842-48). Furthermore, identification of anti-Gmd immunity was recently recognized as the “Holy Grail of Infection Prevention” (Chen A F. J Bone Joint Surg Am. 2020, 102(21):e122).

Presently, there are no long-term prophylactic treatments that can protect high-risk patients from MRSA, most notably the aging “baby boomers” who account for most of the 1.5 million TJR performed annually in the United States. A vaccine that would decrease the MRSA incidence by 50-80% would not only reduce the number one complication of joint replacement and open fracture repair procedures, but also cut the healthcare burden by a similar amount.

Thus, there is a need in the art for improved compositions and methods for treating or preventing S. aureus infection. This invention satisfies this unmet need.

### SUMMARY OF THE DISCLOSURE

In one embodiment, the present disclosure relates to a composition for inducing an immune response against Staphylococcus aureus (S. aureus) in a subject, the composition comprising one or more nucleic acid molecules encoding one or more S. aureus antigenic polypeptide, immunogenic variant or fragment thereof. In some embodiments, the one or more nucleic acid molecules is contained within one or more vector (e.g., a RNA viral vector, a DNA viral vector, and a plasmid). In some embodiments, the composition is a vaccine and optionally further comprises an adjuvant.

In one embodiment, the present disclosure relates to a method for inducing an antigen specific immune response in a subject, comprising administering to the subject one or more composition comprising one or more nucleic acid molecule encoding one or more S. aureus antigenic polypeptide, immunogenic variant or fragment of the present disclosure in an amount effective to produce an antigen specific immune response. In some embodiments, the antigen specific antibody titre in the blood or serum of the subject against the administered antigenic polypeptide, immunogenic variant or fragment reaches >0.2 μg/ml by 30 days following administration. In some embodiments, the method treats or prevents S. aureus infection. In some embodiments, the method treats or prevents one or more disease or disorder associated with S. aureus infection. In some embodiments, the disease or disorder is osteomyelitis.

In some embodiments of the presently disclosed composition(s) and/or method(s), the one or more S. aureus antigenic polypeptide, immunogenic variant or fragment corresponds to an S. aureus protein selected from: autolysin (Atl), N-acetylmuramyl-L-alanine amidase (Amd), endo-β-N-acetylglucosaminidase (Gmd), alpha-hemolysin (Hla), chemotaxis inhibiting protein of S. aureus (CHIPS), Staphylococcal complement inhibitor (SCIN), and coproporphyrinogen III oxidase (CgoX).

In some embodiments of the presently disclosed composition(s) and/or method(s), the one or more S. aureus antigenic polypeptide, immunogenic variant or fragment corresponds to an S. aureus lytic transglycosylase protein. In some embodiments, the one or more S. aureus antigenic polypeptide, immunogenic variant or fragment corresponds to IsaA or SceD.

In some embodiments of the presently disclosed composition(s) and/or method(s): (a) the one or more nucleic acid molecule comprises at least one of: (i) a polynucleotide sequence that does not occur in nature; (ii) a polynucleotide sequence codon optimized for expression in cells of a mammalian subject; and, (iii) a modified nucleoside; (b) the one or more nucleic acid molecules is encapsulated by a lipid nanoparticle; and/or (c) the one or more nucleic acid molecules is contained within one or more vector (e.g., an RNA viral vector, a DNA viral vector, and a plasmid).

In some embodiments of the presently disclosed composition(s) and/or method(s): the one or more nucleic acid molecules comprises one or more selected from: (a) a polynucleotide sequence encoding an Atl polypeptide comprising the amino acid sequence of SEQ ID NO: 1 (e.g., the nucleic acid sequence of SEQ ID NO:2 or the RNA sequence of SEQ ID NO: 3) or SEQ ID NO:72 (e.g., the nucleic acid sequence of SEQ ID NO:73 or the RNA sequence of SEQ ID NO: 74), or an immunogenic variant or fragment thereof; (b) a polynucleotide sequence encoding an Amd polypeptide comprising the amino acid sequence of SEQ ID NO: 4 (e.g., the nucleic acid sequence of SEQ ID NO:5 or the RNA sequence of SEQ ID NO: 6), or an immunogenic variant or fragment thereof; (c) a polynucleotide sequence encoding an Amd polypeptide fragment comprising: (i) the amino acid sequence of SEQ ID NO: 7 (e.g., the nucleic acid sequence of SEQ ID NO:8 or the RNA sequence of SEQ ID NO:9), or an immunogenic variant or fragment thereof; (ii) the amino acid sequence of SEQ ID NO: 10 (e.g., the nucleic acid sequence of SEQ ID NO:11 or the RNA sequence of SEQ ID NO:12), or an immunogenic variant or fragment thereof; (iii) the amino acid sequence of SEQ ID NO: 13 (e.g., the nucleic acid sequence of SEQ ID NO:14 or the RNA sequence of SEQ ID NO:15), or an immunogenic variant or fragment thereof; and/or (iv) the amino acid sequence of SEQ ID NO: 16 (e.g., the nucleic acid sequence of SEQ ID NO:17 or the RNA sequence of SEQ ID NO:18), or an immunogenic variant or fragment thereof; (d) a polynucleotide sequence encoding an Gmd polypeptide comprising the amino acid sequence of SEQ ID NO: 19 (e.g., the nucleic acid sequence of SEQ ID NO:20 or the RNA sequence of SEQ ID NO: 21), or an immunogenic variant or fragment thereof; and (e) a polynucleotide sequence encoding an Gmd polypeptide fragment comprising: (i) the amino acid sequence of SEQ ID NO: 22 (e.g., the nucleic acid sequence of SEQ ID NO:23 or the RNA sequence of SEQ ID NO:24), or an immunogenic variant or fragment thereof; (ii) the amino acid sequence of SEQ ID NO: 25 (e.g., the nucleic acid sequence of SEQ ID NO:26 or the RNA sequence of SEQ ID NO:27), or an immunogenic variant or fragment thereof; and/or (iii) the amino acid sequence of SEQ ID NO: 28 (e.g., the nucleic acid sequence of SEQ ID NO:29 or the RNA sequence of SEQ ID NO:30), or an immunogenic variant or fragment thereof.

In some embodiments of the presently disclosed composition(s) and/or method(s): the one or more nucleic acid molecules comprises one or more selected from: (a) a polynucleotide sequence encoding a CHIPs polypeptide comprising the amino acid sequence of SEQ ID NO: 31 (e.g., the nucleic acid sequence of SEQ ID NO:32 or the RNA sequence of SEQ ID NO: 33), or SEQ ID NO: 34 (e.g., the nucleic acid sequence of SEQ ID NO:35 or the RNA sequence of SEQ ID NO: 36), or an immunogenic variant or fragment thereof; (b) a polynucleotide sequence encoding an CHIPs polypeptide fragment comprising the amino acid sequence of SEQ ID NO: 37 (e.g., the nucleic acid sequence of SEQ ID NO:38 or the RNA sequence of SEQ ID NO:39), or an immunogenic variant or fragment thereof; (c) a polynucleotide sequence encoding an SCIN polypeptide comprising the amino acid sequence of SEQ ID NO: 40 (e.g., the nucleic acid sequence of SEQ ID NO:41 or the RNA sequence of SEQ ID NO: 42), or SEQ ID NO: 43 (e.g., the nucleic acid sequence of SEQ ID NO:75 or the RNA sequence of SEQ ID NO: 44), or an immunogenic variant or fragment thereof; (d) a polynucleotide sequence encoding an SCIN polypeptide fragment comprising: (i) the amino acid sequence of SEQ ID NO: 45 (e.g., the nucleic acid sequence of SEQ ID NO:46 or the RNA sequence of SEQ ID NO:47), or an immunogenic variant or fragment thereof; and/or (ii) the amino acid sequence of SEQ ID NO: 48 (e.g., the nucleic acid sequence of SEQ ID NO:49 or the RNA sequence of SEQ ID NO:50,), or an immunogenic variant or fragment thereof; (e) a polynucleotide sequence encoding a Hla polypeptide comprising the amino acid sequence of SEQ ID NO: 51 (e.g., the nucleic acid sequence of SEQ ID NO:52 or the RNA sequence of SEQ ID NO: 53), or SEQ ID NO: 54 (e.g., the nucleic acid sequence of SEQ ID NO:55 or the RNA sequence of SEQ ID NO: 56), or an immunogenic variant or fragment thereof; (f) a polynucleotide sequence encoding an Hla polypeptide fragment comprising the amino acid sequence of SEQ ID NO: 57 (e.g., the nucleic acid sequence of SEQ ID NO:58 or the RNA sequence of SEQ ID NO:59), or an immunogenic variant or fragment thereof; (g) a polynucleotide sequence encoding an CgoX polypeptide comprising the amino acid sequence of SEQ ID NO: 60 (e.g., the nucleic acid sequence of SEQ ID NO:61 or the RNA sequence of SEQ ID NO: 62), or an immunogenic variant or fragment thereof; and/or (h) a polynucleotide sequence encoding an CgoX polypeptide fragment comprising: (i) the amino acid sequence of SEQ ID NO: 63 (e.g., the nucleic acid sequence of SEQ ID NO:64 or the RNA sequence of SEQ ID NO:65) or an immunogenic variant or fragment thereof; (ii) the amino acid sequence of SEQ ID NO: 66 (e.g., the nucleic acid sequence of SEQ ID NO:67 or the RNA sequence of SEQ ID NO:68,) or an immunogenic variant or fragment thereof, or (iii) the amino acid sequence of SEQ ID NO: 69 (e.g., the nucleic acid sequence of SEQ ID NO:70 or the RNA sequence of SEQ ID NO:71), or an immunogenic variant or fragment thereof.

In some embodiments of the presently disclosed composition(s) and/or method(s): the one or more nucleic acid molecules comprises one or more selected from: (a) a polynucleotide sequence encoding an IsaA polypeptide comprising the amino acid sequence of SEQ ID NO: 91 (e.g., the nucleic acid sequence of SEQ ID NO:92 or the RNA sequence of SEQ ID NO: 93), or SEQ ID NO: 94 (e.g., the nucleic acid sequence of SEQ ID NO:95 or the RNA sequence of SEQ ID NO: 96), or an immunogenic variant or fragment thereof; (b) a polynucleotide sequence encoding an IsaA polypeptide fragment comprising: (i) the amino acid sequence of SEQ ID NO: 97 (e.g., the nucleic acid sequence of SEQ ID NO:98 or the RNA sequence of SEQ ID NO:99), or an immunogenic variant or fragment thereof; (ii) the amino acid sequence of SEQ ID NO: 100 (e.g., the nucleic acid sequence of SEQ ID NO:101 or the RNA sequence of SEQ ID NO:102), or an immunogenic variant or fragment thereof; and/or (iii) the amino acid sequence of SEQ ID NO: 103 (e.g., the nucleic acid sequence of SEQ ID NO:104 or the RNA sequence of SEQ ID NO:105), or an immunogenic variant or fragment thereof; (c) a polynucleotide sequence encoding an SceD polypeptide comprising the amino acid sequence of SEQ ID NO: 106 (e.g., the nucleic acid sequence of SEQ ID NO:107 or the RNA sequence of SEQ ID NO: 108), or SEQ ID NO: 109 (e.g., the nucleic acid sequence of SEQ ID NO:110 or the RNA sequence of SEQ ID NO: 111), or an immunogenic variant or fragment thereof; and/or (d) a polynucleotide sequence encoding an SceD polypeptide fragment comprising the amino acid sequence of SEQ ID NO: 112 (e.g., the nucleic acid sequence of SEQ ID NO:113 or the RNA sequence of SEQ ID NO:114), or an immunogenic variant or fragment thereof.

In some embodiments of the presently disclosed composition(s) and/or method(s): the one or more nucleic acid molecules is an mRNA. In one embodiment, the mRNA comprises a 5′ untranslated (UTR) region, an open reading frame, and a 3′ UTR and a polyA tail. In some embodiments, the one or more nucleic acid molecules is encapsulated by a lipid nanoparticle.

### BRIEF DESCRIPTION OF THE DRAWINGS

The following detailed description of embodiments of the disclosure will be better understood when read in conjunction with the appended drawings. It should be understood that the invention is not limited to the precise arrangements and instrumentalities of the embodiments shown in the drawings.

FIG. 1 depicts exemplary results demonstrating that anti-autolysin antibody levels are lower in patients with adverse outcomes due to orthopaedic S. aureus infections. Results show serum IgG levels, represented as median fluorescent intensity, against autolysin (Gmd and Amd) antigens determined via Luminex immunoassay for patients in the AOTrauma CPP Bone Infection Registry with reported S. aureus infections (N=194).

FIG. 2 comprises FIG. 2A through 2B. FIG. 2B depicts exemplary results demonstrating that anti-autolysin and secreted immunotoxin antibody levels are lower in patients with adverse outcomes due to orthopaedic S. aureus infections. Results show serum IgG levels, represented as Median fluorescent intensity, against autolysin (Gmd and Amd), alpha-hemolysin (Hla), chemotaxis inhibiting protein of S. aureus (CHIPS) and the Staphylococcal complement inhibitor (SCIN) antigens determined via Luminex immunoassay for patients in the AOTrauma CPP Bone Infection Registry with reported S. aureus infections (N=194). FIG. 2B depicts exemplary results demonstrating that IsaA antibody levels are lower in patients with adverse outcomes due to orthopaedic S. aureus infections. Results show serum IgG levels, represented as median fluorescent intensity, against IsaA antigens determined via Luminex immunoassay for patients in the AOTrauma CPP Bone Infection Registry with reported S. aureus infections (N=194).

FIG. 3, comprising FIG. 3A through FIG. 3E, depicts exemplary results demonstrating the in vivo validation of S. aureus mRNA nanoparticle vaccines via induction of antibodies against Atl, Hla, and CHIPS in mice. FIG. 3A depicts exemplary results of the size and integrity of the in vitro synthesized mRNAs as assessed via 0.8% agarose gel electrophoresis, and a representative image for each mRNA as shown with their size in nucleotides (nt). Each synthesized mRNA (15 μg/mice) was individually mixed with in vivo-jetRNA transfection reagent and injected into 6-week-old female C57B/6 mice intramuscularly, and the mice were boosted with the same vaccine on day 14. Submandibular bleeding was performed at days 0, 14, 28, 42 post-immunization, and the resulting sera were probed for anti-S. aureus antibodies via Luminex as we have previously described (Nishitani, K, et al., Clin Orthop Relat Res, 2015, 473 (9), 2735-49; Kates, S. L., et al., J Bone Joint Surg Am, 2020; Lee, C. C., et al., Eur Cell Mater, 2020, 39, 96-107; Muthukrishnan, G.; Beck, C. A.; Owen, J. R.; Xie, C.; Kates, S. L., et al., JOrthop Res, 2020). FIGS. 3B-E depict exemplary results of relative IgG antibody levels against: Hla (FIG. 3B), CHIPS (FIG. 3C), Gmd (FIG. 3D), and Amd (FIG. 3E), presented as the median fluorescent intensity+/−SD (N=3-4, *p<0.05, **p<0.01, Mann-Whitney test vs. unimmunized controls that were only bled on days 0, 14 & 28). Note that significant IgG antibody levels were detectable 28 days post-immunization with all mRNA nanoparticle vaccines.

### DETAILED DESCRIPTION

The present disclosure generally relates to compositions comprising nucleic acids encoding at least one antigenic S. aureus protein selected from autolysin (Atl) glucosaminidase (Gmd), amidase (Amd), alpha-hemolysin (Hla), chemotaxis inhibiting protein of Staphylococcus (CHIPs), staphylococcal complement inhibitor (SCIN), and Coproporphyrinogen III Oxidase (CgoX) and to methods of treating, preventing, or otherwise inducing an immune response against S. aureus infection. The present disclosure also generally relates to compositions comprising nucleic acids encoding at least one antigenic S. aureus lytic transglycosylase such as immunodominant staphylococcal antigen A (IsaA) and SACOL2088 (SceD) and to methods of treating, preventing, or otherwise inducing an immune response against S. aureus infection.

### Definitions

Unless defined otherwise, all technical and scientific terms used herein have the same meaning as commonly understood by one of ordinary skill in the art to which this invention belongs.

As used herein, each of the following terms has the meaning associated with it in this section.

A “5′ untranslated region” (5′UTR) refers to a region of an mRNA that is directly upstream (i.e., 5′) from the start codon (i.e., the first codon of an mRNA transcript translated by a ribosome) that does not encode a polypeptide.

A “3′ untranslated region” (3′UTR) refers to a region of an mRNA that is directly downstream (i.e., 3′) from the stop codon (i.e., the codon of an mRNA transcript that signals a termination of translation) that does not encode a polypeptide.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “antigen” or “antigenic” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “antigenic polypeptide” encompasses immunogenic full-length proteins or fragments of the immunogenic protein (i.e. an immunogenic polypeptide fragment that induces or is capable of inducing an immune response to one or more pathogenic species).

As used herein, the terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” if they contain amino acid substitutions, insertions or a combination of substitutions and insertions.

A “consensus sequence”, as used herein, refers to a sequence derived from a sequence alignment of more than one variable sequence. For example, a calculated order of the most frequent residues in the alignment of multiple amino acid sequences of an antigen can be used to define a consensus sequence for that antigen.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

As used herein, a nucleotide sequence is “substantially homologous” to any of the nucleotide sequences described herein when its nucleotide sequence has a sequence identity of degree of identity with an original or reference nucleotide sequence at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

As used herein, an amino acid sequence is “substantially homologous” to any of the amino acid sequences described herein when its amino acid sequence has a degree of identity with an original or reference amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. The identity between two amino acid sequences can be determined by using the BLASTN algorithm (BLAST Manual, Altschul, S., et al., NCBI NLM NIH Bethesda, Md. 20894, Altschul, S., et al., J. Mol. Biol. 215: 403-410, 1990)).

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

The term “immunogen” or “immunogenic” as used herein, is intended to denote a substance of matter, which is capable of inducing an adaptive immune response in an individual, where said adaptive immune response is capable of inducing an immune response which significantly engages pathogenic agents, which share immunological features with the immunogen. “Immunogen” refers to any substance introduced into the body in order to generate an immune response. That substance can a physical molecule, such as a protein, or can be encoded by a vector, such as DNA, mRNA, or a virus.

“Immune response,” as the term is used herein, means a process involving the activation and/or induction of an effector function in, by way of non-limiting examples, a T cell, B cell, natural killer (NK) cell, and/or an antigen-presenting cell (APC). Thus, an immune response, as would be understood by the skilled artisan, includes, but is not limited to, any detectable antigen-specific activation and/or induction of a helper T cell or cytotoxic T cell activity or response, production of antibodies, antigen presenting cell activity or infiltration, macrophage activity or infiltration, neutrophil activity or infiltration, and the like.

As used herein, the terms “immunogenic fragment” or to a fragment of an antigen or a nucleic acid sequence encoding an antigen that, when administered to a subject, provides an increased immune response. Fragments are generally 10 or more amino acids or nucleic acids in length. “Fragment” may mean a polypeptide fragment of an antigen that is capable of eliciting an immune response in a subject. A fragment of an antigen may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antigen, excluding any heterologous signal peptide added. The fragment may comprise a fragment of a polypeptide that is 95% or more, 96% or more, 97% or more, 98% or more or 99% or more identical to the antigen and additionally comprise an N terminal methionine or heterologous signal peptide which is not included when calculating percent identity.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates.

A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

The term “polynucleotide” as used herein is defined as a chain of nucleotides, including ribonucleotides. Furthermore, nucleic acids are polymers of nucleotides or ribonucleotides. Thus, nucleic acids may be used interchangeably herein with the term polynucleotide. One skilled in the art has the general knowledge that (ribo)nucleic acids are polynucleotides, which can be hydrolyzed into the monomeric “(ribo)nucleotides.” The monomeric (ribo)nucleotides can be hydrolyzed into (ribo)nucleosides. As used herein polynucleotides include, but are not limited to, all (ribo)nucleic acid sequences which are obtained by any means available in the art, including, without limitation, recombinant means, i.e., the cloning of (ribo)nucleic acid sequences from a recombinant library or a cell genome, using ordinary cloning technology and PCR™, and the like, and by synthetic means.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The terms “subject,” “patient,” “individual,” and the like are used interchangeably herein, and refer to any animal, or cells thereof whether in vitro or in situ, amenable to the methods described herein. In some non-limiting embodiments, the patient, subject or individual is a mammal, bird, poultry, cattle, pig, horse, sheep, ferret, primate, dog, cat, guinea pig, rabbit, bat, or human.

The term “therapeutic” as used herein means a treatment and/or prophylaxis. A therapeutic effect is obtained by suppression, diminution, remission, prevention, or eradication of at least one sign or symptom of a disease or disorder.

To “treat” a disease as the term is used herein, means to reduce the frequency or severity of at least one sign or symptom of a disease or disorder experienced by a subject.

As used herein, the term “vaccine” refers to a composition that induces an immune response upon inoculation into a subject. In some embodiments, the induced immune response provides protective immunity.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

### Compositions

In some embodiments, the present disclosure provides a composition for inducing an immune response against one or more bacterium. In some embodiments, the bacterium is a gram-positive bacterium. In one embodiment, the gram-positive bacterium comprises Staphylococcus aureus (S. aureus). In some embodiments, the composition comprises one or more nucleic acid molecules. In some embodiments, the one or more nucleic acid molecules encodes one or more S. aureus antigenic polypeptide or immunogenic fragment thereof. In some embodiments, said one or more S. aureus antigenic polypeptide, immunogenic fragment thereof, or antigenic variant thereof comprises a consensus sequence derived from more than one S. aureus genome.

Vaccines

In some embodiments, the composition for inducing an immune response against S. aureus is a vaccine. For a composition to be useful as a vaccine, the composition must induce an immune response against one or more S. aureus antigen in a cell, tissue or subject. In some embodiments, the composition induces an immune response against one or more S. aureus antigen in a cell, tissue or subject. In some instances, the vaccine induces a protective immune response in the subject.

A vaccine of the present disclosure may vary in its composition of nucleic acid and/or cellular components.

In one embodiment, the vaccine comprises at least one (one or more) nucleic acid polynucleotide having an open reading frame encoding at least one S. aureus antigenic polypeptide. In a further embodiment, the vaccine comprises a nucleic acid sequence that is codon optimized. In a further embodiment, the vaccine comprises a nucleic acid sequence that is codon optimized for expression in cells of a mammalian subject (e.g., a human). In a further embodiment, the vaccine comprises at least one (one or more) nucleic acid polynucleotide having an open reading frame encoding at least one S. aureus antigenic polypeptide selected from Atl, Amd, Gmd, Hla, SCIN, CHIPs and CgoX. In another embodiment, the vaccine comprises at least one (one or more) nucleic acid polynucleotide having an open reading frame encoding at least one S. aureus lytic transglycosylase. In a further embodiment, the encoded antigenic polypeptide is IsaA or SceD. In another embodiment, the vaccine comprises at least one (one or more) nucleic acid polynucleotide having an open reading frame encoding at least one S. aureus antigenic polypeptide having an amino acid sequence disclosed in Table 1, or a fragment or variant thereof. In a further embodiment, the vaccine comprises a nucleic acid sequence that is codon optimized. In a further embodiment, the vaccine comprises a nucleic acid sequence that is codon optimized for expression in cells of a mammalian subject (e.g., a human). In some embodiments, at least one nucleic acid of the vaccine encodes an antigenic fragment of an S. aureus polypeptide. In some embodiments, at least one nucleic acid of the vaccine encodes an antigenic fragment of an S. aureus polypeptide selected from Atl, Amd, Gmd, Hla, SCIN, CHIPs and CgoX. In some embodiments, at least one nucleic acid of the vaccine encodes an antigenic fragment of an S. aureus lytic transglycosylase. In further embodiments, at least one nucleic acid of the vaccine encodes an antigenic fragment of IsaA or SceD. In some embodiments, at least one nucleic acid of the vaccine encodes an antigenic fragment of an S. aureus polypeptide having an amino acid sequence disclosed in Table 1. In some embodiments, at least one nucleic acid of the vaccine encodes an antigenic variant of an S. aureus polypeptide. In some embodiments, at least one nucleic acid of the vaccine encodes an antigenic variant of an S. aureus polypeptide selected from Atl, Amd, Gmd, Hla, SCIN, CHIPs and CgoX. In some embodiments, at least one nucleic acid of the vaccine encodes an antigenic variant of an S. aureus lytic transglycosylase. In further embodiments, at least one nucleic acid of the vaccine encodes an antigenic variant of IsaA or SceD. In some embodiments, at least one nucleic acid of the vaccine encodes an antigenic variant of an S. aureus polypeptide having an amino acid sequence disclosed in Table 1. In some embodiments, the at least one nucleic acid of the vaccine encodes at least one consensus S. aureus antigenic polypeptide, fragment or variant thereof.

In some embodiments, at least one nucleic acid of the vaccine encodes an antigenic variant of an S. aureus polypeptide. In some embodiments, at least one nucleic acid of the vaccine encodes antigenic polypeptides from 2-10, 2-9, 2-8, 2-7, 2-6, 2-5, 2-4 S. aureus polypeptides. In some embodiments, at least one nucleic acid of the vaccine encodes antigenic polypeptides from at least 2, 5, 10, or 15 antigenic S. aureus polypeptides. In one embodiment, the vaccine comprises at least one nucleic acid comprising a polynucleotide sequence having a single open reading frame encoding two or more (e.g., two, three, four, five, or more) S. aureus antigenic polypeptides. In one embodiment, the vaccine comprises at least one nucleic acid comprising a polynucleotide sequence having more than one open reading frame (e.g., two, three, four, five or more open reading frames) encoding two, three, four, five or more S. aureus antigenic polypeptides.

In one embodiment, the vaccine comprises at least one (one or more) RNA polynucleotide having an open reading frame encoding at least one S. aureus antigenic polypeptide. In a further embodiment, the vaccine comprises an RNA polynucleotide sequence that is codon optimized. In a further embodiment, the vaccine comprises a nucleic acid sequence that is codon optimized for expression in cells of a mammalian subject (e.g., a human). In a further embodiment, the vaccine comprises at least one (one or more) RNA polynucleotide having an open reading frame encoding at least one S. aureus antigenic polypeptide selected from Atl, Amd, Gmd, Hla, SCIN, CHIPs and CgoX. In another embodiment, the vaccine comprises at least one (one or more) RNA polynucleotide having an open reading frame encoding at least one S. aureus lytic transglycosylase. In a further embodiment, the encoded antigenic polypeptide is IsaA or SceD. In another embodiment, the vaccine comprises at least one (one or more) RNA polynucleotide having an open reading frame encoding at least one S. aureus antigenic polypeptide having an amino acid sequence disclosed in Table 1, or a fragment or variant thereof. In some embodiments, the at least one RNA polynucleotide of the vaccine encodes at least one consensus S. aureus antigenic polypeptide, fragment or variant thereof.

In some embodiments, the vaccine comprises at least one (one or more) RNA polynucleotide that encodes an antigenic fragment of an S. aureus polypeptide. In some embodiments, the vaccine comprises at least one (one or more) RNA polynucleotide that encodes an antigenic fragment of an S. aureus polypeptide selected from Atl, Amd, Gmd, Hla, SCIN, CHIPs and CgoX. In another embodiment, the vaccine comprises at least one (one or more) RNA polynucleotide that encodes an antigenic fragment of at least one S. aureus lytic transglycosylase. In a further embodiment, the vaccine comprises at least one (one or more) RNA polynucleotide that encodes an antigenic fragment of IsaA or SceD. In some embodiments, the vaccine comprises at least one (one or more) RNA polynucleotide that encodes an antigenic fragment of an S. aureus polypeptide having an amino acid sequence disclosed in Table 1. In some embodiments, the vaccine comprises at least one (one or more) RNA polynucleotide that encodes an antigenic variant of an S. aureus polypeptide. In some embodiments, the vaccine comprises at least one (one or more) RNA polynucleotide that encodes an antigenic variant of an S. aureus polypeptide selected from Atl, Amd, Gmd, Hla, SCIN, CHIPs and CgoX. In another embodiment, the vaccine comprises at least one (one or more) RNA polynucleotide that encodes an antigenic variant of at least one S. aureus lytic transglycosylase. In a further embodiment, the vaccine comprises at least one (one or more) RNA polynucleotide that encodes an antigenic variant of IsaA or SceD. In some embodiments, the vaccine comprises at least one (one or more) RNA polynucleotide that encodes an antigenic variant of an S. aureus polypeptide having an amino acid sequence disclosed in Table 1.

In some embodiments, the vaccine comprises at least one (one or more) RNA polynucleotide that encodes an antigenic variant of an S. aureus polypeptide. In some embodiments, the vaccine comprises at least one (one or more) RNA polynucleotide that encodes antigenic polypeptides from 2-10, 2-9, 2-8, 2-7, 2-6, 2-5, 2-4 S. aureus polypeptides. In some embodiments, at least one nucleic acid of the vaccine encodes antigenic polypeptides from at least 2, 5, 10, or 15 antigenic S. aureus polypeptides. In one embodiment, the vaccine comprises at least one (one or more) RNA polynucleotide sequence having a single open reading frame encoding two or more (e.g., two, three, four, five, or more) S. aureus antigenic polypeptides. In one embodiment, the vaccine comprises at least one RNA polynucleotide sequence having more than one open reading frame (e.g., two, three, four, five or more open reading frames) encoding two, three, four, five or more S. aureus antigenic polypeptides.

In one embodiment, the vaccine comprises at least one (one or more) ribonucleic acid (RNA) polynucleotide having an open reading frame encoding at least one S. aureus antigenic polypeptide. In one embodiment, the vaccine comprises a (ribo)nucleic acid encoding an S. aureus antigen. In some embodiments, the vaccine comprises a (ribo)nucleic acid encoding at least one consensus S. aureus antigenic polypeptide, fragment or variant thereof.

In a non-limiting example, a nucleic acid or ribonucleic acid encoding an S. aureus antigen may also be formulated with an adjuvant. Of course, it will be understood that various compositions described herein may further comprise additional components as needed. For example, one or more vaccine components may be comprised in a lipid, liposome, or lipid nanoparticle. In another non-limiting example, a vaccine may comprise one or more adjuvants. A vaccine of the present disclosure, and its various components, may be prepared and/or administered by any method disclosed herein or as would be known to one of ordinary skill in the art, in light of the present disclosure.

In various embodiments, the induction of immunity by the expression of the S. aureus antigen can be detected by observing in vivo or in vitro the response of all or any part of the immune system in the host against the S. aureus antigen.

For example, a method for detecting the induction of cytotoxic T lymphocytes is well known in the art. A foreign substance that enters the living body is presented to T cells and B cells by the action of antigen presenting cells (APCs). Some T cells that respond to the antigen presented by APC in an antigen specific manner differentiate into cytotoxic T cells (also referred to as cytotoxic T lymphocytes or CTLs) due to stimulation by the antigen. These antigen-stimulated cells then proliferate. This process is referred to herein as “activation” of T cells. Therefore, CTL induction by an epitope of a polypeptide or peptide or combinations thereof can be evaluated by presenting an epitope of a polypeptide or peptide or combinations thereof to a T cell by APC, and detecting the induction of CTL. Furthermore, APCs have the effect of activating B cells, CD4+ T cells, CD8+ T cells, macrophages, eosinophils and NK cells.

A method for evaluating the inducing action of CTL using dendritic cells (DCs) as APC is well known in the art. A DC is a representative APC having a robust CTL inducing action among APCs. In the methods of the disclosure, the epitope of a polypeptide or peptide or combinations thereof is initially expressed by the DC and then this DC is contacted with T cells. Detection of T cells having cytotoxic effects against the cells of interest after the contact with DC shows that the epitope of a polypeptide or peptide or combinations thereof has an activity of inducing the cytotoxic T cells. Furthermore, the induced immune response can also be examined by measuring IFN-gamma produced and released by CTL in the presence of antigen-presenting cells that carry immobilized peptide or a combination of peptides by visualizing using anti-IFN-gamma antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

The antigens confirmed to possess CTL-inducing activity by these methods are antigens having DC activation effect and subsequent CTL-inducing activity. Furthermore, CTLs that have acquired cytotoxicity due to presentation of the antigen by APC can be also used as vaccines against antigen-associated disorders.

The induction of immunity by expression of the S. aureus antigen can be further confirmed by observing the induction of antibody production against the S. aureus antigen. For example, when antibodies against an antigen are induced in a laboratory subject immunized with the composition encoding the antigen, and when antigen-associated pathology is suppressed by those antibodies, the composition is determined to induce immunity.

The specificity of the antibody response induced in a subject can include binding to many regions of the delivered antigen, as well as, the induction of neutralization capable antibodies that that prevent infection or reduce disease severity.

The induction of immunity by expression of the S. aureus antigen can be further confirmed by observing the induction of T cells, such as CD4+ T cells, CD8+ T cells, or a combination thereof. For example, CD4+ T cells can also lyse target cells, but mainly supply help in the induction of other types of immune responses, including CTL and antibody generation. The type of CD4+ T cell help can be characterized, as Th1, Th2, Th9, Th17, Tregulatory (Treg), or T follicular helper (Tfh) cells. Each subtype of CD4+ T cell supplies help to certain types of immune responses. In one embodiment, the composition selectively induces T follicular helper cells, which drive potent antibody responses.

### Nucleic Acid Vaccines

In some embodiments, the present disclosure comprises an S. aureus nucleic acid vaccine. Nucleic acid-based vaccines are known to elicit a prominent cell-mediated immune response. See. e.g., Donnely et al., 1997; Rosenberg, S. A., Immunity 10:281, 1999. Thus, the antigenic agent for use in the vaccines of the disclosure can take the form of a polynucleotide that can stimulate an immune response against one or more S. aureus antigenic polypeptide, variant or an immunogenic fragment thereof when administered to a subject.

The form of the nucleic acid used in a vaccine of the disclosure can be any suitable for stimulating an immune response against S. aureus when administered to a subject. For example, the nucleic acid can be in the form of “naked DNA” or it can be incorporated in an expression vector. A description of suitable nucleic acids is presented below. Nucleic acids that are most immunogenic in a subject can be determined by preparing several of the below listed nucleic acids (e.g., those that encode the whole antigen, variants or peptide fragments thereof), administering to the subject (or a series of genetically similar such subjects) such nucleic acids in a vaccine composition (e.g., as naked nucleic acid or in an expression vector in a suitable carrier), and analyzing the subject(s) for the stimulation of an immune response. Those nucleic acids that induce the desired response can then be selected.

Nucleic acid molecules utilized in the present disclosure as an antigenic agent may be in the form of RNA or in the form of DNA (e.g., cDNA, genomic DNA, and synthetic DNA). The DNA may be double-stranded or single-stranded, and if single-stranded may be the coding (sense) strand or non-coding (anti-sense) strand.

The disclosure provides for the use of nucleic acid vaccines to stimulate an immune response against one or more S. aureus antigenic polypeptide, variant or an immunogenic fragment thereof. The use of nucleic acids for stimulating both class I and class II restricted immune responses against a particular protein is known in the art. See. e.g., Rosenberg, S. A., Immunity 10:281, 1999; Ulmer et al., Science, 259:1745, 1993; Donnelly et al., Ann. NY Acad. Sci., 772:40, 1995; Scheurs et al., Cancer Res. 58:2509, 1998; Hurpin et al., Vaccine 16:208, 1998; Lekutis et al., J. Immunol. 158:4471, 1997; Manickan et al., J. Leukoc. Biol. 61:125, 1997. Nucleic acid vaccines can be administered to a subject by any suitable technique. For example, naked DNA can be injected into muscle cells of a subject or naked DNA-coated gold particles can be introduced into skin cells (to be taken up by dendritic cells) of a subject using a gene gun.

In one embodiment, the provided composition comprises one or more nucleic acid molecule comprising a polynucleotide sequence encoding an S. aureus antigen disclosed in Table 1, or an antigenic variant or fragment thereof. In one embodiment, the nucleic acid molecule is not naturally occurring. In one embodiment, the composition comprises a nucleic acid molecule comprising a codon optimized nucleic acid sequence encoding an S. aureus antigen disclosed in Table 1, or an antigenic variant or fragment thereof. In one embodiment, the composition comprises a nucleic acid molecule comprising a codon optimized nucleic acid sequence encoding at least one S. aureus antigenic polypeptide having an amino acid sequence of any one of SEQ ID NO:1, 4, 7, 10, 13, 16, 19, 22, 25, 28, 31, 34, 37, 40, 43, 45, 48, 51, 54, 57, 60, 63, 66, 69, or 72. In one embodiment, the composition comprises a nucleic acid molecule comprising a codon optimized nucleic acid sequence encoding at least one S. aureus antigenic polypeptide having an amino acid sequence of any one of SEQ ID NO: 86 or 89. In one embodiment, the composition comprises a nucleic acid molecule comprising a codon optimized nucleic acid sequence encoding a fragment or variant of at least one S. aureus antigenic polypeptide having an amino acid sequence of any one of SEQ ID NO: 1, 4, 7, 10, 13, 16, 19, 22, 25, 28, 31, 34, 37, 40, 43, 45, 48, 51, 54, 57, 60, 63, 66, 69, or 72. In one embodiment, the composition comprises a nucleic acid molecule comprising a codon optimized nucleic acid sequence encoding a fragment or variant of at least one S. aureus antigenic polypeptide having an amino acid sequence of any one of SEQ ID NO: 86 or 89.

In one embodiment, the composition comprises one or more nucleic acid molecule comprising a polynucleotide sequence disclosed in Table 1, or a fragment or variant thereof that encodes an antigenic polypeptide. In one embodiment, the composition comprises one or more nucleic acid molecule comprising a codon optimized nucleic acid sequence disclosed in Table 1, or an antigenic variant or fragment thereof. In one embodiment, the composition comprises one or more nucleic acid molecule comprising an RNA sequence disclosed in Table 1, or a fragment or variant thereof. In one embodiment, the composition comprises an RNA molecule comprising an RNA sequence of any one of SEQ ID NO: 3, 6, 9, 12, 15, 18, 21, 24, 27, 30, 33, 36, 39, 42, 44, 47, 50, 53, 56, 59, 62, 65, 68, 71, or 74. In one embodiment, the composition comprises an RNA molecule comprising an RNA sequence of any one of SEQ ID NO: 88 or 91.

In one embodiment, the composition comprises one or more nucleic acid molecule comprising a polynucleotide sequence encoding autolysin (Atl), or a variant or fragment thereof. In one embodiment, the composition comprises one or more nucleic acid molecule comprising a polynucleotide sequence encoding the amino acid sequence of SEQ ID NO: 1, or a variant or fragment thereof. In one embodiment, the nucleic acid is not naturally occurring. In one embodiment, the nucleic acid is codon optimized. In a further embodiment, the nucleic acid is codon optimized for expression in cells of a mammalian subject (e.g., a human). In one embodiment, the nucleic acid comprises the sequence of SEQ ID NO:2. In one embodiment, the nucleic acid is an RNA. In a further embodiment, the RNA comprises the sequence of SEQ ID NO: 3.

In one embodiment, the provided composition comprises one or more nucleic acid molecule comprising a polynucleotide sequence encoding an antigenic fragment of Atl. In one embodiment, the composition comprises one or more nucleic acid molecule comprising a polynucleotide sequence encoding an antigenic fragment of Atl having the amino acid sequence of SEQ ID NO:1. In some embodiments, the composition comprises one or more nucleic acid molecule comprising a polynucleotide sequence encoding an antigenic fragment of Atl that is 6-1267, 6-1200, 6-1000, 6-750, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In further embodiments, the composition comprises one or more nucleic acid molecule comprising a polynucleotide sequence encoding an antigenic fragment of an Atl having the amino acid sequence of SEQ ID NO: 72 that is 6-1066, 6-1200, 6-1000, 6-750, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In further embodiments, the composition comprises one or more nucleic acid molecule comprising a polynucleotide sequence encoding an antigenic fragment of an Atl having the amino acid sequence of SEQ ID NO: 1 that is 6-1237, 6-1200, 6-1000, 6-750, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length.

In one embodiment, the provided composition comprises one or more nucleic acid molecule comprising a polynucleotide sequence encoding an Atl variant. In some embodiments, the polynucleotide sequence encodes an Atl variant having sequence identity with the amino acid sequence of SEQ ID NO:1 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the polynucleotide sequence encodes an Atl variant having sequence identity with the amino acid sequence of SEQ ID NO:72 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In one embodiment, the provided composition comprises one or more nucleic acid molecule comprising a polynucleotide sequence encoding an Atl variant having a total of 1-100, 1-75, 1-50, 1-24, 1-30, 1-20, 1-15, 1-10, or 1-5, amino acid substitutions, deletions, and/or insertions compared to a reference sequence of SEQ ID NO:1. In one embodiment, the composition comprises one or more nucleic acid molecule comprising a polynucleotide sequence encoding an Atl variant having a total of 1, 2, 3, 4, 5, 6, 7, 8, 9, 10, or fewer than 15, amino acid substitutions, deletions, and/or insertions compared to a reference sequence of SEQ ID NO:1. In one embodiment, the provided composition comprises one or more nucleic acid molecule comprising a polynucleotide sequence encoding an Atl variant having a total of 1-100, 1-75, 1-50, 1-24, 1-30, 1-20, 1-15, 1-10, or 1-5, amino acid substitutions, deletions, and/or insertions compared to a reference sequence of SEQ ID NO:72. In one embodiment, the composition comprises one or more nucleic acid molecule comprising a polynucleotide sequence encoding an Atl variant having a total of 1, 2, 3, 4, 5, 6, 7, 8, 9, 10, or fewer than 15, amino acid substitutions, deletions, and/or insertions compared to a reference sequence of SEQ ID NO:72. In some embodiments, one or more of the substitutions in the encoded variant are conservative substitutions. In some embodiments, one or more of the deletions in the encoded variant are at the amino and or carboxy terminus of the reference sequence.

In one embodiment, the provided composition comprises one or more nucleic acid molecule comprising a polynucleotide sequence encoding N-acetylmuramyl-L-alanine amidase (Amd), or an antigenic variant or fragment thereof. In one embodiment, the composition comprises one or more nucleic acid molecule comprising a polynucleotide sequence encoding Amd. In one embodiment, the composition comprises one or more nucleic acid molecule comprising a polynucleotide sequence encoding the amino acid sequence of SEQ ID NO: 4, or an antigenic variant or fragment thereof. In one embodiment, the composition comprises one or more nucleic acid molecule comprising a polynucleotide sequence encoding the amino acid sequence of SEQ ID NO: 4. In one embodiment, the nucleic acid comprises the sequence of SEQ ID NO:5. In one embodiment, the nucleic acid is an RNA. In a further embodiment, the RNA comprises the sequence of SEQ ID NO: 6.

In one embodiment, the composition comprises one or more nucleic acid molecule comprising a consensus, codon-optimized polynucleotide sequence encoding a consensus Amd antigenic protein. In one embodiment, the consensus Amd antigenic protein comprises the amino acid sequence of SEQ ID NO: 4. In one embodiment, the consensus, codon-optimized polynucleotide sequence comprises DNA. In one embodiment, the consensus, codon-optimized DNA sequence comprises the sequence of SEQ ID NO: 5. In one embodiment, the consensus, codon-optimized polynucleotide sequence comprises RNA. In one embodiment, the consensus, codon-optimized RNA sequence comprises the sequence of SEQ ID NO: 6.

In one embodiment, the provided composition comprises one or more nucleic acid molecule comprising a polynucleotide sequence encoding an antigenic fragment of Amd. In one embodiment, the composition comprises one or more nucleic acid molecule comprising a polynucleotide sequence encoding an antigenic fragment of Amd having the amino acid sequence of SEQ ID NO:4. In some embodiments, composition comprises one or more nucleic acid molecule comprising a polynucleotide sequence encoding an antigenic fragment of a Amd having the amino acid sequence of SEQ ID NO:4 that is 6-590, 6-575, 6-550, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length.

In one embodiment, the provided composition comprises one or more nucleic acid molecule comprising a polynucleotide sequence encoding an antigenic fragment comprising one or more domains of Amd or an antigenic fragment or variant of one or more domains of Amd. In some embodiments, composition comprises one or more nucleic acid molecule comprising a polynucleotide sequence encoding an antigenic fragment comprising an Amd R1 domain. In some embodiments, composition comprises one or more nucleic acid molecule comprising a polynucleotide sequence encoding an antigenic fragment comprising the amino acid sequence of SEQ ID NO: 7. In some embodiments, composition comprises one or more nucleic acid molecule comprising a polynucleotide sequence encoding a fragment comprising an Amd R1 domain sequence of SEQ ID NO:7 and a fragment of the sequence of SEQ ID NO:4 that is 176-590, 176-500, 176-250, 176-200, or 176-190, amino acids in length. In one embodiment, the nucleic acid comprises the sequence of SEQ ID NO: 8. In one embodiment, the nucleic acid is an RNA. In a further embodiment, the RNA comprises the sequence of SEQ ID NO: 9. In some embodiments, the composition comprises one or more nucleic acid molecule comprising a polynucleotide sequence encoding a fragment of an Amd R1 domain sequence of SEQ ID NO:7 that is 6-175, 6-150, 6-125, 6-100, 6-75, 6-50 or 6-25 amino acids in length.

In some embodiments, composition comprises one or more nucleic acid molecule comprising a polynucleotide sequence encoding an antigenic fragment comprising an Amd R2 domain. In some embodiments, composition comprises one or more nucleic acid molecule comprising a polynucleotide sequence encoding an antigenic fragment comprising the amino acid sequence of SEQ ID NO: 10. In some embodiments, composition comprises one or more nucleic acid molecule comprising a polynucleotide sequence encoding a fragment comprising an Amd R2 domain sequence of SEQ ID NO:10 that is 172-590, 172-500, 172-250, 172-200, or 172-190, amino acids in length. In one embodiment, the nucleic acid comprises the sequence of SEQ ID NO: 11. In one embodiment, the nucleic acid is an RNA. In a further embodiment, the RNA comprises the sequence of SEQ ID NO: 12. In some embodiments, the composition comprises one or more nucleic acid molecule comprising a polynucleotide sequence encoding a fragment of an Amd R2 domain sequence of SEQ ID NO:10 that is 6-193, 6-175, 6-150, 6-125, 6-100, 6-75, 6-50 or 6-25 amino acids in length.

In some embodiments, composition comprises one or more nucleic acid molecule comprising a polynucleotide sequence encoding an antigenic fragment comprising an Amd catalytic domain. In some embodiments, composition comprises one or more nucleic acid molecule comprising a polynucleotide sequence encoding an antigenic fragment comprising the amino acid sequence of SEQ ID NO: 16. In some embodiments, composition comprises one or more nucleic acid molecule comprising a polynucleotide sequence encoding a fragment comprising an Amd catalytic domain sequence of SEQ ID NO:16 that is 224-590, 224-500, 224-250, 224-240, or 224-230, amino acids in length. In one embodiment, the nucleic acid comprises the sequence of SEQ ID NO: 17. In one embodiment, the nucleic acid is an RNA. In a further embodiment, the RNA comprises the sequence of SEQ ID NO: 18. In some embodiments, the composition comprises one or more nucleic acid molecule comprising a polynucleotide sequence encoding a fragment of a catalytic domain sequence of SEQ ID NO:16 that is 6-223, 6-200, 6-175, 6-150, 6-125, 6-100, 6-75, 6-50, 6-25, 6-20, 6-15, or 6-10 amino acids in length.

In one embodiment, the provided composition comprises one or more nucleic acid molecule comprising a polynucleotide sequence encoding an antigenic fragment comprising an Amd R1 and R2 domain. In some embodiments, composition comprises one or more nucleic acid molecule comprising a polynucleotide sequence encoding a fragment comprising an Amd R1 and R2 domain sequence. In some embodiments, composition comprises one or more nucleic acid molecule comprising a polynucleotide sequence encoding a fragment comprising an Amd R1 and R2 domain sequence of SEQ ID NO: 13. In some embodiments, composition comprises one or more nucleic acid molecule comprising a polynucleotide sequence encoding a fragment comprising an Amd R1 and R2 domain sequence of SEQ ID NO:13 that is 366-1180, 366-1000, 366-750, 366-700, 366-600, 366-590, 366-500, 366-450, or 366-40, amino acids in length.

In one embodiment, the provided composition comprises one or more nucleic acid molecule comprising a polynucleotide sequence encoding an Amd variant. In some embodiments, the polynucleotide sequence encodes an Amd variant having sequence identity with respect to the amino acid sequence of SEQ ID NO:4 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In one embodiment, the provided composition comprises one or more nucleic acid molecule comprising a polynucleotide sequence encoding an Amd variant having a total of 1-20, 1-15, 1-10, or 1-5, amino acid substitutions, deletions, and/or insertions compared to a reference sequence of SEQ ID NO: 4. In one embodiment, the provided composition comprises one or more nucleic acid molecule comprising a polynucleotide sequence encoding an Amd variant having a total of 1-20, 1-15, 1-10, or 1-5, amino acid substitutions, deletions, and/or insertions compared to a reference sequence of SEQ ID NO:7, 10, 13, or 16. In one embodiment, the provided composition comprises one or more nucleic acid molecule comprising a polynucleotide sequence encoding an Amd variant having a total of 1, 2, 3, 4, 5, 6, 7, 8, 9, 10, or fewer than 15, amino acid substitutions, deletions, and/or insertions compared to the reference sequence of SEQ ID NO:4. In one embodiment, the provided composition comprises one or more nucleic acid molecule comprising a polynucleotide sequence encoding an Amd variant having a total of 1, 2, 3, 4, 5, 6, 7, 8, 9, 10, or fewer than 15, amino acid substitutions, deletions, and/or insertions compared to the reference sequence of SEQ ID NO: 7, 10, 13, or 16. In some embodiments, one or more of the substitutions in the encoded variant are conservative substitutions. In some embodiments, one or more of the deletions in the encoded variant are at the amino and or carboxy terminus of the reference sequence.

In one embodiment, the provided composition comprises one or more nucleic acid molecule comprising a polynucleotide sequence encoding endo-β-N-acetylglucosaminidase (Gmd), or an antigenic variant or fragment thereof. In one embodiment, the composition comprises one or more nucleic acid molecule comprising a polynucleotide sequence encoding Gmd. In one embodiment, the composition comprises one or more nucleic acid molecule comprising a polynucleotide sequence encoding the amino acid sequence of SEQ ID NO: 19, or an antigenic fragment of variant thereof. In one embodiment, the composition comprises one or more nucleic acid molecule comprising a polynucleotide sequence encoding the amino acid sequence of SEQ ID NO: 19. In one embodiment, the nucleic acid molecule is an RNA encoding Gmd. In one embodiment, the nucleic acid comprises the sequence of SEQ ID NO: 20. In one embodiment, the nucleic acid is an RNA. In a further embodiment, the RNA comprises the sequence of SEQ ID NO:21.

In one embodiment, the composition comprises one or more nucleic acid molecule comprising a consensus, codon-optimized polynucleotide sequence encoding a consensus Gmd antigenic protein. In one embodiment, the consensus Gmd antigenic protein comprises the amino acid sequence of SEQ ID NO: 19. In one embodiment, the consensus, codon-optimized polynucleotide sequence comprises DNA. In one embodiment, the consensus, codon-optimized DNA sequence comprises the sequence of SEQ ID NO: 20. In one embodiment, the consensus, codon-optimized polynucleotide sequence comprises RNA. In one embodiment, the consensus, codon-optimized RNA sequence comprises the sequence of SEQ ID NO: 21.

In one embodiment, the provided composition comprises one or more nucleic acid molecule comprising a polynucleotide sequence encoding an antigenic fragment of Gmd. In one embodiment, the composition comprises one or more nucleic acid molecule comprising a polynucleotide sequence encoding an antigenic fragment of Gmd having the amino acid sequence of SEQ ID NO:19. In further embodiments, composition comprises one or more nucleic acid molecule comprising a polynucleotide sequence encoding an antigenic fragment of a Gmd having the amino acid sequence of SEQ ID NO: 19 that is 6-482, 6-475, 6-450, 6-400, 6-350, 6-300, 6-250, 6-200, 6-175, 6-150, 6-125, 6-100, 6-75, 6-50, 6-25, 6-20, 6-15, or 6-10 amino acids in length.

In one embodiment, the provided composition comprises one or more nucleic acid molecule comprising a polynucleotide sequence encoding an antigenic fragment comprising one or more domains of Gmd or an antigenic fragment or variant of one or more domains of Gmd. In some embodiments, composition comprises one or more nucleic acid molecule comprising a polynucleotide sequence encoding an antigenic fragment comprising a Gmd R3 domain. In some embodiments, composition comprises one or more nucleic acid molecule comprising a polynucleotide sequence encoding an antigenic fragment comprising the amino acid sequence of SEQ ID NO: 22. In some embodiments, composition comprises one or more nucleic acid molecule comprising a polynucleotide sequence encoding a fragment comprising an Gmd R3 domain sequence of SEQ ID NO:22 that is 138-482, 138-450, 138-400, 138-300, 138-200, 138-175, or 138-150, amino acids in length. In one embodiment, the nucleic acid comprises the sequence of SEQ ID NO: 23. In one embodiment, the nucleic acid is an RNA. In a further embodiment, the RNA comprises the sequence of SEQ ID NO: 24. In some embodiments, the composition comprises one or more nucleic acid molecule comprising a polynucleotide sequence encoding a fragment of an Gmd R3 domain sequence of SEQ ID NO:22 that 6-136, 6-125, 6-100, 6-75, 6-50, 6-25, 6-20, 6-15, or 6-10 amino acids in length.

In some embodiments, composition comprises one or more nucleic acid molecule comprising a polynucleotide sequence encoding an antigenic fragment comprising a Gmd catalytic domain. In some embodiments, the composition comprises one or more nucleic acid molecule comprising a polynucleotide sequence encoding an antigenic fragment comprising the amino acid sequence of SEQ ID NO: 25. In some embodiments, composition comprises one or more nucleic acid molecule comprising a polynucleotide sequence encoding a fragment comprising a Gmd catalytic domain sequence of SEQ ID NO:25 that is 337-482, 337-450, 337-400, 337-350, or 337-340 amino acids in length. In one embodiment, the nucleic acid comprises the sequence of SEQ ID NO: 26. In one embodiment, the nucleic acid is an RNA. In a further embodiment, the RNA comprises the sequence of SEQ ID NO: 27. In some embodiments, the composition comprises one or more nucleic acid molecule comprising a polynucleotide sequence encoding a fragment of an Gmd catalytic domain sequence of SEQ ID NO:25 that 6-336, 6-300, 6-250, 6-200, 6-175, 6-150, 6-125, 6-100, 6-75, 6-50, 6-25, 6-20, 6-15, or 6-10 amino acids in length.

In some embodiments, the composition comprises one or more nucleic acid molecule comprising a polynucleotide sequence encoding an antigenic fragment comprising the amino acid sequence of SEQ ID NO:28. In some embodiments, the composition comprises one or more nucleic acid molecule comprising a polynucleotide sequence encoding a polypeptide comprising the amino acid sequence of SEQ ID NO:28 that is 32-482, 32-450, 32-400, 32-300, 32-200, 32-175, 32-150, 32-100, 32-70, 32-50, 32-40, or 32-35, amino acids in length. In one embodiment, the nucleic acid comprises the sequence of SEQ ID NO:29. In one embodiment, the nucleic acid is an RNA. In a further embodiment, the RNA comprises the sequence of SEQ ID NO: 30. In some embodiments, the composition comprises one or more nucleic acid molecule comprising a polynucleotide sequence encoding a fragment of a polypeptide comprising the amino acid sequence of SEQ ID NO:28 that is 32-482, 32-450, 32-400, 32-300, 32-200, 32-175, 32-150, 32-100, 32-70, 32-50, 32-40, or 32-35, amino acids in length.

In one embodiment, the provided composition comprises one or more nucleic acid molecule comprising a polynucleotide sequence encoding a Gmd variant. In some embodiments, the polynucleotide sequence encodes a Gmd variant having sequence identity with respect to the amino acid sequence of SEQ ID NO:19 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In one embodiment, the provided composition comprises one or more nucleic acid molecule comprising a polynucleotide sequence encoding an Gmd variant having a total of 1-20, 1-15, 1-10, or 1-5, amino acid substitutions, deletions, and/or insertions compared to a reference sequence of SEQ ID NO:19. In one embodiment, the provided composition comprises one or more nucleic acid molecule comprising a polynucleotide sequence encoding a Gmd variant having a total of 1-20, 1-15, 1-10, or 1-5, amino acid substitutions, deletions, and/or insertions compared to a reference sequence of SEQ ID NO: 22 or 25. In one embodiment, the provided composition comprises one or more nucleic acid molecule comprising a polynucleotide sequence encoding a Gmd variant having a total of 1, 2, 3, 4, 5, 6, 7, 8, 9, 10, or fewer than 15, amino acid substitutions, deletions, and/or insertions compared to the reference sequence of SEQ ID NO:19. In one embodiment, the provided composition comprises one or more nucleic acid molecule comprising a polynucleotide sequence encoding an Gmd variant having a total of 1, 2, 3, 4, 5, 6, 7, 8, 9, 10, or fewer than 15, amino acid substitutions, deletions, and/or insertions compared to the reference sequence of SEQ ID NO: 22 or 25. In some embodiments, one or more of the substitutions in the encoded variant are conservative substitutions. In some embodiments, one or more of the deletions in the encoded variant are at the amino and or carboxy terminus of the reference sequence.

In one embodiment, the composition comprises one or more nucleic acid molecules comprising nucleic acid sequences encoding Amd and Gmd, or variants or fragments thereof. In one embodiment, the composition comprises one or more nucleic acid molecule comprising a polynucleotide sequence encoding Amd and Gmd. In one embodiment, the nucleic acid molecule comprises one or more nucleic acid sequence encoding the amino acid sequence of SEQ ID NO: 4 and SEQ ID NO: 19, or variants or fragments thereof. In one embodiment, the nucleic acid molecule comprises one or more nucleic acid sequence encoding the amino acid sequence of SEQ ID NO: 4 and SEQ ID NO: 19. In one embodiment, the composition comprises one or more nucleic acid molecules encoding Amd and Gmd. In one embodiment, the nucleic acid molecule comprises the nucleic acid sequences of SEQ ID NO: 5 and SEQ ID NO: 20. In one embodiment, the composition comprises one or more RNA molecules encoding Amd and Gmd. In one embodiment, the nucleic acid molecule comprises the RNA sequences of SEQ ID NO: 6 and SEQ ID NO: 21.

In some embodiments, the composition comprises one or more nucleic acid molecules comprising a nucleic acid sequence encoding one or more secreted immunotoxin of S. aureus, or variants or fragments thereof. In some embodiments, the one or more secreted immunotoxin of S. aureus is selected from: alpha-hemolysin (Hla), chemotaxis inhibiting protein of S. aureus (CHIPs) and the Staphylococcal complement inhibitor (SCIN).

In one embodiment, the provided composition comprises one or more nucleic acid molecule comprising a polynucleotide sequence encoding alpha-hemolysin (Hla), or an antigenic variant or fragment thereof. In one embodiment, the composition comprises one or more nucleic acid molecule comprising a polynucleotide sequence encoding Hla. In one embodiment, the composition comprises one or more nucleic acid molecule comprising a polynucleotide sequence encoding the amino acid sequence of SEQ ID NO:51, or an antigenic variant or fragment thereof. In one embodiment, the composition comprises one or more nucleic acid molecule comprising a polynucleotide sequence encoding the amino acid sequence of SEQ ID NO:51. In one embodiment, the nucleic acid comprises the sequence of SEQ ID NO: 52. In one embodiment, the nucleic acid is an RNA. In a further embodiment, the RNA comprises the sequence of SEQ ID NO: 53.

In one embodiment, the composition comprises one or more nucleic acid molecule comprising a polynucleotide sequence encoding the amino acid sequence of mature Hla. In a further embodiment, the composition comprises one or more nucleic acid molecule comprising a polynucleotide sequence encoding the amino acid sequence of SEQ ID NO: 54. In one embodiment, the nucleic acid comprises the sequence of SEQ ID NO: 55. In one embodiment, the nucleic acid is an RNA. In a further embodiment, the RNA comprises the sequence of SEQ ID NO:56.

In one embodiment, the composition comprises one or more nucleic acid molecule comprising a consensus, codon-optimized polynucleotide sequence encoding a consensus mature Hla antigenic protein. In one embodiment, the consensus mature Hla antigenic protein comprises the amino acid sequence of SEQ ID NO: 54. In one embodiment, the consensus, codon-optimized polynucleotide sequence comprises DNA. In one embodiment, the consensus, codon-optimized DNA sequence comprises the sequence of SEQ ID NO: 52. In one embodiment, the consensus, codon-optimized polynucleotide sequence comprises RNA. In one embodiment, the consensus, codon-optimized RNA sequence comprises the sequence of SEQ ID NO: 53.

In one embodiment, the provided composition comprises one or more nucleic acid molecule comprising a polynucleotide sequence encoding an antigenic fragment of Hla. In one embodiment, the composition comprises one or more nucleic acid molecule comprising a polynucleotide sequence encoding an antigenic fragment of Hla having the amino acid sequence of SEQ ID NO:51. In some embodiments, composition comprises one or more nucleic acid molecule comprising a polynucleotide sequence encoding an antigenic fragment of Hla that is 6-320, 6-310 6-300, 6-294, 6-275, 6-250, 6-200, 6-175, 6-150, 6-125, 6-100, 6-75, 6-50, 6-25, 6-20, 6-15, or 6-10 amino acids in length. In further embodiments, composition comprises one or more nucleic acid molecule comprising a polynucleotide sequence encoding an antigenic fragment of Hla having the amino acid sequence of SEQ ID NO: 54 that is 296-320, 296-310, or 296-300 amino acids in length. In some embodiments, the composition comprises one or more nucleic acid molecule comprising a polynucleotide sequence encoding an antigenic fragment of Hla comprising the amino acid sequence of SEQ ID NO:57. In some embodiments, the polynucleotide sequence of the nucleic acid comprises the sequence of SEQ ID NO: 57 and is 65-320, 65-250, 65-250, 6-200, 6-175, 6-150, 6-125, 6-100, 6-75, 6-50, 6-25, 6-20, 6-15, or 6-10 amino acids in length. In some embodiments, the composition comprises one or more nucleic acid molecule comprising a polynucleotide sequence encoding an antigenic fragment of Hla comprising the amino acid sequence of SEQ ID NO:57.

In one embodiment, the provided composition comprises one or more nucleic acid molecule comprising a polynucleotide sequence encoding an Hla variant. In some embodiments, the polynucleotide sequence encodes an Hla variant having sequence identity with respect to the amino acid sequence of SEQ ID NO:51 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the polynucleotide sequence encodes an Hla variant having sequence identity with respect to Hla having the amino acid sequence of SEQ ID NO:54 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In one embodiment, the provided composition comprises one or more nucleic acid molecule comprising a polynucleotide sequence encoding an Hla variant having a total of 1-20, 1-15, 1-10, or 1-5, amino acid substitutions, deletions, and/or insertions compared to a reference sequence of SEQ ID NO:51. In one embodiment, the provided composition comprises one or more nucleic acid molecule comprising a polynucleotide sequence encoding a mature Hla variant having a total of 1-20, 1-15, 1-10, or 1-5, amino acid substitutions, deletions, and/or insertions compared to a reference sequence of SEQ ID NO:54. In one embodiment, the provided composition comprises one or more nucleic acid molecule comprising a polynucleotide sequence encoding an Hla variant having a total of 1, 2, 3, 4, 5, 6, 7, 8, 9, 10, or fewer than 15, amino acid substitutions, deletions, and/or insertions compared to the reference sequence of SEQ ID NO:51. In one embodiment, the provided composition comprises one or more nucleic acid molecule comprising a polynucleotide sequence encoding a Hla variant having a total of 1, 2, 3, 4, 5, 6, 7, 8, 9, 10, or fewer than 15, amino acid substitutions, deletions, and/or insertions compared to the reference sequence of SEQ ID NO:54. In some embodiments, one or more of the substitutions in the encoded variant are conservative substitutions. In some embodiments, one or more of the deletions in the encoded variant are at the amino and or carboxy terminus of the reference sequence.

In one embodiment, the provided composition comprises one or more nucleic acid molecule comprising a polynucleotide sequence encoding chemotaxis inhibiting protein of S. aureus (CHIPs), or an antigenic variant or fragment thereof. In one embodiment, the composition comprises one or more nucleic acid molecule comprising a polynucleotide sequence encoding CHIPs. In one embodiment, the composition comprises one or more nucleic acid molecule comprising a polynucleotide sequence encoding the amino acid sequence of SEQ ID NO: 31, or an antigenic variant or fragment thereof. In one embodiment, the composition comprises one or more nucleic acid molecule comprising a polynucleotide sequence encoding the amino acid sequence of SEQ ID NO: 31. In one embodiment, the nucleic acid comprises the sequence of SEQ ID NO: 32. In one embodiment, the nucleic acid is an RNA. In a further embodiment, the RNA comprises the sequence of SEQ ID NO: 33.

In one embodiment, the composition comprises one or more nucleic acid molecule comprising a polynucleotide sequence encoding the amino acid sequence of mature CHIPs. In a further embodiment, the composition comprises one or more nucleic acid molecule comprising a polynucleotide sequence encoding the amino acid sequence of SEQ ID NO: 34. In one embodiment, the nucleic acid comprises the sequence of SEQ ID NO: 35. In one embodiment, the nucleic acid is an RNA. In a further embodiment, the RNA comprises the sequence of SEQ ID NO: 36.

In one embodiment, the composition comprises one or more nucleic acid molecule comprising a polynucleotide sequence encoding a CHIPS antigenic protein. In one embodiment, the composition comprises one or more nucleic acid molecule comprising a polynucleotide sequence encoding the amino acid sequence of SEQ ID NO: 86 or 89, or an antigenic variant or fragment thereof. In one embodiment, the composition comprises one or more nucleic acid molecule comprising a polynucleotide sequence encoding the amino acid sequence of SEQ ID NO: 86 or 89. In one embodiment, the nucleic acid comprises the sequence of SEQ ID NO: 87 or 90. In one embodiment, the nucleic acid is an RNA. In a further embodiment, the RNA comprises the sequence of SEQ ID NO: 87 or 91.

In one embodiment, the composition comprises one or more nucleic acid molecule comprising a consensus, codon-optimized polynucleotide sequence encoding a consensus mature CHIPS antigenic protein. In one embodiment, the consensus mature CHIPS antigenic protein comprises the amino acid sequence of SEQ ID NO: 86. In one embodiment, the consensus, codon-optimized polynucleotide sequence comprises DNA. In one embodiment, the consensus, codon-optimized DNA sequence comprises the sequence of SEQ ID NO: 87. In one embodiment, the consensus, codon-optimized polynucleotide sequence comprises RNA. In one embodiment, the consensus, codon-optimized RNA sequence comprises the sequence of SEQ ID NO: 88. In one embodiment, the provided composition comprises one or more nucleic acid molecule comprising a polynucleotide sequence encoding an antigenic fragment of CHIPs. In one embodiment, the composition comprises one or more nucleic acid molecule comprising a polynucleotide sequence encoding an antigenic fragment of CHIPs having the amino acid sequence of SEQ ID NO: 31. In some embodiments, composition comprises one or more nucleic acid molecule comprising a polynucleotide sequence encoding an antigenic fragment of CHIPs that is 6-140, 6-125, 6-100, 6-75, 6-50, 6-25, 6-20, 6-15, or 6-10 amino acids in length. In further embodiments, the composition comprises one or more nucleic acid molecule comprising a polynucleotide sequence encoding an antigenic fragment of CHIPs having the amino acid sequence of SEQ ID NO: 31 that is 6-148, 6-125, 6-100, 6-75, 6-50, 6-25, 6-20, 6-15, or 6-10 amino acids in length. In some embodiments, composition comprises one or more nucleic acid molecule comprising a polynucleotide sequence encoding an antigenic fragment of an CHIPs comprising the amino acid sequence of SEQ ID NO: 34 or 86. In some embodiments, composition comprises one or more nucleic acid molecule comprising a polynucleotide sequence encoding an antigenic fragment of a CHIPs having the amino acid sequence of SEQ ID NO: 34 or 86 that is 6-123, 6-100, 6-75, 6-50, 6-25, 6-20, 6-15, or 6-10 amino acids in length.

In some embodiments, the composition comprises one or more nucleic acid molecule comprising a polynucleotide sequence encoding an antigenic fragment of CHIPs comprising the amino acid sequence of SEQ ID NO: 37 or 89. In some embodiments, the composition comprises one or more nucleic acid molecule comprising a polynucleotide sequence encoding a polypeptide comprising the amino acid sequence of SEQ ID NO: 37 that is 62-120, 62-100, 62-90, or 62-80 amino acids in length. In one embodiment, the nucleic acid comprises the sequence of SEQ ID NO: 38 or 90. In one embodiment, the nucleic acid is an RNA. In a further embodiment, the RNA comprises the sequence of SEQ ID NO: 39 or 91. In some embodiments, the composition comprises one or more nucleic acid molecule comprising a polynucleotide sequence encoding a fragment of a polypeptide comprising the amino acid sequence of SEQ ID NO: 34 or 86 that is 6-100, 6-75, 6-50, 6-25, 6-15, or 6-10 amino acids in length.

In one embodiment, the provided composition comprises one or more nucleic acid molecule comprising a polynucleotide sequence encoding a CHIPs variant. In some embodiments, the polynucleotide sequence encodes a CHIPs variant having sequence identity with respect to the amino acid sequence of SEQ ID NO: 31 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the polynucleotide sequence encodes a CHIPs variant having sequence identity with respect to the amino acid sequence of SEQ ID NO:34 or 86 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In one embodiment, the provided composition comprises one or more nucleic acid molecule comprising a polynucleotide sequence encoding a CHIPs variant having a total of 1-20, 1-15, 1-10, or 1-5, amino acid substitutions, deletions, and/or insertions compared to a reference sequence of SEQ ID NO: 31. In one embodiment, the provided composition comprises one or more nucleic acid molecule comprising a polynucleotide sequence encoding a CHIPs variant having a total of 1-20, 1-15, 1-10, or 1-5, amino acid substitutions, deletions, and/or insertions compared to a reference sequence of SEQ ID NO: 34 or 86. In one embodiment, the provided composition comprises one or more nucleic acid molecule comprising a polynucleotide sequence encoding an CHIPs variant having a total of 1, 2, 3, 4, 5, 6, 7, 8, 9, 10, or fewer than 15, amino acid substitutions, deletions, and/or insertions compared to the reference sequence of SEQ ID NO: 31. In one embodiment, the provided composition comprises one or more nucleic acid molecule comprising a polynucleotide sequence encoding a mature CHIPs variant having a total of 1, 2, 3, 4, 5, 6, 7, 8, 9, 10, or fewer than 15, amino acid substitutions, deletions, and/or insertions compared to the reference sequence of SEQ ID NO: 34 or 86. In some embodiments, one or more of the substitutions in the encoded variant are conservative substitutions. In some embodiments, one or more of the deletions in the encoded variant are at the amino and or carboxy terminus of the reference sequence.

In one embodiment, the provided composition comprises one or more nucleic acid molecule comprising a polynucleotide sequence encoding Staphylococcal complement inhibitor (SCIN), or an antigenic variant or fragment thereof. In one embodiment, the composition comprises one or more nucleic acid molecule comprising a polynucleotide sequence encoding SCIN. In one embodiment, the composition comprises one or more nucleic acid molecule comprising a polynucleotide sequence encoding the amino acid sequence of SEQ ID NO: 40, or an antigenic variant or fragment thereof. In one embodiment, the composition comprises one or more nucleic acid molecule comprising a polynucleotide sequence encoding the amino acid sequence of SEQ ID NO: 40. In one embodiment, the nucleic acid comprises the sequence of SEQ ID NO: 41. In one embodiment, the nucleic acid is an RNA. In a further embodiment, the RNA comprises the sequence of SEQ ID NO: 42.

In one embodiment, the composition comprises one or more nucleic acid molecule comprising a polynucleotide sequence encoding the amino acid sequence of mature SCIN. In a further embodiment, the composition comprises one or more nucleic acid molecule comprising a polynucleotide sequence encoding the amino acid sequence of SEQ ID NO: 43. In one embodiment, the nucleic acid molecule is an RNA encoding mature SCIN. In one embodiment, the composition comprises one or more nucleic acid molecule comprising a polynucleotide sequence encoding the amino acid sequence of SEQ ID NO: 43. In one embodiment, the nucleic acid comprises the sequence of SEQ ID NO:75. In one embodiment, the nucleic acid is an RNA. In a further embodiment, the RNA comprises the sequence of SEQ ID NO:44.

In one embodiment, the provided composition comprises one or more nucleic acid molecule comprising a polynucleotide sequence encoding an antigenic fragment of SCIN. In one embodiment, the composition comprises one or more nucleic acid molecule comprising a polynucleotide sequence encoding an antigenic fragment of SCIN having the amino acid sequence of SEQ ID NO:40. In some embodiments, composition comprises one or more nucleic acid molecule comprising a polynucleotide sequence encoding an antigenic fragment of SCIN that is 50-110, 50-100, or 50-120 amino acids in length. In further embodiments, composition comprises one or more nucleic acid molecule comprising a polynucleotide sequence encoding an antigenic fragment of SCIN having the amino acid sequence of SEQ ID NO: 40 that is 6-45, 6-40, 6-30, 6-25, or 6-20 amino acids in length.

In one embodiment, the provided composition comprises one or more nucleic acid molecule comprising a polynucleotide sequence encoding an antigenic fragment of SCIN. In one embodiment, the composition comprises one or more nucleic acid molecule comprising a polynucleotide sequence encoding an antigenic fragment of SCIN having the amino acid sequence of SEQ ID NO:43. In some embodiments, composition comprises one or more nucleic acid molecule comprising a polynucleotide sequence encoding an antigenic fragment of SCIN that is 50-75, 50-100, or 50-120 amino acids in length. In further embodiments, composition comprises one or more nucleic acid molecule comprising a polynucleotide sequence encoding an antigenic fragment of SCIN having the amino acid sequence of SEQ ID NO: 43 that is 6-45, 6-40, 6-30, 6-25, or 6-20 amino acids in length.

In some embodiments, composition comprises one or more nucleic acid molecule comprising a polynucleotide sequence encoding an antigenic fragment comprising the amino acid sequence of SEQ ID NO:45. In some embodiments, composition comprises one or more nucleic acid molecule comprising a polynucleotide sequence encoding an antigenic fragment of an SCIN comprising the amino acid sequence of SEQ ID NO:45 that is 6-45, 6-40, 6-30, 6-20, 6-15, or 6-10 amino acids in length. In one embodiment, the nucleic acid comprises the sequence of SEQ ID NO: 46. In one embodiment, the nucleic acid is an RNA. In further embodiments, the RNA comprises the sequence of SEQ ID NO:47.

In some embodiments, composition comprises one or more nucleic acid molecule comprising a polynucleotide sequence encoding an antigenic fragment comprising the amino acid sequence of SEQ ID NO:48. In some embodiments, composition comprises one or more nucleic acid molecule comprising a polynucleotide sequence encoding an antigenic fragment of an SCIN comprising the amino acid sequence of SEQ ID NO:48 that is 6-15 or 6-10 amino acids in length. In one embodiment, the nucleic acid comprises the sequence of SEQ ID NO: 49. In further embodiments, composition comprises one or more nucleic acid molecule comprising a polynucleotide sequence encoding an antigenic fragment of SCIN having the amino acid sequence of SEQ ID NO: 48 that is 6-45, 6-40, 6-30, 6-25, or 6-20 amino acids in length. In one embodiment, the nucleic acid is an RNA. In a further embodiment, the RNA comprises the sequence of SEQ ID NO:50.

In further embodiments, composition comprises one or more nucleic acid molecule comprising a polynucleotide sequence encoding an antigenic fragment of an SCIN comprising the amino acid sequence of SEQ ID NO:40. In some embodiments, the composition comprises one or more nucleic acid molecule comprising a polynucleotide sequence encoding an antigenic fragment of an SCIN comprising the amino acid sequence of SEQ ID NO:40 that is 6-100, 6-75, 6-50, 6-25, 6-20, 6-15, or 6-10 amino acids in length. In one embodiment, the nucleic acid comprises the sequence of SEQ ID NO:41. In one embodiment, the nucleic acid is an RNA. In a further embodiment, the RNA comprises the sequence of SEQ ID NO:42.

In one embodiment, the provided composition comprises one or more nucleic acid molecule comprising a polynucleotide sequence encoding an SCIN variant. In some embodiments, the polynucleotide sequence encodes a SCIN variant having sequence identity with respect to the amino acid sequence of SEQ ID NO:40 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the polynucleotide sequence encodes a SCIN variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%, to the sequence of SEQ ID NO:43.

In one embodiment, the provided composition comprises one or more nucleic acid molecule comprising a polynucleotide sequence encoding a SCIN variant having a total of 1-20, 1-15, 1-10, or 1-5, amino acid substitutions, deletions, and/or insertions compared to a reference sequence of SEQ ID NO:40. In one embodiment, the provided composition comprises one or more nucleic acid molecule comprising a polynucleotide sequence encoding a SCIN variant having a total of 1-20, 1-15, 1-10, or 1-5, amino acid substitutions, deletions, and/or insertions compared to a reference sequence of SEQ ID NO:43. In one embodiment, the provided composition comprises one or more nucleic acid molecule comprising a polynucleotide sequence encoding an SCIN variant having a total of 1, 2, 3, 4, 5, 6, 7, 8, 9, 10, or fewer than 15, amino acid substitutions, deletions, and/or insertions compared to the reference sequence of SEQ ID NO:40. In one embodiment, the provided composition comprises one or more nucleic acid molecule comprising a polynucleotide sequence encoding a SCIN variant having a total of 1, 2, 3, 4, 5, 6, 7, 8, 9, 10, or fewer than 15, amino acid substitutions, deletions, and/or insertions compared to the reference sequence of SEQ ID NO:43. In some embodiments, one or more of the substitutions in the encoded variant are conservative substitutions. In some embodiments, one or more of the deletions in the encoded variant are at the amino and or carboxy terminus of the reference sequence.

In one embodiment, the provided composition comprises one or more nucleic acid molecule comprising a polynucleotide sequence encoding an S. aureus lytic transglycosylase protein, or an antigenic variant or fragment thereof. In one embodiment, the composition comprises one or more nucleic acid molecule comprising a polynucleotide sequence encoding IsaA. In one embodiment, the composition comprises one or more nucleic acid molecule comprising a polynucleotide sequence encoding the amino acid sequence of SEQ ID NO: 91, or an antigenic variant or fragment thereof. In one embodiment, the composition comprises one or more nucleic acid molecule comprising a polynucleotide sequence encoding the amino acid sequence of SEQ ID NO: 91. In one embodiment, the nucleic acid comprises the sequence of SEQ ID NO: 92. In one embodiment, the nucleic acid is an RNA. In a further embodiment, the RNA comprises the sequence of SEQ ID NO: 93.

In one embodiment, the composition comprises one or more nucleic acid molecule comprising a polynucleotide sequence encoding the amino acid sequence of mature IsaA. In a further embodiment, the composition comprises one or more nucleic acid molecule comprising a polynucleotide sequence encoding the amino acid sequence of SEQ ID NO: 94. In one embodiment, the nucleic acid molecule is an RNA encoding mature IsaA. In one embodiment, the composition comprises one or more nucleic acid molecule comprising a polynucleotide sequence encoding the amino acid sequence of SEQ ID NO: 94. In one embodiment, the nucleic acid comprises the sequence of SEQ ID NO:95. In one embodiment, the nucleic acid is an RNA. In a further embodiment, the RNA comprises the sequence of SEQ ID NO:96.

In one embodiment, the provided composition comprises one or more nucleic acid molecule comprising a polynucleotide sequence encoding an antigenic fragment of IsaA. In one embodiment, the composition comprises one or more nucleic acid molecule comprising a polynucleotide sequence encoding an antigenic fragment of IsaA having the amino acid sequence of SEQ ID NO:91. In some embodiments, composition comprises one or more nucleic acid molecule comprising a polynucleotide sequence encoding an antigenic fragment of IsaA that is 50-110, 50-100, or 50-120 amino acids in length. In further embodiments, composition comprises one or more nucleic acid molecule comprising a polynucleotide sequence encoding an antigenic fragment of IsaA having the amino acid sequence of SEQ ID NO: 91 that is 6-45, 6-40, 6-30, 6-25, or 6-20 amino acids in length.

In one embodiment, the provided composition comprises one or more nucleic acid molecule comprising a polynucleotide sequence encoding an antigenic fragment of IsaA. In one embodiment, the composition comprises one or more nucleic acid molecule comprising a polynucleotide sequence encoding an antigenic fragment of IsaA having the amino acid sequence of SEQ ID NO:94. In some embodiments, composition comprises one or more nucleic acid molecule comprising a polynucleotide sequence encoding an antigenic fragment of IsaA that is 50-75, 50-100, or 50-120 amino acids in length. In further embodiments, composition comprises one or more nucleic acid molecule comprising a polynucleotide sequence encoding an antigenic fragment of IsaA having the amino acid sequence of SEQ ID NO: 94 that is 6-45, 6-40, 6-30, 6-25, or 6-20 amino acids in length.

In some embodiments, composition comprises one or more nucleic acid molecule comprising a polynucleotide sequence encoding an antigenic fragment comprising the amino acid sequence of SEQ ID NO:97. In some embodiments, composition comprises one or more nucleic acid molecule comprising a polynucleotide sequence encoding an antigenic fragment of an IsaA comprising the amino acid sequence of SEQ ID NO:97 that is 6-45, 6-40, 6-30, 6-20, 6-15, or 6-10 amino acids in length. In one embodiment, the nucleic acid comprises the sequence of SEQ ID NO: 98. In one embodiment, the nucleic acid is an RNA. In further embodiments, the RNA comprises the sequence of SEQ ID NO:99.

In some embodiments, composition comprises one or more nucleic acid molecule comprising a polynucleotide sequence encoding an antigenic fragment comprising the amino acid sequence of SEQ ID NO:100. In some embodiments, composition comprises one or more nucleic acid molecule comprising a polynucleotide sequence encoding an antigenic fragment of an IsaA comprising the amino acid sequence of SEQ ID NO:100 that is 6-45, 6-40, 6-30, 6-20, 6-15, or 6-10 amino acids in length. In one embodiment, the nucleic acid comprises the sequence of SEQ ID NO: 101. In one embodiment, the nucleic acid is an RNA. In further embodiments, the RNA comprises the sequence of SEQ ID NO:102.

In some embodiments, composition comprises one or more nucleic acid molecule comprising a polynucleotide sequence encoding an antigenic fragment comprising the amino acid sequence of SEQ ID NO:103. In some embodiments, composition comprises one or more nucleic acid molecule comprising a polynucleotide sequence encoding an antigenic fragment of an IsaA comprising the amino acid sequence of SEQ ID NO:103 that is 6-45, 6-40, 6-30, 6-20, 6-15, or 6-10 amino acids in length. In one embodiment, the nucleic acid comprises the sequence of SEQ ID NO: 104. In one embodiment, the nucleic acid is an RNA. In further embodiments, the RNA comprises the sequence of SEQ ID NO:105.

In further embodiments, composition comprises one or more nucleic acid molecule comprising a polynucleotide sequence encoding an antigenic fragment of an IsaA comprising the amino acid sequence of SEQ ID NO:91. In some embodiments, the composition comprises one or more nucleic acid molecule comprising a polynucleotide sequence encoding an antigenic fragment of an IsaA comprising the amino acid sequence of SEQ ID NO:91 that is 6-100, 6-75, 6-50, 6-25, 6-20, 6-15, or 6-10 amino acids in length. In one embodiment, the nucleic acid comprises the sequence of SEQ ID NO:92. In one embodiment, the nucleic acid is an RNA. In a further embodiment, the RNA comprises the sequence of SEQ ID NO:93.

In one embodiment, the provided composition comprises one or more nucleic acid molecule comprising a polynucleotide sequence encoding an IsaA variant. In some embodiments, the polynucleotide sequence encodes a IsaA variant having sequence identity with respect to the amino acid sequence of SEQ ID NO:91 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the polynucleotide sequence encodes a IsaA variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%, to the sequence of SEQ ID NO:94.

In one embodiment, the provided composition comprises one or more nucleic acid molecule comprising a polynucleotide sequence encoding a IsaA variant having a total of 1-20, 1-15, 1-10, or 1-5, amino acid substitutions, deletions, and/or insertions compared to a reference sequence of SEQ ID NO:91. In one embodiment, the provided composition comprises one or more nucleic acid molecule comprising a polynucleotide sequence encoding a IsaA variant having a total of 1-20, 1-15, 1-10, or 1-5, amino acid substitutions, deletions, and/or insertions compared to a reference sequence of SEQ ID NO:94. In one embodiment, the provided composition comprises one or more nucleic acid molecule comprising a polynucleotide sequence encoding an IsaA variant having a total of 1, 2, 3, 4, 5, 6, 7, 8, 9, 10, or fewer than 15, amino acid substitutions, deletions, and/or insertions compared to the reference sequence of SEQ ID NO:91. In one embodiment, the provided composition comprises one or more nucleic acid molecule comprising a polynucleotide sequence encoding a IsaA variant having a total of 1, 2, 3, 4, 5, 6, 7, 8, 9, 10, or fewer than 15, amino acid substitutions, deletions, and/or insertions compared to the reference sequence of SEQ ID NO:94. In some embodiments, one or more of the substitutions in the encoded variant are conservative substitutions. In some embodiments, one or more of the deletions in the encoded variant are at the amino and or carboxy terminus of the reference sequence.

In one embodiment, the provided composition comprises one or more nucleic acid molecule comprising a polynucleotide sequence encoding SceD, or an antigenic variant or fragment thereof. In one embodiment, the composition comprises one or more nucleic acid molecule comprising a polynucleotide sequence encoding SceD. In one embodiment, the composition comprises one or more nucleic acid molecule comprising a polynucleotide sequence encoding the amino acid sequence of SEQ ID NO: 106, or an antigenic variant or fragment thereof. In one embodiment, the composition comprises one or more nucleic acid molecule comprising a polynucleotide sequence encoding the amino acid sequence of SEQ ID NO: 106. In one embodiment, the nucleic acid comprises the sequence of SEQ ID NO: 107. In one embodiment, the nucleic acid is an RNA. In a further embodiment, the RNA comprises the sequence of SEQ ID NO: 108.

In one embodiment, the composition comprises one or more nucleic acid molecule comprising a polynucleotide sequence encoding the amino acid sequence of mature SceD. In a further embodiment, the composition comprises one or more nucleic acid molecule comprising a polynucleotide sequence encoding the amino acid sequence of SEQ ID NO: 109. In one embodiment, the nucleic acid molecule is an RNA encoding mature SceD. In one embodiment, the composition comprises one or more nucleic acid molecule comprising a polynucleotide sequence encoding the amino acid sequence of SEQ ID NO: 109. In one embodiment, the nucleic acid comprises the sequence of SEQ ID NO:110. In one embodiment, the nucleic acid is an RNA. In a further embodiment, the RNA comprises the sequence of SEQ ID NO:111.

In one embodiment, the provided composition comprises one or more nucleic acid molecule comprising a polynucleotide sequence encoding an antigenic fragment of SceD. In one embodiment, the composition comprises one or more nucleic acid molecule comprising a polynucleotide sequence encoding an antigenic fragment of SceD having the amino acid sequence of SEQ ID NO:106. In some embodiments, composition comprises one or more nucleic acid molecule comprising a polynucleotide sequence encoding an antigenic fragment of SceD that is 50-110, 50-100, or 50-120 amino acids in length. In further embodiments, composition comprises one or more nucleic acid molecule comprising a polynucleotide sequence encoding an antigenic fragment of SceD having the amino acid sequence of SEQ ID NO: 106 that is 6-45, 6-40, 6-30, 6-25, or 6-20 amino acids in length.

In one embodiment, the provided composition comprises one or more nucleic acid molecule comprising a polynucleotide sequence encoding an antigenic fragment of SceD. In one embodiment, the composition comprises one or more nucleic acid molecule comprising a polynucleotide sequence encoding an antigenic fragment of SceD having the amino acid sequence of SEQ ID NO:109. In some embodiments, composition comprises one or more nucleic acid molecule comprising a polynucleotide sequence encoding an antigenic fragment of SceD that is 50-75, 50-100, or 50-120 amino acids in length. In further embodiments, composition comprises one or more nucleic acid molecule comprising a polynucleotide sequence encoding an antigenic fragment of SceD having the amino acid sequence of SEQ ID NO: 109 that is 6-45, 6-40, 6-30, 6-25, or 6-20 amino acids in length.

In some embodiments, composition comprises one or more nucleic acid molecule comprising a polynucleotide sequence encoding an antigenic fragment comprising the amino acid sequence of SEQ ID NO:112. In some embodiments, composition comprises one or more nucleic acid molecule comprising a polynucleotide sequence encoding an antigenic fragment of an SceD comprising the amino acid sequence of SEQ ID NO:112 that is 6-45, 6-40, 6-30, 6-20, 6-15, or 6-10 amino acids in length. In one embodiment, the nucleic acid comprises the sequence of SEQ ID NO: 113. In one embodiment, the nucleic acid is an RNA. In further embodiments, the RNA comprises the sequence of SEQ ID NO:114.

In further embodiments, composition comprises one or more nucleic acid molecule comprising a polynucleotide sequence encoding an antigenic fragment of an SCIN comprising the amino acid sequence of SEQ ID NO:106. In some embodiments, the composition comprises one or more nucleic acid molecule comprising a polynucleotide sequence encoding an antigenic fragment of an SceD comprising the amino acid sequence of SEQ ID NO:106 that is 6-100, 6-75, 6-50, 6-25, 6-20, 6-15, or 6-10 amino acids in length. In one embodiment, the nucleic acid comprises the sequence of SEQ ID NO:107. In one embodiment, the nucleic acid is an RNA. In a further embodiment, the RNA comprises the sequence of SEQ ID NO:108.

In one embodiment, the provided composition comprises one or more nucleic acid molecule comprising a polynucleotide sequence encoding an SceD variant. In some embodiments, the polynucleotide sequence encodes a SceD variant having sequence identity with respect to the amino acid sequence of SEQ ID NO:106 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the polynucleotide sequence encodes a SceD variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%, to the sequence of SEQ ID NO:109.

In one embodiment, the provided composition comprises one or more nucleic acid molecule comprising a polynucleotide sequence encoding a SceD variant having a total of 1-20, 1-15, 1-10, or 1-5, amino acid substitutions, deletions, and/or insertions compared to a reference sequence of SEQ ID NO:106. In one embodiment, the provided composition comprises one or more nucleic acid molecule comprising a polynucleotide sequence encoding a SceD variant having a total of 1-20, 1-15, 1-10, or 1-5, amino acid substitutions, deletions, and/or insertions compared to a reference sequence of SEQ ID NO:109. In one embodiment, the provided composition comprises one or more nucleic acid molecule comprising a polynucleotide sequence encoding an SceD variant having a total of 1, 2, 3, 4, 5, 6, 7, 8, 9, 10, or fewer than 15, amino acid substitutions, deletions, and/or insertions compared to the reference sequence of SEQ ID NO:106. In one embodiment, the provided composition comprises one or more nucleic acid molecule comprising a polynucleotide sequence encoding a SceD variant having a total of 1, 2, 3, 4, 5, 6, 7, 8, 9, 10, or fewer than 15, amino acid substitutions, deletions, and/or insertions compared to the reference sequence of SEQ ID NO:109. In some embodiments, one or more of the substitutions in the encoded variant are conservative substitutions. In some embodiments, one or more of the deletions in the encoded variant are at the amino and or carboxy terminus of the reference sequence.

In one embodiment, the provided composition comprises one or more nucleic acid molecule comprising a polynucleotide sequence encoding coproporphyrinogen III oxidase (CgoX) of S. aureus, or an antigenic variant or fragment thereof. In one embodiment, the composition comprises one or more nucleic acid molecule comprising a polynucleotide sequence encoding CgoX. In one embodiment, the composition comprises one or more nucleic acid molecule comprising a polynucleotide sequence encoding the amino acid sequence of SEQ ID NO: 60, or an antigenic variant or fragment thereof. In one embodiment, the composition comprises one or more nucleic acid molecule comprising a polynucleotide sequence encoding the amino acid sequence of SEQ ID NO: 60. In one embodiment, the nucleic acid comprises the sequence of SEQ ID NO: 61. In one embodiment, the nucleic acid is an RNA. In a further embodiment, the RNA comprises the sequence of SEQ ID NO: 62.

In one embodiment, the provided composition comprises one or more nucleic acid molecule comprising a polynucleotide sequence encoding an antigenic fragment of CgoX. In one embodiment, the composition comprises one or more nucleic acid molecule comprising a polynucleotide sequence encoding an antigenic fragment of CgoX having the amino acid sequence of SEQ ID NO:60. In some embodiments, composition comprises one or more nucleic acid molecule comprising a polynucleotide sequence encoding an antigenic fragment of CgoX that is 6-100, 6-75, 6-50, 6-25, 6-20, 6-15, or 6-10 amino acids in length.

In some embodiments, composition comprises one or more nucleic acid molecule comprising a polynucleotide sequence encoding an antigenic fragment comprising the amino acid sequence of SEQ ID NO:63. In some embodiments, the polynucleotide sequence comprises the nucleic acid sequence of SEQ ID NO: 64. In one embodiment, the nucleic acid is an RNA. In a further embodiment, the RNA comprises the sequence of SEQ ID NO: 65.

In some embodiments, composition comprises one or more nucleic acid molecule comprising a polynucleotide sequence encoding an antigenic fragment comprising the amino acid sequence of SEQ ID NO:66. In some embodiments, composition comprises one or more nucleic acid molecule comprising a polynucleotide sequence encoding an antigenic fragment comprising the amino acid sequence of SEQ ID NO:69. In some embodiments, the polynucleotide sequence comprises the nucleic acid sequence of SEQ ID NO: 67 or 70. In one embodiment, the nucleic acid is an RNA. In a further embodiment, the RNA comprises the sequence of SEQ ID NO: 68 or 71.

In further embodiments, composition comprises one or more nucleic acid molecule comprising a polynucleotide sequence encoding an antigenic fragment of an CgoX comprising the amino acid sequence of SEQ ID NO:60. In some embodiments, the composition comprises one or more nucleic acid molecule comprising a polynucleotide sequence encoding an antigenic fragment of an CgoX comprising the amino acid sequence of SEQ ID NO:60 that is 6-100, 6-75, 6-50, 6-25, 6-20, 6-15, or 6-10 amino acids in length. In one embodiment, the nucleic acid comprises the sequence of SEQ ID NO:61. In one embodiment, the nucleic acid is an RNA. In a further embodiment, the RNA comprises the sequence of SEQ ID NO:62.

In one embodiment, the provided composition comprises one or more nucleic acid molecule comprising a polynucleotide sequence encoding a CgoX variant. In some embodiments, the polynucleotide sequence encodes a CgoX variant having sequence identity with respect to the amino acid sequence of SEQ ID NO:60 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In one embodiment, the provided composition comprises one or more nucleic acid molecule comprising a polynucleotide sequence encoding a CgoX variant having a total of 1-20, 1-15, 1-10, or 1-5, amino acid substitutions, deletions, and/or insertions compared to a reference sequence of SEQ ID NO:60. In one embodiment, the provided composition comprises one or more nucleic acid molecule comprising a polynucleotide sequence encoding an CgoX variant having a total of 1, 2, 3, 4, 5, 6, 7, 8, 9, 10, or fewer than 15, amino acid substitutions, deletions, and/or insertions compared to the reference sequence of SEQ ID NO:60. In some embodiments, one or more of the substitutions in the encoded variant are conservative substitutions. In some embodiments, one or more of the deletions in the encoded variant are at the amino and or carboxy terminus of the reference sequence.

The invention also provides for the use of expression vector vaccines to stimulate an immune response against one or more S. aureus antigenic polypeptide, variant or an immunogenic fragment thereof. In a typical application of this technique, a nucleic acid encoding one or more peptide or protein antigens of S. aureus is incorporated into a vector that allows expression of the antigen(s) in a host cell (e.g., a cell inside a subject or administered to a subject). The nucleic acid encoding the antigen(s) is generally under the operational control of other sequences contained within the vector such as a promoter sequences (e.g., tissue specific, constitutively active, or inducible) or enhancer sequences. The antigen(s) encoded by the vector are expressed when the vector is introduced into a host cell in a subject. After expression, the antigen(s) can associate with an MHC molecule for presentation to immune system cells such as T lymphocytes, thus stimulating an immune response. See. e.g., Corr et al., J. Exp. Med. 184:1555, 1996.

Vectors for use in the invention can be any capable of expressing an encoded antigen(s) in a subject. For example, vectors derived from bacterial plasmids and viruses may be used. Representative viral vectors include retroviral, adenoviral, and adeno-associated viral vectors. See. e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

Some embodiments of the present disclosure provide RNA (e.g., mRNA) vaccines that include at least one RNA (e.g., mRNA) polynucleotide having an open reading frame encoding at least one antigenic polypeptide, variant or an immunogenic fragment thereof (e.g., an immunogenic fragment capable of inducing an immune response to the antigenic polypeptide). While not wishing to be bound by theory, it is believed that the RNA (e.g., mRNA) vaccines, for example, as mRNA polynucleotides, are better designed to produce the appropriate protein conformation upon translation, as the RNA (e.g., mRNA) vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

Provided herein, in some embodiments, is a ribonucleic acid (RNA) (e.g., mRNA) vaccine, comprising at least one (e.g., at least 2 or 3) RNA (e.g., mRNA) polynucleotides having an open reading frame encoding at least one (e.g., at least 2 or 3) S. aureus antigenic polypeptides or immunogenic fragments thereof. Further provided herein, in some embodiments, is a ribonucleic acid (RNA) (e.g., mRNA) vaccine, comprising at least one (e.g., at least 2 or 3) RNA (e.g., mRNA) polynucleotides having an open reading frame encoding at least one (e.g., at least 2 or 3) S. aureus antigenic polypeptides or immunogenic fragments thereof, linked to a signal peptide.

The polynucleotides provided herein, including their regions and/or parts may be codon optimized. Codon optimization methods are known in the art and may be useful in efforts to achieve one or more of several goals, including but not limited to: to match codon frequencies in target and host organisms to ensure proper folding, bias and/or GC content to increase mRNA stability or reduce secondary structures; to minimize tandem repeat codons or base runs that may impair gene construction or expression; to customize transcriptional and translational control regions; to introduce or remove protein trafficking sequences; to remove or add post translation modification sites in encoded proteins (e.g., glycosylation sites); to add, remove or shuffle protein domains; to insert or delete restriction sites; to modify ribosome binding sites and mRNA degradation sites; to adjust translational rates to allow the various domains of the protein to fold properly, or to reduce or eliminate problematic secondary structures within the polynucleotide. Codon optimization tools, algorithms and services are known in the art.

In some embodiments, the polynucleotides (e.g., DNA and RNA) provided herein are codon optimized for expression in cells of a mammalian subject. In some embodiments, the mammalian subject is a human. In some embodiments, a DNA polynucleotide provided herein comprise one or more codon optimized sequences corresponding to a nucleic acid sequence disclosed in Table 1. In some embodiments, a RNA polynucleotide provided herein comprises one or more codon optimized sequences corresponding to a nucleic acid sequence disclosed in Table 1.

In some embodiments, a codon optimized polynucleotide (e.g., DNA and RNA) provided herein shares less than 95% sequence identity, less than 90% sequence identity, less than 85% sequence identity, less than 80% sequence identity, or less than 75% sequence identity to a naturally-occurring or wild-type sequence (e.g., a naturally-occurring or wild-type mRNA sequence encoding a polypeptide or protein of interest (e.g., an antigenic protein or antigenic polypeptide)). In some embodiments, a polynucleotide (e.g., DNA and RNA) provided herein shares less than 95% sequence identity, less than 90% sequence identity, less than 85% sequence identity, less than 80% sequence identity, or less than 75% sequence identity to a nucleic acid sequence disclosed in Table 2 (e.g., a nucleic acid sequence of at least 25, 30, 50, 75 or 100 nucleotides contained in any of SEQ ID NOs. 76-85).

In some embodiments, a codon optimized polynucleotide (e.g., DNA and RNA) provided herein shares between 65% and 85% (e.g., between about 67% and about 85%, or between about 67% and about 80%) sequence identity to a naturally-occurring sequence or a wild-type sequence (e.g., a naturally-occurring or wild-type mRNA sequence encoding a polypeptide or protein of interest (e.g., an antigenic protein or polypeptide)). In some embodiments, a codon optimized polynucleotide (e.g., DNA and RNA) provided herein shares between 65% and 75%, or about 80% sequence identity to a naturally-occurring sequence or wild-type sequence (e.g., a naturally-occurring or wild-type mRNA sequence encoding a polypeptide or protein of interest (e.g., an antigenic protein or polypeptide)).

In some embodiments, a codon optimized polynucleotide (e.g., DNA and RNA) provided herein shares between 65% and 85% (e.g., between about 67% and about 85%, or between about 67% and about 80%) sequence identity to a nucleic acid sequence disclosed in Table. 2. In some embodiments, a codon optimized polynucleotide (e.g., DNA and RNA) provided herein shares between 65% and 75%, or about 80% sequence identity to a nucleic acid sequence disclosed in Table 2 (e.g., a nucleic acid sequence of at least 25, 30, 50, 75 or 100 nucleotides contained in any of SEQ ID Nos: 76-85).

In one embodiment, the provided composition comprises one or more RNA polynucleotide comprising a nucleic acid sequence encoding at least one S. aureus antigen disclosed in Table 1, or an antigenic variant or fragment thereof. In one embodiment, the composition comprises an RNA polynucleotide comprising a codon optimized nucleic acid sequence encoding an S. aureus antigen disclosed in Table 1, or an antigenic variant or fragment thereof. In one embodiment, the composition comprises one or more RNA molecule comprising a nucleic acid sequence disclosed in Table 1, or a fragment or variant thereof that encodes an antigenic polypeptide.

In one embodiment, the provided composition comprises one or more RNA polynucleotide comprising a nucleic acid sequence encoding at least one Atl, Gmd, and/or Amd antigen associated with an accession number disclosed in Table 3, or an antigenic variant or fragment thereof. In one embodiment, the composition comprises an RNA polynucleotide comprising a codon optimized nucleic acid sequence encoding at least one Atl, Gmd, and/or Amd antigen associated with an accession number disclosed in Table 3, or an antigenic variant or fragment thereof.

In one embodiment, the provided composition comprises one or more RNA polynucleotide comprising a nucleic acid sequence encoding at least one HLA antigen associated with an accession number disclosed in Table 4, or an antigenic variant or fragment thereof. In one embodiment, the composition comprises an RNA polynucleotide comprising a codon optimized nucleic acid sequence encoding at least one HLA antigen associated with an accession number disclosed in Table 4, or an antigenic variant or fragment thereof.

In one embodiment, the provided composition comprises one or more RNA polynucleotide comprising a nucleic acid sequence encoding at least one CHIPS antigen associated with an accession number disclosed in Table 5, or an antigenic variant or fragment thereof. In one embodiment, the composition comprises an RNA polynucleotide comprising a codon optimized nucleic acid sequence encoding at least one CHIPS antigen associated with an accession number disclosed in Table 5, or an antigenic variant or fragment thereof.

In one embodiment, the provided composition comprises one or more RNA polynucleotide comprising a nucleic acid sequence encoding at least one SCIN antigen associated with an accession number disclosed in Table 6, or an antigenic variant or fragment thereof. In one embodiment, the composition comprises an RNA polynucleotide comprising a codon optimized nucleic acid sequence encoding at least one SCIN antigen associated with an accession number disclosed in Table 6, or an antigenic variant or fragment thereof.

In one embodiment, the provided composition comprises one or more RNA polynucleotide comprising a nucleic acid sequence encoding at least one lytic transglycosylase antigen associated with an accession number disclosed in Table 7, or an antigenic variant or fragment thereof. In one embodiment, the composition comprises an RNA polynucleotide comprising a codon optimized nucleic acid sequence encoding at least one lytic transglycosylase antigen associated with an accession number disclosed in Table 7, or an antigenic variant or fragment thereof.

In one embodiment, the provided composition comprises one or more RNA polynucleotide comprising a nucleic acid sequence encoding at least one IsaA antigen associated with an accession number disclosed in Table 7, or an antigenic variant or fragment thereof. In one embodiment, the composition comprises an RNA polynucleotide comprising a codon optimized nucleic acid sequence encoding at least one IsaA antigen associated with an accession number disclosed in Table 7, or an antigenic variant or fragment thereof.

In one embodiment, the provided composition comprises one or more RNA polynucleotide comprising a nucleic acid sequence encoding at least one SceD antigen associated with an accession number disclosed in Table 7, or an antigenic variant or fragment thereof. In one embodiment, the composition comprises an RNA polynucleotide comprising a codon optimized nucleic acid sequence encoding at least one SceD antigen associated with an accession number disclosed in Table 7, or an antigenic variant or fragment thereof.

In one embodiment, the provided composition comprises one or more RNA polynucleotide comprising a sequence that encodes Atl. In one embodiment, the provided composition comprises one or more RNA polynucleotide comprising a sequence that encodes the amino acid sequence of SEQ ID NO: 1. In one embodiment, the provided composition comprises one or more RNA polynucleotide comprising a sequence that encodes the amino acid sequence of SEQ ID NO: 72. In one embodiment, the provided composition comprises one or more RNA polynucleotide comprising a nucleic acid sequence encoding an antigenic variant or fragment of Atl. In one embodiment, the RNA polynucleotide comprises a sequence encoding a polypeptide variant or fragment of SEQ ID NO: 1. In one embodiment, the RNA polynucleotide comprises a sequence encoding a polypeptide variant or fragment of SEQ ID NO: 72. In one embodiment, the one or more RNA polynucleotide of the provided composition is not naturally occurring. In one embodiment, the one or more RNA polynucleotide of the provided composition comprises a coding sequence that is codon optimized. In a further embodiment, the RNA polynucleotide comprises a nucleic acid sequence that is codon optimized for expression in cells of a mammalian subject (e.g., a human). In one embodiment, the RNA polynucleotide comprises the sequence of SEQ ID NO: 3. In one embodiment, the RNA polynucleotide comprises the sequence of SEQ ID NO: 74.

In one embodiment, the provided composition comprises one or more RNA polynucleotide comprising a nucleic acid sequence encoding an antigenic fragment of Atl. In one embodiment, the composition comprises one or more RNA polynucleotide comprising a nucleic acid sequence encoding an antigenic fragment of an Atl polypeptide having the amino acid sequence of SEQ ID NO:1. In some embodiments, the composition comprises a RNA polynucleotide comprising a nucleic acid sequence encoding an antigenic fragment of Atl that is 6-1267, 6-1200, 6-1000, 6-750, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In further embodiments, the composition comprises a RNA polynucleotide comprising a nucleic acid sequence encoding an antigenic fragment of an Atl having the amino acid sequence of SEQ ID NO:72 that is 6-1066, 6-1000, 6-750, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length.

In one embodiment, the provided composition comprises one or more RNA polynucleotide comprising a nucleic acid sequence encoding an Atl variant. In some embodiments, the RNA polynucleotide comprises a nucleic acid sequence that encodes an Atl variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% with the amino acid sequence of SEQ ID NO:1. In some embodiments, the RNA polynucleotide comprises a nucleic acid sequence that encodes an Atl variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% with the amino acid sequence of SEQ ID NO:72.

In one embodiment, the provided composition comprises one or more RNA polynucleotide comprising a nucleic acid sequence encoding an Atl variant having a total of 1-100, 1-75, 1-50, 1-24, 1-30, 1-20, 1-15, 1-10, or 1-5, amino acid substitutions, deletions, and/or insertions compared to a reference Atl sequence of SEQ ID NO:1. In one embodiment, the RNA polynucleotide comprises a nucleic acid sequence encoding an Atl variant having a total of 1, 2, 3, 4, 5, 6, 7, 8, 9, 10, or fewer than 15, amino acid substitutions, deletions, and/or insertions compared to a reference Atl sequence of SEQ ID NO:1. In some embodiments, one or more of the substitutions in the Atl variant encoded by the RNA polynucleotide is a conservative substitution. In some embodiments, one or more of the deletions in the Atl variant encoded by the RNA polynucleotide is at the amino terminus and or carboxy terminus of the reference Atl sequence.

In one embodiment, the provided composition comprises one or more RNA polynucleotide comprising a nucleic acid sequence encoding an Atl variant having a total of 1-100, 1-75, 1-50, 1-24, 1-30, 1-20, 1-15, 1-10, or 1-5, amino acid substitutions, deletions, and/or insertions compared to a reference Atl sequence of SEQ ID NO:72. In one embodiment, the RNA polynucleotide comprises a nucleic acid sequence encoding an Atl variant having a total of 1, 2, 3, 4, 5, 6, 7, 8, 9, 10, or fewer than 15, amino acid substitutions, deletions, and/or insertions compared to a reference Atl sequence of SEQ ID NO:72. In some embodiments, one or more of the substitutions in the Atl variant encoded by the RNA polynucleotide is a conservative substitution. In some embodiments, one or more of the deletions in the Atl variant encoded by the RNA polynucleotide is at the amino terminus and or carboxy terminus of the reference Atl sequence.

In one embodiment, the provided composition comprises one or more RNA polynucleotide comprising a sequence that encodes N-acetylmuramyl-L-alanine amidase (Amd). In one embodiment, the provided composition comprises one or more RNA polynucleotide comprising a sequence that encodes the amino acid sequence of SEQ ID NO: 4. In one embodiment, the provided composition comprises one or more RNA polynucleotide comprising a nucleic acid sequence encoding an antigenic variant or fragment of Amd. In one embodiment, the RNA polynucleotide comprises a sequence encoding a polypeptide variant or fragment of SEQ ID NO: 4. In one embodiment, the one or more RNA polynucleotide of the provided composition is not naturally occurring. In one embodiment, the one or more RNA polynucleotide of the provided composition comprises a coding sequence that is codon optimized. In a further embodiment, the RNA polynucleotide comprises a nucleic acid sequence that is codon optimized for expression in cells of a mammalian subject (e.g., a human). In one embodiment, the RNA polynucleotide comprises the sequence of SEQ ID NO: 6.

In one embodiment, the provided composition comprises one or more RNA polynucleotide comprising a nucleic acid sequence encoding an antigenic fragment of Amd. In one embodiment, the composition comprises one or more RNA polynucleotide comprising a nucleic acid sequence encoding an antigenic fragment of an Amd polypeptide having the amino acid sequence of SEQ ID NO:4. In some embodiments, the composition comprises a RNA polynucleotide comprising a nucleic acid sequence encoding an antigenic fragment of Amd that is 6-590, 6-575, 6-550, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In further embodiments, the composition comprises an RNA polynucleotide comprising a nucleic acid sequence encoding an antigenic fragment of an Amd having the amino acid sequence of SEQ ID NO: 4 that is 6-590, 6-575, 6-550, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length.

In one embodiment, the provided composition comprises one or more RNA polynucleotide comprising a nucleic acid sequence encoding an antigenic fragment comprising one or more domains of Amd. In some embodiments, one or more RNA polynucleotide comprises a nucleic acid sequence encoding an antigenic fragment comprising an Amd R1 domain. In some embodiments, one or more RNA polynucleotide comprises a nucleic acid sequence encoding a fragment comprising an Amd R1 domain sequence of SEQ ID NO:7 that is 175-590, 175-575, 175-550, 175-500, 175-450, 175-400, 175-350, 175-300, 175-250, 175-200, or 175-190, amino acids in length. In some embodiments, one or more RNA polynucleotide comprises a nucleic acid sequence encoding an antigenic fragment comprising an Amd R2 domain. In some embodiments, one or more RNA polynucleotide comprises a nucleic acid sequence encoding a fragment comprising an Amd R2 domain sequence of SEQ ID NO:10 that is 194-590, 194-575, 194-550, 194-500, 194-450, 194-400, 194-350, 194-300, 194-250, or 194-200, amino acids in length. In some embodiments, one or more RNA polynucleotide comprises a nucleic acid sequence encoding an antigenic fragment comprising an Amd catalytic domain. In some embodiments, one or more RNA polynucleotide comprises a nucleic acid sequence encoding a fragment comprising an Amd catalytic domain sequence of SEQ ID NO:16 that is 224-590, 224-575, 224-550, 224-500, 224-450, 224-400, 224-350, 224-300, or 224-250, amino acids in length.

In one embodiment, the provided composition comprises one or more RNA polynucleotide comprising a nucleic acid sequence encoding an antigenic fragment comprising an Amd R1 and R2 domain. In some embodiments, one or more RNA polynucleotide comprises a nucleic acid sequence encoding a fragment comprising an Amd R1 and R2 domain sequence. In some embodiments, the one or more RNA polynucleotide comprises a nucleic acid sequence encoding a fragment comprising an Amd R1 and R2 domain sequence of SEQ ID NO:13. In some embodiments, the one or more RNA polynucleotide comprises a nucleic acid sequence encoding a fragment comprising an Amd R1 and R2 domain sequence of SEQ ID NO:13 that is 369-1180, 369-1000, 369-750, 369-500, 369-450, or 369-400, amino acids in length.

In one embodiment, the provided composition comprises one or more RNA polynucleotide comprising a nucleic acid sequence encoding an Amd variant. In some embodiments, one or more RNA polynucleotide comprises a nucleic acid encoding an Amd variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%, with the amino acid sequence of SEQ ID NO:4.

In one embodiment, the provided composition comprises one or more RNA polynucleotide comprising a nucleic acid sequence encoding an Amd variant having a total of 1-100, 1-75, 1-50, 1-24, 1-30, 1-20, 1-15, 1-10, or 1-5, amino acid substitutions, deletions, and/or insertions compared to a reference Amd sequence of SEQ ID NO:4. In one embodiment, the RNA polynucleotide comprises a nucleic acid sequence encoding an Amd variant having a total of 1, 2, 3, 4, 5, 6, 7, 8, 9, 10, or fewer than 15, amino acid substitutions, deletions, and/or insertions compared to a reference Amd sequence of SEQ ID NO:4. In some embodiments, one or more of the substitutions in the Amd variant encoded by the RNA polynucleotide is a conservative substitution. In some embodiments, one or more of the deletions in the Amd variant encoded by the RNA polynucleotide is at the amino terminus and/or the carboxy terminus of the reference Amd sequence.

In one embodiment, the provided composition comprises one or more RNA polynucleotide comprising a sequence that encodes one or more RNA polynucleotide comprising a nucleic acid sequence encoding endo-β-N-acetylglucosaminidase (Gmd). In one embodiment, the provided composition comprises one or more RNA polynucleotide comprising a sequence that encodes the amino acid sequence of SEQ ID NO: 19. In one embodiment, the provided composition comprises one or more RNA polynucleotide comprising a nucleic acid sequence encoding an antigenic variant or fragment of Gmd. In one embodiment, the RNA polynucleotide comprises a sequence encoding a polypeptide variant or fragment of SEQ ID NO: 19. In one embodiment, the one or more RNA polynucleotide of the provided composition is not naturally occurring. In one embodiment, the one or more RNA polynucleotide of the provided composition comprises a coding sequence that is codon optimized. In a further embodiment, the RNA polynucleotide comprises a nucleic acid sequence that is codon optimized for expression in cells of a mammalian subject (e.g., a human). In one embodiment, the RNA polynucleotide comprises the sequence of SEQ ID NO: 21.

In one embodiment, the provided composition comprises one or more RNA polynucleotide comprising a nucleic acid sequence encoding an antigenic fragment of Gmd. In one embodiment, the composition comprises one or more RNA polynucleotide comprising a nucleic acid sequence encoding an antigenic fragment of an Gmd polypeptide having the amino acid sequence of SEQ ID NO:19. In some embodiments, the composition comprises a RNA polynucleotide comprising a nucleic acid sequence encoding an antigenic fragment of Gmd that is 6-482, 6-475, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In further embodiments, the composition comprises an RNA polynucleotide comprising a nucleic acid sequence encoding an antigenic fragment of an Gmd having the amino acid sequence of SEQ ID NO: 19 that is 6-482, 6-475, 6-450, 6-400, 6-350, 6-300, 6-250, 6-200, 6-175, 6-150, 6-125, 6-100, 6-75, 6-50, 6-25, 6-20, 6-15, or 6-10 amino acids in length.

In one embodiment, the provided composition comprises one or more RNA polynucleotide comprising a nucleic acid sequence encoding an antigenic fragment comprising one or more domains of Gmd. In some embodiments, one or more RNA polynucleotide comprises a nucleic acid sequence encoding an antigenic fragment comprising a Gmd R3 domain. In some embodiments, one or more RNA polynucleotide comprises a nucleic acid sequence encoding a fragment comprising an Gmd R3 domain sequence of SEQ ID NO:22 that is 137-482, 137-450, 137-500, 137-450, 137-400, 137-350, 137-300, 137-250, 137-200, 137-175, or 1137-150, amino acids in length, amino acids in length. In some embodiments, one or more RNA polynucleotide comprises a nucleic acid sequence encoding an antigenic fragment comprising an Gmd R3 domain. In some embodiments, one or more RNA polynucleotide comprises a nucleic acid sequence encoding a fragment comprising an Gmd R3 domain sequence of SEQ ID NO:22 that is 137-482, 137-450, 137-500, 137-450, 137-400, 137-350, 137-300, 137-250, 137-200, 137-175, or 1137-150, amino acids in length. In some embodiments, one or more RNA polynucleotide comprises a nucleic acid sequence encoding an antigenic fragment comprising a Gmd catalytic domain. In some embodiments, one or more RNA polynucleotide comprises a nucleic acid sequence encoding a fragment comprising an Gmd catalytic domain sequence of SEQ ID NO:25 that is 6-500, 6-450, 6-400, 6-350, 6-300, 6-250, 6-200, 6-175, 6-150, 6-125, 6-100, 6-75, 6-50, 6-25, 6-20, 6-15, or 6-10, amino acids in length.

In one embodiment, the provided composition comprises one or more RNA polynucleotide comprising a nucleic acid sequence encoding an antigenic fragment of Gmd comprising the amino acid sequence of SEQ ID NO: 28. In some embodiments, one or more RNA polynucleotide comprises a nucleic acid sequence encoding a fragment comprising the amino acid sequence of SEQ ID NO:28 that is 32-482, 32-450, 32-400, 32-300, 32-200, 32-175, 32-150, 32-100, 32-70, 32-50, 32-40, or 32-35 amino acids in length.

In one embodiment, the provided composition comprises one or more RNA polynucleotide comprising a nucleic acid sequence encoding a Gmd variant. In some embodiments, one or more RNA polynucleotide comprises a nucleic acid encoding an Gmd variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%, with the amino acid sequence of SEQ ID NO:19.

In one embodiment, the provided composition comprises one or more RNA polynucleotide comprising a nucleic acid sequence encoding an Gmd variant having a total of 1-100, 1-75, 1-50, 1-24, 1-30, 1-20, 1-15, 1-10, or 1-5, amino acid substitutions, deletions, and/or insertions compared to a reference Gmd sequence of SEQ ID NO:19. In one embodiment, the RNA polynucleotide comprises a nucleic acid sequence encoding an Gmd variant having a total of 1, 2, 3, 4, 5, 6, 7, 8, 9, 10, or fewer than 15, amino acid substitutions, deletions, and/or insertions compared to a reference Gmd sequence of SEQ ID NO:19. In some embodiments, one or more of the substitutions in the Gmd variant encoded by the RNA polynucleotide is a conservative substitution. In some embodiments, one or more of the deletions in the Gmd variant encoded by the RNA polynucleotide is at the amino terminus and/or the carboxy terminus of the reference Gmd sequence.

In some embodiments, the composition comprises one or more RNA polynucleotides comprising a nucleic acid sequence encoding one or more secreted immunotoxin of S. aureus, or variants or fragments thereof. In some embodiments, the one or more secreted immunotoxin of S. aureus is selected from: alpha-hemolysin (Hla), chemotaxis inhibiting protein of S. aureus (CHIPs) and the Staphylococcal complement inhibitor (SCIN).

In one embodiment, the provided composition comprises one or more RNA polynucleotide comprising a nucleic acid sequence that encodes alpha-hemolysin (Hla). In one embodiment, one or more RNA polynucleotide comprises a nucleic acid sequence encoding the amino acid sequence of SEQ ID NO:51. In one embodiment, one or more RNA polynucleotide comprises a nucleic acid sequence encoding the amino acid sequence of SEQ ID NO:54. In one embodiment, one or more RNA polynucleotide comprises a nucleic acid sequence encoding an antigenic variant or fragment of Hla. In one embodiment, one or more RNA polynucleotide comprises a nucleic acid sequence encoding an antigenic variant or fragment of Hla having the sequence of SEQ ID NO:51. In one embodiment, one or more RNA polynucleotide comprises a nucleic acid sequence encoding an antigenic variant or fragment of Hla having the sequence of SEQ ID NO:54. In one embodiment, one or more RNA polynucleotide of the provided composition is not naturally occurring. In one embodiment, one or more RNA polynucleotide of the provided composition comprises a coding sequence that is codon optimized. In a further embodiment, the RNA polynucleotide comprises a nucleic acid sequence that is codon optimized for expression in cells of a mammalian subject (e.g., a human). In one embodiment, one or more RNA polynucleotide comprises the sequence of SEQ ID NO: 53. In one embodiment, one or more RNA polynucleotide comprises the sequence of SEQ ID NO:56.

In one embodiment, the provided composition comprises one or more RNA polynucleotide comprising a nucleic acid sequence encoding an antigenic fragment of Hla. In one embodiment, the composition comprises one or more RNA polynucleotide comprising a nucleic acid sequence encoding an antigenic fragment of an Hla polypeptide having the amino acid sequence of SEQ ID NO:51. In one embodiment, the composition comprises one or more RNA polynucleotide comprising a nucleic acid sequence encoding an antigenic fragment of an Hla polypeptide having the amino acid sequence of SEQ ID NO:54. In some embodiments, the composition comprises a RNA polynucleotide comprising a nucleic acid sequence encoding an antigenic fragment of Hla that is 6-320 6-300, 6-294, 6-275, 6-250, 6-200, 6-175, 6-150, 6-125, 6-100, 6-75, 6-50, 6-25, 6-20, 6-15, or 6-10 amino acids in length. In further embodiments, the composition comprises an RNA polynucleotide comprising a nucleic acid sequence encoding an antigenic fragment of an Hla having the amino acid sequence of SEQ ID NO:51 that is 6-320 6-300, 6-294, 6-275, 6-250, 6-200, 6-175, 6-150, 6-125, 6-100, 6-75, 6-50, 6-25, 6-20, 6-15, or 6-10 amino acids in length. In one embodiment, the composition comprises an RNA polynucleotide comprising a nucleic acid sequence encoding an antigenic fragment of an Hla having the amino acid sequence of SEQ ID NO:54 that is 6-482, 6-475, 6-450, 6-400, 6-350, 6-300, 6-250, 6-200, 6-175, 6-150, 6-125, 6-100, 6-75, 6-50, 6-25, 6-20, 6-15, or 6-10 amino acids in length.

In one embodiment, the provided composition comprises one or more RNA polynucleotide comprising a nucleic acid sequence encoding an antigenic fragment of Hla comprising the amino acid sequence of SEQ ID NO:57. In some embodiments, one or more RNA polynucleotide comprises a nucleic acid sequence encoding a fragment of Hla that comprises the amino acid sequence of SEQ ID NO:57 and that is 66-294, 66-200, 66-150, 66-100, 66-75, or 66-70 amino acids in length.

In one embodiment, the provided composition comprises one or more RNA polynucleotide comprising a nucleic acid sequence encoding a Hla variant. In some embodiments, one or more RNA polynucleotide comprises a nucleic acid encoding an Hla variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%, with the amino acid sequence of SEQ ID NO:51, 54, or 57.

In one embodiment, the provided composition comprises one or more RNA polynucleotide comprising a nucleic acid sequence encoding an Hla variant having a total of 1-100, 1-75, 1-50, 1-24, 1-30, 1-20, 1-15, 1-10, or 1-5, amino acid substitutions, deletions, and/or insertions compared to a reference Hla sequence of SEQ ID NO:51. In one embodiment, the RNA polynucleotide comprises a nucleic acid sequence encoding an Hla variant having a total of 1, 2, 3, 4, 5, 6, 7, 8, 9, 10, or fewer than 15, amino acid substitutions, deletions, and/or insertions compared to a reference Hla sequence of SEQ ID NO:51. In one embodiment, the provided composition comprises one or more RNA polynucleotide comprising a nucleic acid sequence encoding an Hla variant having a total of 1-100, 1-75, 1-50, 1-24, 1-30, 1-20, 1-15, 1-10, or 1-5, amino acid substitutions, deletions, and/or insertions compared to a reference Hla sequence of SEQ ID NO:54. In one embodiment, the RNA polynucleotide comprises a nucleic acid sequence encoding an Hla variant having a total of 1, 2, 3, 4, 5, 6, 7, 8, 9, 10, or fewer than 15, amino acid substitutions, deletions, and/or insertions compared to a reference Hla sequence of SEQ ID NO:54. In one embodiment, the provided composition comprises one or more RNA polynucleotide comprising a nucleic acid sequence encoding an Hla variant having a total of 1-100, 1-75, 1-50, 1-24, 1-30, 1-20, 1-15, 1-10, or 1-5, amino acid substitutions, deletions, and/or insertions compared to a reference Hla sequence of SEQ ID NO:57. In one embodiment, the RNA polynucleotide comprises a nucleic acid sequence encoding an Hla variant having a total of 1, 2, 3, 4, 5, 6, 7, 8, 9, 10, or fewer than 15, amino acid substitutions, deletions, and/or insertions compared to a reference Hla sequence of SEQ ID NO:57. In some embodiments, one or more of the substitutions in the Hla variant encoded by the RNA polynucleotide is a conservative substitution. In some embodiments, one or more of the deletions in the Hla variant encoded by the RNA polynucleotide is at the amino terminus and/or the carboxy terminus of the reference Hla sequence.

In one embodiment, the provided composition comprises one or more RNA polynucleotide comprising a nucleic acid sequence that encodes coproporphyrinogen III oxidase (CgoX). In one embodiment, one or more RNA polynucleotide comprises a nucleic acid sequence encoding the amino acid sequence of SEQ ID NO: 60. In one embodiment, one or more RNA polynucleotide comprises a nucleic acid sequence encoding the amino acid sequence of SEQ ID NO: 60. In one embodiment, one or more RNA polynucleotide comprises a nucleic acid sequence encoding an antigenic variant or fragment of CgoX. In one embodiment, one or more RNA polynucleotide comprises a nucleic acid sequence encoding an antigenic variant or fragment of CgoX having the nucleic acid sequence of SEQ ID NO:61. In one embodiment, one or more RNA polynucleotide of the provided composition is not naturally occurring. In one embodiment, one or more RNA polynucleotide of the provided composition comprises a coding sequence that is codon optimized. In a further embodiment, the RNA polynucleotide comprises a nucleic acid sequence that is codon optimized for expression in cells of a mammalian subject (e.g., a human). In one embodiment, one or more RNA polynucleotide comprises the sequence of SEQ ID NO: 62.

In one embodiment, the provided composition comprises one or more RNA polynucleotide comprising a nucleic acid sequence encoding an antigenic fragment of CgoX. In one embodiment, the composition comprises one or more RNA polynucleotide comprising a nucleic acid sequence encoding an antigenic fragment of an CgoX polypeptide having the amino acid sequence of SEQ ID NO: 60. In one embodiment, the composition comprises one or more RNA polynucleotide comprising a nucleic acid sequence encoding an antigenic fragment of an CgoX polypeptide having the amino acid sequence of SEQ ID NO: 60. In some embodiments, the composition comprises a RNA polynucleotide comprising a nucleic acid sequence encoding an antigenic fragment of CgoX that is 6-465 6-450, 6-400, 6-350 6-300, 6-250, 6-200, 6-175, 6-150, 6-125, 6-100, 6-75, 6-50, 6-25, 6-20, 6-15, or 6-10 amino acids in length.

In one embodiment, the provided composition comprises one or more RNA polynucleotide comprising a nucleic acid sequence encoding an antigenic fragment of CgoX comprising the amino acid sequence of SEQ ID NO: 63. In some embodiments, one or more RNA polynucleotide comprises a nucleic acid sequence encoding a fragment of CgoX that comprises the amino acid sequence of SEQ ID NO: 63 and that is 13-465, 13-450, 13-400, 13-350, 13-300, 13-250, 13-200, 13-150, 13-100, 13-75, 13-50, 13-25, 13-20, or 13-15, amino acids in length. In one embodiment, the provided composition comprises one or more RNA polynucleotide comprising a nucleic acid sequence encoding an antigenic fragment of CgoX comprising the amino acid sequence of SEQ ID NO:66. In some embodiments, one or more RNA polynucleotide comprises a nucleic acid sequence encoding a fragment of CgoX that comprises the amino acid sequence of SEQ ID NO:69.

In one embodiment, the provided composition comprises one or more RNA polynucleotide comprising a nucleic acid sequence encoding a CgoX variant. In some embodiments, one or more RNA polynucleotide comprises a nucleic acid encoding an CgoX variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%, with the amino acid sequence of SEQ ID NO: 60, 63, 66, or 69.

In one embodiment, the provided composition comprises one or more RNA polynucleotide comprising a nucleic acid sequence encoding an CgoX variant having a total of 1-100, 1-75, 1-50, 1-24, 1-30, 1-20, 1-15, 1-10, or 1-5, amino acid substitutions, deletions, and/or insertions compared to a reference CgoX sequence of SEQ ID NO:54.

In one embodiment, the RNA polynucleotide comprises a nucleic acid sequence encoding an CgoX variant having a total of 1, 2, 3, 4, 5, 6, 7, 8, 9, 10, or fewer than 15, amino acid substitutions, deletions, and/or insertions compared to a reference CgoX sequence of SEQ ID NO:54. In some embodiments, one or more of the substitutions in the CgoX variant encoded by the RNA polynucleotide is a conservative substitution. In some embodiments, one or more of the deletions in the CgoX variant encoded by the RNA polynucleotide is at the amino terminus and/or the carboxy terminus of the reference CgoX sequence.

In one embodiment, the provided composition comprises one or more RNA polynucleotide comprising a nucleic acid sequence that encodes chemotaxis inhibiting protein of S. aureus (CHIPs). In one embodiment, one or more RNA polynucleotide comprises a nucleic acid sequence encoding the amino acid sequence of SEQ ID NO: 31. In one embodiment, one or more RNA polynucleotide comprises a nucleic acid sequence encoding the amino acid sequence of SEQ ID NO: 34. In one embodiment, one or more RNA polynucleotide comprises a nucleic acid sequence encoding an antigenic variant or fragment of CHIPs. In one embodiment, one or more RNA polynucleotide comprises a nucleic acid sequence encoding an antigenic variant or fragment of CHIPs having the sequence of SEQ ID NO: 31. In one embodiment, one or more RNA polynucleotide comprises a nucleic acid sequence encoding an antigenic variant or fragment of CHIPs having the sequence of SEQ ID NO: 34. In one embodiment, one or more RNA polynucleotide of the provided composition is not naturally occurring. In one embodiment, one or more RNA polynucleotide of the provided composition comprises a coding sequence that is codon optimized. In a further embodiment, the RNA polynucleotide comprises a nucleic acid sequence that is codon optimized for expression in cells of a mammalian subject (e.g., a human). In one embodiment, one or more RNA polynucleotide comprises the sequence of SEQ ID NO: 33. In one embodiment, one or more RNA polynucleotide comprises the sequence of SEQ ID NO: 36.

In one embodiment, the provided composition comprises one or more RNA polynucleotide comprising a nucleic acid sequence encoding an antigenic fragment of CHIPs. In one embodiment, the composition comprises one or more RNA polynucleotide comprising a nucleic acid sequence encoding an antigenic fragment of an CHIPs polypeptide having the amino acid sequence of SEQ ID NO: 31. In one embodiment, the composition comprises one or more RNA polynucleotide comprising a nucleic acid sequence encoding an antigenic fragment of an CHIPs polypeptide having the amino acid sequence of SEQ ID NO:34. In some embodiments, the composition comprises a RNA polynucleotide comprising a nucleic acid sequence encoding an antigenic fragment of CHIPs that is 6-148, 6-140, 6-125, 6-100, 6-75, 6-50, 6-25, 6-20, 6-15, or 6-10 amino acids in length. In further embodiments, the composition comprises an RNA polynucleotide comprising a nucleic acid sequence encoding an antigenic fragment of CHIPs having the amino acid sequence of SEQ ID NO: 31 that is 6-120, 6-100, 6-75, 6-50, 6-25, 6-20, 6-15, or 6-10 amino acids in length. In one embodiment, the provided composition comprises one or more RNA polynucleotide comprising a nucleic acid sequence encoding an antigenic fragment of CHIPs comprising the amino acid sequence of SEQ ID NO: 37. In some embodiments, one or more RNA polynucleotide comprises a nucleic acid sequence encoding a fragment of CHIPs that comprises the amino acid sequence of SEQ ID NO: 37 and that is 62-148, 62-125, 62-100, 62-75, or 62-70 amino acids in length.

In one embodiment, the provided composition comprises one or more RNA polynucleotide comprising a nucleic acid sequence encoding a CHIPs variant. In some embodiments, one or more RNA polynucleotide comprises a nucleic acid encoding an CHIPs variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%, with the amino acid sequence of SEQ ID NO:31, 34, or 37.

In one embodiment, the provided composition comprises one or more RNA polynucleotide comprising a nucleic acid sequence encoding an CHIPs variant having a total of 1-100, 1-75, 1-50, 1-24, 1-30, 1-20, 1-15, 1-10, or 1-5, amino acid substitutions, deletions, and/or insertions compared to a reference CHIPs sequence of SEQ ID NO: 31. In one embodiment, the RNA polynucleotide comprises a nucleic acid sequence encoding an CHIPs variant having a total of 1, 2, 3, 4, 5, 6, 7, 8, 9, 10, or fewer than 15, amino acid substitutions, deletions, and/or insertions compared to a reference CHIPs sequence of SEQ ID NO: 31. In one embodiment, the provided composition comprises one or more RNA polynucleotide comprising a nucleic acid sequence encoding an CHIPs variant having a total of 1-100, 1-75, 1-50, 1-24, 1-30, 1-20, 1-15, 1-10, or 1-5, amino acid substitutions, deletions, and/or insertions compared to a reference CHIPs sequence of SEQ ID NO:34. In one embodiment, the RNA polynucleotide comprises a nucleic acid sequence encoding an CHIPs variant having a total of 1, 2, 3, 4, 5, 6, 7, 8, 9, 10, or fewer than 15, amino acid substitutions, deletions, and/or insertions compared to a reference CHIPs sequence of SEQ ID NO:34. In one embodiment, the provided composition comprises one or more RNA polynucleotide comprising a nucleic acid sequence encoding an CHIPs variant having a total of 1-100, 1-75, 1-50, 1-24, 1-30, 1-20, 1-15, 1-10, or 1-5, amino acid substitutions, deletions, and/or insertions compared to a reference CHIPs sequence of SEQ ID NO: 37. In one embodiment, the RNA polynucleotide comprises a nucleic acid sequence encoding an CHIPs variant having a total of 1, 2, 3, 4, 5, 6, 7, 8, 9, 10, or fewer than 15, amino acid substitutions, deletions, and/or insertions compared to a reference CHIPs sequence of SEQ ID NO: 37. In some embodiments, one or more of the substitutions in the CHIPs variant encoded by the RNA polynucleotide is a conservative substitution. In some embodiments, one or more of the deletions in the CHIPs variant encoded by the RNA polynucleotide is at the amino terminus and/or the carboxy terminus of the reference CHIPs sequence.

In one embodiment, the provided composition comprises one or more RNA polynucleotide comprising a nucleic acid sequence that encodes Staphylococcal complement inhibitor (SCIN). In one embodiment, one or more RNA polynucleotide comprises a nucleic acid sequence encoding the amino acid sequence of SEQ ID NO: 40. In one embodiment, one or more RNA polynucleotide comprises a nucleic acid sequence encoding the amino acid sequence of SEQ ID NO: 43. In one embodiment, one or more RNA polynucleotide comprises a nucleic acid sequence encoding an antigenic variant or fragment of SCIN. In one embodiment, one or more RNA polynucleotide comprises a nucleic acid sequence encoding an antigenic variant or fragment of SCIN having the sequence of SEQ ID NO: 40. In one embodiment, one or more RNA polynucleotide comprises a nucleic acid sequence encoding an antigenic variant or fragment of SCIN having the sequence of SEQ ID NO: 43. In one embodiment, one or more RNA polynucleotide of the provided composition is not naturally occurring. In one embodiment, one or more RNA polynucleotide of the provided composition comprises a coding sequence that is codon optimized. In a further embodiment, the RNA polynucleotide comprises a nucleic acid sequence that is codon optimized for expression in cells of a mammalian subject (e.g., a human). In one embodiment, one or more RNA polynucleotide comprises the sequence of SEQ ID NO: 42. In one embodiment, one or more RNA polynucleotide comprises the sequence of SEQ ID NO: 44.

In one embodiment, the provided composition comprises one or more RNA polynucleotide comprising a nucleic acid sequence encoding an antigenic fragment of SCIN. In one embodiment, the composition comprises one or more RNA polynucleotide comprising a nucleic acid sequence encoding an antigenic fragment of an SCIN polypeptide having the amino acid sequence of SEQ ID NO:45. In one embodiment, the composition comprises one or more RNA polynucleotide comprising a nucleic acid sequence encoding an antigenic fragment of an SCIN polypeptide having the amino acid sequence of SEQ ID NO:48. In some embodiments, the composition comprises a RNA polynucleotide comprising a nucleic acid sequence encoding an antigenic fragment of SCIN that is 6-115, 6-100, 6-75, 6-50, 6-25, 6-20, 6-15, or 6-10 amino acids in length. In further embodiments, the composition comprises an RNA polynucleotide comprising a nucleic acid sequence encoding an antigenic fragment of an SCIN having the amino acid sequence of SEQ ID NO: 40 that is 6-115, 6-100, 6-75, 6-50, 6-25, 6-20, 6-15, or 6-10 amino acids in length. In one embodiment, the composition comprises a RNA polynucleotide comprising a nucleic acid sequence encoding an antigenic fragment of an SCIN having the amino acid sequence of SEQ ID NO: 43 that is 6-115, 6-100, 6-75, 6-50, 6-25, 6-20, 6-15, or 6-10 amino acids in length.

In one embodiment, the provided composition comprises one or more RNA polynucleotide comprising a nucleic acid sequence encoding an antigenic fragment of SCIN comprising the amino acid sequence of SEQ ID NO: 45. In some embodiments, one or more RNA polynucleotide comprises a nucleic acid sequence encoding a fragment of SCIN that comprises the amino acid sequence of SEQ ID NO: 45 and that is 51-115, 51-100, 51-75, or 51-70, amino acids in length.

In one embodiment, the provided composition comprises one or more RNA polynucleotide comprising a nucleic acid sequence encoding an antigenic fragment of SCIN comprising the amino acid sequence of SEQ ID NO: 48. In some embodiments, one or more RNA polynucleotide comprises a nucleic acid sequence encoding a fragment of SCIN that comprises the amino acid sequence of SEQ ID NO: 48 and that is 1-18, 3-15, 5-10, or 6-9, amino acids in length.

In one embodiment, the provided composition comprises one or more RNA polynucleotide comprising a nucleic acid sequence encoding a SCIN variant. In some embodiments, one or more RNA polynucleotide comprises a nucleic acid encoding an SCIN variant having sequence identity of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%, with the amino acid sequence of SEQ ID NO:40, 43, 45 or 48.

In one embodiment, the provided composition comprises one or more RNA polynucleotide comprising a nucleic acid sequence encoding an SCIN variant having a total of 1400, 1-75, 1-50, 1-24, 1-30, 1-20, 1-15, 1-10, or 1-5, amino acid substitutions, deletions, and/or insertions compared to a reference SCIN sequence of SEQ ID NO:40. In one embodiment, the RNA polynucleotide comprises a nucleic acid sequence encoding an SCIN variant having a total of 1, 2, 3, 4, 5, 6, 7, 8, 9, 10, or fewer than 15, amino acid substitutions, deletions, and/or insertions compared to a reference SCIN sequence of SEQ ID NO:40. In one embodiment, the provided composition comprises one or more RNA polynucleotide comprising a nucleic acid sequence encoding an SCIN variant having a total of 1-100, 1-75, 1-50, 1-24, 1-30, 1-20, 1-15, 1-10, or 1-5, amino acid substitutions, deletions, and/or insertions compared to a reference SCIN sequence of SEQ ID NO:43. In one embodiment, the RNA polynucleotide comprises a nucleic acid sequence encoding an SCIN variant having a total of 1, 2, 3, 4, 5, 6, 7, 8, 9, 10, or fewer than 15, amino acid substitutions, deletions, and/or insertions compared to a reference SCIN sequence of SEQ ID NO:43. In one embodiment, the provided composition comprises one or more RNA polynucleotide comprising a nucleic acid sequence encoding an SCIN variant having a total of 1-100, 1-75, 1-50, 1-24, 1-30, 1-20, 1-15, 1-10, or 1-5, amino acid substitutions, deletions, and/or insertions compared to a reference SCIN sequence of SEQ ID NO:45. In one embodiment, the RNA polynucleotide comprises a nucleic acid sequence encoding an SCIN variant having a total of 1, 2, 3, 4, 5, 6, 7, 8, 9, 10, or fewer than 15, amino acid substitutions, deletions, and/or insertions compared to a reference SCIN sequence of SEQ ID NO:45. In one embodiment, the provided composition comprises one or more RNA polynucleotide comprising a nucleic acid sequence encoding an SCIN variant having a total of 1-100, 1-75, 1-50, 1-24, 1-30, 1-20, 1-15, 1-10, or 1-5, amino acid substitutions, deletions, and/or insertions compared to a reference SCIN sequence of SEQ ID NO:48. In one embodiment, the RNA polynucleotide comprises a nucleic acid sequence encoding an SCIN variant having a total of 1, 2, 3, 4, 5, 6, 7, 8, 9, 10, or fewer than 15, amino acid substitutions, deletions, and/or insertions compared to a reference CHIPs sequence of SEQ ID NO:48. In some embodiments, one or more of the substitutions in the SCIN variant encoded by the RNA polynucleotide is a conservative substitution. In some embodiments, one or more of the deletions in the SCIN variant encoded by the RNA polynucleotide is at the amino terminus and/or the carboxy terminus of the reference SCIN sequence.

In one embodiment, the composition comprises one or more RNA polynucleotide comprising a nucleic acid sequence encoding N-acetylmuramyl-L-alanine amidase (Amd), or a variant or fragment thereof. In one embodiment, the sequence encodes the amino acid sequence of SEQ ID NO: 4, or a variant or fragment thereof. In one embodiment, the one or more RNA polynucleotide comprises the nucleic acid sequence of SEQ ID NO: 6.

In one embodiment, the composition comprises one or more RNA polynucleotide comprising a nucleic acid sequence encoding endo-β-N-acetylglucosaminidase (Gmd), or a variant or fragment thereof. In one embodiment, the sequence encodes the amino acid sequence of SEQ ID NO: 19, or a variant or fragment thereof. In one embodiment, the one or more RNA polynucleotide comprises the nucleic acid sequence of SEQ ID NO:21.

In one embodiment, the composition comprises one or more RNA polynucleotide comprising a nucleic acid sequence encoding autolysin (Atl), or a variant or fragment thereof. In one embodiment, the sequence encodes the amino acid sequence of SEQ ID NO: 1, or a variant or fragment thereof. In one embodiment, the one or more RNA polynucleotide comprises the nucleic acid sequence of SEQ ID NO: 3.

In one embodiment, the composition comprises one or more RNA polynucleotides comprising nucleic acid sequences encoding Amd and Gmd, or variants or fragments thereof. In one embodiment, the sequences encode the amino acid sequences of SEQ ID NO: 4 and SEQ ID NO: 19, or variants or fragments thereof. In one embodiment, the nucleic acid sequence encoding Amd comprises the nucleic acid sequence of SEQ ID NO: 6 and the nucleic acid sequence encoding Gmd comprises the nucleic acid sequence of SEQ ID NO:21.

In some embodiments, the composition comprises one or more RNA polynucleotide comprising a nucleic acid sequence encoding one or more secreted immunotoxin of S. aureus. In some embodiments, said one or more secreted immunotoxin of S. aureus is selected from the group consisting of: alpha-hemolysin (Hla), chemotaxis inhibiting protein of S. aureus (CHIPS) and the Staphylococcal complement inhibitor (SCIN).

In some embodiments, the composition comprises one or more RNA polynucleotide comprising a nucleic acid sequence encoding one or more lytic transglycosylase of S. aureus. In some embodiments, said one or more lytic transglycosylase is IsaA or SceD.

In some embodiments, the composition comprises one or more RNA polynucleotide comprising a nucleic acid sequence encoding one or more secreted immunotoxin of S. aureus. In some embodiments, said one or more secreted immunotoxin of S. aureus is selected from the group consisting of: alpha-hemolysin (Hla), chemotaxis inhibiting protein of S. aureus (CHIPS) and the Staphylococcal complement inhibitor (SCIN).

In one embodiment, the composition comprises one or more RNA polynucleotide comprising a nucleic acid sequence encoding Hla immunotoxin, or a variant or fragment thereof. In one embodiment, the sequence encodes the amino acid sequence of SEQ ID NO:51, or a variant or fragment thereof. In one embodiment, the one or more RNA polynucleotide comprises the nucleic acid sequence of SEQ ID NO: 53.

In one embodiment, the composition comprises one or more RNA polynucleotide comprising a nucleic acid sequence encoding CHIPS immunotoxin, or a variant or fragment thereof. In one embodiment, the sequence encodes the amino acid sequence of SEQ ID NO: 31, or a variant or fragment thereof. In one embodiment, the one or more RNA polynucleotide comprises the nucleic acid sequence of SEQ ID NO: 33.

In one embodiment, the composition comprises one or more RNA polynucleotide comprising a nucleic acid sequence encoding SCIN immunotoxin, or a variant or fragment thereof. In one embodiment, the sequence encodes the amino acid sequence of SEQ ID NO: 40, or a variant or fragment thereof. In one embodiment, the one or more RNA polynucleotide comprises the nucleic acid sequence of SEQ ID NO: 42.

In some embodiments of the present disclosure, one or more nucleic acid molecules encoding one or more S. aureus antigenic polypeptide or immunogenic variant or fragment thereof comprises a nucleic acid sequence that is substantially homologous to the nucleic acid sequences described herein, such that the expressed antigenic polypeptides or fragments thereof retain the immunogenic function of the original or reference amino acid sequences. For example, in some embodiments, the nucleic acid sequence encoding the S. aureus antigenic polypeptides or immunogenic fragments thereof has a degree of identity with respect to the original or reference nucleic acid sequences of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In some embodiments of the present disclosure, one or more nucleic acid molecules encoding one or more S. aureus antigenic polypeptide or immunogenic variant or fragment thereof comprises a nucleic acid sequence that is a portion of the nucleic acid sequence encoding an S. aureus antigenic protein described, such that the expressed antigenic polypeptides or immunogenic variants thereof retain the immunogenic function of the original or reference amino acid sequences. For example, in some embodiments, the nucleic acid sequence encoding the S. aureus antigen or immunogenic variant thereof has a length with respect to the original or reference amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In some embodiments, one or more nucleic acid molecules encoding one or more S. aureus antigenic polypeptide or immunogenic variant or fragment thereof comprises a nucleic acid sequence that is substantially homologous to the nucleic acid sequence encoding the S. aureus antigen or immunogenic fragment thereof, is a portion of the nucleic acid sequence encoding the S. aureus antigen or immunogenic variant thereof, and the expressed antigenic polypeptides, variants or fragments thereof retain the immunogenic function of the original or reference amino acid sequences. For example, in some embodiments, the nucleic acid sequence encoding one or more S. aureus antigen, immunogenic variant, or immunogenic fragment thereof has a degree of identity with respect to the original or reference nucleic acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% and/or a length with respect to the original or reference nucleic acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

S. aureus vaccines of the present disclosure comprising at least one RNA polynucleotide, such as a mRNA may be transcribed in vitro from template DNA, referred to as an “in vitro transcription template.” The source of the DNA can be, for example, genomic DNA, plasmid DNA, phage DNA, cDNA, synthetic DNA sequence or any other appropriate source of DNA. In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability of mRNA in the cell. On a circular DNA template, for instance, plasmid DNA, RNA polymerase produces a long concatameric product, which is not suitable for expression in eukaryotic cells. The transcription of plasmid DNA linearized at the end of the 3′ UTR results in normal sized mRNA, which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36, 1985; Nacheva and Berzal-Herranz, Eur. J. Biochem., 270:1485-65 (2003)).

The conventional method of integration of polyA/T stretches into a DNA template is molecular cloning. However, polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (E-PAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA. 5′ caps also provide stability to mRNA molecules. In one embodiment, RNAs produced by the methods include a 5′ cap1 structure. Such cap1 structure can be generated using Vaccinia capping enzyme and 2′-O-methyltransferase enzymes (CellScript, Madison, WI). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

### Chemical Modification

Polynucleotides (e.g., DNA or RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphdioester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

Modifications of polynucleotides (e.g., RNA polynucleotides, such as mRNA polynucleotides) that are useful in the vaccines of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-0 Me-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyo sine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}] propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluro-cytidine; 2′ methyl, 2′amino, 2′azido, 2′fluro-adenine; 2′methyl, 2′amino, 2′azido, 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, polynucleotides (e.g., RNA polynucleotides, such as mRNA polynucleotides) include a combination of at least two (e.g., 2, 3, 4 or more) of the aforementioned modified nucleobases.

Antigenic Polypeptides

In some embodiments, the nucleic acid molecules of the present disclosure encode and express one or more antigenic polypeptide or immunogenic variant or fragment thereof. In some embodiments, said one or more antigenic polypeptide or immunogenic variant or fragment thereof derives from one or more S. aureus antigenic proteins. In some embodiments, said one or more S. aureus antigenic polypeptide, immunogenic fragment thereof, or antigenic variant thereof comprises a consensus sequence derived from more than one S. aureus genome. In some embodiments, said one or more S. aureus antigenic polypeptide or immunogenic variant or fragment thereof further comprises a signal peptide. In some embodiments, said signal peptide is cleaved during cellular processing and export, resulting in an extracellular mature antigenic polypeptide or immunogenic variant or fragment thereof absent a signal peptide.

In some embodiments, the one or more S. aureus antigenic polypeptide has an amino acid sequence disclosed in Table 1, or an antigenic fragment or variant thereof. In some embodiments, the one or more S. aureus antigenic polypeptide comprises a fragment of a polypeptide having an amino acid sequence disclosed in Table 1. In a further embodiment, the one or more S. aureus antigenic polypeptide comprises a fragment of a polypeptide having an amino acid sequence disclosed in Table 1 wherein the fragment is 6-1267, 6-1200, 6-1000, 6-750, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In some embodiments, the one or more S. aureus antigenic polypeptide fragment is at least 10, 25, 50, 75, 100 or 125 amino acids in length. In some embodiments, the one or more S. aureus antigenic polypeptide comprises an amino acid sequence having a sequence identity with a reference amino acid sequence disclosed in Table 1 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more S. aureus antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to a reference sequence in Table 1. In one embodiment, the one or more S. aureus antigenic polypeptide has a total of 1, 2, 3, 4, 5, 6, 7, 8, 9, 10, or fewer than 15, amino acid substitutions, deletions, and/or insertions compared to a reference sequence in Table 1. In one embodiment, the one or more S. aureus antigenic polypeptide has a total of 1, 2, 3, 4, 5, 6, 7, 8, 9, 10, or fewer than 15, amino acid substitutions compared to a reference sequence in Table 1. In a further embodiment, the one or more S. aureus antigenic polypeptide has a total of 1, 2, 3, 4, 5, 6, 7, 8, 9, 10, or fewer than 15, conservative amino acid substitutions compared to a reference sequence in Table 1.

In some embodiments, the one or more S. aureus Atl, Amd, and/or Gmd antigenic polypeptide encoded by the nucleic acid has an amino acid sequence disclosed in Table 1, or an antigenic fragment or variant thereof. In some embodiments, the one or more S. aureus antigenic Atl, Amd, and/or Gmd polypeptide comprises a fragment of a polypeptide having an amino acid sequence disclosed in Table 1. In a further embodiment, the one or more S. aureus antigenic polypeptide comprises a Atl, Amd, and/or Gmd fragment of a polypeptide having an amino acid sequence disclosed in Table 1 wherein the Atl, Amd, and/or Gmd fragment is 6-1267, 6-1200, 6-1000, 6-750, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In some embodiments, the one or more S. aureus antigenic Atl, Amd, and/or Gmd polypeptide fragment is at least 10, 25, 50, 75, 100 or 125 amino acids in length. In some embodiments, the one or more S. aureus Atl, Amd, and/or Gmd antigenic polypeptide comprises an Atl, Amd, and/or Gmd amino acid sequence having a sequence identity with a reference Atl, Amd, and/or Gmd amino acid sequence disclosed in Table 1 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more S. aureus antigenic polypeptide has an Atl, Amd, and/or Gmd amino acid sequence that contains a total of 1-100, 1-75, 1-50, 1-24, 1-30, 1-20, 1-15, 1-10, or 1-5, amino acid substitutions, deletions, and/or insertions compared to a corresponding reference sequence in Table 1. In one embodiment, the one or more S. aureus antigenic Atl, Amd, and/or Gmd polypeptide has a total of 1, 2, 3, 4, 5, 6, 7, 8, 9, 10, or fewer than 15, amino acid substitutions, deletions, and/or insertions compared to a corresponding reference Atl, Amd, and/or Gmd sequence in Table 1. In one embodiment, the one or more S. aureus antigenic Atl, Amd, and/or Gmd polypeptide has a total of 1, 2, 3, 4, 5, 6, 7, 8, 9, 10, or fewer than 15, amino acid substitutions compared to a corresponding reference sequence in Table 1. In a further embodiment, the one or more S. aureus antigenic polypeptide has a total of 1, 2, 3, 4, 5, 6, 7, 8, 9, 10, or fewer than 15, conservative amino acid substitutions compared to a corresponding reference sequence in Table 1.

In some embodiments, the one or more S. aureus Atl, Amd, and/or Gmd antigenic polypeptide encoded by the nucleic acid has an amino acid sequence corresponding to an accession number disclosed in Table 3, or an antigenic fragment or variant thereof. In some embodiments, the one or more S. aureus antigenic Atl, Amd, and/or Gmd polypeptide comprises a fragment of a polypeptide having an amino acid sequence corresponding to an accession number disclosed in Table 3 (e.g., amino acids 198-772 or amino acid 773 to the carboxyl terminus of the sequence associated with the accession number). In a further embodiment, the one or more S. aureus antigenic polypeptide comprises a Atl, Amd, and/or Gmd fragment of a polypeptide having an amino acid sequence corresponding to an accession number disclosed in Table 3 wherein the Atl, Amd, and/or Gmd fragment is 6-1267, 6-1200, 6-1000, 6-750, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In some embodiments, the one or more S. aureus antigenic Atl, Amd, and/or Gmd polypeptide fragment is at least 10, 25, 50, 75, 100 or 125 amino acids in length. In some embodiments, the one or more S. aureus Atl, Amd, and/or Gmd antigenic polypeptide comprises an Atl, Amd, and/or Gmd amino acid sequence having a sequence identity with a reference Atl, Amd, and/or Gmd amino acid sequence corresponding to an accession number disclosed in Table 3 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide comprises an amino acid sequence having a sequence identity with a reference amino acid sequence corresponding to amino acids 198-772 or amino acid 773 to the carboxyl terminus of the sequence associated with an accession number disclosed in Table 3 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more S. aureus antigenic polypeptide has an Atl, Amd, and/or Gmd amino acid sequence that contains a total of 1-100, 1-75, 1-50, 1-24, 1-30, 1-20, 1-15, 1-10, or 1-5, amino acid substitutions, deletions, and/or insertions compared to a reference amino acid sequence (e.g., amino acids 198-772 or amino acid 773 to the carboxyl terminus) associated with an accession number disclosed in Table 3. In one embodiment, the one or more S. aureus antigenic Atl, Amd, and/or Gmd polypeptide has a total of 1, 2, 3, 4, 5, 6, 7, 8, 9, 10, or fewer than 15, amino acid substitutions, deletions, and/or insertions compared to a reference amino acid sequence (e.g., amino acids 198-772 or amino acid 773 to the carboxyl terminus) associated with an accession number disclosed in Table 3. In one embodiment, the one or more S. aureus antigenic Atl, Amd, and/or Gmd polypeptide has a total of 1, 2, 3, 4, 5, 6, 7, 8, 9, 10, or fewer than 15, amino acid substitutions compared to a reference amino acid sequence (e.g., amino acids 198-772 or amino acid 773 to the carboxyl terminus) associated with an accession number disclosed in Table 3. In a further embodiment, the one or more S. aureus antigenic polypeptide has a total of 1, 2, 3, 4, 5, 6, 7, 8, 9, 10, or fewer than 15, conservative amino acid substitutions compared to a reference amino acid sequence (e.g., amino acids 198-772 or amino acid 773 to the carboxyl terminus) associated with an accession number disclosed in Table 3.

In some embodiments, said one or more S. aureus antigenic proteins is selected from the group consisting of: Amd, Gmd and Atl. In one embodiment, said Amd antigenic protein comprises the amino acid sequence of SEQ ID NO: 4. In one embodiment, said Gmd antigenic protein comprises the amino acid sequence of SEQ ID NO: 19. In one embodiment, said Atl antigenic protein comprises the amino acid sequence of SEQ ID NO: 1. In one embodiment, said Atl antigenic protein comprises the amino acid sequence of SEQ ID NO: 72.

In some embodiments, the one or more S. aureus antigenic polypeptide is an Atl polypeptide comprising the amino acid sequence of SEQ ID NO: 1. In some embodiments, the one or more S. aureus antigenic polypeptide comprises a fragment of an Atl polypeptide having the amino acid sequence of SEQ ID NO: 1. In a further embodiment, the one or more S. aureus antigenic polypeptide comprises a fragment of a polypeptide having an amino acid sequence of SEQ ID NO:1 wherein the fragment is 6-1267, 6-1200, 6-1000, 6-750, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In some embodiments, the one or more S. aureus antigenic polypeptide fragment comprises the amino acid sequence of SEQ ID NO:13. In some embodiments, the one or more S. aureus antigenic polypeptide fragment comprises the amino acid sequence of SEQ ID NO:13 and a fragment of the sequence of SEQ ID NO:1 that is 365-1180, 365-1000, 365-750, 365-700, 365-600, 365-590, 365-500, 365-450, or 365-400, amino acids in length.

In some embodiments, the one or more S. aureus antigenic polypeptide fragment is at least 10, 25, 50, 75, 100 or 125 amino acids in length. In some embodiments, the antigenic polypeptide comprises an amino acid sequence having a sequence identity with the sequence of SEQ ID NO: 1, 13, or 72 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to the amino acid sequence of SEQ ID NO:1, 13, or 72. In one embodiment, the one or more antigenic polypeptide has a total of 1, 2, 3, 4, 5, 6, 7, 8, 9, 10, or fewer than 15, amino acid substitutions, deletions, and/or insertions compared to the sequence of SEQ ID NO:1. In one embodiment, the one or more antigenic polypeptide has a total of 1, 2, 3, 4, 5, 6, 7, 8, 9, 10, or fewer than 15, amino acid substitutions compared to the sequence of SEQ ID NO:1, 13 or 72. In a further embodiment, the one or more antigenic polypeptide has a total of 1, 2, 3, 4, 5, 6, 7, 8, 9, 10, or fewer than 15, conservative amino acid substitutions compared to the sequence of SEQ ID NO:1, 13, or 72.

In some embodiments, the one or more S. aureus antigenic polypeptide is an Amd polypeptide comprising the amino acid sequence of SEQ ID NO: 4. In some embodiments, the one or more S. aureus antigenic polypeptide comprises a fragment of an Amd polypeptide having the amino acid sequence of SEQ ID NO: 4.

In a further embodiment, the one or more S. aureus antigenic polypeptide comprises a fragment of a Amd polypeptide having an amino acid sequence of SEQ ID NO:4, wherein the fragment is 6-590, 6-575, 6-550, 6-500, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In some embodiments, the one or more S. aureus antigenic polypeptide fragment comprises the Amd R1 domain amino acid sequence of SEQ ID NO:7. In some embodiments, the one or more S. aureus antigenic polypeptide comprises a fragment of SEQ ID NO:7 that is 6-175, 6-150, 6-100, 6-75, 6-50, 6-40, 6-30, 6-25, 6-20, or 6-15, amino acids in length. In some embodiments, the one or more S. aureus antigenic polypeptide fragment comprises the amino acid sequence of SEQ ID NO:7 and a fragment of the sequence of SEQ ID NO:4 that is 176-590, 176-500, 176-250, 176-200, or 176-190, amino acids in length. In some embodiments, the one or more S. aureus antigenic polypeptide fragment comprises the Amd R2 domain amino acid sequence of SEQ ID NO:10 In some embodiments, the one or more S. aureus antigenic polypeptide comprises a fragment of SEQ ID NO:10 that is 6-170, 6-150, 6-100, 6-75, 6-50, 6-40, 6-30, 6-20, or 6-15, amino acids in length. In some embodiments, the one or more S. aureus antigenic polypeptide fragment comprises the amino acid sequence of SEQ ID NO:10 and a fragment of the sequence of SEQ ID NO:4 that is 172-590, 172-500, 172-250, 172-200, or 172-190, amino acids in length. In some embodiments, the one or more S. aureus antigenic polypeptide fragment comprises the Amd catalytic domain amino acid sequence of SEQ ID NO:16. In some embodiments, the one or more S. aureus antigenic polypeptide comprises a fragment of SEQ ID NO:16 that is 6-223, 6-200, 6-175, 6-150, 6-125, 6-100, 6-75, 6-50, 6-25, 6-20, 6-15, or 6-10, amino acids in length. In some embodiments, the one or more S. aureus antigenic polypeptide fragment comprises the amino acid sequence of SEQ ID NO:16 and a fragment of the sequence of SEQ ID NO:4 that is 224-590, 224-500, 224-250, 224-240, or 224-230, amino acids in length.

In some embodiments, the antigenic polypeptide comprises an amino acid sequence having a sequence identity with the sequence of SEQ ID NO:4, 7, 10, or 16 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to the amino acid sequence of SEQ ID NO: 4, 7, 10, or 16. In one embodiment, the one or more antigenic polypeptide has a total of 1, 2, 3, 4, 5, 6, 7, 8, 9, 10, or fewer than 15, amino acid substitutions, deletions, and/or insertions compared to the sequence of SEQ ID NO: 4, 7, 10, or 16. In one embodiment, the one or more antigenic polypeptide has a total of 1, 2, 3, 4, 5, 6, 7, 8, 9, 10, or fewer than 15, amino acid substitutions compared to the sequence of SEQ ID NO: 4, 7, 10, or 16. In a further embodiment, the one or more antigenic polypeptide has a total of 1, 2, 3, 4, 5, 6, 7, 8, 9, 10, or fewer than 15, conservative amino acid substitutions compared to the sequence of SEQ ID NO: 4, 7, 10, or 16.

In some embodiments, the one or more S. aureus antigenic polypeptide is an Gmd polypeptide comprising the amino acid sequence of SEQ ID NO: 19. In some embodiments, the one or more S. aureus antigenic polypeptide comprises a fragment of an Gmd polypeptide having the amino acid sequence of SEQ ID NO: 19.

In a further embodiment, the one or more S. aureus antigenic polypeptide comprises a fragment of a Gmd polypeptide having an amino acid sequence of SEQ ID NO:19, wherein the fragment is 6-1268, 6-1000, 6-750, 6-500, 6-482, 6-475, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In some embodiments, the one or more S. aureus antigenic polypeptide fragment comprises the Gmd R3 domain amino acid sequence of SEQ ID NO:22. In some embodiments, the one or more S. aureus antigenic polypeptide comprises a fragment of SEQ ID NO:22 that is 6-136, 6-125, 6-100, 6-75, 6-50, 6-25, 6-20, 6-15, or 6-10 amino acids in length. In some embodiments, the one or more S. aureus antigenic polypeptide fragment comprises the amino acid sequence of SEQ ID NO:22 and a fragment of the sequence of SEQ ID NO:19 that is 138-482, 138-450, 138-400, 138-300, 138-200, 138-175, or 138-150, amino acids in length. In some embodiments, the one or more S. aureus antigenic polypeptide fragment comprises the amino acid sequence of SEQ ID NO:28. In some embodiments, the one or more S. aureus antigenic polypeptide fragment comprises the amino acid sequence of SEQ ID NO:28 and a fragment of the sequence of SEQ ID NO:19 that is 32-482, 32-450, 32-400, 32-300, 32-200, 32-175, 32-150, 32-100, 32-70, 32-50, 32-40, or 32-35, amino acids in length. In some embodiments, the one or more S. aureus antigenic polypeptide fragment comprises the Gmd catalytic domain amino acid sequence of SEQ ID NO:25. In some embodiments, the one or more S. aureus antigenic polypeptide comprises a fragment of SEQ ID NO:25 that is 6-336, 6-300, 6-250, 6-200, 6-175, 6-150, 6-125, 6-100, 6-75, 6-50, 6-25, 6-20, 6-15, or 6-10 amino acids in length. In some embodiments, the one or more S. aureus antigenic polypeptide fragment comprises the amino acid sequence of SEQ ID NO:25 and a fragment of the sequence of SEQ ID NO:19 that is 337-482, 337-450, 337-400, 337-350, or 337-340, amino acids in length.

In some embodiments, the antigenic polypeptide comprises an amino acid sequence having a sequence identity with the sequence of SEQ ID NO:19, 22, or 25, of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to the amino acid sequence of SEQ ID NO: 19, 22, or 25. In one embodiment, the one or more antigenic polypeptide has a total of 1, 2, 3, 4, 5, 6, 7, 8, 9, 10, or fewer than 15, amino acid substitutions, deletions, and/or insertions compared to the sequence of SEQ ID NO: 19, 22, or 25. In one embodiment, the one or more antigenic polypeptide has a total of 1, 2, 3, 4, 5, 6, 7, 8, 9, 10, or fewer than 15, amino acid substitutions compared to the sequence of SEQ ID NO: 19, 22, or 25. In a further embodiment, the one or more antigenic polypeptide has a total of 1, 2, 3, 4, 5, 6, 7, 8, 9, 10, or fewer than 15, conservative amino acid substitutions compared to the sequence of SEQ ID NO: 19, 22, or 25.

In some embodiments, said one or more S. aureus antigenic protein comprises one or more secreted immunotoxin of S. aureus. In some embodiments, one or more secreted immunotoxin of S. aureus is selected from the group consisting of: alpha-hemolysin (Hla), chemotaxis inhibiting protein of S. aureus (CHIPS) and the Staphylococcal complement inhibitor (SCIN). In one embodiment, said Hla immunotoxin comprises the amino acid sequence of SEQ ID NO:51. In one embodiment, said CHIPS immunotoxin comprises the amino acid sequence of SEQ ID NO: 31. In one embodiment, said SCIN comprises the amino acid sequence of SEQ ID NO: 40.

In some embodiments, the one or more antigenic HLA polypeptide encoded by the nucleic acid has an amino acid sequence disclosed in Table 1, or an antigenic fragment or variant thereof. In some embodiments, the one or more polypeptide comprises a fragment of a HLA polypeptide having an amino acid sequence disclosed in Table 1. In a further embodiment, the one or more antigenic polypeptide comprises a fragment of a HLA polypeptide having an amino acid sequence disclosed in Table 1, wherein the fragment is 6-320 6-300, 6-294, 6-275, 6-250, 6-200, 6-175, 6-150, 6-125, 6-100, 6-75, 6-50, 6-25, 6-20, 6-15, or 6-10 amino acids in length. In some embodiments, the one or more antigenic HLA polypeptide fragment is at least 10, 25, 50, 75, 100 or 125 amino acids in length. In some embodiments, the one or more antigenic HLA polypeptide comprises an amino acid sequence having a sequence identity with a corresponding reference HLA amino acid sequence disclosed in Table 1 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic HLA polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference HLA sequence in Table 1. In one embodiment, the one or more antigenic HLA polypeptide has a total of 1, 2, 3, 4, 5, 6, 7, 8, 9, 10, or fewer than 15, amino acid substitutions, deletions, and/or insertions compared to a corresponding reference HLA sequence in Table 1. In one embodiment, the one or more antigenic HLA polypeptide has a total of 1, 2, 3, 4, 5, 6, 7, 8, 9, 10, or fewer than 15, amino acid substitutions compared to a corresponding reference HLA sequence in Table 1. In a further embodiment, the one or more antigenic polypeptide has a total of 1, 2, 3, 4, 5, 6, 7, 8, 9, 10, or fewer than 15, conservative amino acid substitutions compared to a corresponding reference HLA sequence in Table 1.

In some embodiments, the one or more antigenic HLA polypeptide encoded by the nucleic acid has an amino acid sequence associated with an accession number disclosed in Table 4, or an antigenic fragment or variant thereof. In some embodiments, the one or more antigenic polypeptide comprises a fragment of a HLA polypeptide having an amino acid sequence associated with an accession number disclosed in Table 4 (e.g., amino acid 30 to the carboxyl terminus of the sequence associated with the accession number). In a further embodiment, the one or more antigenic polypeptide comprises a fragment of a HLA polypeptide having an amino acid sequence associated with an accession number disclosed in Table 4, wherein the fragment is 6-320 6-300, 6-294, 6-275, 6-250, 6-200, 6-175, 6-150, 6-125, 6-100, 6-75, 6-50, 6-25, 6-20, 6-15, or 6-10 amino acids in length. In some embodiments, the one or more antigenic HLA polypeptide fragment is at least 10, 25, 50, 75, 100 or 125 amino acids in length. In some embodiments, the one or more antigenic HLA polypeptide comprises an HLA amino acid sequence having a sequence identity with a corresponding reference HLA amino acid sequence associated with an accession number disclosed in Table 4 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide comprises an amino acid sequence having a sequence identity with a reference amino acid sequence corresponding to amino acid 30 to the carboxyl terminus of the sequence associated with an accession number disclosed in Table 4 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference HLA sequence (e.g., amino acid 30 to the carboxyl terminus) associated with an accession number disclosed in Table 4. In one embodiment, the one or more antigenic HLA polypeptide has a total of 1, 2, 3, 4, 5, 6, 7, 8, 9, 10, or fewer than 15, amino acid substitutions, deletions, and/or insertions compared to a corresponding reference HLA sequence (e.g., amino acid 30 to the carboxyl terminus) associated with an accession number disclosed in Table 4. In one embodiment, the one or more antigenic HLA polypeptide has a total of 1, 2, 3, 4, 5, 6, 7, 8, 9, 10, or fewer than 15, amino acid substitutions compared to a corresponding reference HLA sequence (e.g., amino acid 30 to the carboxyl terminus) associated with an accession number disclosed in Table 4. In a further embodiment, the one or more antigenic polypeptide has a total of 1, 2, 3, 4, 5, 6, 7, 8, 9, 10, or fewer than 15, conservative amino acid substitutions compared to a corresponding reference HLA sequence (e.g., amino acid 30 to the carboxyl terminus) associated with an accession number disclosed in Table 4.

In some embodiments, the one or more S. aureus antigenic polypeptide is an alpha-hemolysin (Hla) polypeptide comprising the amino acid sequence of SEQ ID NO:51. In some embodiments, the one or more S. aureus antigenic polypeptide comprises the amino acid sequence of SEQ ID NO:54. In some embodiments, the one or more S. aureus antigenic polypeptide comprises a fragment of an Hla polypeptide having the amino acid sequence of SEQ ID NO: 51 or 54.

In one embodiment, the one or more S. aureus antigenic polypeptide comprises a fragment of a Hla polypeptide having an amino acid sequence of SEQ ID NO:51, wherein the fragment is 6-320 6-300, 6-294, 6-275, 6-250, 6-200, 6-175, 6-150, 6-125, 6-100, 6-75, 6-50, 6-25, 6-20, 6-15, or 6-10 amino acids in length. In one embodiment, the one or more S. aureus antigenic polypeptide comprises a fragment of a Hla polypeptide having an amino acid sequence of SEQ ID NO:54, wherein the fragment 6-482, 6-475, 6-450, 6-400, 6-350, 6-300, 6-250, 6-200, 6-175, 6-150, 6-125, 6-100, 6-75, 6-50, 6-25, 6-20, 6-15, or 6-10 amino acids in length. In some embodiments, the one or more S. aureus antigenic polypeptide fragment comprises the amino acid sequence of SEQ ID NO:57. In some embodiments, the one or more S. aureus antigenic polypeptide fragment comprises the amino acid sequence of SEQ ID NO:57 and a fragment of the sequence of SEQ ID NO:51 that is 66-294, 66-200, 66-150, 66-100, 66-75, or 66-70, amino acids in length.

In some embodiments, the antigenic polypeptide comprises an amino acid sequence having a sequence identity with the sequence of SEQ ID NO:51, 54, or 57, of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to the amino acid sequence of SEQ ID NO: 51, 54, or 57. In one embodiment, the one or more antigenic polypeptide has a total of 1, 2, 3, 4, 5, 6, 7, 8, 9, 10, or fewer than 15, amino acid substitutions, deletions, and/or insertions compared to the sequence of SEQ ID NO:51, 54, or 57. In one embodiment, the one or more antigenic polypeptide has a total of 1, 2, 3, 4, 5, 6, 7, 8, 9, 10, or fewer than 15, amino acid substitutions compared to the sequence of SEQ ID NO:51, 54, or 57. In a further embodiment, the one or more antigenic polypeptide has a total of 1, 2, 3, 4, 5, 6, 7, 8, 9, 10, or fewer than 15, conservative amino acid substitutions compared to the sequence of SEQ ID NO: 51, 54, or 57.

In some embodiments, the one or more antigenic CHIPS polypeptide encoded by the nucleic acid has an amino acid sequence disclosed in Table 1, or an antigenic fragment or variant thereof. In some embodiments, the one or more polypeptide comprises a fragment of a CHIPS polypeptide having an amino acid sequence disclosed in Table 1. In a further embodiment, the one or more antigenic polypeptide comprises a fragment of a CHIPS polypeptide having an amino acid sequence disclosed in Table 1, wherein the fragment is 6-148, 6-140, 6-125, 6-100, 6-75, 6-50, 6-25, 6-20, 6-15, or 6-10 amino acids in length. In some embodiments, the one or more antigenic CHIPS polypeptide fragment is at least 10, 25, 50, 75, 100 or 125 amino acids in length. In some embodiments, the one or more antigenic CHIPS polypeptide comprises an amino acid sequence having a sequence identity with a corresponding reference CHIPS amino acid sequence disclosed in Table 1 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic CHIPS polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference CHIPS sequence in Table 1. In one embodiment, the one or more antigenic CHIPS polypeptide has a total of 1, 2, 3, 4, 5, 6, 7, 8, 9, 10, or fewer than 15, amino acid substitutions, deletions, and/or insertions compared to a corresponding reference CHIPS sequence in Table 1. In one embodiment, the one or more antigenic CHIPS polypeptide has a total of 1, 2, 3, 4, 5, 6, 7, 8, 9, 10, or fewer than 15, amino acid substitutions compared to a corresponding reference CHIPS sequence in Table 1. In a further embodiment, the one or more antigenic polypeptide has a total of 1, 2, 3, 4, 5, 6, 7, 8, 9, 10, or fewer than 15, conservative amino acid substitutions compared to a corresponding reference CHIPS sequence in Table 1.

In some embodiments, the one or more antigenic CHIPS polypeptide encoded by the nucleic acid has an amino acid sequence associated with an accession number disclosed in Table 5, or an antigenic fragment or variant thereof. In some embodiments, the one or more antigenic polypeptide comprises a fragment of a CHIPS polypeptide having an amino acid sequence associated with an accession number disclosed in Table 5 (e.g., amino acid 30 to the carboxyl terminus of the sequence associated with the accession number). In a further embodiment, the one or more antigenic polypeptide comprises a fragment of a CHIPS polypeptide having an amino acid sequence associated with an accession number disclosed in Table 5, wherein the fragment is 6-148, 6-140, 6-125, 6-100, 6-75, 6-50, 6-25, 6-20, 6-15, or 6-10 amino acids in length. In some embodiments, the one or more antigenic CHIPS polypeptide fragment is at least 10, 25, 50, 75, 100 or 125 amino acids in length. In some embodiments, the one or more antigenic CHIPS polypeptide comprises an CHIPS amino acid sequence having a sequence identity with a corresponding reference CHIPS amino acid sequence associated with an accession number disclosed in Table 5 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide comprises an amino acid sequence having a sequence identity with a reference amino acid sequence corresponding to amino acid 30 to the carboxyl terminus of the sequence associated with an accession number disclosed in Table 5 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference CHIPS sequence (e.g., amino acid 30 to the carboxyl terminus) associated with an accession number disclosed in Table 5. In one embodiment, the one or more antigenic CHIPS polypeptide has a total of 1, 2, 3, 4, 5, 6, 7, 8, 9, 10, or fewer than 15, amino acid substitutions, deletions, and/or insertions compared to a corresponding reference CHIPS sequence (e.g., amino acid 30 to the carboxyl terminus) associated with an accession number disclosed in Table 5. In one embodiment, the one or more antigenic CHIPS polypeptide has a total of 1, 2, 3, 4, 5, 6, 7, 8, 9, 10, or fewer than 15, amino acid substitutions compared to a corresponding reference CHIPS sequence (e.g., amino acid 30 to the carboxyl terminus) associated with an accession number disclosed in Table 5. In a further embodiment, the one or more antigenic polypeptide has a total of 1, 2, 3, 4, 5, 6, 7, 8, 9, 10, or fewer than 15, conservative amino acid substitutions compared to a corresponding reference CHIPS sequence (e.g., amino acid 30 to the carboxyl terminus) associated with an accession number disclosed in Table 5.

In some embodiments, the one or more S. aureus antigenic polypeptide is an is an chemotaxis inhibiting protein of S. aureus (CHIPs) polypeptide comprising the amino acid sequence of SEQ ID NO: 31. In some embodiments, the one or more S. aureus antigenic polypeptide comprises the amino acid sequence of SEQ ID NO: 34. In some embodiments, the one or more S. aureus antigenic polypeptide comprises a fragment of a CHIPs polypeptide having the amino acid sequence of SEQ ID NO: 31 or 34.

In one embodiment, the one or more S. aureus antigenic polypeptide comprises a fragment of a CHIPs polypeptide having an amino acid sequence of SEQ ID NO: 31, wherein the fragment is 6-148, 6-140, 6-125, 6-100, 6-75, 6-50, 6-25, 6-20, 6-15, or 6-10 amino acids in length. In one embodiment, the one or more S. aureus antigenic polypeptide comprises a fragment of a CHIPs polypeptide having an amino acid sequence of SEQ ID NO:34, wherein the fragment is 6-120, 6-100, 6-75, 6-50, 6-25, 6-20, 6-15, or 6-10 amino acids in length. In some embodiments, the one or more S. aureus antigenic polypeptide fragment comprises the amino acid sequence of SEQ ID NO: 37. In some embodiments, the one or more S. aureus antigenic polypeptide fragment comprises the amino acid sequence of SEQ ID NO:37 and a fragment of the sequence of SEQ ID NO:31 that is 62-148, 62-125, 62-100, 62-75, or 62-70, amino acids in length.

In some embodiments, the antigenic polypeptide comprises an amino acid sequence having a sequence identity with the sequence of SEQ ID NO:31, 34, or 37, of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to the amino acid sequence of SEQ ID NO: 31, 34, or 37. In one embodiment, the one or more antigenic polypeptide has a total of 1, 2, 3, 4, 5, 6, 7, 8, 9, 10, or fewer than 15, amino acid substitutions, deletions, and/or insertions compared to the sequence of SEQ ID NO: 31, 34, or 37. In one embodiment, the one or more antigenic polypeptide has a total of 1, 2, 3, 4, 5, 6, 7, 8, 9, 10, or fewer than 15, amino acid substitutions compared to the sequence of SEQ ID NO: 31, 34, or 37. In a further embodiment, the one or more antigenic polypeptide has a total of 1, 2, 3, 4, 5, 6, 7, 8, 9, 10, or fewer than 15, conservative amino acid substitutions compared to the sequence of SEQ ID NO: 31, 34, or 37.

In some embodiments, the one or more antigenic SCIN polypeptide encoded by the nucleic acid has an amino acid sequence disclosed in Table 1, or an antigenic fragment or variant thereof. In some embodiments, the one or more polypeptide comprises a fragment of a SCIN polypeptide having an amino acid sequence disclosed in Table 1. In a further embodiment, the one or more antigenic polypeptide comprises a fragment of a SCIN polypeptide having an amino acid sequence disclosed in Table 1, wherein the fragment is 6-115, 6-100, 6-75, 6-50, 6-25, 6-20, 6-15, or 6-10 amino acids in length. In some embodiments, the one or more antigenic SCIN polypeptide fragment is at least 10, 25, 50, 75, 100 or 125 amino acids in length. In some embodiments, the one or more antigenic SCIN polypeptide comprises an amino acid sequence having a sequence identity with a corresponding reference SCIN amino acid sequence disclosed in Table 1 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic SCIN polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference SCIN sequence in Table 1. In one embodiment, the one or more antigenic SCIN polypeptide has a total of 1, 2, 3, 4, 5, 6, 7, 8, 9, 10, or fewer than 15, amino acid substitutions, deletions, and/or insertions compared to a corresponding reference SCIN sequence in Table 1. In one embodiment, the one or more antigenic SCIN polypeptide has a total of 1, 2, 3, 4, 5, 6, 7, 8, 9, 10, or fewer than 15, amino acid substitutions compared to a corresponding reference SCIN sequence in Table 1. In a further embodiment, the one or more antigenic polypeptide has a total of 1, 2, 3, 4, 5, 6, 7, 8, 9, 10, or fewer than 15, conservative amino acid substitutions compared to a corresponding reference SCIN sequence in Table 1.

In some embodiments, the one or more antigenic SCIN polypeptide encoded by the nucleic acid has an amino acid sequence associated with an accession number disclosed in Table 6, or an antigenic fragment or variant thereof. In some embodiments, the one or more antigenic polypeptide comprises a fragment of a SCIN polypeptide having an amino acid sequence associated with an accession number disclosed in Table 6. In a further embodiment, the one or more antigenic polypeptide comprises a fragment of a SCIN polypeptide having an amino acid sequence associated with an accession number disclosed in Table 6, wherein the fragment is 6-115, 6-100, 6-75, 6-50, 6-25, 6-20, 6-15, or 6-10 amino acids in length. In some embodiments, the one or more antigenic SCIN polypeptide fragment is at least 10, 25, 50, 75, 100 or 125 amino acids in length. In some embodiments, the one or more antigenic SCIN polypeptide comprises an SCIN amino acid sequence having a sequence identity with a corresponding reference SCIN amino acid sequence associated with an accession number disclosed in Table 6 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide comprises an amino acid sequence having a sequence identity with a reference amino acid sequence corresponding to amino acid 32 to the carboxyl terminus of the sequence associated with an accession number disclosed in Table 6 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference SCIN sequence (e.g., amino acid 32 to the carboxyl terminus) associated with an accession number disclosed in Table 6. In one embodiment, the one or more antigenic SCIN polypeptide has a total of 1, 2, 3, 4, 5, 6, 7, 8, 9, 10, or fewer than 15, amino acid substitutions, deletions, and/or insertions compared to a corresponding reference SCIN sequence (e.g., amino acid 32 to the carboxyl terminus) associated with an accession number disclosed in Table 6. In one embodiment, the one or more antigenic SCIN polypeptide has a total of 1, 2, 3, 4, 5, 6, 7, 8, 9, 10, or fewer than 15, amino acid substitutions compared to a corresponding reference SCIN sequence (e.g., amino acid 32 to the carboxyl terminus) associated with an accession number disclosed in Table 6. In a further embodiment, the one or more antigenic polypeptide has a total of 1, 2, 3, 4, 5, 6, 7, 8, 9, 10, or fewer than 15, conservative amino acid substitutions compared to a corresponding reference SCIN sequence (e.g., amino acid 32 to the carboxyl terminus) associated with an accession number disclosed in Table 6.

In some embodiments, the one or more S. aureus antigenic polypeptide is a Staphylococcal complement inhibitor (SCIN) polypeptide comprising the amino acid sequence of SEQ ID NO: 40. In some embodiments, the one or more S. aureus antigenic polypeptide comprises the amino acid sequence of SEQ ID NO: 43. In some embodiments, the one or more S. aureus antigenic polypeptide comprises a fragment of a SCIN polypeptide having the amino acid sequence of SEQ ID NO: 40 or 43.

In one embodiment, the one or more S. aureus antigenic polypeptide comprises a fragment of a SCIN polypeptide having an amino acid sequence of SEQ ID NO:40, wherein the fragment is 6-115, 6-100, 6-75, 6-50, 6-25, 6-20, 6-15, or 6-10 amino acids in length. In one embodiment, the one or more S. aureus antigenic polypeptide comprises a fragment of a SCIN polypeptide having an amino acid sequence of SEQ ID NO:43. In some embodiments, the one or more S. aureus antigenic polypeptide fragment comprises the amino acid sequence of SEQ ID NO:45. In some embodiments, the one or more S. aureus antigenic polypeptide fragment comprises the amino acid sequence of SEQ ID NO:45 and a fragment of the sequence of SEQ ID NO:40 that is 51-115, 51-100, 51-75, or 51-70, amino acids in length. In some embodiments, the one or more S. aureus antigenic polypeptide fragment comprises the amino acid sequence of SEQ ID NO:48. In some embodiments, the one or more S. aureus antigenic polypeptide comprises a fragment of SEQ ID NO:48 that is 6-17, 6-15, or 6-10 amino acids in length. In some embodiments, the one or more S. aureus antigenic polypeptide fragment comprises the amino acid sequence of SEQ ID NO:48 and a fragment of the sequence of SEQ ID NO:40 that is 19-115, 19-100, 19-75, or 19-70, amino acids in length.

In additional embodiments, the one or more S. aureus antigenic polypeptide is a lytic transglycosylase. In some embodiments, the lytic transglycosylase encoded by the nucleic acid has an amino acid sequence associated with a sequence accession number disclosed in Table 7, or an antigenic fragment or variant there. In some embodiments, the lytic transglycosylase encoded by the nucleic acid has an amino acid sequence associated with a sequence disclosed in Table 1, or an antigenic fragment or variant thereof.

In additional embodiments, the one or more S. aureus antigenic polypeptide is an IsaA. In some embodiments, the IsaA encoded by the nucleic acid has an amino acid sequence associated with a sequence accession number disclosed in Table 7, or an antigenic fragment or variant there.

In additional embodiments, the one or more S. aureus antigenic polypeptide is an SceD. In some embodiments, the SceD encoded by the nucleic acid has an amino acid sequence associated with a sequence accession number disclosed in Table 7, or an antigenic fragment or variant there.

In some embodiments, the one or more antigenic lytic transglycosylase polypeptide encoded by the nucleic acid has an amino acid sequence disclosed in Table 7, or an antigenic fragment or variant thereof. In some embodiments, the one or more polypeptide comprises a fragment of a lytic transglycosylase polypeptide having an amino acid sequence disclosed in Table 7. In a further embodiment, the one or more antigenic polypeptide comprises a fragment of a lytic transglycosylase polypeptide having an amino acid sequence disclosed in Table 7, wherein the fragment is 6-115, 6-100, 6-75, 6-50, 6-25, 6-20, 6-15, or 6-10 amino acids in length. In some embodiments, the one or more antigenic lytic transglycosylase polypeptide fragment is at least 10, 25, 50, 75, 100 or 125 amino acids in length. In some embodiments, the one or more antigenic lytic transglycosylase polypeptide comprises an amino acid sequence having a sequence identity with a corresponding reference lytic transglycosylase amino acid sequence disclosed in Table 7 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic lytic transglycosylase polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference lytic transglycosylase sequence in Table 7. In one embodiment, the one or more antigenic lytic transglycosylase polypeptide has a total of 1, 2, 3, 4, 5, 6, 7, 8, 9, 10, or fewer than 15, amino acid substitutions, deletions, and/or insertions compared to a corresponding reference lytic transglycosylase sequence in Table 7. In one embodiment, the one or more antigenic lytic transglycosylase polypeptide has a total of 1, 2, 3, 4, 5, 6, 7, 8, 9, 10, or fewer than 15, amino acid substitutions compared to a corresponding reference lytic transglycosylase sequence in Table 7. In a further embodiment, the one or more antigenic polypeptide has a total of 1, 2, 3, 4, 5, 6, 7, 8, 9, 10, or fewer than 15, conservative amino acid substitutions compared to a corresponding reference lytic transglycosylase sequence in Table 7.

In some embodiments, the one or more antigenic IsaA polypeptide encoded by the nucleic acid has an amino acid sequence disclosed in Table 1 or an IsaA polypeptide sequence associated with an accession number disclosed in Table 7, or an antigenic fragment or variant thereof. In some embodiments, the one or more antigenic IsaA polypeptide encoded by the nucleic acid has an amino acid sequence disclosed in Table 1, or an antigenic fragment or variant thereof. In some embodiments, the one or more polypeptide comprises a fragment of a IsaA polypeptide having an amino acid sequence disclosed in Table 1. In a further embodiment, the one or more antigenic polypeptide comprises a fragment of a IsaA polypeptide having an amino acid sequence disclosed in Table 1, wherein the fragment is 6-115, 6-100, 6-75, 6-50, 6-25, 6-20, 6-15, or 6-10 amino acids in length. In some embodiments, the one or more antigenic IsaA polypeptide fragment is at least 10, 25, 50, 75, 100 or 125 amino acids in length. In some embodiments, the one or more antigenic IsaA polypeptide comprises an amino acid sequence having a sequence identity with a corresponding reference IsaA amino acid sequence disclosed in Table 1 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic IsaA polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference IsaA sequence in Table 1. In one embodiment, the one or more antigenic IsaA polypeptide has a total of 1, 2, 3, 4, 5, 6, 7, 8, 9, 10, or fewer than 15, amino acid substitutions, deletions, and/or insertions compared to a corresponding reference IsaA sequence in Table 1. In one embodiment, the one or more antigenic IsaA polypeptide has a total of 1, 2, 3, 4, 5, 6, 7, 8, 9, 10, or fewer than 15, amino acid substitutions compared to a corresponding reference IsaA sequence in Table 1. In a further embodiment, the one or more antigenic polypeptide has a total of 1, 2, 3, 4, 5, 6, 7, 8, 9, 10, or fewer than 15, conservative amino acid substitutions compared to a corresponding reference IsaA sequence in Table 1.

In some embodiments, the one or more antigenic IsaA polypeptide encoded by the nucleic acid has an amino acid sequence associated with an accession number disclosed in Table 7, or an antigenic fragment or variant thereof. In some embodiments, the one or more antigenic polypeptide comprises a fragment of a IsaA polypeptide having an amino acid sequence associated with an accession number disclosed in Table 7. In a further embodiment, the one or more antigenic polypeptide comprises a fragment of a IsaA polypeptide having an amino acid sequence associated with an accession number disclosed in Table 7, wherein the fragment is 6-115, 6-100, 6-75, 6-50, 6-25, 6-20, 6-15, or 6-10 amino acids in length. In some embodiments, the one or more antigenic IsaA polypeptide fragment is at least 10, 25, 50, 75, 100 or 125 amino acids in length. In some embodiments, the one or more antigenic IsaA polypeptide comprises an IsaA amino acid sequence having a sequence identity with a corresponding reference IsaA amino acid sequence associated with an accession number disclosed in Table 7 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide comprises an amino acid sequence having a sequence identity with a reference amino acid sequence corresponding to amino acid 32 to the carboxyl terminus of the sequence associated with an accession number disclosed in Table 7 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference IsaA sequence (e.g., amino acid 32 to the carboxyl terminus) associated with an accession number disclosed in Table 7. In one embodiment, the one or more antigenic IsaA polypeptide has a total of 1, 2, 3, 4, 5, 6, 7, 8, 9, 10, or fewer than 15, amino acid substitutions, deletions, and/or insertions compared to a corresponding reference IsaA sequence (e.g., amino acid 32 to the carboxyl terminus) associated with an accession number disclosed in Table 7. In one embodiment, the one or more antigenic IsaA polypeptide has a total of 1, 2, 3, 4, 5, 6, 7, 8, 9, 10, or fewer than 15, amino acid substitutions compared to a corresponding reference IsaA sequence (e.g., amino acid 32 to the carboxyl terminus) associated with an accession number disclosed in Table 7. In a further embodiment, the one or more antigenic polypeptide has a total of 1, 2, 3, 4, 5, 6, 7, 8, 9, 10, or fewer than 15, conservative amino acid substitutions compared to a corresponding reference IsaA sequence (e.g., amino acid 32 to the carboxyl terminus) associated with an accession number disclosed in Table 7.

In some embodiments, the one or more S. aureus antigenic polypeptide is an Staphylococcal complement inhibitor (IsaA) polypeptide comprising the amino acid sequence of SEQ ID NO: 91. In some embodiments, the one or more S. aureus antigenic polypeptide comprises the amino acid sequence of SEQ ID NO: 94. In some embodiments, the one or more S. aureus antigenic polypeptide comprises a fragment of a IsaA polypeptide having the amino acid sequence of SEQ ID NO: 91 or 94.

In one embodiment, the one or more S. aureus antigenic polypeptide comprises a fragment of a IsaA polypeptide having an amino acid sequence of SEQ ID NO:91, wherein the fragment is 6-115, 6-100, 6-75, 6-50, 6-25, 6-20, 6-15, or 6-10 amino acids in length. In one embodiment, the one or more S. aureus antigenic polypeptide comprises a fragment of a IsaA polypeptide having an amino acid sequence of SEQ ID NO:94. In some embodiments, the one or more S. aureus antigenic polypeptide fragment comprises the amino acid sequence of SEQ ID NO:97. In some embodiments, the one or more S. aureus antigenic polypeptide fragment comprises the amino acid sequence of SEQ ID NO:97 and a fragment of the sequence of SEQ ID NO:91 that is 51-115, 51-100, 51-75, or 51-70, amino acids in length. In some embodiments, the one or more S. aureus antigenic polypeptide fragment comprises the amino acid sequence of SEQ ID NO:100. In some embodiments, the one or more S. aureus antigenic polypeptide comprises a fragment of SEQ ID NO:100 that is 6-17, 6-15, or 6-10 amino acids in length. In some embodiments, the one or more S. aureus antigenic polypeptide fragment comprises the amino acid sequence of SEQ ID NO:100 and a fragment of the sequence of SEQ ID NO:91 that is 19-115, 19-100, 19-75, or 19-70, amino acids in length. In some embodiments, the one or more S. aureus antigenic polypeptide fragment comprises the amino acid sequence of SEQ ID NO:103. In some embodiments, the one or more S. aureus antigenic polypeptide comprises a fragment of SEQ ID NO:103 that is 6-17, 6-15, or 6-10 amino acids in length. In some embodiments, the one or more S. aureus antigenic polypeptide fragment comprises the amino acid sequence of SEQ ID NO:103 and a fragment of the sequence of SEQ ID NO:91 that is 19-115, 19-100, 19-75, or 19-70, amino acids in length.

In some embodiments, the one or more antigenic SceD polypeptide encoded by the nucleic acid has an amino acid sequence disclosed in Table 1 or an SceD polypeptide sequence associated with an accession number disclosed in Table 7, or an antigenic fragment or variant thereof. In some embodiments, the one or more antigenic SceD polypeptide encoded by the nucleic acid has an amino acid sequence disclosed in Table 1, or an antigenic fragment or variant thereof. In some embodiments, the one or more polypeptide comprises a fragment of a SceD polypeptide having an amino acid sequence disclosed in Table 1. In a further embodiment, the one or more antigenic polypeptide comprises a fragment of a SceD polypeptide having an amino acid sequence disclosed in Table 1, wherein the fragment is 6-115, 6-100, 6-75, 6-50, 6-25, 6-20, 6-15, or 6-10 amino acids in length. In some embodiments, the one or more antigenic SceD polypeptide fragment is at least 10, 25, 50, 75, 100 or 125 amino acids in length. In some embodiments, the one or more antigenic SceD polypeptide comprises an amino acid sequence having a sequence identity with a corresponding reference SceD amino acid sequence disclosed in Table 1 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic SceD polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference SceD sequence in Table 1. In one embodiment, the one or more antigenic SceD polypeptide has a total of 1, 2, 3, 4, 5, 6, 7, 8, 9, 10, or fewer than 15, amino acid substitutions, deletions, and/or insertions compared to a corresponding reference SceD sequence in Table 1. In one embodiment, the one or more antigenic SceD polypeptide has a total of 1, 2, 3, 4, 5, 6, 7, 8, 9, 10, or fewer than 15, amino acid substitutions compared to a corresponding reference SceD sequence in Table 1. In a further embodiment, the one or more antigenic polypeptide has a total of 1, 2, 3, 4, 5, 6, 7, 8, 9, 10, or fewer than 15, conservative amino acid substitutions compared to a corresponding reference SceD sequence in Table 1.

In some embodiments, the one or more antigenic SceD polypeptide encoded by the nucleic acid has an amino acid sequence associated with an accession number disclosed in Table 7, or an antigenic fragment or variant thereof. In some embodiments, the one or more antigenic polypeptide comprises a fragment of a SceD polypeptide having an amino acid sequence associated with an accession number disclosed in Table 7. In a further embodiment, the one or more antigenic polypeptide comprises a fragment of a SceD polypeptide having an amino acid sequence associated with an accession number disclosed in Table 7, wherein the fragment is 6-115, 6-100, 6-75, 6-50, 6-25, 6-20, 6-15, or 6-10 amino acids in length. In some embodiments, the one or more antigenic SceD polypeptide fragment is at least 10, 25, 50, 75, 100 or 125 amino acids in length. In some embodiments, the one or more antigenic SceD polypeptide comprises an SceD amino acid sequence having a sequence identity with a corresponding reference SceD amino acid sequence associated with an accession number disclosed in Table 7 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide comprises an amino acid sequence having a sequence identity with a reference amino acid sequence corresponding to amino acid 32 to the carboxyl terminus of the sequence associated with an accession number disclosed in Table 7 of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to a corresponding reference SceD sequence (e.g., amino acid 32 to the carboxyl terminus) associated with an accession number disclosed in Table 7. In one embodiment, the one or more antigenic SceD polypeptide has a total of 1, 2, 3, 4, 5, 6, 7, 8, 9, 10, or fewer than 15, amino acid substitutions, deletions, and/or insertions compared to a corresponding reference SceD sequence (e.g., amino acid 32 to the carboxyl terminus) associated with an accession number disclosed in Table 7. In one embodiment, the one or more antigenic SceD polypeptide has a total of 1, 2, 3, 4, 5, 6, 7, 8, 9, 10, or fewer than 15, amino acid substitutions compared to a corresponding reference SceD sequence (e.g., amino acid 32 to the carboxyl terminus) associated with an accession number disclosed in Table 7. In a further embodiment, the one or more antigenic polypeptide has a total of 1, 2, 3, 4, 5, 6, 7, 8, 9, 10, or fewer than 15, conservative amino acid substitutions compared to a corresponding reference SceD sequence (e.g., amino acid 32 to the carboxyl terminus) associated with an accession number disclosed in Table 7.

In some embodiments, the one or more S. aureus antigenic polypeptide is an Staphylococcal complement inhibitor (SceD) polypeptide comprising the amino acid sequence of SEQ ID NO: 106. In some embodiments, the one or more S. aureus antigenic polypeptide comprises the amino acid sequence of SEQ ID NO: 109. In some embodiments, the one or more S. aureus antigenic polypeptide comprises a fragment of a SceD polypeptide having the amino acid sequence of SEQ ID NO: 106 or 109.

In one embodiment, the one or more S. aureus antigenic polypeptide comprises a fragment of a SceD polypeptide having an amino acid sequence of SEQ ID NO:106, wherein the fragment is 6-115, 6-100, 6-75, 6-50, 6-25, 6-20, 6-15, or 6-10 amino acids in length. In one embodiment, the one or more S. aureus antigenic polypeptide comprises a fragment of a SceD polypeptide having an amino acid sequence of SEQ ID NO:109. In some embodiments, the one or more S. aureus antigenic polypeptide fragment comprises the amino acid sequence of SEQ ID NO:112. In some embodiments, the one or more S. aureus antigenic polypeptide fragment comprises the amino acid sequence of SEQ ID NO:112 and a fragment of the sequence of SEQ ID NO:106 that is 51-115, 51-100, 51-75, or 51-70, amino acids in length. In some embodiments, the one or more S. aureus antigenic polypeptide fragment comprises the amino acid sequence of SEQ ID NO:112.

In some embodiments, the one or more S. aureus antigenic polypeptide comprises a fragment of SEQ ID NO: 112 that is 6-17, 6-15, or 6-10 amino acids in length. In some embodiments, the one or more S. aureus antigenic polypeptide fragment comprises the amino acid sequence of SEQ ID NO:112 and a fragment of the sequence of SEQ ID NO:106 that is 19-115, 19-100, 19-75, or 19-70, amino acids in length.

In some embodiments, the one or more S. aureus antigenic polypeptide is CgoX protein of S. aureus comprising the amino acid sequence of SEQ ID NO: 60. In some embodiments, the one or more S. aureus antigenic polypeptide comprises a fragment of a CgoX polypeptide having the amino acid sequence of SEQ ID NO: 60.

In one embodiment, the one or more S. aureus antigenic polypeptide comprises a fragment of a CgoX polypeptide having an amino acid sequence of SEQ ID NO: 60, wherein the fragment is 6-465 6-450, 6-400, 6-350 6-300, 6-250, 6-200, 6-175, 6-150, 6-125, 6-100, 6-75, 6-50, 6-25, 6-20, 6-15, or 6-10 amino acids in length. In some embodiments, the one or more S. aureus antigenic polypeptide fragment comprises the amino acid sequence of SEQ ID NO: 63. In some embodiments, the one or more S. aureus antigenic polypeptide fragment comprises the amino acid sequence of SEQ ID NO:63 and a fragment of the sequence of SEQ ID NO:60 that is 13-465, 13-450, 13-400, 13-350, 13-300, 13-250, 13-200, 13-150, 13-100, 13-75, 13-50, 13-25, 13-20, or 13-15, amino acids in length. In some embodiments, the one or more S. aureus antigenic polypeptide fragment comprises the amino acid sequence of SEQ ID NO: 66 or 69. In some embodiments, the one or more S. aureus antigenic polypeptide fragment comprises the amino acid sequence of SEQ ID NO:66 or 69 and a fragment of the sequence of SEQ ID NO:60 that is 13-465, 13-450, 13-400, 13-350, 13-300, 13-250, 13-200, 13-150, 13-100, 13-75, 13-50, 13-25, 13-20, or 13-15, amino acids in length.

In some embodiments, the antigenic polypeptide comprises an amino acid sequence having a sequence identity with the sequence of SEQ ID NO: 60 or 63, of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. In some embodiments, the one or more antigenic polypeptide has an amino acid sequence that contains a total of 1-100, 1-75, 1-50, 1-24, 1-30, 1-20, 1-15, 1-10, or 1-5, amino acid substitutions, deletions, and/or insertions compared to the amino acid sequence of SEQ ID NO: 60. In one embodiment, the one or more antigenic polypeptide has a total of 1, 2, 3, 4, 5, 6, 7, 8, 9, 10, or fewer than 15, amino acid substitutions, deletions, and/or insertions compared to the sequence of SEQ ID NO: 60.

In some embodiments, the S. aureus antigenic polypeptide or immunogenic variant or fragment thereof comprises an amino acid sequence that is substantially homologous to the amino acid sequence of an S. aureus antigenic protein described herein and retains the immunogenic function of the original or reference amino acid sequence. For example, in some embodiments, the amino acid sequence of the S. aureus antigen or immunogenic variant thereof has a degree of identity with respect to the original or reference amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In some embodiments, the S. aureus antigenic polypeptide or immunogenic variant or fragment thereof comprises an amino acid sequence that is a portion of the amino acid sequence of an S. aureus antigenic protein described herein and retains the immunogenic function of the original or reference amino acid sequence. For example, in some embodiments, the amino acid sequence of the S. aureus antigen or immunogenic fragment thereof has a length with respect to the original or reference amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In some embodiments, the S. aureus antigenic polypeptide or immunogenic variant or fragment thereof comprises an amino acid sequence that is substantially homologous to the amino acid sequence of an S. aureus antigenic protein described herein, is a portion of the amino acid sequence of an S. aureus antigenic protein described herein, and retains the immunogenic function of the original or reference amino acid sequence. For example, in some embodiments, the amino acid sequence of the S. aureus antigen, immunogenic variant, or immunogenic fragment thereof has a degree of identity with respect to the original or reference amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5% and/or a length with respect to the original or reference amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

Signal Peptides

In some embodiments, antigenic polypeptides encoded by nucleic acid molecules comprise a signal peptide. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. Signal peptides generally include three regions: an N-terminal region of differing length, which usually comprises positively charged amino acids; a hydrophobic region; and a short carboxy-terminal peptide region. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane. The signal peptide, however, is not responsible for the final destination of the mature protein. Secretory proteins devoid of additional address tags in their sequence are by default secreted to the external environment. During recent years, a more advanced view of signal peptides has evolved, showing that the functions and immunodominance of certain signal peptides are much more versatile than previously anticipated.

S. aureus vaccines of the present disclosure may comprise, for example, nucleic acid molecules encoding an artificial signal peptide, wherein the signal peptide coding sequence is operably linked to and is in frame with the coding sequence of the antigenic polypeptide. Thus, S. aureus vaccines of the present disclosure, in some embodiments, produce an antigenic polypeptide comprising an antigenic polypeptide (e.g. Amd, Gmd, Atl, CHIPs, SCIN, Hla, or CgoX, or a fragment or variant thereof described herein) fused to a signal peptide. In some embodiments, S. aureus vaccines of the present disclosure produce an antigenic polypeptide comprising an antigenic lytic transglycosylase polypeptide (e.g., IsaA or SceD, or a fragment or variant thereof described herein) fused to a signal peptide. In some embodiments, a signal peptide is fused to the N-terminus of the antigenic polypeptide. In some embodiments, a signal peptide is fused to the C-terminus of the antigenic polypeptide.

In some embodiments, the signal peptide fused to the antigenic polypeptide is an artificial signal peptide. For example, in some embodiments, an artificial signal peptide fused to the antigenic polypeptide encoded by the RNA (e.g., mRNA) vaccine is obtained from an immunoglobulin protein, e.g., an IgE signal peptide or an IgG signal peptide. In some embodiments, a signal peptide fused to the antigenic polypeptide encoded by a RNA (e.g., mRNA) vaccine is an Ig heavy chain epsilon-1 signal peptide (IgE HC SP). In some embodiments, a signal peptide fused to the antigenic polypeptide encoded by the (e.g., mRNA) RNA (e.g., mRNA) vaccine is an IgGk chain V-III region HAH signal peptide (IgGk SP). In some embodiments, the signal peptide is selected from: Japanese encephalitis PRM signal sequence, VSVg protein signal sequence and Japanese encephalitis JEV signal sequence.

The examples disclosed herein are not meant to be limiting and any signal peptide that is known in the art to facilitate targeting of a protein to ER for processing and/or targeting of a protein to the cell membrane may be used in accordance with the present disclosure. A signal peptide is typically cleaved from the nascent polypeptide at the cleavage junction during ER processing. Therefore, in some embodiments, the mature antigenic polypeptide produced by a S. aureus RNA (e.g., mRNA) vaccine of the present disclosure does not comprise a signal peptide.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Nanoparticles

In some embodiments, the present disclosure provides a nucleic acid vaccine of any one of the preceding paragraphs (e.g., an S. aureus Amd vaccine, an S. aureus Gmd vaccine, an S. aureus Atl vaccine, S. aureus CHIPs vaccine, an S. aureus SCIN vaccine, an S. aureus Hla vaccine, an S. aureus CgoX vaccine, an S. aureus IsaA vaccine, an S. aureus SceD vaccine or any combination of two or more of the foregoing vaccines), formulated in a nanoparticle (e.g., a lipid nanoparticle).

In some embodiments, S. aureus nucleic acid vaccines are formulated in a lipid nanoparticle. In some embodiments, S. aureus nucleic acid vaccines are formulated in a lipid-polycation complex, referred to as a cationic lipid nanoparticle. As a non-limiting example, the polycation may include a cationic peptide or a polypeptide such as, but not limited to, polylysine, polyornithine and/or polyarginine. In some embodiments, S. aureus nucleic acid vaccines are formulated in a lipid nanoparticle that includes a non-cationic lipid such as, but not limited to, cholesterol or dioleoyl phosphatidyl-ethanolamine (DOPE). In some embodiments, the lipid nanoparticle comprises at least one ionizable cationic lipid, at least one non-cationic lipid, at least one sterol, and/or at least one polyethylene glycol (PEG)-modified lipid.

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200).

In some embodiments, lipid nanoparticle formulations may comprise 35 to 45% cationic lipid, 40% to 50% cationic lipid, 50% to 60% cationic lipid and/or 55% to 65% cationic lipid. In some embodiments, the ratio of lipid to RNA (e.g., mRNA) in lipid nanoparticles may be 5:1 to 20:1, 10:1 to 25:1, 15:1 to 30:1 and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, an S. aureus nucleic acid vaccine formulation is a nanoparticle that comprises at least one lipid selected from, but not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-lol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, a lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% cationic lipid:5-25% neutral lipid: 25-55% sterol; 0.5-15% PEG-lipid.

In some embodiments, a lipid nanoparticle formulation includes 25% to 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., 35 to 65%, 45 to 65%, 60%, 57.5%, 50% or 40% on a molar basis.

In some embodiments, a lipid nanoparticle formulation includes 0.5% to 15% on a molar basis of the neutral lipid, e.g., 3 to 12%, 5 to 10% or 15%, 10%, or 7.5% on a molar basis. Examples of neutral lipids include, without limitation, DSPC, POPC, DPPC, DOPE and SM. In some embodiments, the formulation includes 5% to 50% on a molar basis of the sterol (e.g., 15 to 45%, 20 to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5 to 10%, 0.5 to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety).

In some embodiments, the molar lipid ratio is 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., Si: 8529-8533; and Maier et al. (2013) Molecular Therapy 21, 1570-1578 (the contents of each of which are incorporated herein by reference in their entirety).

In some embodiments, lipid nanoparticle formulations may comprise a cationic lipid, a PEG lipid and a structural lipid and optionally comprise a non-cationic lipid. As a non-limiting example, a lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, a lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, the lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may comprise a cationic lipid, a non-cationic lipid, a PEG lipid and a structural lipid. As a non-limiting example, the lipid nanoparticle comprise 50% of the cationic lipid DLin-KC2-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DMG and 38.5% of the structural lipid cholesterol. As yet another non-limiting example, the lipid nanoparticle comprise 55% of the cationic lipid L319, 10% of the non-cationic lipid DSPC, 2.5% of the PEG lipid PEG-DMG and 32.5% of the structural lipid cholesterol.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm or 80-200 nm.

Nucleic Acid Vectors

In some embodiments, the polynucleotide encoding one or more S. aureus antigenic polypeptide, variant or immunogenic fragment or one or more consensus S. aureus antigenic polypeptide, variant or immunogenic fragment thereof can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

In one embodiment, the disclosure provides a vector comprising a regulatory element operable in a eukaryotic cell (e.g., a mammalian cell such as a human cell) operably linked to a nucleic acid described herein. In some embodiments, the vector comprises a DNA or DNA plasmid vector.

In one embodiment, the vector comprises an RNA or mRNA vector.

In some embodiments, the vector comprises a regulatory element operable in a eukaryotic cell operably linked to the nucleic acid molecule. The vector can be any vector as herein discussed, including that the vector can comprise a viral vector, such as AAV, VSV, or a chimeric vector (e.g., VSV or another virus expressing the RBD or surface glycoprotein described herein on the surface of the virus).

The nucleotide sequences described herein can be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

In some embodiments it can be preferred that the vectors used for these in vivo applications are attenuated to prevent the vector from amplifying in the subject. For example, if plasmid vectors are used, preferably they will lack an origin of replication that functions in the subject so as to enhance safety for in vivo use in the subject. If viral vectors are used, preferably they are attenuated or replication-defective in the subject, again, so as to enhance safety for in vivo use in the subject.

In some embodiments described herein viral vectors are used. Viral expression vectors are well known to those skilled in the art and include, for example, viruses such as adenoviruses, adeno-associated viruses (AAV), alphaviruses, herpesviruses, retroviruses and poxviruses, including avipox viruses, attenuated poxviruses, vaccinia viruses, and particularly, the modified vaccinia Ankara virus (MVA; ATCC Accession No. VR-1566). Vesicular stomatitis viruses (VSV) are also contemplated, especially if the VSV G protein is substituted with another protein, such as the fusion polypeptides described herein. Such viruses, when used as expression vectors are innately non-pathogenic in the selected subjects such as humans or have been modified to render them non-pathogenic in the selected subjects. For example, replication-defective adenoviruses and alphaviruses are well known and can be used as gene delivery vectors.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components. The one or more vectors may be circular plasmid, which may transform a target cell by integration into the cellular genome or exist extrachromosomally (e.g., autonomous replicating plasmid with an origin of replication).

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered. The plasmid may also comprise a mammalian origin of replication in order to maintain the plasmid extrachromosomally and produce multiple copies of the plasmid in a cell.

The one or more vectors can be a linear nucleic acid, or linear expression cassette (“LEC”), that is capable of being efficiently delivered to a subject via electroporation and expressing one or more S. aureus antigenic polypeptide, variant or immunogenic fragment thereof encoded by the polynucleotide sequence. The LEC may be any linear DNA devoid of any phosphate backbone. The LEC may not contain any antibiotic resistance genes and/or a phosphate backbone. The LEC may not contain other nucleic acid sequences unrelated to the desired gene expression.

In one embodiment, viral vectors are provided herein which are capable of delivering a polynucleotide of the present disclosure to a cell. The expression vector may be provided to a cell in the form of a viral vector. Viral vector technology is well known in the art and is described, for example, in Sambrook et al. (2001), and in Ausubel et al., 1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See. e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In one embodiment, the composition of the present disclosure comprises a DNA viral vector. In one embodiment, the DNA viral vector comprises one or more nucleic acid comprising a sequence encoding N-acetylmuramyl-L-alanine amidase (Amd). In one embodiment, the one or more nucleic acid comprises the sequence of SEQ ID NO:5.

In one embodiment, the DNA viral vector comprises one or more nucleic acid comprising a sequence encoding endo-β-N-acetylglucosaminidase (Gmd). In one embodiment, the one or more nucleic acid comprises the sequence of SEQ ID NO: 20.

In one embodiment, the DNA viral vector comprises one or more nucleic acid comprising a sequence encoding autolysin (Atl). In one embodiment, the one or more nucleic acid comprises the nucleic acid sequence of SEQ ID NO: 2.

In one embodiment, the DNA viral vector comprises one or more nucleic acid comprising sequences encoding Amd and Gmd. In one embodiment, the nucleic acid sequence encoding Amd comprises the sequence of SEQ ID NO:5 and the nucleic acid sequence encoding Gmd comprises the sequence of SEQ ID NO:20.

In one embodiment, the DNA viral vector comprises one or more nucleic acid comprising a sequence encoding one or more secreted immunotoxin of S. aureus. In some embodiments, said one or more secreted immunotoxin of S. aureus is selected from the group consisting of: alpha-hemolysin (Hla), chemotaxis inhibiting protein of S. aureus (CHIPS) and the Staphylococcal complement inhibitor (SCIN). In one embodiment, the nucleic acid sequence encoding Hla comprises the sequence of SEQ ID NO: 55. In one embodiment, the nucleic acid sequence encoding CHIPS comprises the sequence of SEQ ID NO: 32. In one embodiment, the nucleic acid sequence encoding SCIN comprises the sequence of SEQ ID NO: 41.

In one embodiment, the DNA viral vector comprises one or more nucleic acid comprising a sequence encoding one or more S. aureus lytic transglycosylase protein. In some embodiments, the one or more lytic transglycosylase is IsaA. In one embodiment, the nucleic acid sequence encoding IsaA comprises the sequence of SEQ ID NO: 94. In some embodiments, the one or more lytic transglycosylase is SceD. In one embodiment, the nucleic acid sequence encoding SceD comprises the sequence of SEQ ID NO: 109.

In one embodiment, the DNA viral vector comprises one or more nucleic acid comprising a sequence encoding CgoX of S. aureus. In one embodiment, the nucleic acid sequence encoding CgoX comprises the sequence of SEQ ID NO: 61. In one embodiment, the nucleic acid sequence encoding CgoX comprises the sequence of SEQ ID NO: 64.

In some embodiments of the present disclosure, RNA polynucleotides encoding one or more S. aureus antigenic polypeptide, variant or immunogenic fragment thereof are delivered to the desired cells via RNA viral vectors. Thus, in certain embodiments, the composition comprises an RNA viral vector comprising one or more RNA polynucleotides, as described herein. One of skill in the art shall recognize that any known engineered RNA virus can be used in the compositions or methods of the present disclosure, if such an RNA virus is capable infecting cells, inducing production of one or more antigenic polypeptide or immunogenic fragment thereof, and inducing a host immune response against said one or more antigenic polypeptide or immunogenic fragment thereof.

In one embodiment, the RNA viral vector comprises one or more RNA polynucleotide comprising a nucleic acid sequence encoding N-acetylmuramyl-L-alanine amidase (Amd). In one embodiment, the one or more RNA polynucleotide comprises the nucleic acid sequence of SEQ ID NO: 6.

In one embodiment, the RNA viral vector comprises one or more RNA polynucleotide comprising a nucleic acid sequence encoding endo-β-N-acetylglucosaminidase (Gmd). In one embodiment, the one or more RNA polynucleotide comprises the nucleic acid sequence of SEQ ID NO:21.

In one embodiment, the RNA viral vector comprises one or more RNA polynucleotide comprising a nucleic acid sequence encoding autolysin (Atl). In one embodiment, the one or more RNA polynucleotide comprises the nucleic acid sequence of SEQ ID NO: 3. In some embodiments, the RNA viral vector comprises one or more RNA polynucleotide comprising a nucleic acid sequence encoding Atl (e.g., SEQ ID NO:1) or an antigenic fragment thereof (e.g., a polypeptide having the sequence of SEQ ID NO:72).

In one embodiment, the RNA viral vector comprises one or more RNA polynucleotides comprising nucleic acid sequences encoding Amd and Gmd. In one embodiment, the RNA sequence encoding Amd comprises the nucleic acid sequence of SEQ ID NO: 6 and the RNA sequence encoding Gmd comprises the nucleic acid sequence of SEQ ID NO:21. In some embodiments, the RNA viral vector comprises one or more RNA polynucleotide comprising a nucleic acid sequence encoding Amd (e.g., SEQ ID NO:4) or an antigenic fragment thereof (e.g., a polypeptide having the sequence of SEQ ID NO:7, 10, 13, or 16). In some embodiments, the RNA viral vector comprises one or more RNA polynucleotide comprising a nucleic acid sequence encoding Gmd (e.g., SEQ ID NO:19) or an antigenic fragment thereof (e.g., a polypeptide having the sequence of SEQ ID NO:22, 25, or 28).

In some embodiments, the RNA viral vector comprises one or more RNA polynucleotide comprising a nucleic acid sequence encoding one or more secreted immunotoxin of S. aureus. In some embodiments, said one or more secreted immunotoxin of S. aureus is selected from the group consisting of: alpha-hemolysin (Hla), chemotaxis inhibiting protein of S. aureus (CHIPS) and the Staphylococcal complement inhibitor (SCIN). In one embodiment, the nucleic acid sequence encoding Hla comprises the sequence of SEQ ID NO: 53. In some embodiments, the RNA viral vector comprises one or more RNA polynucleotide comprising a nucleic acid sequence encoding Hla (e.g., SEQ ID NO:51) or an antigenic fragment thereof (e.g., a polypeptide having the sequence of SEQ ID NO:54 or 57). In one embodiment, the nucleic acid sequence encoding CHIPS comprises the sequence of SEQ ID NO: 33. In some embodiments, the RNA viral vector comprises one or more RNA polynucleotide comprising a nucleic acid sequence encoding CHIPs (e.g., SEQ ID NO:31) or an antigenic fragment thereof (e.g., a polypeptide having the sequence of SEQ ID NO:34, or 37). In one embodiment, the nucleic acid sequence encoding SCIN comprises the sequence of SEQ ID NO: 42. In some embodiments, the RNA viral vector comprises one or more RNA polynucleotide comprising a nucleic acid sequence encoding SCIN (e.g., SEQ ID NO:40) or an antigenic fragment thereof (e.g., a polypeptide having the sequence of SEQ ID NO:43, 45, or 48).

In some embodiments, the RNA viral vector comprises one or more RNA polynucleotide comprising a nucleic acid sequence encoding one or more lytic transglycosylase of S. aureus. In some embodiments, said one or more lytic transglycosylase is IsaA or SceD. In some embodiments, the RNA viral vector comprises one or more RNA polynucleotide comprising a nucleic acid sequence encoding IsaA (e.g., SEQ ID NO:94) or an antigenic fragment thereof (e.g., a polypeptide having the sequence of SEQ ID NO:97, 100, or 103). In one embodiment, the nucleic acid sequence encoding IsaA comprises the sequence of SEQ ID NO: 96. In some embodiments, the RNA viral vector comprises one or more RNA polynucleotide comprising a nucleic acid sequence encoding SceD (e.g., SEQ ID NO:109) or an antigenic fragment thereof (e.g., a polypeptide having the sequence of SEQ ID NO: 112). In one embodiment, the nucleic acid sequence encoding SceD comprises the sequence of SEQ ID NO: 111.

In some embodiments, the RNA viral vector comprises one or more RNA polynucleotide comprising a nucleic acid sequence encoding CgoX and comprises the sequence of SEQ ID NO:62. In some embodiments, the RNA viral vector comprises one or more RNA polynucleotide comprising a nucleic acid sequence encoding CgoX (e.g., SEQ ID NO: 60) or an antigenic fragment thereof (e.g., a polypeptide having the sequence of SEQ ID NO: 63, 66, or 69).

In some embodiments, the RNA viral vector is capable of infecting vertebrate cells. In some embodiments, said vertebrate is a mammal. In some embodiments, said mammal is a human. In some embodiments, no specific cell is targeted. In some embodiments, an antigen-presenting cell (APC) is specifically targeted. In some embodiments, the APC is a dendritic cell (DC).

In some embodiments, the RNA viral vector is capable of inducing production of one or more antigenic polypeptide or immunogenic fragment thereof directly from the engineered RNA viral genome, which can act as functional mRNA translated by host cellular machinery. In some embodiments, the RNA viral vector is a positive-strand RNA virus. In some embodiments, the positive-strand RNA virus belongs to one or more family selected from the group consisting of (Virion morphology/genome type displayed in parentheses): Arteriviridae (Enveloped, helical nucleocapsid/Unsegmented); Astroviridae (Nonenveloped, icosahedral/Unsegmented); Caliciviridae (Nonenveloped, icosahedral/Unsegmented); Coronaviridae (Enveloped, helical nucleocapsid/Unsegmented); Flaviviridae (Enveloped, icosahedral nucleocapsid/Unsegmented); Hepeviridae (Nonenveloped, icosahedral/Unsegmented); Nodaviridae (Nonenveloped, icosahedral/Bisegmented); Picornaviridae (Nonenveloped, icosahedral/Unsegmented); Togaviridae (Enveloped, icosahedral nucleocapsid/Unsegmented).

In some embodiments, the RNA viral vector is packaged with viral proteins that transcribe the engineered RNA viral genome into functional mRNA, which can then be translated into one or more antigenic polypeptide or immunogenic fragment thereof by host cellular machinery. In some embodiments, the RNA viral vector is a negative-strand RNA virus. In some embodiments, the negative-strand RNA virus belongs to one or more family selected from the group consisting of (Virion morphology/genome type displayed in parentheses): Arenaviridae (Enveloped, helical nucleocapsid/Bisegmented; Bornaviridaeb (Enveloped, helical nucleocapsid/Unsegmented); Bunyaviridae (Enveloped, helical nucleocapsid/Trisegmented); Filoviridaeb (Enveloped, helical nucleocapsid/Unsegmented); Nymaviridaeb (Enveloped, helical nucleocapsid/Unsegmented); Orthmyxoviridae (Enveloped, helical nucleocapsid/Segmented); Paramyxoviridaeb (Enveloped, helical nucleocapsid/Unsegmented); Pneumoviridaeb (Enveloped, helical nucleocapsid/Unsegmented) Rhabdoviridaeb (Enveloped, helical nucleocapsid/Unsegmented).

In some embodiments, the RNA viral vector is one or more selected from the group consisting of: retrovirus, lentivirus, alphavirus, flavivirus, rhabdovirus, measles virus, Newcastle disease virus, and picornavirus.

In some embodiments, the retrovirus is one or more selected from the group consisting: Moloney Murine Leukemia Virus (MMLV), Moloney Murine Sarcoma Virus (MMSV), and Murine Stem Cell Virus (MSCV).

In some embodiments, the lentivirus is one more selected from the group consisting of: Human Immunodeficiency Virus 1 (HIV-1), Human Immunodeficiency Virus 2 (HIV-2) and Equine Infectious Anemia Virus (EIAV).

In some embodiments, the alphavirus is one or more selected from the group consisting of: Semliki Forest Virus (SFV), Sindbis virus (SIN), Venezuelan Equine Encephalitis virus (VEE), and M1 alphavirus.

In some embodiments, the flavivirus is one or more selected from the group consisting of: Kunjin virus (KUN), West Nile virus, Yellow Fever Virus (YFV), and Dengue virus.

In some embodiments, the rhabdovirus is one or more selected from the group consisting of: Rabies virus and Vesicular Stomatitis Virus (VSV).

In some embodiments, the measles virus comprises measles virus Edmonston strain (MV-Edm).

In some embodiments, the Newcastle disease virus is one or more strain selected from the group consisting of: LaSota and Hitchner B1.

In some embodiments, the picornavirus comprises Coxsackievirus. In some embodiments, the Coxsackievirus is Coxsackievirus B3 (CVB3) strain.

### Methods

In some embodiments, the present disclosure comprises a method of inducing an immune response against S. aureus in a subject. In some embodiments, the disclosure provides a method of treating or preventing S. aureus infection in a subject. In some embodiments, the disclosure provides a method that comprises administering to a subject a composition comprising one or more nucleic acid encoding one or more S. aureus antigenic polypeptide or immunogenic fragment thereof. In some embodiments, the disclosure provides a method that comprises administering to a subject a composition comprising one or more nucleic acid encoding one or more consensus S. aureus antigenic polypeptide or immunogenic fragment thereof. In some embodiments, the S. aureus antigenic polypeptide comprises one or more selected from Amd, Gmd and Atl, or an immunogenic fragment or variant thereof. In some embodiments, the S. aureus antigenic polypeptide comprises one or more selected from CHIPs, SCIN, and Hla, or an immunogenic fragment or variant thereof. In some embodiments, the S. aureus antigenic polypeptide comprises a lytic transglycosylase, or an immunogenic fragment or variant thereof. In some embodiments, the S. aureus antigenic polypeptide comprises IsaA and/or SceD, or an immunogenic fragment or variant thereof. In some embodiments, the S. aureus antigenic polypeptide comprises CgoX, or an immunogenic fragment or variant thereof.

In some embodiments, the disclosure provides a method of inducing an antigen specific immune response in a subject, that comprises administering to the subject an effective amount of a composition comprising one or more nucleic acid molecules encoding one or more S. aureus antigenic polypeptide, immunogenic variant or fragment thereof. In some embodiments, the encoded S. aureus antigenic polypeptide, immunogenic variant or fragment corresponds to an S. aureus protein selected from: autolysin (Atl), N-acetylmuramyl-L-alanine amidase (Amd), endo-β-N-acetylglucos-aminidase (Gmd), alpha-hemolysin (Hla), chemotaxis inhibiting protein of S. aureus (CHIPS), Staphylococcal complement inhibitor (SCIN), and coproporphyrinogen III oxidase (CgoX), as provided herein. In some embodiments, the encoded S. aureus antigenic polypeptide, immunogenic variant or fragment corresponds to an S. aureus lytic transglycosylase. In some embodiments, the encoded S. aureus antigenic polypeptide, immunogenic variant or fragment corresponds to IsaA or SceD, as provided herein. In some embodiments, the administered amount is an effective amount to elicit an antigen specific antibody titre (i.e., titer of antibodies that bind the encoded antigenic polypeptide, immunogenic variant or fragment) in the blood or serum of the subject of >0.1 μg/ml, >0.2 μg/ml, >0.35 μg/ml, or >0.5 μg/ml, by 20 days after administration, 25 days, days after administration or 30 days after administration. An “antigen specific antibody” is a serum antibody the binds specifically to the antigenic polypeptide encoded by the administered compositions provided herein. In some embodiments, the administered amount is effective to elicit an antigen-specific antibody titre in the blood or serum of the subject (against the administered antigenic polypeptide, immunogenic variant or fragment) of >0.2 μg/ml by 20 days after administration. In exemplary embodiments, the antigen-specific antibody is produced or reached following a single dose of vaccine administered to the subject. In other embodiments, the concentration of antigen-specific antibody is produced or reached following multiple doses e.g., following a first and a second dose (e.g., a booster dose). Antibody concentration can routinely be determined using techniques known in the art in some exemplary embodiments, antibody concentration is determined or measured by enzyme-linked immunosorbent assay (ELISA). In exemplary embodiments, antibody level or concentration is determined or measured by neutralization assay, e.g., by microneutralization assay.

Treatment and Prevention

The present disclosure provides methods of inducing an adaptive immune response in a subject comprising administering an effective amount of a composition comprising one or more nucleic acid molecules (e.g., a ribonucleic acid) encoding one or more S. aureus antigenic polypeptide, immunogenic variant or fragment thereof, and wherein an effective amount is a dose is effective to induce an adaptive immune response in the subject. In some embodiments, the encoded S. aureus antigenic polypeptide, immunogenic variant or fragment corresponds to an S. aureus protein selected from: autolysin (Atl), N-acetylmuramyl-L-alanine amidase (Amd), endo-β-N-acetylglucos-aminidase (Gmd), alpha-hemolysin (Hla), chemotaxis inhibiting protein of S. aureus (CHIPS), Staphylococcal complement inhibitor (SCIN), and coproporphyrinogen III oxidase (CgoX), as provided herein. In some embodiments, the encoded S. aureus antigenic polypeptide, immunogenic variant or fragment comprises a consensus S. aureus antigenic polypeptide, immunogenic variant or fragment thereof. In some embodiments, the encoded S. aureus antigenic polypeptide, immunogenic variant or fragment corresponds to a lytic transglycosylase. In some embodiments, the encoded S. aureus antigenic polypeptide, immunogenic variant or fragment corresponds to a IsaA or SceD, as provided herein.

In one embodiment, the method provides immunity in the subject to an infection, disease, or disorder associated with an antigen. The present disclosure thus provides a method of treating or preventing the infection, disease, or disorder associated with the antigen. For example, the method may be used to treat or prevent S. aureus infection, or diseases or disorders associated with S. aureus infection.

In one embodiment, the composition is administered to a subject having an infection, disease, or disorder associated with the antigen. In one embodiment, the composition is administered to a subject at risk for developing the infection, disease, or disorder associated with the antigen. For example, the composition may be administered to a subject who is at risk for developing S. aureus infection.

In one embodiment, the method comprises treating osteomyelitis by administering an effective amount of a composition comprising one or more nucleic acid molecules (e.g., a ribonucleic acid) encoding one or more S. aureus antigenic polypeptide, immunogenic variant or fragment thereof, to a subject in need thereof. In some embodiments, the encoded S. aureus antigenic polypeptide, immunogenic variant or fragment corresponds to an S. aureus protein selected from: autolysin (Atl), N-acetylmuramyl-L-alanine amidase (Amd), endo-β-N-acetylglucos-aminidase (Gmd), alpha-hemolysin (Hla), chemotaxis inhibiting protein of S. aureus (CHIPS), Staphylococcal complement inhibitor (SCIN), and coproporphyrinogen III oxidase (CgoX), as provided herein. In some embodiments, the encoded S. aureus antigenic polypeptide, immunogenic variant or fragment corresponds to an S. aureus lytic transglycoslyase sequence. In some embodiments, the encoded S. aureus antigenic polypeptide, immunogenic variant or fragment corresponds to IsaA or SceD protein, as provided herein.

In one embodiment, the method comprises treating osteomyelitis by administering an effective amount of a composition provided herein to a subject having an S. aureus bone or joint infection. In some embodiments, the disclosure provides a method of treating osteomyelitis that comprises administering a nucleic acid (e.g., a ribonucleic acid molecule) encoding one or more S. aureus antigens to a subject having an S. aureus bone or joint infection. Administration of these agents or compositions can be carried out using any of the routes described herein.

In one embodiment, the present disclosure comprises a method of introducing an orthopedic implant which includes administering to the subject in need of the orthopedic implant an effective amount of a composition provided herein. In some embodiments, the disclosure provides a method of introducing an orthopedic implant which includes a nucleic acid (e.g., a ribonucleic acid molecule) encoding one or more S. aureus antigens to the subject in need of the orthopedic implant. The orthopedic implant can be a joint prosthesis, graft or synthetic implant. Exemplary joint prosthetics includes, without limitation, a knee prosthetic, hip prosthetic, finger prosthetic, elbow prosthetic, shoulder prosthetic, temperomandibular prosthetic, and ankle prosthetic. Other prosthetics can also be used. Exemplary grafts or synthetic implants include, without limitation, an artificial intervertebral disk, meniscal implant, or a synthetic or allograft anterior cruciate ligament, medial collateral ligament, lateral collateral ligament, posterior cruciate ligament, Achilles tendon, and rotator cuff. Other grafts or implants can also be used.

In some embodiments, the provided method comprises administering nucleoside-modified nucleic acid which provides stable expression of the antigen described herein. In some embodiments, administration of nucleoside-modified nucleic acid results in little to no innate immune response, while inducing an effective adaptive immune response.

Administration of the compositions of the disclosure in a method of treatment can be achieved in a number of different ways, using methods known in the art. In one embodiment, the method of the disclosure comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

It will be appreciated that the composition of the disclosure may be administered to a subject either alone, or in conjunction with a second therapeutic agent. In some embodiment, the second therapeutic agent is an antibiotic agent or immunotherapeutic agent. Exemplary antibiotic agents include, without limitation, vancomycin, tobramycin, cefazolin, erythromycin, clindamycin, rifampin, gentamycin, fusidic acid, minocycline, co-trimoxazole, clindamycin, linezolid, quinupristin-dalfopristin, daptomycin, tigecycline, dalbavancin, telavancin, oritavancin, ceftobiprole, ceftaroline, iclaprim, the carbapenem CS-023/RO-4908463, and combinations thereof. Exemplary immunotherapeutic agents include, without limitation, tefibazumab, BSYX-A110, Aurexis™, and combinations thereof. The above lists of antibiotic agents and immunotherapeutic agents are intended to be non-limiting examples; thus, other antibiotic agents or immunotherapeutic agents are also contemplated. Combinations or mixtures of the second therapeutic agent can also be used for the purposes of the present disclosure. These agents can be administered contemporaneously or as a single formulation.

The therapeutic and prophylactic methods of the disclosure thus encompass the use of pharmaceutical compositions encoding an antigen, described herein to practice the methods of the disclosure. The pharmaceutical compositions useful for practicing the invention may be administered to deliver a dose of between 1 ng/kg/day and 100 mg/kg/day. In one embodiment, the invention envisions administration of a dose which results in a concentration of the compound of the present disclosure from 10 nM and 10 μM in a mammal.

Typically, dosages which may be administered in a method of the disclosure to a mammal, for example a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 0.1 μg to about 10 mg per kilogram of body weight of the mammal. In some embodiments, the dosage will vary from about 1 kg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of an immunogenic composition or vaccine of the present disclosure may be performed by single administration or boosted by multiple administrations.

In one embodiment, the invention includes a method comprising administering one or more compositions encoding one or more antigens or adjuvants described herein. In certain embodiments, the method has an additive effect, wherein the overall effect of the administering the combination is approximately equal to the sum of the effects of administering each antigen or adjuvant. In other embodiments, the method has a synergistic effect, wherein the overall effect of administering the combination is greater than the sum of the effects of administering each antigen or adjuvant.

Therapeutic Formulations

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences, 1985, Genaro, ed., Mack Publishing Co., Easton, PA), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present disclosure and practice the claimed methods. The following working examples therefore are not to be construed as limiting in any way the remainder of the disclosure.

### Example 1: Anti-Autolysin Antibodies Correlate with S. aureus Infection Resolution

Previous work has indicated that passive immunization with anti-Amd and anti-Gmd antibodies induces immune responses capable of treating S. aureus infection and associated diseases or disorders. However, passive immunization strategies are transient, limiting their long-term ability to protect against infection. Thus, there remains a need for compositions and methods for providing both transient therapeutic treatments and long-term prevention for S. aureus infection.

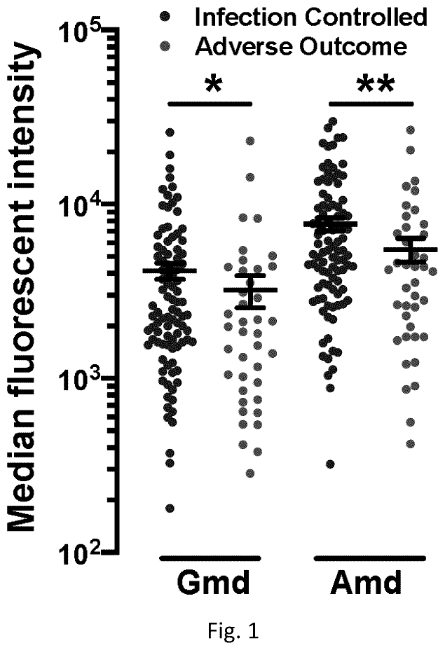

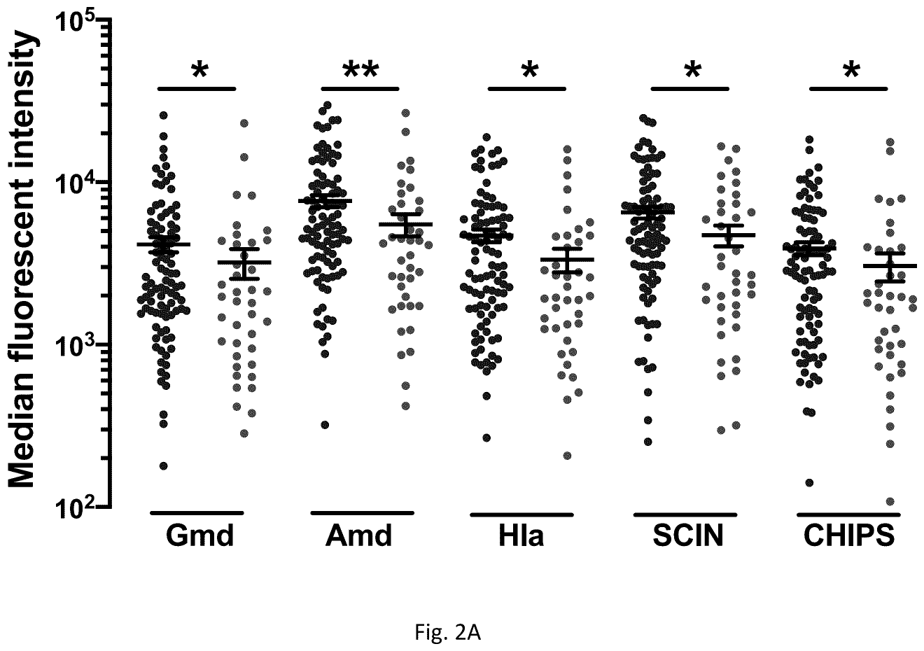

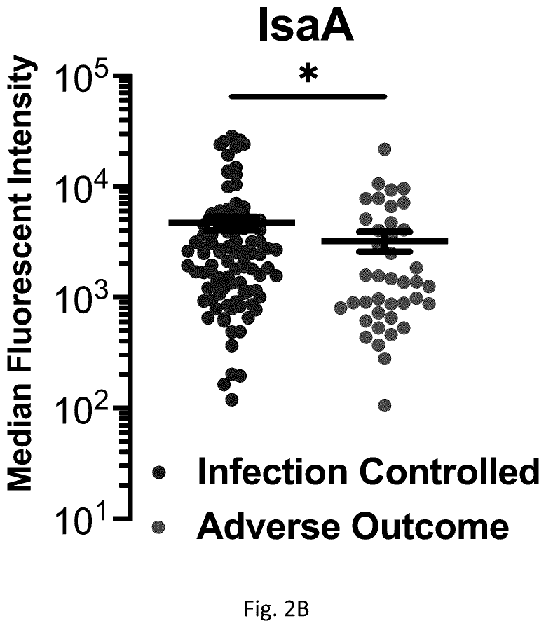

Examination of anti-autolysin serum IgG levels in a cohort of 194 patients in the AOTrauma CPP Bone Infection Registry with confirmed S. aureus infections demonstrates that endogenously produced anti-Amd and anti-Gmd levels were positively correlated with successful resolution of infection absent complications (FIG. 1). This, coupled with the successful strategies involving passive immunization, suggest that active immunization with Amd, Gmd or both (in the form of the precursor Atl) will be effective in the treatment or prevention S. aureus infection. Furthermore, serum IgG levels of S. aureus secreted immunotoxins alpha-hemolysin (Hla), chemotaxis inhibiting protein of S. aureus (CHIPS) and the Staphylococcal complement inhibitor (SCIN) in the same cohort were also positively correlated with successful resolution of infection absent complications (FIG. 2A). Additionally, serum IgG levels against S. aureus lytic transglycosylase protein IsaA positively correlated with successful resolution of infection absent complications (FIG. 2B). FIG. 2B depicts exemplary results of relative IgG antibody levels in patients against IsaA presented as the median fluorescent intensity+/−SD. This suggests that immunization against secreted immunotoxins and S. aureus lytic transglycosylases may also be effective in the treatment or prevention of S. aureus infection, and that co-immunization against one or more autolysin protein may further improve the effectiveness.

Thus, Atl is expressed in the form of mRNA with its codon usage optimized for expression in human cells and encoding an N-terminal leader sequence (i.e. signal peptide) that will mediate its secretion from the producing cells (SEQ ID NO. 3). Further, each Atl subunit-encoding mRNA is expressed separately in an Amd-specific (SEQ ID NO: 6) and a Gmd-specific (SEQ ID NO:21) vehicle so that they can be examined independently or in combination in evaluation experiments. Codon optimized mRNA that encodes selected S. aureus antigens are delivered into the human cells by one of several mechanisms (e.g. lipid nanoparticles, RNA viruses, etc.) where it is transiently expressed. While not being bound by particular theory, it is believed that this will yield significant levels of S. aureus proteins (e.g., SEQ ID NO: 4, SEQ ID NO:19, SEQ ID NO: 1) that will elicit protective humoral and cellular immune response.

### Example 2: Antibody Response to Consensus S. aureus Antigenic Polypeptides

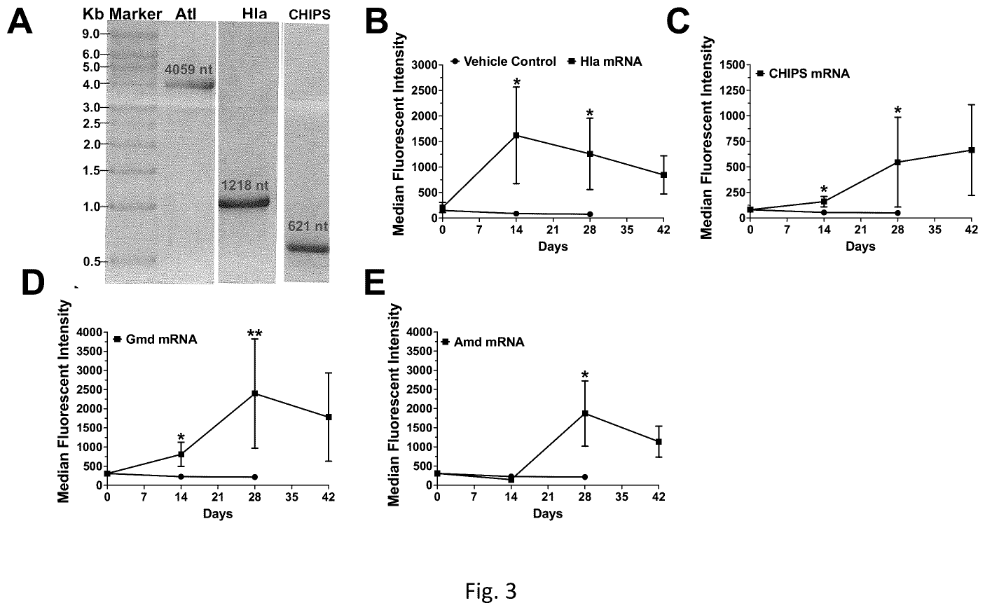

To generate consensus sequences for S. aureus antigenic polypeptides, 238 completed/closed S. aureus genomes were searched using blastp to identify the accessioned protein products with sequences similar to those of Atl (contains both Gmd and Amd on a single transcript), Hla, CHIPS, IsaA and SceD from the USA300 genome. Clustal-Omega (1.2.4) was used to perform multiple sequence alignment of the top results for each assembly and derive the consensus sequence for full-length Atl (containing Gmd and Amd), Hla, and CHIPS. Jalview (2.11.1.4). Subsequently, the consensus mRNA sequences for each antigen were entered into the GenScript GenSmart Codon Optimization program for human codon optimization. The resulting human codon optimized mRNA sequences were given to TriLink Biotechnologies Inc., who completed in vitro mRNA synthesis (>2 mg each) and quality control for all antigens. FIG. 3A depicts the size and integrity of the in vitro synthesized mRNAs as was assessed via 0.8% agarose gel electrophoresis, and a representative image for each mRNA is shown with their size in nucleotides (nt). Each synthesized mRNA (15 μg/mice) was individually mixed with in vivo-jetRNA transfection reagent and injected into 6-week-old female C57B/6 mice intramuscularly, and the mice were boosted with the same vaccine on day 14. Submandibular bleeding was performed at days 0, 14, 28, 42 post-immunization, and the resulting sera were probed for anti-S. aureus antibodies via Luminex. FIGS. 3B-E depict exemplary results of relative IgG antibody levels against: Hla (FIG. 3B), CHIPS (FIG. 3C), Gmd (FIG. 3D), and Amd (FIG. 3E), presented as the median fluorescent intensity+/−SD. Significant IgG antibody levels were detectable 28 days post-immunization with all mRNA nanoparticle vaccines, suggesting robust immune induction against the majority of the consensus S. aureus antigenic polypeptides expressed.

### Example 3: Exemplary S. aureus Antigen Protein, RNA, and DNA Sequences

TABLE 1
Exemplary S. aureus antigen and codon optimized sequences

| | Sequence |
|---|---|
| Atl, | MAKKFNYKLPSMVALTLVGSAVTAHQVQAAETTQDQTTNKNVLDSNKVKATTEQAKAEVKNPTQNISGTQVYQDP |
| FL, | AIVQPKTANNKTGNAQVSQKVDTAQVNGDTRANQSATTNNTQPVAKSTSTTAPKTNTNVTNAGYSLVDDEDDNSA |
| PP | NTENQINPELIKSAAKPAALETQYKAAAPKAATTSAPKAKTEATPKVTTFSASAQPRSVAATPKTSLPKYKPQVN |
| | SSINDYIRKNNLKAPKIEEDYTSYFPKYAYRNGVGRPEGIVVHDTANDRSTINGEISYMKNNYQNAFVHAFVDGD |
| | RIIETAPTDYLSWGVGAVGNPRFINVEIVHTHDYASFARSMNNYADYAATQLQYYGLKPDSAEYDGNGTVWTHYA |
| | VSKYLGGTDHADPHGYLRSHNYSYDQLYDLINEKYLIKMGKVAPWGTQSTTTPTTPSKPTTPSTPSKPSKPSTGK |
| | LTVAANNGVAQIKPTNSGLYTTVYDKTGKATNEVQKTFAVSKTATLGNQKFYLVQDYNSGNKFGWVKEGDVVYNT |
| | AKSPVNVNQSYSIKPGTKLYTVPWGTSKQVAGSVSGSGNQTFKASKQQQIDKSIYLYGSVNGKSGWVSKAYLVDT |
| | AKPTPTPTPKPSTPTTNNKLTVSSLNGVAQINAKNNGLFTTVYDKTGKPTKEVQKTFAVTKEASLGGNKFYLVKD |
| | YNSPTLIGWVKQGDVIYNNAKSPVNVMQTYTVKPGTKLYSVPWGTYKQEAGAVSGTGNQTFKATKQQQIDKSIYL |
| | FGTVNGKSGWVSKAYLAVPAAPKKAVAQPKTAVKAYAVTKPQTTQTTQTVSKIAQVKPNNTGIRASVYEKTAKNG |
| | AKYADRTFYVTKERAHGNETYVLLNNTSHNIPLGWFNVKDLNVQNLGKEVKTTQKYTVNKSNNGLSMVPWGTKNQ |
| | VILTGNNIAQGTFNATKQVSVGKDVYLYGTINNRTGWVNAKDLTAPTAVKPTTSAAKDYNYTYVIKNGNGYYYVT |
| | PNSDTAKYSLKAFNEQPFAVVKEQVINGQTWYYGKLSNGKLAWIKSTDLAKELIKYNQTGMTLNQVAQIQAGLQY |
| | KPQVQRVPGKWTDANFNDVKHAMDTKRLAQDPALKYQFLRLDQPQNISIDKINQFLKGKGVLENQGAAFNKAAQM |
| | YGINEVYLISHALLETGNGTSQLAKGADVVNNKVVTNSNTKYHNVFGIAAYDNDPLREGIKYAKQAGWDTVSKAI |
| | VGGAKFIGNSYVKAGQNTLYKMRWNPAHPGTHQYATDVDWANINAKIIKGYYDKIGEVGKYFDIPQYK (SEQ |
| | ID NO: 1) |
| Atl, | ATGGCCAAGAAATTCAATTATAAGCTGCCTAGCATGGTGGCCCTGACACTGGTGGGAAGCGCCGTGACAGCCCAC |
| FL, DNA | CAGGTCCAGGCCGCTGAAACCACCCAGGACCAGACGACGAACAAGAACGTGCTGGACAGCAATAAAGTCAAGGCC |
| | ACCACCGAGCAGGCCAAAGCTGAGGTGAAGAATCCCACCCAAAACATCTCTGGCACCCAGGTGTACCAGGACCCT |
| | GCCATCGTGCAGCCTAAGACCGCCAACAACAAGACTGGCAACGCCCAGGTGTCCCAGAAGGTGGATACCGCACAA |
| | GTGAACGGCGACACCAGAGCCAATCAGAGCGCCACAACCAACAACACCCAGCCTGTGGCCAAGAGCACCTCCACA |
| | ACTGCCCCCAAGACAAATACTAATGTGACAAACGCCGGTTATTCTCTGGTCGATGACGAGGATGATAACTCTGCC |
| | AATACCGAGAACCAGATCAACCCAGAGCTGATCAAAAGCGCCGCCAAACCTGCCGCCCTGGAAACACAGTATAAG |
| | GCCGCTGCTCCAAAGGCCGCCACCACCAGCGCCCCAAAGGCCAAAACCGAAGCCACTCCTAAGGTGACAACATTT |
| | TCTGCCAGCGCCCAGCCAAGAAGCGTAGCCGCCACCCCTAAGACGAGCCTGCCCAAATACAAGCCCCAGGTGAAT |
| | AGCAGCATTAACGACTACATCAGAAAGAATAATCTGAAGGCCCCAAAGATCGAGGAAGATTACACCAGCTACTTC |
| | CCCAAGTACGCCTATAGAAACGGCGTGGGCAGACCTGAGGGCATCGTGGTTCACGACACAGCTAATGACCGGAGC |
| | ACCATCAACGGCGAAATCAGCTACATGAAGAACAACTACCAGAATGCCTTCGTGCACGCCTTCGTGGATGGCGAC |
| | CGGATCATCGAGACCGCTCCTACCGACTACCTGAGCTGGGGCGTGGGCGCCGTGGGCAACCCCAGATTCATCAAC |
| | GTGGAAATCGTGCATACACACGACTACGCCAGCTTCGCTAGAAGCATGAACAACTACGCCGATTACGCCGCTACA |
| | CAACTGCAATACTACGGCCTGAAGCCTGATAGCGCCGAGTATGACGGCAACGGCACAGTTTGGACCCACTACGCC |
| | GTGAGCAAGTACCTCGGCGGCACAGACCACGCCGATCCTCACGGCTACCTGCGGTCCCACAACTACTCTTACGAT |
| | CAGCTGTACGACCTGATCAACGAAAAGTATCTTATCAAAATGGGCAAGGTGGCACCTTGGGGCACCCAGAGCACC |
| | ACCACACCTACAACACCTTCCAAGCCTACAACCCCTTCTACCCCTTCTAAGCCCTCTAAGCCCAGCACCGGGAAA |
| | CTGACAGTGGCCGCTAACAACGGAGTAGCCCAGATCAAACCTACCAACAGCGGCCTGTACACCACCGTCTACGAC |
| | AAGACAGGCAAGGCTACCAACGAGGTGCAGAAGACCTTTGCCGTGTCCAAAACAGCAACGCTGGGCAACCAGAAG |
| | TTCTACCTGGTGCAGGACTACAACAGTGGCAACAAGTTCGGCTGGGTGAAGGAAGGAGATGTGGTGTACAACACC |
| | GCCAAGAGCCCTGTGAATGTGAACCAGTCTTATAGCATCAAGCCCGGCACAAAGCTGTACACCGTTCCTTGGGGA |
| | ACCTCCAAACAGGTGGCCGGCAGTGTGAGTGGCTCCGGCAATCAGACCTTCAAGGCCAGCAAGCAGCAGCAGATC |
| | GACAAATCTATCTACCTGTATGGAAGTGTGAACGGGAAGTCCGGCTGGGTATCCAAGGCATACCTAGTTGATACC |
| | GCCAAGCCCACCCCTACCCCGACACCTAAACCTAGCACACCCACAACCAACAACAAGCTGACCGTGTCTTCTCTG |
| | AACGGAGTGGCCCAGATCAACGCCAAGAACAATGGCCTGTTCACCACAGTGTACGACAAGACAGGCAAGCCTACC |
| | AAGGAGGTGCAGAAGACCTTCGCCGTGACCAAGGAAGCAAGCCTGGGAGGCAACAAGTTCTACCTCGTGAAGGAC |
| | TACAATAGCCCTACCCTGATTGGCTGGGTGAAGCAGGGAGATGTAATCTACAACAACGCTAAGAGCCCCGTGAAC |
| | GTGATGCAGACCTACACCGTGAAGCCTGGCACAAAGCTGTACAGCGTGCCCTGGGGCACATACAAGCAGGAGGCC |
| | GGCGCCGTGAGCGGAACCGGCAACCAGACCTTTAAGGCCACCAAGCAGCAACAGATCGACAAGAGTATATACCTG |
| | TTCGGCACTGTAAACGGTAAGTCCGGCTGGGTGAGCAAAGCGTACCTGGCCGTTCCTGCTGCCCCTAAAAAGGCC |
| | GTGGCTCAACCGAAAACCGCCGTGAAAGCCTACGCCGTCACCAAACCTCAGACAACACAGACAACTCAAACCGTA |
| | AGCAAGATCGCCCAGGTGAAGCCCAACAACACCGGAATTCGGGCCAGCGTGTACGAGAAGACCGCCAAGAACGGC |
| | GCCAAGTACGCTGATAGAACATTCTACGTGACCAAGGAACGGGCCCACGGTAACGAGACATACGTGCTGCTGAAC |
| | AACACCTCCCACAACATCCCCCTGGGATGGTTCAACGTGAAAGATCTGAACGTGCAGAACCTGGGCAAAGAGGTG |
| | AAGACCACACAGAAATACACCGTGAACAAGAGTAACAACGGCCTCAGCATGGTGCCTTGGGGCACCAAGAACCAG |
| | GTGATCCTGACCGGCAACAATATCGCCCAAGGCACCTTCAACGCCACCAAGCAGGTGAGCGTGGGCAAGGACGTG |
| | TACCTGTACGGCACAATCAACAACAGAACCGGATGGGTCAACGCCAAGGACCTGACCGCCCCTACTGCCGTGAAA |
| | CCAACAACCAGCGCCGCCAAGGACTACAATTACACCTACGTGATTAAGAATGGAAATGGCTACTACTACGTGACA |
| | CCAAATAGTGATACCGCCAAATATAGCCTGAAGGCCTTCAACGAGCAGCCATTCGCGGTGGTGAAGGAGCAGGTG |
| | ATCAACGGACAGACCTGGTACTACGGCAAGCTGAGCAACGGCAAACTGGCCTGGATCAAGAGCACTGACCTGGCC |
| | AAAGAGCTGATTAAATACAACCAGACGGGCATGACCCTTAACCAAGTGGCCCAGATCCAGGCCGGACTGCAGTAC |
| | AAGCCTCAGGTGCAGAGAGTGCCAGGGAAGTGGACCGACGCCAACTTCAACGACGTGAAGCACGCTATGGACACA |
| | AAGCGCCTGGCCCAGGATCCTGCTCTGAAGTACCAGTTTCTGAGACTGGACCAGCCTCAGAACATCTCCATCGAC |
| | AAGATCAACCAGTTCCTGAAAGGCAAGGGCGTTCTGGAGAACCAAGGCGCCGCCTTCAACAAAGCTGCTCAGATG |
| | TACGGCATCAATGAGGTGTACCTGATCAGCCACGCCCTGCTGGAAACCGGCAACGGCACCAGCCAGCTGGCTAAG |
| | GGCGCCGACGTGGTGAACAACAAGGTGGTGACCAACTCTAATACCAAGTATCACAACGTCTTTGGAATCGCCGCC |
| | TATGACAACGACCCCCTGAGGGAAGGCATCAAATACGCCAAGCAGGCCGGCTGGGATACAGTGAGCAAGGCTATC |
| | GTGGGGGGCGCTAAGTTCATCGGAAATTCTTACGTGAAGGCTGGCCAGAACACCCTGTACAAGATGCGGTGGAAC |
| | CCTGCTCATCCTGGCACCCACCAATACGCCACGGACGTGGACTGGGCCAACATCAACGCCAAGATCATCAAGGGT |
| | TACTACGACAAGATTGGAGAGGTGGGCAAGTACTTCGACATCCCTCAGTACAAG (SEQ ID NO: 2) |
| Atl, | AUGGCCAAGAAAUUCAAUUAUAAGCUGCCUAGCAUGGUGGCCCUGACACUGGUGGGAAGCGCCGUGACAGCCCAC |
| RNA | ACCACCGAGCAGGCCAAAGCUGAGGUGAAGAAUCCCACCCAAAACAUCUCUGGCACCCAGGUGUACCAGGACCCU |
| FL, | CAGGUCCAGGCCGCUGAAACCACCCAGGACCAGACGACGAACAAGAACGUGCUGGACAGCAAUAAAGUCAAGGCC |
| | GCCAUCGUGCAGCCUAAGACCGCCAACAACAAGACUGGCAACGCCCAGGUGUCCCAGAAGGUGGAUACCGCACAA |
| | GUGAACGGCGACACCAGAGCCAAUCAGAGCGCCACAACCAACAACACCCAGCCUGUGGCCAAGAGCACCUCCACA |
| | ACUGCCCCCAAGACAAAUACUAAUGUGACAAACGCCGGUUAUUCUCUGGUCGAUGACGAGGAUGAUAACUCUGCC |
| | AAUACCGAGAACCAGAUCAACCCAGAGCUGAUCAAAAGCGCCGCCAAACCUGCCGCCCUGGAAACACAGUAUAAG |
| | GCCGCUGCUCCAAAGGCCGCCACCACCAGCGCCCCAAAGGCCAAAACCGAAGCCACUCCUAAGGUGACAACAUUU |
| | UCUGCCAGCGCCCAGCCAAGAAGCGUAGCCGCCACCCCUAAGACGAGCCUGCCCAAAUACAAGCCCCAGGUGAAU |
| | AGCAGCAUUAACGACUACAUCAGAAAGAAUAAUCUGAAGGCCCCAAAGAUCGAGGAAGAUUACACCAGCUACUUC |
| | CCCAAGUACGCCUAUAGAAACGGCGUGGGCAGACCUGAGGGCAUCGUGGUUCACGACACAGCUAAUGACCGGAGC |
| | ACCAUCAACGGCGAAAUCAGCUACAUGAAGAACAACUACCAGAAUGCCUUCGUGCACGCCUUCGUGGAUGGCGAC |
| | CGGAUCAUCGAGACCGCUCCUACCGACUACCUGAGCUGGGGCGUGGGCGCCGUGGGCAACCCCAGAUUCAUCAAC |
| | GUGGAAAUCGUGCAUACACACGACUACGCCAGCUUCGCUAGAAGCAUGAACAACUACGCCGAUUACGCCGCUACA |
| | CAACUGCAAUACUACGGCCUGAAGCCUGAUAGCGCCGAGUAUGACGGCAACGGCACAGUUUGGACCCACUACGCC |
| | GUGAGCAAGUACCUCGGCGGCACAGACCACGCCGAUCCUCACGGCUACCUGCGGUCCCACAACUACUCUUACGAU |
| | CAGCUGUACGACCUGAUCAACGAAAAGUAUCUUAUCAAAAUGGGCAAGGUGGCACCUUGGGGCACCCAGAGCACC |
| | ACCACACCUACAACACCUUCCAAGCCUACAACCCCUUCUACCCCUUCUAAGCCCUCUAAGCCCAGCACCGGGAAA |
| | CUGACAGUGGCCGCUAACAACGGAGUAGCCCAGAUCAAACCUACCAACAGCGGCCUGUACACCACCGUCUACGAC |
| | AAGACAGGCAAGGCUACCAACGAGGUGCAGAAGACCUUUGCCGUGUCCAAAACAGCAACGCUGGGCAACCAGAAG |
| | UUCUACCUGGUGCAGGACUACAACAGUGGCAACAAGUUCGGCUGGGUGAAGGAAGGAGAUGUGGUGUACAACACC |
| | GCCAAGAGCCCUGUGAAUGUGAACCAGUCUUAUAGCAUCAAGCCCGGCACAAAGCUGUACACCGUUCCUUGGGGA |
| | ACCUCCAAACAGGUGGCCGGCAGUGUGAGUGGCUCCGGCAAUCAGACCUUCAAGGCCAGCAAGCAGCAGCAGAUC |
| | GACAAAUCUAUCUACCUGUAUGGAAGUGUGAACGGGAAGUCCGGCUGGGUAUCCAAGGCAUACCUAGUUGAUACC |
| | GCCAAGCCCACCCCUACCCCGACACCUAAACCUAGCACACCCACAACCAACAACAAGCUGACCGUGUCUUCUCUG |
| | AACGGAGUGGCCCAGAUCAACGCCAAGAACAAUGGCCUGUUCACCACAGUGUACGACAAGACAGGCAAGCCUACC |
| | AAGGAGGUGCAGAAGACCUUCGCCGUGACCAAGGAAGCAAGCCUGGGAGGCAACAAGUUCUACCUCGUGAAGGAC |
| | UACAAUAGCCCUACCCUGAUUGGCUGGGUGAAGCAGGGAGAUGUAAUCUACAACAACGCUAAGAGCCCCGUGAAC |
| | GUGAUGCAGACCUACACCGUGAAGCCUGGCACAAAGCUGUACAGCGUGCCCUGGGGCACAUACAAGCAGGAGGCC |
| | GGCGCCGUGAGCGGAACCGGCAACCAGACCUUUAAGGCCACCAAGCAGCAACAGAUCGACAAGAGUAUAUACCUG |
| | UUCGGCACUGUAAACGGUAAGUCCGGCUGGGUGAGCAAAGCGUACCUGGCCGUUCCUGCUGCCCCUAAAAAGGCC |
| | GUGGCUCAACCGAAAACCGCCGUGAAAGCCUACGCCGUCACCAAACCUCAGACAACACAGACAACUCAAACCGUA |
| | AGCAAGAUCGCCCAGGUGAAGCCCAACAACACCGGAAUUCGGGCCAGCGUGUACGAGAAGACCGCCAAGAACGGC |
| | GCCAAGUACGCUGAUAGAACAUUCUACGUGACCAAGGAACGGGCCCACGGUAACGAGACAUACGUGCUGCUGAAC |
| | AACACCUCCCACAACAUCCCCCUGGGAUGGUUCAACGUGAAAGAUCUGAACGUGCAGAACCUGGGCAAAGAGGUG |
| | AAGACCACACAGAAAUACACCGUGAACAAGAGUAACAACGGCCUCAGCAUGGUGCCUUGGGGCACCAAGAACCAG |
| | GUGAUCCUGACCGGCAACAAUAUCGCCCAAGGCACCUUCAACGCCACCAAGCAGGUGAGCGUGGGCAAGGACGUG |
| | UACCUGUACGGCACAAUCAACAACAGAACCGGAUGGGUCAACGCCAAGGACCUGACCGCCCCUACUGCCGUGAAA |
| | CCAACAACCAGCGCCGCCAAGGACUACAAUUACACCUACGUGAUUAAGAAUGGAAAUGGCUACUACUACGUGACA |
| | CCAAAUAGUGAUACCGCCAAAUAUAGCCUGAAGGCCUUCAACGAGCAGCCAUUCGCGGUGGUGAAGGAGCAGGUG |
| | AUCAACGGACAGACCUGGUACUACGGCAAGCUGAGCAACGGCAAACUGGCCUGGAUCAAGAGCACUGACCUGGCC |
| | AAAGAGCUGAUUAAAUACAACCAGACGGGCAUGACCCUUAACCAAGUGGCCCAGAUCCAGGCCGGACUGCAGUAC |
| | AAGCCUCAGGUGCAGAGAGUGCCAGGGAAGUGGACCGACGCCAACUUCAACGACGUGAAGCACGCUAUGGACACA |
| | AAGAUCAACCAGUUCCUGAAAGGCAAGGGCGUUCUGGAGAACCAAGGCGCCGCCUUCAACAAAGCUGCUCAGAUG |
| | AAGCGCCUGGCCCAGGAUCCUGCUCUGAAGUACCAGUUUCUGAGACUGGACCAGCCUCAGAACAUCUCCAUCGAC |
| | AAGAUCAACCAGUUCCUGAAAGGCAAGGGCGUUCUGGAGAACCAAGGCGCCGCCUUCAACAAAGCUGCUCAGAUG |
| | UACGGCAUCAAUGAGGUGUACCUGAUCAGCCACGCCCUGCUGGAAACCGGCAACGGCACCAGCCAGCUGGCUAAG |
| | GGCGCCGACGUGGUGAACAACAAGGUGGUGACCAACUCUAAUACCAAGUAUCACAACGUCUUUGGAAUCGCCGCC |
| | UAUGACAACGACCCCCUGAGGGAAGGCAUCAAAUACGCCAAGCAGGCCGGCUGGGAUACAGUGAGCAAGGCUAUC |
| | GUGGGGGGCGCUAAGUUCAUCGGAAAUUCUUACGUGAAGGCUGGCCAGAACACCCUGUACAAGAUGCGGUGGAAC |
| | CCUGCUCAUCCUGGCACCCACCAAUACGCCACGGACGUGGACUGGGCCAACAUCAACGCCAAGAUCAUCAAGGGU |
| | UACUACGACAAGAUUGGAGAGGUGGGCAAGUACUUCGACAUCCCUCAGUACAAG (SEQ ID NO: 3) |
| At1, | SASAQPRSVAATPKTSLPKYKPQVNSSINDYIRKNNLKAPKIEEDYTSYFPKYAYRNGVGRPEGIVVHDTANDRS |
| Mat., | TINGEISYMKNNYQNAFVHAFVDGDRIIETAPTDYLSWGVGAVGNPRFINVEIVHTHDYASFARSMNNYADYAAT |
| PP | QLQYYGLKPDSAEYDGNGTVWTHYAVSKYLGGTDHADPHGYLRSHNYSYDQLYDLINEKYLIKMGKVAPWGTQST |
| | TTPTTPSKPTTPSTPSKPSKPSTGKLTVAANNGVAQIKPTNSGLYTTVYDKTGKATNEVQKTFAVSKTATLGNQK |
| | FYLVQDYNSGNKFGWVKEGDVVYNTAKSPVNVNQSYSIKPGTKLYTVPWGTSKQVAGSVSGSGNQTFKASKQQQI |
| | DKSIYLYGSVNGKSGWVSKAYLVDTAKPTPTPTPKPSTPTTNNKLTVSSLNGVAQINAKNNGLFTTVYDKTGKPT |
| | KEVQKTFAVTKEASLGGNKFYLVKDYNSPTLIGWVKQGDVIYNNAKSPVNVMQTYTVKPGTKLYSVPWGTYKQEA |
| | GAVSGTGNQTFKATKQQQIDKSIYLFGTVNGKSGWVSKAYLAVPAAPKKAVAQPKTAVKAYAVTKPQTTQTTQTV |
| | SKIAQVKPNNTGIRASVYEKTAKNGAKYADRTFYVTKERAHGNETYVLLNNTSHNIPLGWFNVKDLNVQNLGKEV |
| | KTTQKYTVNKSNNGLSMVPWGTKNQVILTGNNIAQGTFNATKQVSVGKDVYLYGTINNRTGWVNAKDLTAPTAVK |
| | PTTSAAKDYNYTYVIKNGNGYYYVTPNSDTAKYSLKAFNEQPFAVVKEQVINGQTWYYGKLSNGKLAWIKSTDLA |
| | KELIKYNQTGMTLNQVAQIQAGLQYKPQVQRVPGKWTDANFNDVKHAMDTKRLAQDPALKYQFLRLDQPQNISID |
| | KINQFLKGKGVLENQGAAFNKAAQMYGINEVYLISHALLETGNGTSQLAKGADVVNNKVVTNSNTKYHNVFGIAA |
| | YDNDPLREGIKYAKQAGWDTVSKAIVGGAKFIGNSYVKAGQNTLYKMRWNPAHPGTHQYATDVDWANINAKIIKG |
| | YYDKIGEVGKYFDIPQYK (SEQ ID NO: 72) |
| Atl, | TTCTGCCAGCGCCCAGCCAAGAAGCGTAGCCGCCACCCCTAAGACGAGCCTGCCCAAATACAAGCCCCAGGTGAA |
| Mat., | TAGCAGCATTAACGACTACATCAGAAAGAATAATCTGAAGGCCCCAAAGATCGAGGAAGATTACACCAGCTACTT |
| DNA | CCCCAAGTACGCCTATAGAAACGGCGTGGGCAGACCTGAGGGCATCGTGGTTCACGACACAGCTAATGACCGGAG |
| | CACCATCAACGGCGAAATCAGCTACATGAAGAACAACTACCAGAATGCCTTCGTGCACGCCTTCGTGGATGGCGA |
| | CCGGATCATCGAGACCGCTCCTACCGACTACCTGAGCTGGGGCGTGGGCGCCGTGGGCAACCCCAGATTCATCAA |
| | CGTGGAAATCGTGCATACACACGACTACGCCAGCTTCGCTAGAAGCATGAACAACTACGCCGATTACGCCGCTAC |
| | ACAACTGCAATACTACGGCCTGAAGCCTGATAGCGCCGAGTATGACGGCAACGGCACAGTTTGGACCCACTACGC |
| | CGTGAGCAAGTACCTCGGCGGCACAGACCACGCCGATCCTCACGGCTACCTGCGGTCCCACAACTACTCTTACGA |
| | TCAGCTGTACGACCTGATCAACGAAAAGTATCTTATCAAAATGGGCAAGGTGGCACCTTGGGGCACCCAGAGCAC |
| | CACCACACCTACAACACCTTCCAAGCCTACAACCCCTTCTACCCCTTCTAAGCCCTCTAAGCCCAGCACCGGGAA |
| | ACTGACAGTGGCCGCTAACAACGGAGTAGCCCAGATCAAACCTACCAACAGCGGCCTGTACACCACCGTCTACGA |
| | CAAGACAGGCAAGGCTACCAACGAGGTGCAGAAGACCTTTGCCGTGTCCAAAACAGCAACGCTGGGCAACCAGAA |
| | GTTCTACCTGGTGCAGGACTACAACAGTGGCAACAAGTTCGGCTGGGTGAAGGAAGGAGATGTGGTGTACAACAC |
| | CGCCAAGAGCCCTGTGAATGTGAACCAGTCTTATAGCATCAAGCCCGGCACAAAGCTGTACACCGTTCCTTGGGG |
| | AACCTCCAAACAGGTGGCCGGCAGTGTGAGTGGCTCCGGCAATCAGACCTTCAAGGCCAGCAAGCAGCAGCAGAT |
| | CGACAAATCTATCTACCTGTATGGAAGTGTGAACGGGAAGTCCGGCTGGGTATCCAAGGCATACCTAGTTGATAC |
| | CGCCAAGCCCACCCCTACCCCGACACCTAAACCTAGCACACCCACAACCAACAACAAGCTGACCGTGTCTTCTCT |
| | GAACGGAGTGGCCCAGATCAACGCCAAGAACAATGGCCTGTTCACCACAGTGTACGACAAGACAGGCAAGCCTAC |
| | CAAGGAGGTGCAGAAGACCTTCGCCGTGACCAAGGAAGCAAGCCTGGGAGGCAACAAGTTCTACCTCGTGAAGGA |
| | CTACAATAGCCCTACCCTGATTGGCTGGGTGAAGCAGGGAGATGTAATCTACAACAACGCTAAGAGCCCCGTGAA |
| | CGTGATGCAGACCTACACCGTGAAGCCTGGCACAAAGCTGTACAGCGTGCCCTGGGGCACATACAAGCAGGAGGC |
| | CGGCGCCGTGAGCGGAACCGGCAACCAGACCTTTAAGGCCACCAAGCAGCAACAGATCGACAAGAGTATATACCT |
| | GTTCGGCACTGTAAACGGTAAGTCCGGCTGGGTGAGCAAAGCGTACCTGGCCGTTCCTGCTGCCCCTAAAAAGGC |
| | CGTGGCTCAACCGAAAACCGCCGTGAAAGCCTACGCCGTCACCAAACCTCAGACAACACAGACAACTCAAACCGT |
| | AAGCAAGATCGCCCAGGTGAAGCCCAACAACACCGGAATTCGGGCCAGCGTGTACGAGAAGACCGCCAAGAACGG |
| | CGCCAAGTACGCTGATAGAACATTCTACGTGACCAAGGAACGGGCCCACGGTAACGAGACATACGTGCTGCTGAA |
| | CAACACCTCCCACAACATCCCCCTGGGATGGTTCAACGTGAAAGATCTGAACGTGCAGAACCTGGGCAAAGAGGT |
| | GAAGACCACACAGAAATACACCGTGAACAAGAGTAACAACGGCCTCAGCATGGTGCCTTGGGGCACCAAGAACCA |
| | GGTGATCCTGACCGGCAACAATATCGCCCAAGGCACCTTCAACGCCACCAAGCAGGTGAGCGTGGGCAAGGACGT |
| | GTACCTGTACGGCACAATCAACAACAGAACCGGATGGGTCAACGCCAAGGACCTGACCGCCCCTACTGCCGTGAA |
| | ACCAACAACCAGCGCCGCCAAGGACTACAATTACACCTACGTGATTAAGAATGGAAATGGCTACTACTACGTGAC |
| | ACCAAATAGTGATACCGCCAAATATAGCCTGAAGGCCTTCAACGAGCAGCCATTCGCGGTGGTGAAGGAGCAGGT |
| | GATCAACGGACAGACCTGGTACTACGGCAAGCTGAGCAACGGCAAACTGGCCTGGATCAAGAGCACTGACCTGGC |
| | CAAAGAGCTGATTAAATACAACCAGACGGGCATGACCCTTAACCAAGTGGCCCAGATCCAGGCCGGACTGCAGTA |
| | CAAGCCTCAGGTGCAGAGAGTGCCAGGGAAGTGGACCGACGCCAACTTCAACGACGTGAAGCACGCTATGGACAC |
| | AAAGCGCCTGGCCCAGGATCCTGCTCTGAAGTACCAGTTTCTGAGACTGGACCAGCCTCAGAACATCTCCATCGA |
| | CAAGATCAACCAGTTCCTGAAAGGCAAGGGCGTTCTGGAGAACCAAGGCGCCGCCTTCAACAAAGCTGCTCAGAT |
| | GTACGGCATCAATGAGGTGTACCTGATCAGCCACGCCCTGCTGGAAACCGGCAACGGCACCAGCCAGCTGGCTAA |
| | GGGCGCCGACGTGGTGAACAACAAGGTGGTGACCAACTCTAATACCAAGTATCACAACGTCTTTGGAATCGCCGC |
| | CTATGACAACGACCCCCTGAGGGAAGGCATCAAATACGCCAAGCAGGCCGGCTGGGATACAGTGAGCAAGGCTAT |
| | CGTGGGGGGCGCTAAGTTCATCGGAAATTCTTACGTGAAGGCTGGCCAGAACACCCTGTACAAGATGCGGTGGAA |
| | CCCTGCTCATCCTGGCACCCACCAATACGCCACGGACGTGGACTGGGCCAACATCAACGCCAAGATCATCAAGGG |
| | TTACTACGACAAGATTGGAGAGGTGGGCAAGTACTTCGACATCCCTCAGTACAAG (SEQ ID NO: 73) |
| At1, | UUCUGCCAGCGCCCAGCCAAGAAGCGUAGCCGCCACCCCUAAGACGAGCCUGCCCAAAUACAAGCCCCAGGUGAA |
| Mat., | UAGCAGCAUUAACGACUACAUCAGAAAGAAUAAUCUGAAGGCCCCAAAGAUCGAGGAAGAUUACACCAGCUACUU |
| RNA | CCCCAAGUACGCCUAUAGAAACGGCGUGGGCAGACCUGAGGGCAUCGUGGUUCACGACACAGCUAAUGACCGGAG |
| | CACCAUCAACGGCGAAAUCAGCUACAUGAAGAACAACUACCAGAAUGCCUUCGUGCACGCCUUCGUGGAUGGCGA |
| | CCGGAUCAUCGAGACCGCUCCUACCGACUACCUGAGCUGGGGCGUGGGCGCCGUGGGCAACCCCAGAUUCAUCAA |
| | CGUGGAAAUCGUGCAUACACACGACUACGCCAGCUUCGCUAGAAGCAUGAACAACUACGCCGAUUACGCCGCUAC |
| | ACAACUGCAAUACUACGGCCUGAAGCCUGAUAGCGCCGAGUAUGACGGCAACGGCACAGUUUGGACCCACUACGC |
| | CGUGAGCAAGUACCUCGGCGGCACAGACCACGCCGAUCCUCACGGCUACCUGCGGUCCCACAACUACUCUUACGA |
| | UCAGCUGUACGACCUGAUCAACGAAAAGUAUCUUAUCAAAAUGGGCAAGGUGGCACCUUGGGGCACCCAGAGCAC |
| | CACCACACCUACAACACCUUCCAAGCCUACAACCCCUUCUACCCCUUCUAAGCCCUCUAAGCCCAGCACCGGGAA |
| | ACUGACAGUGGCCGCUAACAACGGAGUAGCCCAGAUCAAACCUACCAACAGCGGCCUGUACACCACCGUCUACGA |
| | CAAGACAGGCAAGGCUACCAACGAGGUGCAGAAGACCUUUGCCGUGUCCAAAACAGCAACGCUGGGCAACCAGAA |
| | GUUCUACCUGGUGCAGGACUACAACAGUGGCAACAAGUUCGGCUGGGUGAAGGAAGGAGAUGUGGUGUACAACAC |
| | CGCCAAGAGCCCUGUGAAUGUGAACCAGUCUUAUAGCAUCAAGCCCGGCACAAAGCUGUACACCGUUCCUUGGGG |
| | AACCUCCAAACAGGUGGCCGGCAGUGUGAGUGGCUCCGGCAAUCAGACCUUCAAGGCCAGCAAGCAGCAGCAGAU |
| | CGACAAAUCUAUCUACCUGUAUGGAAGUGUGAACGGGAAGUCCGGCUGGGUAUCCAAGGCAUACCUAGUUGAUAC |
| | CGCCAAGCCCACCCCUACCCCGACACCUAAACCUAGCACACCCACAACCAACAACAAGCUGACCGUGUCUUCUCU |
| | GAACGGAGUGGCCCAGAUCAACGCCAAGAACAAUGGCCUGUUCACCACAGUGUACGACAAGACAGGCAAGCCUAC |
| | CAAGGAGGUGCAGAAGACCUUCGCCGUGACCAAGGAAGCAAGCCUGGGAGGCAACAAGUUCUACCUCGUGAAGGA |
| | CUACAAUAGCCCUACCCUGAUUGGCUGGGUGAAGCAGGGAGAUGUAAUCUACAACAACGCUAAGAGCCCCGUGAA |
| | CGUGAUGCAGACCUACACCGUGAAGCCUGGCACAAAGCUGUACAGCGUGCCCUGGGGCACAUACAAGCAGGAGGC |
| | CGGCGCCGUGAGCGGAACCGGCAACCAGACCUUUAAGGCCACCAAGCAGCAACAGAUCGACAAGAGUAUAUACCU |
| | GUUCGGCACUGUAAACGGUAAGUCCGGCUGGGUGAGCAAAGCGUACCUGGCCGUUCCUGCUGCCCCUAAAAAGGC |
| | CGUGGCUCAACCGAAAACCGCCGUGAAAGCCUACGCCGUCACCAAACCUCAGACAACACAGACAACUCAAACCGU |
| | AAGCAAGAUCGCCCAGGUGAAGCCCAACAACACCGGAAUUCGGGCCAGCGUGUACGAGAAGACCGCCAAGAACGG |
| | CGCCAAGUACGCUGAUAGAACAUUCUACGUGACCAAGGAACGGGCCCACGGUAACGAGACAUACGUGCUGCUGAA |
| | CAACACCUCCCACAACAUCCCCCUGGGAUGGUUCAACGUGAAAGAUCUGAACGUGCAGAACCUGGGCAAAGAGGU |
| | GAAGACCACACAGAAAUACACCGUGAACAAGAGUAACAACGGCCUCAGCAUGGUGCCUUGGGGCACCAAGAACCA |
| | GGUGAUCCUGACCGGCAACAAUAUCGCCCAAGGCACCUUCAACGCCACCAAGCAGGUGAGCGUGGGCAAGGACGU |
| | GUACCUGUACGGCACAAUCAACAACAGAACCGGAUGGGUCAACGCCAAGGACCUGACCGCCCCUACUGCCGUGAA |
| | ACCAACAACCAGCGCCGCCAAGGACUACAAUUACACCUACGUGAUUAAGAAUGGAAAUGGCUACUACUACGUGAC |
| | ACCAAAUAGUGAUACCGCCAAAUAUAGCCUGAAGGCCUUCAACGAGCAGCCAUUCGCGGUGGUGAAGGAGCAGGU |
| | GAUCAACGGACAGACCUGGUACUACGGCAAGCUGAGCAACGGCAAACUGGCCUGGAUCAAGAGCACUGACCUGGC |
| | CAAAGAGCUGAUUAAAUACAACCAGACGGGCAUGACCCUUAACCAAGUGGCCCAGAUCCAGGCCGGACUGCAGUA |
| | CAAGCCUCAGGUGCAGAGAGUGCCAGGGAAGUGGACCGACGCCAACUUCAACGACGUGAAGCACGCUAUGGACAC |
| | AAAGCGCCUGGCCCAGGAUCCUGCUCUGAAGUACCAGUUUCUGAGACUGGACCAGCCUCAGAACAUCUCCAUCGA |
| | CAAGAUCAACCAGUUCCUGAAAGGCAAGGGCGUUCUGGAGAACCAAGGCGCCGCCUUCAACAAAGCUGCUCAGAU |
| | GUACGGCAUCAAUGAGGUGUACCUGAUCAGCCACGCCCUGCUGGAAACCGGCAACGGCACCAGCCAGCUGGCUAA |
| | GGGCGCCGACGUGGUGAACAACAAGGUGGUGACCAACUCUAAUACCAAGUAUCACAACGUCUUUGGAAUCGCCGC |
| | CUAUGACAACGACCCCCUGAGGGAAGGCAUCAAAUACGCCAAGCAGGCCGGCUGGGAUACAGUGAGCAAGGCUAU |
| | CGUGGGGGGCGCUAAGUUCAUCGGAAAUUCUUACGUGAAGGCUGGCCAGAACACCCUGUACAAGAUGCGGUGGAA |
| | CCCUGCUCAUCCUGGCACCCACCAAUACGCCACGGACGUGGACUGGGCCAACAUCAACGCCAAGAUCAUCAAGGG |
| | UUACUACGACAAGAUUGGAGAGGUGGGCAAGUACUUCGACAUCCCUCAGUACAAG (SEQ ID NO: 74) |
| Amd, | MSASAQPRSVAATPKTSLPKYKPQVNSSINDYIRKNNLKAPKIEEDYTSYFPKYAYRNGVGRPEGIVVHDTANDR |
| FL, | STINGEISYMKNNYQNAFVHAFVDGDRIIETAPTDYLSWGVGAVGNPRFINVEIVHTHDYASFARSMNNYADYAA |
| PP | TQLQYYGLKPDSAEYDGNGTVWTHYAVSKYLGGTDHADPHGYLRSHNYSYDQLYDLINEKYLIKMGKVAPWGTQS |
| | TTTPTTPSKPTTPSTPSKPSKPSTGKLTVAANNGVAQIKPTNSGLYTTVYDKTGKATNEVQKTFAVSKTATLGNQ |
| | KFYLVQDYNSGNKFGWVKEGDVVYNTAKSPVNVNQSYSIKPGTKLYTVPWGTSKQVAGSVSGSGNQTFKASKQQQ |
| | IDKSIYLYGSVNGKSGWVSKAYLVDTAKPTPTPTPKPSTPTTNNKLTVSSLNGVAQINAKNNGLFTTVYDKTGKP |
| | TKEVQKTFAVTKEASLGGNKFYLVKDYNSPTLIGWVKQGDVIYNNAKSPVNVMQTYTVKPGTKLYSVPWGTYKQE |
| | AGAVSGTGNQTFKATKQQQIDKSIYLFGTVNGKSGWVSKAYLAVPAAPKKAVAQPKTAVKAYAVTK (SEQ ID |
| | NO: 4) |
| Amd, | ATGTCTGCCAGCGCCCAGCCAAGAAGCGTAGCCGCCACCCCTAAGACGAGCCTGCCCAAATACAAGCCCCAGGTG |
| FL, | AATAGCAGCATTAACGACTACATCAGAAAGAATAATCTGAAGGCCCCAAAGATCGAGGAAGATTACACCAGCTAC |
| DNA | TTCCCCAAGTACGCCTATAGAAACGGCGTGGGCAGACCTGAGGGCATCGTGGTTCACGACACAGCTAATGACCGG |
| | AGCACCATCAACGGCGAAATCAGCTACATGAAGAACAACTACCAGAATGCCTTCGTGCACGCCTTCGTGGATGGC |
| | GACCGGATCATCGAGACCGCTCCTACCGACTACCTGAGCTGGGGCGTGGGCGCCGTGGGCAACCCCAGATTCATC |
| | AACGTGGAAATCGTGCATACACACGACTACGCCAGCTTCGCTAGAAGCATGAACAACTACGCCGATTACGCCGCT |
| | ACACAACTGCAATACTACGGCCTGAAGCCTGATAGCGCCGAGTATGACGGCAACGGCACAGTTTGGACCCACTAC |
| | GCCGTGAGCAAGTACCTCGGCGGCACAGACCACGCCGATCCTCACGGCTACCTGCGGTCCCACAACTACTCTTAC |
| | GATCAGCTGTACGACCTGATCAACGAAAAGTATCTTATCAAAATGGGCAAGGTGGCACCTTGGGGCACCCAGAGC |
| | ACCACCACACCTACAACACCTTCCAAGCCTACAACCCCTTCTACCCCTTCTAAGCCCTCTAAGCCCAGCACCGGG |
| | AAACTGACAGTGGCCGCTAACAACGGAGTAGCCCAGATCAAACCTACCAACAGCGGCCTGTACACCACCGTCTAC |
| | GACAAGACAGGCAAGGCTACCAACGAGGTGCAGAAGACCTTTGCCGTGTCCAAAACAGCAACGCTGGGCAACCAG |
| | AAGTTCTACCTGGTGCAGGACTACAACAGTGGCAACAAGTTCGGCTGGGTGAAGGAAGGAGATGTGGTGTACAAC |
| | ACCGCCAAGAGCCCTGTGAATGTGAACCAGTCTTATAGCATCAAGCCCGGCACAAAGCTGTACACCGTTCCTTGG |
| | GGAACCTCCAAACAGGTGGCCGGCAGTGTGAGTGGCTCCGGCAATCAGACCTTCAAGGCCAGCAAGCAGCAGCAG |
| | ATCGACAAATCTATCTACCTGTATGGAAGTGTGAACGGGAAGTCCGGCTGGGTATCCAAGGCATACCTAGTTGAT |
| | ACCGCCAAGCCCACCCCTACCCCGACACCTAAACCTAGCACACCCACAACCAACAACAAGCTGACCGTGTCTTCT |
| | CTGAACGGAGTGGCCCAGATCAACGCCAAGAACAATGGCCTGTTCACCACAGTGTACGACAAGACAGGCAAGCCT |
| | ACCAAGGAGGTGCAGAAGACCTTCGCCGTGACCAAGGAAGCAAGCCTGGGAGGCAACAAGTTCTACCTCGTGAAG |
| | GACTACAATAGCCCTACCCTGATTGGCTGGGTGAAGCAGGGAGATGTAATCTACAACAACGCTAAGAGCCCCGTG |
| | AACGTGATGCAGACCTACACCGTGAAGCCTGGCACAAAGCTGTACAGCGTGCCCTGGGGCACATACAAGCAGGAG |
| | GCCGGCGCCGTGAGCGGAACCGGCAACCAGACCTTTAAGGCCACCAAGCAGCAACAGATCGACAAGAGTATATAC |
| | CTGTTCGGCACTGTAAACGGTAAGTCCGGCTGGGTGAGCAAAGCGTACCTGGCCGTTCCTGCTGCCCCTAAAAAG |
| | GCCGTGGCTCAACCGAAAACCGCCGTGAAAGCCTACGCCGTCACCAAATGA (SEQ ID NO: 5) |
| Amd, | AUGUCUGCCAGCGCCCAGCCAAGAAGCGUAGCCGCCACCCCUAAGACGAGCCUGCCCAAAUACAAGCCCCAGGUG |
| FL, | AAUAGCAGCAUUAACGACUACAUCAGAAAGAAUAAUCUGAAGGCCCCAAAGAUCGAGGAAGAUUACACCAGCUAC |
| RNA | UUCCCCAAGUACGCCUAUAGAAACGGCGUGGGCAGACCUGAGGGCAUCGUGGUUCACGACACAGCUAAUGACCGG |
| | AGCACCAUCAACGGCGAAAUCAGCUACAUGAAGAACAACUACCAGAAUGCCUUCGUGCACGCCUUCGUGGAUGGC |
| | GACCGGAUCAUCGAGACCGCUCCUACCGACUACCUGAGCUGGGGCGUGGGCGCCGUGGGCAACCCCAGAUUCAUC |
| | AACGUGGAAAUCGUGCAUACACACGACUACGCCAGCUUCGCUAGAAGCAUGAACAACUACGCCGAUUACGCCGCU |
| | ACACAACUGCAAUACUACGGCCUGAAGCCUGAUAGCGCCGAGUAUGACGGCAACGGCACAGUUUGGACCCACUAC |
| | GCCGUGAGCAAGUACCUCGGCGGCACAGACCACGCCGAUCCUCACGGCUACCUGCGGUCCCACAACUACUCUUAC |
| | GAUCAGCUGUACGACCUGAUCAACGAAAAGUAUCUUAUCAAAAUGGGCAAGGUGGCACCUUGGGGCACCCAGAGC |
| | ACCACCACACCUACAACACCUUCCAAGCCUACAACCCCUUCUACCCCUUCUAAGCCCUCUAAGCCCAGCACCGGG |
| | AAACUGACAGUGGCCGCUAACAACGGAGUAGCCCAGAUCAAACCUACCAACAGCGGCCUGUACACCACCGUCUAC |
| | GACAAGACAGGCAAGGCUACCAACGAGGUGCAGAAGACCUUUGCCGUGUCCAAAACAGCAACGCUGGGCAACCAG |
| | AAGUUCUACCUGGUGCAGGACUACAACAGUGGCAACAAGUUCGGCUGGGUGAAGGAAGGAGAUGUGGUGUACAAC |
| | ACCGCCAAGAGCCCUGUGAAUGUGAACCAGUCUUAUAGCAUCAAGCCCGGCACAAAGCUGUACACCGUUCCUUGG |
| | GGAACCUCCAAACAGGUGGCCGGCAGUGUGAGUGGCUCCGGCAAUCAGACCUUCAAGGCCAGCAAGCAGCAGCAG |
| | AUCGACAAAUCUAUCUACCUGUAUGGAAGUGUGAACGGGAAGUCCGGCUGGGUAUCCAAGGCAUACCUAGUUGAU |
| | ACCGCCAAGCCCACCCCUACCCCGACACCUAAACCUAGCACACCCACAACCAACAACAAGCUGACCGUGUCUUCU |
| | CUGAACGGAGUGGCCCAGAUCAACGCCAAGAACAAUGGCCUGUUCACCACAGUGUACGACAAGACAGGCAAGCCU |
| | ACCAAGGAGGUGCAGAAGACCUUCGCCGUGACCAAGGAAGCAAGCCUGGGAGGCAACAAGUUCUACCUCGUGAAG |
| | GACUACAAUAGCCCUACCCUGAUUGGCUGGGUGAAGCAGGGAGAUGUAAUCUACAACAACGCUAAGAGCCCCGUG |
| | AACGUGAUGCAGACCUACACCGUGAAGCCUGGCACAAAGCUGUACAGCGUGCCCUGGGGCACAUACAAGCAGGAG |
| | GCCGGCGCCGUGAGCGGAACCGGCAACCAGACCUUUAAGGCCACCAAGCAGCAACAGAUCGACAAGAGUAUAUAC |
| | CUGUUCGGCACUGUAAACGGUAAGUCCGGCUGGGUGAGCAAAGCGUACCUGGCCGUUCCUGCUGCCCCUAAAAAG |
| | GCCGUGGCUCAACCGAAAACCGCCGUGAAAGCCUACGCCGUCACCAAAUGA (SEQ ID NO: 6) |
| Amd, | TTTPTTPSKPTTPSTPSKPSKPSTGKLTVAANNGVAQIKPTNSGLYTTVYDKTGKATNEVQKTFAVSKTATLGNQ |
| R1 | KFYLVQDYNSGNKFGWVKEGDVVYNTAKSPVNVNQSYSIKPGTKLYTVPWGTSKQVAGSVSGSGNQTFKASKQQQ |
| Dom., | IDKSIYLYGSVNGKSGWVSKAYLVD (SEQ ID NO: 7) |
| PP | |
| Amd, | ACCACCACACCTACAACACCTTCCAAGCCTACAACCCCTTCTACCCCTTCTAAGCCCTCTAAGCCCAGCACCGGG |
| R1 | AAACTGACAGTGGCCGCTAACAACGGAGTAGCCCAGATCAAACCTACCAACAGCGGCCTGTACACCACCGTCTAC |
| Dom., | GACAAGACAGGCAAGGCTACCAACGAGGTGCAGAAGACCTTTGCCGTGTCCAAAACAGCAACGCTGGGCAACCAG |
| DNA | AAGTTCTACCTGGTGCAGGACTACAACAGTGGCAACAAGTTCGGCTGGGTGAAGGAAGGAGATGTGGTGTACAAC |
| | ACCGCCAAGAGCCCTGTGAATGTGAACCAGTCTTATAGCATCAAGCCCGGCACAAAGCTGTACACCGTTCCTTGG |
| | GGAACCTCCAAACAGGTGGCCGGCAGTGTGAGTGGCTCCGGCAATCAGACCTTCAAGGCCAGCAAGCAGCAGCAG |
| | ATCGACAAATCTATCTACCTGTATGGAAGTGTGAACGGGAAGTCCGGCTGGGTATCCAAGGCATACCTAGTTGAT |
| | (SEQ ID NO: 8) |
| Amd, | ACCACCACACCUACAACACCUUCCAAGCCUACAACCCCUUCUACCCCUUCUAAGCCCUCUAAGCCCAGCACCGGG |
| R1 | AAACUGACAGUGGCCGCUAACAACGGAGUAGCCCAGAUCAAACCUACCAACAGCGGCCUGUACACCACCGUCUAC |
| Dom., | GACAAGACAGGCAAGGCUACCAACGAGGUGCAGAAGACCUUUGCCGUGUCCAAAACAGCAACGCUGGGCAACCAG |
| RNA | AAGUUCUACCUGGUGCAGGACUACAACAGUGGCAACAAGUUCGGCUGGGUGAAGGAAGGAGAUGUGGUGUACAAC |
| | ACCGCCAAGAGCCCUGUGAAUGUGAACCAGUCUUAUAGCAUCAAGCCCGGCACAAAGCUGUACACCGUUCCUUGG |
| | GGAACCUCCAAACAGGUGGCCGGCAGUGUGAGUGGCUCCGGCAAUCAGACCUUCAAGGCCAGCAAGCAGCAGCAG |
| | AUCGACAAAUCUAUCUACCUGUAUGGAAGUGUGAACGGGAAGUCCGGCUGGGUAUCCAAGGCAUACCUAGUUGAU |
| | (SEQ ID NO: 9) |
| Amd | TAKPTPTPTPKPSTPTTNNKLTVSSLNGVAQINAKNNGLFTTVYDKTGKPTKEVQKTFAVTKEASLGGNKFYLVK |
| R2 | DYNSPTLIGWVKQGDVIYNNAKSPVNVMQTYTVKPGTKLYSVPWGTYKQEAGAVSGTGNQTFKATKQQQIDKSIY |
| Dom. | LFGTVNGKSGWVSKAYLAVPAAPKKAVAQPKTAVKAYAVTKQTT (SEQ ID NO: 10) |
| PP | |
| Amd, | ACCGCCAAGCCCACCCCTACCCCGACACCTAAACCTAGCACACCCACAACCAACAACAAGCTGACCGTGTCTTCT |
| R2 | CTGAACGGAGTGGCCCAGATCAACGCCAAGAACAATGGCCTGTTCACCACAGTGTACGACAAGACAGGCAAGCCT |
| Dom., | ACCAAGGAGGTGCAGAAGACCTTCGCCGTGACCAAGGAAGCAAGCCTGGGAGGCAACAAGTTCTACCTCGTGAAG |
| DNA | GACTACAATAGCCCTACCCTGATTGGCTGGGTGAAGCAGGGAGATGTAATCTACAACAACGCTAAGAGCCCCGTG |
| | AACGTGATGCAGACCTACACCGTGAAGCCTGGCACAAAGCTGTACAGCGTGCCCTGGGGCACATACAAGCAGGAG |
| | GCCGGCGCCGTGAGCGGAACCGGCAACCAGACCTTTAAGGCCACCAAGCAGCAACAGATCGACAAGAGTATATAC |
| | CTGTTCGGCACTGTAAACGGTAAGTCCGGCTGGGTGAGCAAAGCGTACCTGGCCGTTCCTGCT (SEQ ID |
| | NO: 11) |
| Amd, | ACCGCCAAGCCCACCCCUACCCCGACACCUAAACCUAGCACACCCACAACCAACAACAAGCUGACCGUGUCUUCU |
| R2 | CUGAACGGAGUGGCCCAGAUCAACGCCAAGAACAAUGGCCUGUUCACCACAGUGUACGACAAGACAGGCAAGCCU |
| Dom., | ACCAAGGAGGUGCAGAAGACCUUCGCCGUGACCAAGGAAGCAAGCCUGGGAGGCAACAAGUUCUACCUCGUGAAG |
| RNA | GACUACAAUAGCCCUACCCUGAUUGGCUGGGUGAAGCAGGGAGAUGUAAUCUACAACAACGCUAAGAGCCCCGUG |
| | AACGUGAUGCAGACCUACACCGUGAAGCCUGGCACAAAGCUGUACAGCGUGCCCUGGGGCACAUACAAGCAGGAG |
| | GCCGGCGCCGUGAGCGGAACCGGCAACCAGACCUUUAAGGCCACCAAGCAGCAACAGAUCGACAAGAGUAUAUAC |
| | CUGUUCGGCACUGUAAACGGUAAGUCCGGCUGGGUGAGCAAAGCGUACCUGGCCGUUCCUGCU (SEQ ID |
| | NO: 12) |
| Amd, | TTPTTPSKPTTPSTPSKPSKPSTGKLTVAANNGVAQIKPTNSGLYTTVYDKTGKATNEVQKTFAVSKTATLGNQK |
| R1 +R2 | FYLVQDYNSGNKFGWVKEGDVVYNTAKSPVNVNQSYSIKPGTKLYTVPWGTSKQVAGSVSGSGNQTFKASKQQQI |
| Dom., | DKSIYLYGSVNGKSGWVSKAYLVDTAKPTPTPTPKPSTPTTNNKLTVSSLNGVAQINAKNNGLFTTVYDKTGKPT |
| PP | KEVQKTFAVTKEASLGGNKFYLVKDYNSPTLIGWVKQGDVIYNNAKSPVNVMQTYTVKPGTKLYSVPWGTYKQEA |
| | GAVSGTGNQTFKATKQQQIDKSIYLFGTVNGKSGWVSKAYLAVPAAPKKAVAQPKTAVKAYAVTK (SEQ ID |
| | NO: 13) |
| Amd, | ACCACCACACCTACAACACCTTCCAAGCCTACAACCCCTTCTACCCCTTCTAAGCCCTCTAAGCCCAGCACCGGG |
| R1 +R2 | AAACTGACAGTGGCCGCTAACAACGGAGTAGCCCAGATCAAACCTACCAACAGCGGCCTGTACACCACCGTCTAC |
| Dom., | GACAAGACAGGCAAGGCTACCAACGAGGTGCAGAAGACCTTTGCCGTGTCCAAAACAGCAACGCTGGGCAACCAG |
| DNA | AAGTTCTACCTGGTGCAGGACTACAACAGTGGCAACAAGTTCGGCTGGGTGAAGGAAGGAGATGTGGTGTACAAC |
| | ACCGCCAAGAGCCCTGTGAATGTGAACCAGTCTTATAGCATCAAGCCCGGCACAAAGCTGTACACCGTTCCTTGG |
| | GGAACCTCCAAACAGGTGGCCGGCAGTGTGAGTGGCTCCGGCAATCAGACCTTCAAGGCCAGCAAGCAGCAGCAG |
| | ATCGACAAATCTATCTACCTGTATGGAAGTGTGAACGGGAAGTCCGGCTGGGTATCCAAGGCATACCTAGTTGAT |
| | ACCGCCAAGCCCACCCCTACCCCGACACCTAAACCTAGCACACCCACAACCAACAACAAGCTGACCGTGTCTTCT |
| | CTGAACGGAGTGGCCCAGATCAACGCCAAGAACAATGGCCTGTTCACCACAGTGTACGACAAGACAGGCAAGCCT |
| | ACCAAGGAGGTGCAGAAGACCTTCGCCGTGACCAAGGAAGCAAGCCTGGGAGGCAACAAGTTCTACCTCGTGAAG |
| | GACTACAATAGCCCTACCCTGATTGGCTGGGTGAAGCAGGGAGATGTAATCTACAACAACGCTAAGAGCCCCGTG |
| | AACGTGATGCAGACCTACACCGTGAAGCCTGGCACAAAGCTGTACAGCGTGCCCTGGGGCACATACAAGCAGGAG |
| | GCCGGCGCCGTGAGCGGAACCGGCAACCAGACCTTTAAGGCCACCAAGCAGCAACAGATCGACAAGAGTATATAC |
| | CTGTTCGGCACTGTAAACGGTAAGTCCGGCTGGGTGAGCAAAGCGTACCTGGCCGTTCCTGCT (SEQ ID |
| | NO: 14) |
| Amd, | ACCACCACACCUACAACACCUUCCAAGCCUACAACCCCUUCUACCCCUUCUAAGCCCUCUAAGCCCAGCACCGGG |
| R1 +R2 | AAACUGACAGUGGCCGCUAACAACGGAGUAGCCCAGAUCAAACCUACCAACAGCGGCCUGUACACCACCGUCUAC |
| Dom., | GACAAGACAGGCAAGGCUACCAACGAGGUGCAGAAGACCUUUGCCGUGUCCAAAACAGCAACGCUGGGCAACCAG |
| RNA | AAGUUCUACCUGGUGCAGGACUACAACAGUGGCAACAAGUUCGGCUGGGUGAAGGAAGGAGAUGUGGUGUACAAC |
| | ACCGCCAAGAGCCCUGUGAAUGUGAACCAGUCUUAUAGCAUCAAGCCCGGCACAAAGCUGUACACCGUUCCUUGG |
| | GGAACCUCCAAACAGGUGGCCGGCAGUGUGAGUGGCUCCGGCAAUCAGACCUUCAAGGCCAGCAAGCAGCAGCAG |
| | AUCGACAAAUCUAUCUACCUGUAUGGAAGUGUGAACGGGAAGUCCGGCUGGGUAUCCAAGGCAUACCUAGUUGAU |
| | ACCGCCAAGCCCACCCCUACCCCGACACCUAAACCUAGCACACCCACAACCAACAACAAGCUGACCGUGUCUUCU |
| | CUGAACGGAGUGGCCCAGAUCAACGCCAAGAACAAUGGCCUGUUCACCACAGUGUACGACAAGACAGGCAAGCCU |
| | ACCAAGGAGGUGCAGAAGACCUUCGCCGUGACCAAGGAAGCAAGCCUGGGAGGCAACAAGUUCUACCUCGUGAAG |
| | GACUACAAUAGCCCUACCCUGAUUGGCUGGGUGAAGCAGGGAGAUGUAAUCUACAACAACGCUAAGAGCCCCGUG |
| | AACGUGAUGCAGACCUACACCGUGAAGCCUGGCACAAAGCUGUACAGCGUGCCCUGGGGCACAUACAAGCAGGAG |
| | GCCGGCGCCGUGAGCGGAACCGGCAACCAGACCUUUAAGGCCACCAAGCAGCAACAGAUCGACAAGAGUAUAUAC |
| | CUGUUCGGCACUGUAAACGGUAAGUCCGGCUGGGUGAGCAAAGCGUACCUGGCCGUUCCUGCU (SEQ ID |
| | NO: 15) |
| Amd, | MSASAQPRSVAATPKTSLPKYKPQVNSSINDYIRKNNLKAPKIEEDYTSYFPKYAYRNGVGRPEGIVVHDTANDR |
| Cat. | STINGEISYMKNNYQNAFVHAFVDGDRIIETAPTDYLSWGVGAVGNPRFINVEIVHTHDYASFARSMNNYADYAA |
| Dom., | TQLQYYGLKPDSAEYDGNGTVWTHYAVSKYLGGTDHADPHGYLRSHNYSYDQLYDLINEKYLIKMGKVAPWGTQ |
| PP | (SEQ ID NO: 16) |
| Amd, | ATGTCTGCCAGCGCCCAGCCAAGAAGCGTAGCCGCCACCCCTAAGACGAGCCTGCCCAAATACAAGCCCCAGGTG |
| Cat. | AATAGCAGCATTAACGACTACATCAGAAAGAATAATCTGAAGGCCCCAAAGATCGAGGAAGATTACACCAGCTAC |
| Dom., | TTCCCCAAGTACGCCTATAGAAACGGCGTGGGCAGACCTGAGGGCATCGTGGTTCACGACACAGCTAATGACCGG |
| DNA | AGCACCATCAACGGCGAAATCAGCTACATGAAGAACAACTACCAGAATGCCTTCGTGCACGCCTTCGTGGATGGC |
| | GACCGGATCATCGAGACCGCTCCTACCGACTACCTGAGCTGGGGCGTGGGCGCCGTGGGCAACCCCAGATTCATC |
| | AACGTGGAAATCGTGCATACACACGACTACGCCAGCTTCGCTAGAAGCATGAACAACTACGCCGATTACGCCGCT |
| | ACACAACTGCAATACTACGGCCTGAAGCCTGATAGCGCCGAGTATGACGGCAACGGCACAGTTTGGACCCACTAC |
| | GCCGTGAGCAAGTACCTCGGCGGCACAGACCACGCCGATCCTCACGGCTACCTGCGGTCCCACAACTACTCTTAC |
| | GATCAGCTGTACGACCTGATCAACGAAAAGTATCTTATCAAAATGGGCAAGGTGGCACCTTGGGGCACCCAGA |
| | (SEQ ID NO: 17) |
| Amd, | AUGUCUGCCAGCGCCCAGCCAAGAAGCGUAGCCGCCACCCCUAAGACGAGCCUGCCCAAAUACAAGCCCCAGGUG |
| Cat. | AAUAGCAGCAUUAACGACUACAUCAGAAAGAAUAAUCUGAAGGCCCCAAAGAUCGAGGAAGAUUACACCAGCUAC |
| Dom., | UUCCCCAAGUACGCCUAUAGAAACGGCGUGGGCAGACCUGAGGGCAUCGUGGUUCACGACACAGCUAAUGACCGG |
| RNA | AGCACCAUCAACGGCGAAAUCAGCUACAUGAAGAACAACUACCAGAAUGCCUUCGUGCACGCCUUCGUGGAUGGC |
| | GACCGGAUCAUCGAGACCGCUCCUACCGACUACCUGAGCUGGGGCGUGGGCGCCGUGGGCAACCCCAGAUUCAUC |
| | AACGUGGAAAUCGUGCAUACACACGACUACGCCAGCUUCGCUAGAAGCAUGAACAACUACGCCGAUUACGCCGCU |
| | ACACAACUGCAAUACUACGGCCUGAAGCCUGAUAGCGCCGAGUAUGACGGCAACGGCACAGUUUGGACCCACUAC |
| | GCCGUGAGCAAGUACCUCGGCGGCACAGACCACGCCGAUCCUCACGGCUACCUGCGGUCCCACAACUACUCUUAC |
| | GAUCAGCUGUACGACCUGAUCAACGAAAAGUAUCUUAUCAAAAUGGGCAAGGUGGCACCUUGGGGCACCCAGA |
| | (SEQ ID NO: 18) |
| Gmd, | MAVTKPQTTQTTQTVSKIAQVKPNNTGIRASVYEKTAKNGAKYADRTFYVTKERAHGNETYVLLNNTSHNIPLGW |
| FL, | FNVKDLNVQNLGKEVKTTQKYTVNKSNNGLSMVPWGTKNQVILTGNNIAQGTFNATKQVSVGKDVYLYGTINNRT |
| PP | GWVNAKDLTAPTAVKPTTSAAKDYNYTYVIKNGNGYYYVTPNSDTAKYSLKAFNEQPFAVVKEQVINGQTWYYGK |
| | LSNGKLAWIKSTDLAKELIKYNQTGMTLNQVAQIQAGLQYKPQVQRVPGKWTDANFNDVKHAMDTKRLAQDPALK |
| | YQFLRLDQPQNISIDKINQFLKGKGVLENQGAAFNKAAQMYGINEVYLISHALLETGNGTSQLAKGADVVNNKVV |
| | TNSNTKYHNVFGIAAYDNDPLREGIKYAKQAGWDTVSKAIVGGAKFIGNSYVKAGQNTLYKMRWNPAHPGTHQYA |
| | TDVDWANINAKIIKGYYDKIGEVGKYFDIPQYK (SEQ ID NO: 19) |
| Gmd, | ATGGCCGTCACCAAACCTCAGACAACACAGACAACTCAAACCGTAAGCAAGATCGCCCAGGTGAAGCCCAACAAC |
| FL, | ACCGGAATTCGGGCCAGCGTGTACGAGAAGACCGCCAAGAACGGCGCCAAGTACGCTGATAGAACATTCTACGTG |
| DNA | ACCAAGGAACGGGCCCACGGTAACGAGACATACGTGCTGCTGAACAACACCTCCCACAACATCCCCCTGGGATGG |
| | TTCAACGTGAAAGATCTGAACGTGCAGAACCTGGGCAAAGAGGTGAAGACCACACAGAAATACACCGTGAACAAG |
| | AGTAACAACGGCCTCAGCATGGTGCCTTGGGGCACCAAGAACCAGGTGATCCTGACCGGCAACAATATCGCCCAA |
| | GGCACCTTCAACGCCACCAAGCAGGTGAGCGTGGGCAAGGACGTGTACCTGTACGGCACAATCAACAACAGAACC |
| | GGATGGGTCAACGCCAAGGACCTGACCGCCCCTACTGCCGTGAAACCAACAACCAGCGCCGCCAAGGACTACAAT |
| | TACACCTACGTGATTAAGAATGGAAATGGCTACTACTACGTGACACCAAATAGTGATACCGCCAAATATAGCCTG |
| | AAGGCCTTCAACGAGCAGCCATTCGCGGTGGTGAAGGAGCAGGTGATCAACGGACAGACCTGGTACTACGGCAAG |
| | CTGAGCAACGGCAAACTGGCCTGGATCAAGAGCACTGACCTGGCCAAAGAGCTGATTAAATACAACCAGACGGGC |
| | ATGACCCTTAACCAAGTGGCCCAGATCCAGGCCGGACTGCAGTACAAGCCTCAGGTGCAGAGAGTGCCAGGGAAG |
| | TGGACCGACGCCAACTTCAACGACGTGAAGCACGCTATGGACACAAAGCGCCTGGCCCAGGATCCTGCTCTGAAG |
| | TACCAGTTTCTGAGACTGGACCAGCCTCAGAACATCTCCATCGACAAGATCAACCAGTTCCTGAAAGGCAAGGGC |
| | GTTCTGGAGAACCAAGGCGCCGCCTTCAACAAAGCTGCTCAGATGTACGGCATCAATGAGGTGTACCTGATCAGC |
| | CACGCCCTGCTGGAAACCGGCAACGGCACCAGCCAGCTGGCTAAGGGCGCCGACGTGGTGAACAACAAGGTGGTG |
| | ACCAACTCTAATACCAAGTATCACAACGTCTTTGGAATCGCCGCCTATGACAACGACCCCCTGAGGGAAGGCATC |
| | AAATACGCCAAGCAGGCCGGCTGGGATACAGTGAGCAAGGCTATCGTGGGGGGCGCTAAGTTCATCGGAAATTCT |
| | TACGTGAAGGCTGGCCAGAACACCCTGTACAAGATGCGGTGGAACCCTGCTCATCCTGGCACCCACCAATACGCC |
| | ACGGACGTGGACTGGGCCAACATCAACGCCAAGATCATCAAGGGTTACTACGACAAGATTGGAGAGGTGGGCAAG |
| | TACTTCGACATCCCTCAGTACAAGTGA (SEQ ID NO: 20) |
| Gmd, | AUGGCCGUCACCAAACCUCAGACAACACAGACAACUCAAACCGUAAGCAAGAUCGCCCAGGUGAAGCCCAACAAC |
| FL, | ACCGGAAUUCGGGCCAGCGUGUACGAGAAGACCGCCAAGAACGGCGCCAAGUACGCUGAUAGAACAUUCUACGUG |
| RNA | ACCAAGGAACGGGCCCACGGUAACGAGACAUACGUGCUGCUGAACAACACCUCCCACAACAUCCCCCUGGGAUGG |
| | UUCAACGUGAAAGAUCUGAACGUGCAGAACCUGGGCAAAGAGGUGAAGACCACACAGAAAUACACCGUGAACAAG |
| | AGUAACAACGGCCUCAGCAUGGUGCCUUGGGGCACCAAGAACCAGGUGAUCCUGACCGGCAACAAUAUCGCCCAA |
| | GGCACCUUCAACGCCACCAAGCAGGUGAGCGUGGGCAAGGACGUGUACCUGUACGGCACAAUCAACAACAGAACC |
| | GGAUGGGUCAACGCCAAGGACCUGACCGCCCCUACUGCCGUGAAACCAACAACCAGCGCCGCCAAGGACUACAAU |
| | UACACCUACGUGAUUAAGAAUGGAAAUGGCUACUACUACGUGACACCAAAUAGUGAUACCGCCAAAUAUAGCCUG |
| | AAGGCCUUCAACGAGCAGCCAUUCGCGGUGGUGAAGGAGCAGGUGAUCAACGGACAGACCUGGUACUACGGCAAG |
| | CUGAGCAACGGCAAACUGGCCUGGAUCAAGAGCACUGACCUGGCCAAAGAGCUGAUUAAAUACAACCAGACGGGC |
| | AUGACCCUUAACCAAGUGGCCCAGAUCCAGGCCGGACUGCAGUACAAGCCUCAGGUGCAGAGAGUGCCAGGGAAG |
| | UGGACCGACGCCAACUUCAACGACGUGAAGCACGCUAUGGACACAAAGCGCCUGGCCCAGGAUCCUGCUCUGAAG |
| | UACCAGUUUCUGAGACUGGACCAGCCUCAGAACAUCUCCAUCGACAAGAUCAACCAGUUCCUGAAAGGCAAGGGC |
| | GUUCUGGAGAACCAAGGCGCCGCCUUCAACAAAGCUGCUCAGAUGUACGGCAUCAAUGAGGUGUACCUGAUCAGC |
| | CACGCCCUGCUGGAAACCGGCAACGGCACCAGCCAGCUGGCUAAGGGCGCCGACGUGGUGAACAACAAGGUGGUG |
| | ACCAACUCUAAUACCAAGUAUCACAACGUCUUUGGAAUCGCCGCCUAUGACAACGACCCCCUGAGGGAAGGCAUC |
| | AAAUACGCCAAGCAGGCCGGCUGGGAUACAGUGAGCAAGGCUAUCGUGGGGGGCGCUAAGUUCAUCGGAAAUUCU |
| | UACGUGAAGGCUGGCCAGAACACCCUGUACAAGAUGCGGUGGAACCCUGCUCAUCCUGGCACCCACCAAUACGCC |
| | ACGGACGUGGACUGGGCCAACAUCAACGCCAAGAUCAUCAAGGGUUACUACGACAAGAUUGGAGAGGUGGGCAAG |
| | UACUUCGACAUCCCUCAGUACAAGUGA (SEQ ID NO: 21) |
| Gmd, | QTTQTVSKIAQVKPNNTGIRASVYEKTAKNGAKYADRTFYVTKERAHGNETYVLLNNTSHNIPLGWFNVKDLNVQ |
| R3 | NLGKEVKTTQKYTVNKSNNGLSMVPWGTKNQVILTGNNIAQGTFNATKOVSVGKDVYLYGTI (SEQ ID |
| Dom., | NO: 22) |
| PP | |
| Gmd, | CAGACAACACAGACAACTCAAACCGTAAGCAAGATCGCCCAGGTGAAGCCCAACAACACCGGAATTCGGGCCAGC |
| R3 | GTGTACGAGAAGACCGCCAAGAACGGCGCCAAGTACGCTGATAGAACATTCTACGTGACCAAGGAACGGGCCCAC |
| Dom., | GGTAACGAGACATACGTGCTGCTGAACAACACCTCCCACAACATCCCCCTGGGATGGTTCAACGTGAAAGATCTG |
| DNA | AACGTGCAGAACCTGGGCAAAGAGGTGAAGACCACACAGAAATACACCGTGAACAAGAGTAACAACGGCCTCAGC |
| | ATGGTGCCTTGGGGCACCAAGAACCAGGTGATCCTGACCGGCAACAATATCGCCCAAGGCACCTTCAACGCCACC |
| | AAGCAGGTGAGCGTGGGCAAGGACGTGTACCTGTACGGCACAATC (SEQ ID NO: 23) |
| Gmd, | CAGACAACACAGACAACUCAAACCGUAAGCAAGAUCGCCCAGGUGAAGCCCAACAACACCGGAAUUCGGGCCAGC |
| R3 | GUGUACGAGAAGACCGCCAAGAACGGCGCCAAGUACGCUGAUAGAACAUUCUACGUGACCAAGGAACGGGCCCAC |
| Dom., | GGUAACGAGACAUACGUGCUGCUGAACAACACCUCCCACAACAUCCCCCUGGGAUGGUUCAACGUGAAAGAUCUG |
| RNA | AACGUGCAGAACCUGGGCAAAGAGGUGAAGACCACACAGAAAUACACCGUGAACAAGAGUAACAACGGCCUCAGC |
| | AUGGUGCCUUGGGGCACCAAGAACCAGGUGAUCCUGACCGGCAACAAUAUCGCCCAAGGCACCUUCAACGCCACC |
| | AAGCAGGUGAGCGUGGGCAAGGACGUGUACCUGUACGGCACAAUC (SEQ ID NO: 24) |
| Gmd, | NNRTGWVNAKDLTAPTAVKPTTSAAKDYNYTYVIKNGNGYYYVTPNSDTAKYSLKAFNEQPFAVVKEQVINGQTW |
| Cat. | YYGKLSNGKLAWIKSTDLAKELIKYNQTGMTLNQVAQIQAGLQYKPQVQRVPGKWTDANFNDVKHAMDTKRLAQD |
| Dom., | PALKYQFLRLDQPQNISIDKINQFLKGKGVLENQGAAFNKAAQMYGINEVYLISHALLETGNGTSQLAKGADVVN |
| PP | NKVVTNSNTKYHNVFGIAAYDNDPLREGIKYAKQAGWDTVSKAIVGGAKFIGNSYVKAGQNTLYKMRWNPAHPGT |
| | HQYATDVDWANINAKIIKGYYDKIGEVGKYFDIPQYK (SEQ ID NO: 25) |
| Gmd, | AACAACAGAACCGGATGGGTCAACGCCAAGGACCTGACCGCCCCTACTGCCGTGAAACCAACAACCAGCGCCGCC |
| Cat. | AAGGACTACAATTACACCTACGTGATTAAGAATGGAAATGGCTACTACTACGTGACACCAAATAGTGATACCGCC |
| Dom., | AAATATAGCCTGAAGGCCTTCAACGAGCAGCCATTCGCGGTGGTGAAGGAGCAGGTGATCAACGGACAGACCTGG |
| DNA | TACTACGGCAAGCTGAGCAACGGCAAACTGGCCTGGATCAAGAGCACTGACCTGGCCAAAGAGCTGATTAAATAC |
| | AACCAGACGGGCATGACCCTTAACCAAGTGGCCCAGATCCAGGCCGGACTGCAGTACAAGCCTCAGGTGCAGAGA |
| | GTGCCAGGGAAGTGGACCGACGCCAACTTCAACGACGTGAAGCACGCTATGGACACAAAGCGCCTGGCCCAGGAT |
| | CCTGCTCTGAAGTACCAGTTTCTGAGACTGGACCAGCCTCAGAACATCTCCATCGACAAGATCAACCAGTTCCTG |
| | AAAGGCAAGGGCGTTCTGGAGAACCAAGGCGCCGCCTTCAACAAAGCTGCTCAGATGTACGGCATCAATGAGGTG |
| | TACCTGATCAGCCACGCCCTGCTGGAAACCGGCAACGGCACCAGCCAGCTGGCTAAGGGCGCCGACGTGGTGAAC |
| | AACAAGGTGGTGACCAACTCTAATACCAAGTATCACAACGTCTTTGGAATCGCCGCCTATGACAACGACCCCCTG |
| | AGGGAAGGCATCAAATACGCCAAGCAGGCCGGCTGGGATACAGTGAGCAAGGCTATCGTGGGGGGCGCTAAGTTC |
| | ATCGGAAATTCTTACGTGAAGGCTGGCCAGAACACCCTGTACAAGATGCGGTGGAACCCTGCTCATCCTGGCACC |
| | CACCAATACGCCACGGACGTGGACTGGGCCAACATCAACGCCAAGATCATCAAGGGTTACTACGACAAGATTGGA |
| | GAGGTGGGCAAGTACTTCGACATCCCTCAGTACAAG (SEQ ID NO: 26) |
| Gmd, | AACAACAGAACCGGAUGGGUCAACGCCAAGGACCUGACCGCCCCUACUGCCGUGAAACCAACAACCAGCGCCGCC |
| Cat. | AAGGACUACAAUUACACCUACGUGAUUAAGAAUGGAAAUGGCUACUACUACGUGACACCAAAUAGUGAUACCGCC |
| Dom., | AAAUAUAGCCUGAAGGCCUUCAACGAGCAGCCAUUCGCGGUGGUGAAGGAGCAGGUGAUCAACGGACAGACCUGG |
| RNA | UACUACGGCAAGCUGAGCAACGGCAAACUGGCCUGGAUCAAGAGCACUGACCUGGCCAAAGAGCUGAUUAAAUAC |
| | AACCAGACGGGCAUGACCCUUAACCAAGUGGCCCAGAUCCAGGCCGGACUGCAGUACAAGCCUCAGGUGCAGAGA |
| | GUGCCAGGGAAGUGGACCGACGCCAACUUCAACGACGUGAAGCACGCUAUGGACACAAAGCGCCUGGCCCAGGAU |
| | CCUGCUCUGAAGUACCAGUUUCUGAGACUGGACCAGCCUCAGAACAUCUCCAUCGACAAGAUCAACCAGUUCCUG |
| | AAAGGCAAGGGCGUUCUGGAGAACCAAGGCGCCGCCUUCAACAAAGCUGCUCAGAUGUACGGCAUCAAUGAGGUG |
| | UACCUGAUCAGCCACGCCCUGCUGGAAACCGGCAACGGCACCAGCCAGCUGGCUAAGGGCGCCGACGUGGUGAAC |
| | AACAAGGUGGUGACCAACUCUAAUACCAAGUAUCACAACGUCUUUGGAAUCGCCGCCUAUGACAACGACCCCCUG |
| | AGGGAAGGCAUCAAAUACGCCAAGCAGGCCGGCUGGGAUACAGUGAGCAAGGCUAUCGUGGGGGGCGCUAAGUUC |
| | AUCGGAAAUUCUUACGUGAAGGCUGGCCAGAACACCCUGUACAAGAUGCGGUGGAACCCUGCUCAUCCUGGCACC |
| | CACCAAUACGCCACGGACGUGGACUGGGCCAACAUCAACGCCAAGAUCAUCAAGGGUUACUACGACAAGAUUGGA |
| | GAGGUGGGCAAGUACUUCGACAUCCCUCAGUACAAG (SEQ ID NO: 27) |
| Gmd, | NIPLGWFNVKDLNVQNLGKEVKTTQKYTVNK (SEQ ID NO: 28) |
| Ant., | |
| PP | |
| Gmd, | AACATCCCCCTGGGATGGTTCAACGTGAAAGATCTGAACGTGCAGAACCTGGGCAAAGAGGTGAAGACCACACAG |
| Ant., | AAATACACCGTGAACAAG (SEQ ID NO: 29) |
| DNA | |
| Gmd, | AACAUCCCCCUGGGAUGGUUCAACGUGAAAGAUCUGAACGUGCAGAACCUGGGCAAAGAGGUGAAGACCACACAG |
| Ant., | AAAUACACCGUGAACAAG (SEQ ID NO: 30) |
| RNA | |
| CHIPS, | MLPSSEMKKKLATTDPVLALSFLTAGLSTHHPHSAKAFFTFEPFPTNEEIESNKKLLEKEKKDDERIDKAYKESF |
| FL, | KNSGLPTTLGKLDERLRNYLKKGTKGSAKQAEKMVILTENKGYYTFYLNTPLAEDRKNIELLGKMYKTYFFKKGE |
| PP | SKSSYVINGPGKTNEYAY (SEQ ID NO: 31) |
| CHIPS, | ATGCTCCCCAGCAGCGAGATGAAGAAAAAACTGGCTACAACCGATCCTGTGCTGGCCCTGAGCTTCCTGACCGCC |
| FL, | GGCCTGTCTACACACCACCCCCACAGCGCCAAGGCCTTCTTCACCTTTGAGCCTTTCCCAACAAATGAGGAAATC |
| DNA | GAGAGCAACAAGAAGCTGCTGGAAAAAGAAAAGAAGGACGACGAGCGGATCGACAAGGCTTATAAGGAATCTTTT |
| | AAGAACAGCGGCCTGCCTACCACCCTGGGAAAACTGGACGAAAGACTGAGAAACTACCTGAAGAAGGGAACAAAG |
| | GGCTCCGCCAAGCAGGCCGAGAAGATGGTGATCCTGACAGAGAACAAGGGCTACTACACCTTCTACCTGAACACC |
| | CCTCTGGCCGAAGATAGAAAGAATATCGAGCTGCTGGGCAAGATGTACAAGACCTACTTCTTCAAGAAAGGCGAG |
| | AGCAAGTCCAGCTACGTGATCAACGGCCCTGGCAAAACCAACGAGTACGCCTACTGA (SEQ ID NO: 32) |
| CHIPS, | AUGCUCCCCAGCAGCGAGAUGAAGAAAAAACUGGCUACAACCGAUCCUGUGCUGGCCCUGAGCUUCCUGACCGCC |
| FL, | GGCCUGUCUACACACCACCCCCACAGCGCCAAGGCCUUCUUCACCUUUGAGCCUUUCCCAACAAAUGAGGAAAUC |
| RNA | GAGAGCAACAAGAAGCUGCUGGAAAAAGAAAAGAAGGACGACGAGCGGAUCGACAAGGCUUAUAAGGAAUCUUUU |
| | AAGAACAGCGGCCUGCCUACCACCCUGGGAAAACUGGACGAAAGACUGAGAAACUACCUGAAGAAGGGAACAAAG |
| | GGCUCCGCCAAGCAGGCCGAGAAGAUGGUGAUCCUGACAGAGAACAAGGGCUACUACACCUUCUACCUGAACACC |
| | CCUCUGGCCGAAGAUAGAAAGAAUAUCGAGCUGCUGGGCAAGAUGUACAAGACCUACUUCUUCAAGAAAGGCGAG |
| | AGCAAGUCCAGCUACGUGAUCAACGGCCCUGGCAAAACCAACGAGUACGCCUACUGA (SEQ ID NO: 33) |
| CHIPS, | FTFEPFPTNEEIESNKKLLEKEKKDDERIDKAYKESFKNSGLPTTLGKLDERLRNYLKKGTKGSAKQAEKMVILT |
| Mat., | ENKGYYTFYLNTPLAEDRKNIELLGKMYKTYFFKKGESKSSYVINGPGKTNEYAY (SEQ ID NO: 34) |
| PP | |
| CHIPS, | TTCACCTTTGAGCCTTTCCCAACAAATGAGGAAATCGAGAGCAACAAGAAGCTGCTGGAAAAAGAAAAGAAGGAC |
| Mat., | GACGAGCGGATCGACAAGGCTTATAAGGAATCTTTTAAGAACAGCGGCCTGCCTACCACCCTGGGAAAACTGGAC |
| DNA | GAAAGACTGAGAAACTACCTGAAGAAGGGAACAAAGGGCTCCGCCAAGCAGGCCGAGAAGATGGTGATCCTGACA |
| | GAGAACAAGGGCTACTACACCTTCTACCTGAACACCCCTCTGGCCGAAGATAGAAAGAATATCGAGCTGCTGGGC |
| | AAGATGTACAAGACCTACTTCTTCAAGAAAGGCGAGAGCAAGTCCAGCTACGTGATCAACGGCCCTGGCAAAACC |
| | AACGAGTACGCCTACTGA (SEQ ID NO: 35) |
| CHIPS, | UUCACCUUUGAGCCUUUCCCAACAAAUGAGGAAAUCGAGAGCAACAAGAAGCUGCUGGAAAAAGAAAAGAAGGAC |
| Mat., | GACGAGCGGAUCGACAAGGCUUAUAAGGAAUCUUUUAAGAACAGCGGCCUGCCUACCACCCUGGGAAAACUGGAC |
| RNA | GAAAGACUGAGAAACUACCUGAAGAAGGGAACAAAGGGCUCCGCCAAGCAGGCCGAGAAGAUGGUGAUCCUGACA |
| | GAGAACAAGGGCUACUACACCUUCUACCUGAACACCCCUCUGGCCGAAGAUAGAAAGAAUAUCGAGCUGCUGGGC |
| | AAGAUGUACAAGACCUACUUCUUCAAGAAAGGCGAGAGCAAGUCCAGCUACGUGAUCAACGGCCCUGGCAAAACC |
| | AACGAGUACGCCUACUGA (SEQ ID NO: 36) |
| CHIPS | FTFEPFPTNEEIESNKKMLEKEKAYKESFKNSGLPTTLGKLDERLRNYLKKGTKNSAQFEKMVILTENKGYYTVY |
| 2, | LNTPLAEDRKNVELLGKMYKTYFFKKGESKSSYVINGPGKTNEYAY (SEQ ID NO: 86) |
| Mat., | |
| PP | |
| CHIPS | TTCACCTTTGAGCCTTTCCCCACCAATGAGGAAATCGAGAGCAACAAAAAGATGCTGGAAAAAGAGAAGGCCTAT |
| 2, | AAGGAATCTTTTAAGAATAGCGGCCTGCCTACAACACTGGGCAAGCTGGACGAGCGGCTGAGAAACTACCTCAAG |
| Mat., | AAAGGAACCAAGAACAGCGCCCAGTTCGAGAAGATGGTCATCCTGACCGAGAACAAGGGCTACTACACCGTGTAC |
| DNA | CTGAACACCCCTCTGGCTGAAGATAGAAAGAACGTGGAACTGCTGGGAAAAATGTACAAGACCTACTTCTTCAAG |
| | AAGGGCGAGAGCAAGTCCAGCTACGTGATCAACGGCCCAGGCAAGACAAACGAGTACGCCTACTGA (SEQ ID |
| | NO: 87) |
| CHIPS | UUCACCUUUGAGCCUUUCCCCACCAAUGAGGAAAUCGAGAGCAACAAAAAGAUGCUGGAAAAAGAGAAGGCCUAU |
| 2, | AAGGAAUCUUUUAAGAAUAGCGGCCUGCCUACAACACUGGGCAAGCUGGACGAGCGGCUGAGAAACUACCUCAAG |
| Mat., | AAAGGAACCAAGAACAGCGCCCAGUUCGAGAAGAUGGUCAUCCUGACCGAGAACAAGGGCUACUACACCGUGUAC |
| RNA | CUGAACACCCCUCUGGCUGAAGAUAGAAAGAACGUGGAACUGCUGGGAAAAAUGUACAAGACCUACUUCUUCAAG |
| | AAGGGCGAGAGCAAGUCCAGCUACGUGAUCAACGGCCCAGGCAAGACAAACGAGUACGCCUACUGA (SEQ ID |
| | NO: 88) |
| CHIPS, | LRNYLKKGTKGSAKQAEKMVILTENKGYYTFYLNTPLAEDRKNIELLGKMYKTYFFKKGESK (SEQ ID |
| Ant1, | NO: 37) |
| PP | |
| CHIPS, | CUGAGAAACUACCUGAAGAAGGGAACAAAGGGCUCCGCCAAGCAGGCCGAGAAGAUGGUGAUCCUGACAGAGAAC |
| Ant1, | AAGGGCUACUACACCUUCUACCUGAACACCCCUCUGGCCGAAGAUAGAAAGAAUAUCGAGCUGCUGGGCAAGAUG |
| RNA | UACAAGACCUACUUCUUCAAGAAAGGCGAGAGCAAG ((SEQ ID NO: 38) |
| CHIPS, | CTGAGAAACTACCTGAAGAAGGGAACAAAGGGCTCCGCCAAGCAGGCCGAGAAGATGGTGATCCTGACAGAGAAC |
| Ant1, | AAGGGCTACTACACCTTCTACCTGAACACCCCTCTGGCCGAAGATAGAAAGAATATCGAGCTGCTGGGCAAGATG |
| DNA | TACAAGACCTACTTCTTCAAGAAAGGCGAGAGCAAG (SEQ ID NO: 39) |
| CHIPS, | LRNYLKKGTKNSAQFEKMVILTENKGYYTVYLNTPLAEDRKNVELLGKMYKTYFFKKGESK (SEQ ID |
| Ant2, | NO: 89) |
| PP | |
| CHIPS, | CTGAGAAACTACCTCAAGAAAGGAACCAAGAACAGCGCCCAGTTCGAGAAGATGGTCATCCTGACCGAGAACAAG |
| Ant2, | GGCTACTACACCGTGTACCTGAACACCCCTCTGGCTGAAGATAGAAAGAACGTGGAACTGCTGGGAAAAATGTAC |
| DNA | AAGACCTACTTCTTCAAGAAGGGCGAGAGCAAG (SEQ ID NO: 90) |
| CHIPS, | CUGAGAAACUACCUCAAGAAAGGAACCAAGAACAGCGCCCAGUUCGAGAAGAUGGUCAUCCUGACCGAGAACAAG |
| Ant2, | GGCUACUACACCGUGUACCUGAACACCCCUCUGGCUGAAGAUAGAAAGAACGUGGAACUGCUGGGAAAAAUGUAC |
| RNA | AAGACCUACUUCUUCAAGAAGGGCGAGAGCAAG (SEQ ID NO: 119) |
| SCIN, | MKIRKSILAGTLAIVLASPLVTNLDKNEAQASASTSLPTSNEYQNEKLANELKSLLDELNVNELATGSLNTYYKR |
| FL, | TIKISGQKAMYALKSKDFKKMSEAKYQLQKIYNEIDEALKSKY (SEQ ID NO: 40) |
| PP | |
| SCIN, | ATGAAAATTAGAAAGAGCATCCTGGCCGGAACCCTGGCCATCGTGCTGGCCAGCCCCCTGGTGACCAACCTGGAT |
| FL, | AAGAACGAGGCCCAGGCCAGCGCCTCCACCAGCCTGCCTACAAGCAACGAGTACCAGAACGAGAAGCTGGCTAAT |
| DNA | GAACTGAAGTCTCTGCTGGACGAGCTGAACGTGAACGAACTCGCTACAGGCAGCCTGAATACCTACTACAAGCGG |
| | ACCATCAAGATCAGCGGCCAGAAGGCCATGTACGCCCTGAAGTCCAAGGACTTCAAGAAAATGAGCGAGGCTAAG |
| | TACCAACTGCAGAAAATCTACAACGAAATCGACGAGGCCCTGAAATCTAAGTATTGA (SEQ ID |
| | NO: 41) |
| SCIN, | AUGAAAAUUAGAAAGAGCAUCCUGGCCGGAACCCUGGCCAUCGUGCUGGCCAGCCCCCUGGUGACCAACCUGGAU |
| FL, | AAGAACGAGGCCCAGGCCAGCGCCUCCACCAGCCUGCCUACAAGCAACGAGUACCAGAACGAGAAGCUGGCUAAU |
| RNA | GAACUGAAGUCUCUGCUGGACGAGCUGAACGUGAACGAACUCGCUACAGGCAGCCUGAAUACCUACUACAAGCGG |
| | ACCAUCAAGAUCAGCGGCCAGAAGGCCAUGUACGCCCUGAAGUCCAAGGACUUCAAGAAAAUGAGCGAGGCUAAG |
| | UACCAACUGCAGAAAAUCUACAACGAAAUCGACGAGGCCCUGAAAUCUAAGUAUUGA (SEQ ID |
| | NO: 42) |
| SCIN, | STSLPTSNEYQNEKLANELKSLLDELNVNELATGSLNTYYKRTIKISGQKAMYALKSKDFKKMSEAKYQLQKIYN |
| Mat., | EIDEALKSKY (SEQ ID NO: 43) |
| PP | |
| SCIN, | TCCACCAGCCTGCCTACAAGCAACGAGTACCAGAACGAGAAGCTGGCTAATGAACTGAAGTCTCTGCTGGACGAG |
| Mat., | CTGAACGTGAACGAACTCGCTACAGGCAGCCTGAATACCTACTACAAGCGGACCATCAAGATCAGCGGCCAGAAG |
| DNA | GCCATGTACGCCCTGAAGTCCAAGGACTTCAAGAAAATGAGCGAGGCTAAGTACCAACTGCAGAAAATCTACAAC |
| | GAAATCGACGAGGCCCTGAAATCTAAGTATTGA (SEQ ID NO: 75) |
| SCIN, | UCCACCAGCCUGCCUACAAGCAACGAGUACCAGAACGAGAAGCUGGCUAAUGAACUGAAGUCUCUGCUGGACGAG |
| Mat., | CUGAACGUGAACGAACUCGCUACAGGCAGCCUGAAUACCUACUACAAGCGGACCAUCAAGAUCAGCGGCCAGAAG |
| RNA | GCCAUGUACGCCCUGAAGUCCAAGGACUUCAAGAAAAUGAGCGAGGCUAAGUACCAACUGCAGAAAAUCUACAAC |
| | GAAAUCGACGAGGCCCUGAAAUCUAAGUAUUGA (SEQ ID NO: 44) |
| SCIN | MSTSLPTSNEYQNEKLANELKSLLDELNVNELATGSLNTYYKRTIKISGQKAMYALKSKDFKKMSEAKYQLQKIY |
| 2, | NEIDEALKSKY (SEQ ID NO: 120) |
| Mat., | |
| PP | |
| SCIN | ATGTCTACAAGCCTGCCTACCAGCAACGAGTACCAGAACGAGAAGCTGGCTAATGAACTCAAGTCCCTGCTGGAT |
| 2, | GAGCTGAACGTGAACGAACTGGCCACAGGCAGCCTGAATACCTACTACAAGAGAACCATCAAGATCAGCGGCCAG |
| Mat., | AAGGCCATGTACGCCCTGAAGAGCAAGGACTTCAAGAAAATGAGCGAGGCCAAGTACCAACTGCAGAAAATCTAC |
| DNA | AACGAAATCGACGAGGCCCTGAAATCTAAGTATTGA (SEQ ID NO: 121) |
| SCIN | AUGUCUACAAGCCUGCCUACCAGCAACGAGUACCAGAACGAGAAGCUGGCUAAUGAACUCAAGUCCCUGCUGGAU |
| 2, | GAGCUGAACGUGAACGAACUGGCCACAGGCAGCCUGAAUACCUACUACAAGAGAACCAUCAAGAUCAGCGGCCAG |
| Mat., | AAGGCCAUGUACGCCCUGAAGAGCAAGGACUUCAAGAAAAUGAGCGAGGCCAAGUACCAACUGCAGAAAAUCUAC |
| RNA | AACGAAAUCGACGAGGCCCUGAAAUCUAAGUAUUGA (SEQ ID NO: 122) |
| SCIN, | STSLPTSNEYQNEKLANELKSLLDELNVNELATGSLNTYYKRTIKISGQK (SEQ ID NO: 45) |
| Ant1, | |
| PP | |
| SCIN, | TCCACCAGCCTGCCTACAAGCAACGAGTACCAGAACGAGAAGCTGGCTAATGAACTGAAGTCTCTGCTGGACGAG |
| Ant1, | CTGAACGTGAACGAACTCGCTACAGGCAGCCTGAATACCTACTACAAGCGGACCATCAAGATCAGCGGCCAGAAG |
| DNA | (SEQ ID NO: 46) |
| SCIN, | UCCACCAGCCUGCCUACAAGCAACGAGUACCAGAACGAGAAGCUGGCUAAUGAACUGAAGUCUCUGCUGGACGAG |
| Ant1, | CUGAACGUGAACGAACUCGCUACAGGCAGCCUGAAUACCUACUACAAGCGGACCAUCAAGAUCAGCGGCCAGAAG |
| RNA | (SEQ ID NO: 47) |
| SCIN, | LATGSLNTYYKRTIKISG (SEQ ID NO: 48) |
| Ant2, | |
| PP | |
| SCIN, | CTCGCTACAGGCAGCCTGAATACCTACTACAAGCGGACCATCAAGATCAGCGGC (SEQ ID NO: 49) |
| Ant2, | |
| DNA | |
| SCIN, | CUCGCUACAGGCAGCCUGAAUACCUACUACAAGCGGACCAUCAAGAUCAGCGGC (SEQ ID NO: 50) |
| Ant2, | |
| RNA | |
| Hla, | MKTRIVSSVTTTLLLGSILMNPVANAADSDINIKTGTTDKIGSNTTVKTGDLVTYDKENGMHKKVFYSFIDDKNH |
| FL, | NKKLLVIRTKGTIAGQYRVYSEEGANKSGLAWPSAFKVQLQLPDNEVAQISDYYPRNSIDTKEYMSTLTYGFNGN |
| PP | VTGDDTGKIGGLIGANVSIGHTLKYVQPDFKTILESPTTDKKVGWKVIFNNMVNQNWGPYDRDSWNPVYGNQLFM |
| | KTRNGSMKAADNFLDPNKASSLLSSGFSPDFATVITMDRKASKQQTNIDVIYERVRDDYQLHWTSTNWKGTNTKD |
| | KWTDRSSERYKIDWEKEEMTN (SEQ ID NO: 51) |
| Hla, | ATGAAAACAAGAATCGTGTCCAGCGTGACGACCACACTGCTGCTCGGCAGCATCCTGATGAACCCCGTGGCCAAT |
| FL, | GCCGCTGATAGCGATATCAACATCAAGACCGGCACCACAGATAAGATCGGCTCTAATACCACCGTGAAGACCGGC |
| DNA | GACCTGGTGACCTACGACAAGGAGAACGGCATGCACAAGAAGGTGTTCTACAGCTTCATCGACGATAAGAACCAC |
| | AACAAGAAACTGCTGGTCATCAGAACAAAAGGCACCATCGCCGGCCAGTACAGAGTGTATTCTGAAGAGGGCGCC |
| | AACAAGAGCGGACTGGCCTGGCCTTCCGCCTTTAAGGTGCAGCTGCAACTGCCTGATAACGAGGTGGCCCAGATT |
| | AGCGACTACTACCCCAGAAACAGCATCGACACCAAGGAATACATGAGCACCCTGACCTACGGCTTCAACGGAAAT |
| | GTGACAGGAGATGATACAGGCAAGATTGGCGGCCTGATCGGCGCTAACGTGTCCATCGGACACACACTGAAGTAT |
| | GTGCAACCTGACTTCAAGACCATCCTGGAATCTCCCACCACAGACAAGAAGGTCGGATGGAAGGTGATCTTCAAC |
| | AACATGGTGAACCAGAACTGGGGCCCTTACGACAGGGACAGCTGGAACCCTGTGTACGGCAATCAGCTGTTCATG |
| | AAGACCCGCAACGGTAGCATGAAGGCCGCCGATAACTTCCTGGACCCAAACAAAGCCAGCAGCCTGCTGAGCTCC |
| | GGCTTCAGCCCTGATTTTGCCACAGTTATCACCATGGACAGAAAGGCTTCTAAACAGCAGACCAACATCGACGTG |
| | ATCTACGAGAGAGTGCGGGACGACTACCAGCTGCATTGGACAAGCACCAATTGGAAAGGCACAAACACCAAGGAC |
| | AAATGGACCGACCGGAGCTCTGAGCGGTACAAGATCGACTGGGAGAAGGAGGAAATGACCAATTGA (SEQ |
| | ID NO: 52) |
| Hla, | AUGAAAACAAGAAUCGUGUCCAGCGUGACGACCACACUGCUGCUCGGCAGCAUCCUGAUGAACCCCGUGGCCAAU |
| FL, | GCCGCUGAUAGCGAUAUCAACAUCAAGACCGGCACCACAGAUAAGAUCGGCUCUAAUACCACCGUGAAGACCGGC |
| RNA | GACCUGGUGACCUACGACAAGGAGAACGGCAUGCACAAGAAGGUGUUCUACAGCUUCAUCGACGAUAAGAACCAC |
| | AACAAGAAACUGCUGGUCAUCAGAACAAAAGGCACCAUCGCCGGCCAGUACAGAGUGUAUUCUGAAGAGGGCGCC |
| | AACAAGAGCGGACUGGCCUGGCCUUCCGCCUUUAAGGUGCAGCUGCAACUGCCUGAUAACGAGGUGGCCCAGAUU |
| | AGCGACUACUACCCCAGAAACAGCAUCGACACCAAGGAAUACAUGAGCACCCUGACCUACGGCUUCAACGGAAAU |
| | GUGACAGGAGAUGAUACAGGCAAGAUUGGCGGCCUGAUCGGCGCUAACGUGUCCAUCGGACACACACUGAAGUAU |
| | GUGCAACCUGACUUCAAGACCAUCCUGGAAUCUCCCACCACAGACAAGAAGGUCGGAUGGAAGGUGAUCUUCAAC |
| | AACAUGGUGAACCAGAACUGGGGCCCUUACGACAGGGACAGCUGGAACCCUGUGUACGGCAAUCAGCUGUUCAUG |
| | AAGACCCGCAACGGUAGCAUGAAGGCCGCCGAUAACUUCCUGGACCCAAACAAAGCCAGCAGCCUGCUGAGCUCC |
| | GGCUUCAGCCCUGAUUUUGCCACAGUUAUCACCAUGGACAGAAAGGCUUCUAAACAGCAGACCAACAUCGACGUG |
| | AUCUACGAGAGAGUGCGGGACGACUACCAGCUGCAUUGGACAAGCACCAAUUGGAAAGGCACAAACACCAAGGAC |
| | AAAUGGACCGACCGGAGCUCUGAGCGGUACAAGAUCGACUGGGAGAAGGAGGAAAUGACCAAUUGA (SEQ |
| | ID NO: 53) |
| Hla | ADSDINIKTGTTDKIGSNTTVKTGDLVTYDKENGMHKKVFYSFIDDKNHNKKLLVIRTKGTIAGQYRVYSEEGAN |
| Mat., | KSGLAWPSAFKVQLQLPDNEVAQISDYYPRNSIDTKEYMSTLTYGFNGNVTGDDTGKIGGLIGANVSIGHTLKYV |
| PP | QPDFKTILESPTTDKKVGWKVIFNNMVNQNWGPYDRDSWNPVYGNQLFMKTRNGSMKAADNFLDPNKASSLLSSG |
| | FSPDFATVITMDRKASKQQTNIDVIYERVRDDYQLHWTSTNWKGTNTKDKWTDRSSERYKIDWEKEEMTN |
| | (SEQ ID NO: 54) |
| Hla, | GCTGATAGCGATATCAACATCAAGACCGGCACCACAGATAAGATCGGCTCTAATACCACCGTGAAGACCGGCGAC |
| Mat., | CTGGTGACCTACGACAAGGAGAACGGCATGCACAAGAAGGTGTTCTACAGCTTCATCGACGATAAGAACCACAAC |
| DNA | AAGAAACTGCTGGTCATCAGAACAAAAGGCACCATCGCCGGCCAGTACAGAGTGTATTCTGAAGAGGGCGCCAAC |
| | AAGAGCGGACTGGCCTGGCCTTCCGCCTTTAAGGTGCAGCTGCAACTGCCTGATAACGAGGTGGCCCAGATTAGC |
| | GACTACTACCCCAGAAACAGCATCGACACCAAGGAATACATGAGCACCCTGACCTACGGCTTCAACGGAAATGTG |
| | ACAGGAGATGATACAGGCAAGATTGGCGGCCTGATCGGCGCTAACGTGTCCATCGGACACACACTGAAGTATGTG |
| | CAACCTGACTTCAAGACCATCCTGGAATCTCCCACCACAGACAAGAAGGTCGGATGGAAGGTGATCTTCAACAAC |
| | ATGGTGAACCAGAACTGGGGCCCTTACGACAGGGACAGCTGGAACCCTGTGTACGGCAATCAGCTGTTCATGAAG |
| | ACCCGCAACGGTAGCATGAAGGCCGCCGATAACTTCCTGGACCCAAACAAAGCCAGCAGCCTGCTGAGCTCCGGC |
| | TTCAGCCCTGATTTTGCCACAGTTATCACCATGGACAGAAAGGCTTCTAAACAGCAGACCAACATCGACGTGATC |
| | TACGAGAGAGTGCGGGACGACTACCAGCTGCATTGGACAAGCACCAATTGGAAAGGCACAAACACCAAGGACAAA |
| | TGGACCGACCGGAGCTCTGAGCGGTACAAGATCGACTGGGAGAAGGAGGAAATGACCAATTGA (SEQ ID |
| | NO: 55) |
| Hla, | GCUGAUAGCGAUAUCAACAUCAAGACCGGCACCACAGAUAAGAUCGGCUCUAAUACCACCGUGAAGACCGGCGAC |
| Mat., | CUGGUGACCUACGACAAGGAGAACGGCAUGCACAAGAAGGUGUUCUACAGCUUCAUCGACGAUAAGAACCACAAC |
| RNA | AAGAAACUGCUGGUCAUCAGAACAAAAGGCACCAUCGCCGGCCAGUACAGAGUGUAUUCUGAAGAGGGCGCCAAC |
| | AAGAGCGGACUGGCCUGGCCUUCCGCCUUUAAGGUGCAGCUGCAACUGCCUGAUAACGAGGUGGCCCAGAUUAGC |
| | GACUACUACCCCAGAAACAGCAUCGACACCAAGGAAUACAUGAGCACCCUGACCUACGGCUUCAACGGAAAUGUG |
| | ACAGGAGAUGAUACAGGCAAGAUUGGCGGCCUGAUCGGCGCUAACGUGUCCAUCGGACACACACUGAAGUAUGUG |
| | CAACCUGACUUCAAGACCAUCCUGGAAUCUCCCACCACAGACAAGAAGGUCGGAUGGAAGGUGAUCUUCAACAAC |
| | AUGGUGAACCAGAACUGGGGCCCUUACGACAGGGACAGCUGGAACCCUGUGUACGGCAAUCAGCUGUUCAUGAAG |
| | ACCCGCAACGGUAGCAUGAAGGCCGCCGAUAACUUCCUGGACCCAAACAAAGCCAGCAGCCUGCUGAGCUCCGGC |
| | UUCAGCCCUGAUUUUGCCACAGUUAUCACCAUGGACAGAAAGGCUUCUAAACAGCAGACCAACAUCGACGUGAUC |
| | UACGAGAGAGUGCGGGACGACUACCAGCUGCAUUGGACAAGCACCAAUUGGAAAGGCACAAACACCAAGGACAAA |
| | UGGACCGACCGGAGCUCUGAGCGGUACAAGAUCGACUGGGAGAAGGAGGAAAUGACCAAUUGA (SEQ ID |
| | NO: 56) |
| Hla, | ADSDINIKTGTTDIGSNTTVKTGDLVTYDKENGMHKKVFYSFIDDKNHNKKLLVIRTKGTIAGQY |
| Ant, | (SEQ ID NO: 57) |
| PP | |
| Hla | GCTGATAGCGATATCAACATCAAGACCGGCACCACAGATAAGATCGGCTCTAATACCACCGTGAAGACCGGCGAC |
| Ant, | CTGGTGACCTACGACAAGGAGAACGGCATGCACAAGAAGGTGTTCTACAGCTTCATCGACGATAAGAACCACAAC |
| DNA | AAGAAACTGCTGGTCATCAGAACAAAAGGCACCATCGCCGGCCAGTAC (SEQ ID NO: 58) |
| Hla, | GCUGAUAGCGAUAUCAACAUCAAGACCGGCACCACAGAUAAGAUCGGCUCUAAUACCACCGUGAAGACCGGCGAC |
| Ant, | CUGGUGACCUACGACAAGGAGAACGGCAUGCACAAGAAGGUGUUCUACAGCUUCAUCGACGAUAAGAACCACAAC |
| RNA | AAGAAACUGCUGGUCAUCAGAACAAAAGGCACCAUCGCCGGCCAGUAC (SEQ ID NO: 59) |
| CgoX, | MTKSVAIIGAGITGLSSAYFLKQQDPNIDVTIFEASNRPGGKIQSYRKDGYMIELGPESYLGRKTIMTELAKDIG |
| FL, | LEQDIVTNTTGQSYIFAKNKLYPIPGGSIMGIPTDIKPFVTTKLISPLGKLRAGFDLLKKPTQMQDGDISVGAFF |
| PP | RARLGNEVLENLIEPLMGGIYGTDIDKLSLMSTFPNFKEKEEAFGSLIKGMKDEKNKRLKQRQLYPGAPKGQFKQ |
| | FKHGLSSFIEALEQDVKNKGVTIRYNTSVDDIITSQKQYKIVYNDQLEEVYDGVLVTTPHQVFLNWFGQDPAFDY |
| | FKTMDSTTVATVVLAFDEKDIENTHDGTGFVIARTSDTDITACTWTSKKWPFTTPEGKVLIRAYVGKPGDTVVDD |
| | HTDNELVSIVRRDLSQMMTFKGDPEFTIVNRLPKSMPQYHVGHIQQIRQIQAHIKQTYPRLRVTGASFEAVGLPD |
| | CITQGKVAAEEVIAEL (SEQ ID NO: 60) |
| CgoX, | ATGACAAAGAGCGTAGCAATCATTGGTGCAGGCATTACCGGCCTGAGCTCCGCTTACTTCCTGAAACAACAAGAC |
| FL, | CCTAATATCGATGTAACCATCTTCGAAGCCTCCAATCGCCCTGGGGGCAAGATCCAGTCTTATAGAAAAGACGGT |
| DNA | TACATGATAGAGCTGGGGCCCGAAAGCTATCTCGGTCGAAAAACGATCATGACCGAGCTTGCCAAAGACATTGGC |
| | CTGGAGCAGGACATTGTGACAAACACCACGGGACAAAGCTATATATTCGCTAAGAATAAGCTCTATCCAATCCCG |
| | GGCGGGTCAATTATGGGCATTCCCACGGATATCAAACCATTCGTAACCACAAAATTGATCTCTCCCTTGGGGAAG |
| | CTGAGAGCTGGGTTTGACCTGCTGAAGAAACCAACCCAGATGCAAGACGGGGACATCTCTGTGGGGGCCTTCTTT |
| | AGAGCTAGGCTGGGAAACGAAGTGCTGGAGAACCTGATCGAGCCCCTCATGGGCGGCATCTATGGGACTGATATT |
| | GACAAGCTTAGCCTGATGTCTACTTTCCCAAACTTTAAGGAGAAGGAAGAAGCATTCGGCTCCCTGATTAAGGGG |
| | ATGAAGGACGAAAAGAACAAGCGGCTGAAGCAGAGACAACTGTATCCTGGGGCCCCCAAGGGACAGTTCAAGCAA |
| | TTCAAGCATGGGCTGAGTTCCTTCATTGAGGCTCTGGAGCAGGACGTGAAGAACAAGGGCGTGACCATCAGATAT |
| | AATACATCTGTGGATGACATTATTACCTCACAGAAACAGTATAAGATCGTGTACAATGACCAGTTGGAGGAAGTG |
| | TACGACGGGGTCCTTGTGACAACCCCACACCAGGTTTTCCTCAATTGGTTTGGGCAGGATCCCGCGTTTGACTAC |
| | TTTAAGACCATGGATTCAACCACAGTGGCCACCGTCGTATTGGCATTCGACGAGAAGGACATTGAAAATACTCAC |
| | GATGGCACGGGATTTGTGATCGCTCGGACGTCAGACACAGACATCACCGCGTGCACTTGGACATCTAAGAAATGG |
| | CCCTTCACTACACCAGAGGGAAAAGTATTGATTAGGGCCTACGTCGGGAAGCCTGGGGACACGGTTGTGGACGAT |
| | CACACAGACAACGAACTGGTGTCAATTGTGAGACGAGATCTCTCTCAGATGATGACTTTCAAAGGGGACCCAGAA |
| | TTCACTATCGTGAATCGGCTTCCCAAGAGCATGCCCCAGTACCACGTGGGGCACATCCAGCAGATCCGGCAGATC |
| | CAAGCCCATATAAAGCAAACATATCCACGCCTTCGGGTTACTGGCGCCTCTTTTGAGGCTGTAGGGCTGCCTGAT |
| | TGTATAACACAGGGCAAGGTGGCAGCTGAGGAGGTGATTGCCGAACTG (SEQ ID NO: 61) |
| CgoX, | AUGACAAAGAGCGUAGCAAUCAUUGGUGCAGGCAUUACCGGCCUGAGCUCCGCUUACUUCCUGAAACAACAAGAC |
| FL, | CCUAAUAUCGAUGUAACCAUCUUCGAAGCCUCCAAUCGCCCUGGGGGCAAGAUCCAGUCUUAUAGAAAAGACGGU |
| RNA | UACAUGAUAGAGCUGGGGCCCGAAAGCUAUCUCGGUCGAAAAACGAUCAUGACCGAGCUUGCCAAAGACAUUGGC |
| | CUGGAGCAGGACAUUGUGACAAACACCACGGGACAAAGCUAUAUAUUCGCUAAGAAUAAGCUCUAUCCAAUCCCG |
| | GGCGGGUCAAUUAUGGGCAUUCCCACGGAUAUCAAACCAUUCGUAACCACAAAAUUGAUCUCUCCCUUGGGGAAG |
| | CUGAGAGCUGGGUUUGACCUGCUGAAGAAACCAACCCAGAUGCAAGACGGGGACAUCUCUGUGGGGGCCUUCUUU |
| | AGAGCUAGGCUGGGAAACGAAGUGCUGGAGAACCUGAUCGAGCCCCUCAUGGGCGGCAUCUAUGGGACUGAUAUU |
| | GACAAGCUUAGCCUGAUGUCUACUUUCCCAAACUUUAAGGAGAAGGAAGAAGCAUUCGGCUCCCUGAUUAAGGGG |
| | AUGAAGGACGAAAAGAACAAGCGGCUGAAGCAGAGACAACUGUAUCCUGGGGCCCCCAAGGGACAGUUCAAGCAA |
| | UUCAAGCAUGGGCUGAGUUCCUUCAUUGAGGCUCUGGAGCAGGACGUGAAGAACAAGGGCGUGACCAUCAGAUAU |
| | AAUACAUCUGUGGAUGACAUUAUUACCUCACAGAAACAGUAUAAGAUCGUGUACAAUGACCAGUUGGAGGAAGUG |
| | UACGACGGGGUCCUUGUGACAACCCCACACCAGGUUUUCCUCAAUUGGUUUGGGCAGGAUCCCGCGUUUGACUAC |
| | UUUAAGACCAUGGAUUCAACCACAGUGGCCACCGUCGUAUUGGCAUUCGACGAGAAGGACAUUGAAAAUACUCAC |
| | GAUGGCACGGGAUUUGUGAUCGCUCGGACGUCAGACACAGACAUCACCGCGUGCACUUGGACAUCUAAGAAAUGG |
| | CCCUUCACUACACCAGAGGGAAAAGUAUUGAUUAGGGCCUACGUCGGGAAGCCUGGGGACACGGUUGUGGACGAU |
| | CACACAGACAACGAACUGGUGUCAAUUGUGAGACGAGAUCUCUCUCAGAUGAUGACUUUCAAAGGGGACCCAGAA |
| | UUCACUAUCGUGAAUCGGCUUCCCAAGAGCAUGCCCCAGUACCACGUGGGGCACAUCCAGCAGAUCCGGCAGAUC |
| | CAAGCCCAUAUAAAGCAAACAUAUCCACGCCUUCGGGUUACUGGCGCCUCUUUUGAGGCUGUAGGGCUGCCUGAU |
| | UGUAUAACACAGGGCAAGGUGGCAGCUGAGGAGGUGAUUGCCGAACUG (SEQ ID NO: 62) |
| Cgox, | TDNELVSIVRRD (SEQ ID NO: 63) |
| Ant1, | |
| PP | |
| CgoX, | ACCGACAATGAGCTGGTGAGTATCGTGAGGAGGGAC (SEQ ID NO: 64) |
| Ant1, | |
| DNA | |
| CgoX, | ACCGACAAUGAGCUGGUGAGUAUCGUGAGGAGGGAC (SEQ ID NO: 65) |
| Ant1, | |
| RNA | |
| CgoX, | TDDKLVSIVRRD (SEQ ID NO: 66) |
| Ant2, | |
| PP | |
| Cgox, | ACCGACGATAAGCTGGTCTCCATCGTCCGGCGCGAC (SEQ ID NO: 67) |
| Ant2, | |
| DNA | |
| CgoX, | ACCGACGAUAAGCUGGUCUCCAUCGUCCGGCGCGAC (SEQ ID NO: 68) |
| Ant2, | |
| RNA | |
| CgoX, | TDDELVSIVRRD (SEQ ID NO: 69) |
| Ant3, | |
| PP | |
| CgoX, | ACTGACGATGAACTGGTCAGCATCGTGCGGCGGGAC (SEQ ID NO: 70) |
| Ant3, | |
| DNA | |
| CgoX, | ACUGACGAUGAACUGGUCAGCAUCGUGCGGCGGGAC (SEQ ID NO: 71) |
| Ant3, | |
| RNA | |
| IsaA, | MKKTIMASSLAVALGVTGYAAGTGHQAHAAEVNVDQAHLVDLAHNHQDQLNAAPIKDGAYDIHFVKDG |
| FL, | FQYNFTSNGTTWSWSYEAANGQTAGFSNVAGADYTTSYNQGSNVQSVSYNAQSSNSNVEAVSAPTYHN |
| PP | YSTSTTSSSVRLSNGNTAGATGSSAAQIMAQRTGVSASTWAAIIARESNGQVNAYNPSGASGLFQTMP |
| | GWGPTNTVDQQINAAVKAYKAQGLGAWGF (SEQ ID NO: 91) |
| IsaA, | ATGAAAAAGACCATCATGGCCTCTAGCCTGGCCGTCGCCCTGGGCGTGACAGGCTACGCCGCTGGTAC |
| FL, | AGGCCACCAGGCCCACGCCGCCGAAGTGAACGTGGATCAAGCCCATCTGGTGGACCTGGCCCACAACC |
| DNA | ACCAGGACCAGCTGAACGCCGCTCCAATCAAGGATGGCGCCTACGACATCCACTTCGTGAAGGACGGC |
| | TTCCAATACAACTTCACCTCTAATGGCACCACATGGTCCTGGAGCTACGAGGCCGCCAACGGCCAGAC |
| | AGCCGGATTTAGCAATGTCGCCGGCGCAGATTACACCACCAGCTACAACCAGGGCAGCAACGTGCAGT |
| | CCGTGTCTTACAATGCCCAGAGCAGCAACAGCAACGTGGAAGCCGTGTCCGCTCCTACCTACCACAAC |
| | TACAGCACCAGCACCACATCTAGCAGCGTGCGGCTGAGCAACGGCAACACCGCCGGCGCCACCGGCTC |
| | TTCTGCTGCCCAGATCATGGCCCAAAGAACCGGCGTGTCCGCCAGCACATGGGCCGCTATCATCGCCA |
| | GAGAGAGCAATGGACAGGTGAACGCCTACAACCCCAGCGGCGCTAGCGGCCTGTTCCAGACCATGCCT |
| | GGCTGGGGCCCTACAAACACCGTGGACCAGCAGATTAACGCCGCTGTTAAGGCCTATAAGGCCCAGGG |
| | ACTCGGAGCTTGGGGATTCTGA (SEQ ID NO: 92) |
| IsaA, | AUGAAAAAGACCAUCAUGGCCUCUAGCCUGGCCGUCGCCCUGGGCGUGACAGGCUACGCCGCUGGUAC |
| FL, | AGGCCACCAGGCCCACGCCGCCGAAGUGAACGUGGAUCAAGCCCAUCUGGUGGACCUGGCCCACAACC |
| RNA | ACCAGGACCAGCUGAACGCCGCUCCAAUCAAGGAUGGCGCCUACGACAUCCACUUCGUGAAGGACGGC |
| | UUCCAAUACAACUUCACCUCUAAUGGCACCACAUGGUCCUGGAGCUACGAGGCCGCCAACGGCCAGAC |
| | AGCCGGAUUUAGCAAUGUCGCCGGCGCAGAUUACACCACCAGCUACAACCAGGGCAGCAACGUGCAGU |
| | CCGUGUCUUACAAUGCCCAGAGCAGCAACAGCAACGUGGAAGCCGUGUCCGCUCCUACCUACCACAAC |
| | UACAGCACCAGCACCACAUCUAGCAGCGUGCGGCUGAGCAACGGCAACACCGCCGGCGCCACCGGCUC |
| | UUCUGCUGCCCAGAUCAUGGCCCAAAGAACCGGCGUGUCCGCCAGCACAUGGGCCGCUAUCAUCGCCA |
| | GAGAGAGCAAUGGACAGGUGAACGCCUACAACCCCAGCGGCGCUAGCGGCCUGUUCCAGACCAUGCCU |
| | GGCUGGGGCCCUACAAACACCGUGGACCAGCAGAUUAACGCCGCUGUUAAGGCCUAUAAGGCCCAGGG |
| | ACUCGGAGCUUGGGGAUUCUGA (SEQ ID NO: 93) |
| IsaA, | AEVNVDQAHLVDLAHNHQDQLNAAPIKDGAYDIHFVKDGFQYNFTSNGTTWSWSYEAANGQTAGFSNV |
| Mat., | AGADYTTSYNQGSNVQSVSYNAQSSNSNVEAVSAPTYHNYSTSTTSSSVRLSNGNTAGATGSSAAQIM |
| PP | AQRTGVSASTWAAIIARESNGQVNAYNPSGASGLFQTMPGWGPTNTVDQQINAAVKAYKAQGLGAWGF |
| | (SEQ ID NO: 94) |
| IsaA, | GCCGAAGTGAACGTGGATCAAGCCCATCTGGTGGACCTGGCCCACAACCACCAGGACCAGCTGAACGC |
| Mat., | CGCTCCAATCAAGGATGGCGCCTACGACATCCACTTCGTGAAGGACGGCTTCCAATACAACTTCACCT |
| DNA | CTAATGGCACCACATGGTCCTGGAGCTACGAGGCCGCCAACGGCCAGACAGCCGGATTTAGCAATGTC |
| | GCCGGCGCAGATTACACCACCAGCTACAACCAGGGCAGCAACGTGCAGTCCGTGTCTTACAATGCCCA |
| | GAGCAGCAACAGCAACGTGGAAGCCGTGTCCGCTCCTACCTACCACAACTACAGCACCAGCACCACAT |
| | CTAGCAGCGTGCGGCTGAGCAACGGCAACACCGCCGGCGCCACCGGCTCTTCTGCTGCCCAGATCATG |
| | GCCCAAAGAACCGGCGTGTCCGCCAGCACATGGGCCGCTATCATCGCCAGAGAGAGCAATGGACAGGT |
| | GAACGCCTACAACCCCAGCGGCGCTAGCGGCCTGTTCCAGACCATGCCTGGCTGGGGCCCTACAAACA |
| | CCGTGGACCAGCAGATTAACGCCGCTGTTAAGGCCTATAAGGCCCAGGGACTCGGAGCTTGGGGATTC |
| | TGA (SEQ ID NO: 95) |
| IsaA, | GCCGAAGUGAACGUGGAUCAAGCCCAUCUGGUGGACCUGGCCCACAACCACCAGGACCAGCUGAACGC |
| Mat., | CGCUCCAAUCAAGGAUGGCGCCUACGACAUCCACUUCGUGAAGGACGGCUUCCAAUACAACUUCACCU |
| RNA | CUAAUGGCACCACAUGGUCCUGGAGCUACGAGGCCGCCAACGGCCAGACAGCCGGAUUUAGCAAUGUC |
| | GCCGGCGCAGAUUACACCACCAGCUACAACCAGGGCAGCAACGUGCAGUCCGUGUCUUACAAUGCCCA |
| | GAGCAGCAACAGCAACGUGGAAGCCGUGUCCGCUCCUACCUACCACAACUACAGCACCAGCACCACAU |
| | CUAGCAGCGUGCGGCUGAGCAACGGCAACACCGCCGGCGCCACCGGCUCUUCUGCUGCCCAGAUCAUG |
| | GCCCAAAGAACCGGCGUGUCCGCCAGCACAUGGGCCGCUAUCAUCGCCAGAGAGAGCAAUGGACAGGU |
| | GAACGCCUACAACCCCAGCGGCGCUAGCGGCCUGUUCCAGACCAUGCCUGGCUGGGGCCCUACAAACA |
| | CCGUGGACCAGCAGAUUAACGCCGCUGUUAAGGCCUAUAAGGCCCAGGGACUCGGAGCUUGGGGAUUC |
| | UGA (SEQ ID NO: 96) |
| IsaA | MAEVNVDQAHLVDLAHNHQDQLNAAPIKDGAYDIHFVKDGFQYNFTSNGTTWSWSYEAANGQTAGFSNVAGADYT |
| 2, | TSYNQGSNVQSVSYNAQSSNSNVEAVSAPTYHNYSTSTTSSSVRLSNGNTAGATGSSAAQIMAQRTGVPASTWAA |
| Mat., | IIARESNGQVNAYNPSGASGLFQTMPGWGPTNTVDQQINAAVKAYKAQGLGAWGF (SEQ ID NO: 123) |
| PP | |
| IsaA | ATGGCTGAAGTAAACGTTGATCAAGCACACTTAGTTGACTTAGCGCATAATCATCAAGATCAATTAAATGCAGCT |
| 2, | CCAATCAAAGATGGTGCATATGACATCCACTTTGTAAAAGATGGTTTCCAATATAACTTTACTTCAAATGGTACT |
| Mat., | ACATGGTCATGGAGCTATGAAGCAGCTAATGGTCAAACTGCTGGTTTCTCAAACGTTGCAGGTGCAGACTACACT |
| DNA | ACTTCATACAACCAAGGTTCAAATGTACAATCAGTAAGCTACAATGCACAATCAAGTAACTCAAACGTTGAAGCT |
| | GTTTCAGCTCCAACTTACCATAACTACAGCACTTCAACTACTTCAAGTTCAGTGAGATTAAGCAATGGTAATACT |
| | GCAGGTGCTACTGGTTCATCAGCAGCTCAAATCATGGCTCAACGTACTGGTGTTCCAGCTTCTACATGGGCTGCA |
| | ATCATCGCTCGTGAATCAAATGGTCAAGTAAATGCTTACAACCCATCAGGTGCTTCAGGTTTATTCCAAACTATG |
| | CCAGGTTGGGGCCCAACAAACACTGTTGACCAACAAATCAACGCAGCTGTTAAAGCATACAAAGCACAAGGTTTA |
| | GGTGCTTGGGGATTCTAA (SEQ ID NO: 124) |
| IsaA | AUGGCUGAAGUAAACGUUGAUCAAGCACACUUAGUUGACUUAGCGCAUAAUCAUCAAGAUCAAUUAAAUGCAGCU |
| 2, | CCAAUCAAAGAUGGUGCAUAUGACAUCCACUUUGUAAAAGAUGGUUUCCAAUAUAACUUUACUUCAAAUGGUACU |
| Mat., | ACAUGGUCAUGGAGCUAUGAAGCAGCUAAUGGUCAAACUGCUGGUUUCUCAAACGUUGCAGGUGCAGACUACACU |
| RNA | ACUUCAUACAACCAAGGUUCAAAUGUACAAUCAGUAAGCUACAAUGCACAAUCAAGUAACUCAAACGUUGAAGCU |
| | GUUUCAGCUCCAACUUACCAUAACUACAGCACUUCAACUACUUCAAGUUCAGUGAGAUUAAGCAAUGGUAAUACU |
| | GCAGGUGCUACUGGUUCAUCAGCAGCUCAAAUCAUGGCUCAACGUACUGGUGUUCCAGCUUCUACAUGGGCUGCA |
| | AUCAUCGCUCGUGAAUCAAAUGGUCAAGUAAAUGCUUACAACCCAUCAGGUGCUUCAGGUUUAUUCCAAACUAUG |
| | CCAGGUUGGGGCCCAACAAACACUGUUGACCAACAAAUCAACGCAGCUGUUAAAGCAUACAAAGCACAAGGUUUA |
| | GGUGCUUGGGGAUUCUAA (SEQ ID NO: 125) |
| IsaA, | AEVNVDQAHLVDLAHNHQDQLNAAPIKDGAYDIHFVKDGFQYNFTSNGTTWSWSY (SEQ ID |
| Ant1, | NO: 97) |
| PP | |
| IsaA, | GCCGAAGTGAACGTGGATCAAGCCCATCTGGTGGACCTGGCCCACAACCACCAGGACCAGCTGAACG |
| Ant1, | CCGCTCCAATCAAGGATGGCGCCTACGACATCCACTTCGTGAAGGACGGCTTCCAATACAACTTCAC |
| DNA | CTCTAATGGCACCACATGGTCCTGGAGCTAC |
| | (SEQ ID NO: 98) |
| IsaA, | GCCGAAGUGAACGUGGAUCAAGCCCAUCUGGUGGACCUGGCCCACAACCACCAGGACCAGCUGAACGC |
| Ant1, | CGCUCCAAUCAAGGAUGGCGCCUACGACAUCCACUUCGUGAAGGACGGCUUCCAAUACAACUUCACCU |
| RNA | CUAAUGGCACCACAUGGUCCUGGAGCUAC (SEQ ID NO: 99) |
| IsaA, | SNVAGADYTTSYNQGS (SEQ ID NO: 100) |
| Ant2, | |
| PP | |
| IsaA, | AGCAATGTCGCCGGCGCAGATTACACCACCAGCTACAACCAGGGCAGCAACGTGCAGTCC (SEQ ID |
| Ant2, | NO: 101) |
| DNA | |
| IsaA, | AGCAAUGUCGCCGGCGCAGAUUACACCACCAGCUACAACCAGGGCAGCAACGUGCAGUCC (SEQ |
| Ant2, | ID NO: 102) |
| RNA | |
| IsaA, | SGASGLFQTMPGWGPTNTVDQQINAAVKAYKAQGLGAW (SEQ ID NO: 103) |
| Ant3, | |
| PP | |
| IsaA, | AGCGGCGCTAGCGGCCTGTTCCAGACCATGCCTGGCTGGGGCCCTACAAACACCGTGGACCAGCAGAT |
| Ant3, | TAACGCCGCTGTTAAGGCCTATAAGGCCCAGGGACTCGGAGCT (SEQ ID NO: 104) |
| DNA | |
| IsaA, | AGCGGCGCUAGCGGCCUGUUCCAGACCAUGCCUGGCUGGGGCCCUACAAACACCGUGGACCAGCAGAU |
| Ant3, | UAACGCCGCUGUUAAGGCCUAUAAGGCCCAGGGACUCGGAGCU (SEQ ID NO: 105) |
| RNA | |
| SceD, | MKKTLLASSLAVGLGIVAGNAGHEAHASEADLNKASLAQMAQSNDQTLNQKPIEAGAYNYTFDYEGFT |
| FL, | YHFESDGTHFAWNYHATGANGADMSAQAPATNNVAPSAVQANQANQVQSQEVEAPQNAQTQQPQASTS |
| PP | NNSQVTATPTESKASEGSSVNVNAHLKQIAQRESGGNIHAVNPTSGAAGKYQFLQSTWDSVAPAKYKG |
| | VSPANAPESVODAAAVKLYNTGGAGHWVTA (SEQ ID NO: 106) |
| SceD, | ATGAAAAAGACCCTGCTGGCCTCCAGCCTGGCTGTGGGCCTGGGCATCGTGGCCGGCAACGCCGGCCA |
| FL, | CGAGGCTCACGCCTCTGAAGCCGACCTGAACAAGGCCAGCCTGGCCCAGATGGCCCAATCTAACGACC |
| DNA | AGACACTCAACCAGAAACCTATCGAGGCCGGAGCTTATAATTACACCTTCGACTACGAGGGCTTCACC |
| | TACCACTTCGAGTCTGATGGCACCCACTTTGCCTGGAACTACCACGCAACAGGCGCCAATGGCGCTGA |
| | TATGAGCGCCCAAGCTCCTGCCACAAACAACGTGGCTCCATCTGCCGTGCAGGCCAACCAGGCCAACC |
| | AGGTGCAGAGCCAGGAGGTGGAAGCCCCTCAGAACGCCCAGACCCAGCAGCCCCAGGCCAGCACCAGC |
| | AACAACAGCCAGGTCACAGCCACCCCTACAGAGAGCAAGGCCAGCGAAGGCAGCAGCGTCAACGTGAA |
| | CGCCCATCTGAAGCAGATCGCCCAAAGAGAATCCGGCGGAAATATCCACGCCGTGAACCCCACCAGCG |
| | GCGCCGCCGGAAAGTACCAGTTCCTGCAGTCTACCTGGGACAGCGTGGCCCCCGCCAAGTACAAGGGC |
| | GTGTCCCCTGCTAATGCCCCTGAGAGCGTGCAGGACGCCGCTGCTGTTAAGCTGTACAACACCGGAGG |
| | CGCCGGCCACTGGGTGACAGCCTGA (SEQ ID NO: 107) |
| SceD, | AUGAAAAAGACCCUGCUGGCCUCCAGCCUGGCUGUGGGCCUGGGCAUCGUGGCCGGCAACGCCGGCCA |
| FL, | CGAGGCUCACGCCUCUGAAGCCGACCUGAACAAGGCCAGCCUGGCCCAGAUGGCCCAAUCUAACGACC |
| RNA | AGACACUCAACCAGAAACCUAUCGAGGCCGGAGCUUAUAAUUACACCUUCGACUACGAGGGCUUCACC |
| | UACCACUUCGAGUCUGAUGGCACCCACUUUGCCUGGAACUACCACGCAACAGGCGCCAAUGGCGCUGA |
| | UAUGAGCGCCCAAGCUCCUGCCACAAACAACGUGGCUCCAUCUGCCGUGCAGGCCAACCAGGCCAACC |
| | AGGUGCAGAGCCAGGAGGUGGAAGCCCCUCAGAACGCCCAGACCCAGCAGCCCCAGGCCAGCACCAGC |
| | AACAACAGCCAGGUCACAGCCACCCCUACAGAGAGCAAGGCCAGCGAAGGCAGCAGCGUCAACGUGAA |
| | CGCCCAUCUGAAGCAGAUCGCCCAAAGAGAAUCCGGCGGAAAUAUCCACGCCGUGAACCCCACCAGCG |
| | GCGCCGCCGGAAAGUACCAGUUCCUGCAGUCUACCUGGGACAGCGUGGCCCCCGCCAAGUACAAGGGC |
| | GUGUCCCCUGCUAAUGCCCCUGAGAGCGUGCAGGACGCCGCUGCUGUUAAGCUGUACAACACCGGAGG |
| | CGCCGGCCACUGGGUGACAGCCUGA (SEQ ID NO: 108) |
| SceD, | SEADLNKASLAQMAQSNDQTLNQKPIEAGAYNYTFDYEGFTYHFESDGTHFAWNYHATGANGADMSAQ |
| Mat., | APATNNVAPSAVQANQANQVQSQEVEAPQNAQTQQPQASTSNNSQVTATPTESKASEGSSVNVNAHLK |
| PP | QIAQRESGGNIHAVNPTSGAAGKYQFLQSTWDSVAPAKYKGVSPANAPESVQDAAAVKLYNTGGAGHW |
| | VTA (SEQ ID NO: 109) |
| SceD, | TCTGAAGCCGACCTGAACAAGGCCAGCCTGGCCCAGATGGCCCAATCTAACGACCAGACACTCAACC |
| Mat., | AGAAACCTATCGAGGCCGGAGCTTATAATTACACCTTCGACTACGAGGGCTTCACCTACCACTTCGA |
| DNA | GTCTGATGGCACCCACTTTGCCTGGAACTACCACGCAACAGGCGCCAATGGCGCTGATATGAGCGCC |
| | CAAGCTCCTGCCACAAACAACGTGGCTCCATCTGCCGTGCAGGCCAACCAGGCCAACCAGGTGCAGA |
| | GCCAGGAGGTGGAAGCCCCTCAGAACGCCCAGACCCAGCAGCCCCAGGCCAGCACCAGCAACAACAG |
| | CCAGGTCACAGCCACCCCTACAGAGAGCAAGGCCAGCGAAGGCAGCAGCGTCAACGTGAACGCCCAT |
| | CTGAAGCAGATCGCCCAAAGAGAATCCGGCGGAAATATCCACGCCGTGAACCCCACCAGCGGCGCCG |
| | CCGGAAAGTACCAGTTCCTGCAGTCTACCTGGGACAGCGTGGCCCCCGCCAAGTACAAGGGCGTGTC |
| | CCCTGCTAATGCCCCTGAGAGCGTGCAGGACGCCGCTGCTGTTAAGCTGTACAACACCGGAGGCGCC |
| | GGCCACTGGGTGACAGCCTGA |
| | (SEQ ID NO: 110) |
| SceD, | UCUGAAGCCGACCUGAACAAGGCCAGCCUGGCCCAGAUGGCCCAAUCUAACGACCAGACACUCAACCA |
| Mat., | GAAACCUAUCGAGGCCGGAGCUUAUAAUUACACCUUCGACUACGAGGGCUUCACCUACCACUUCGAGU |
| RNA | CUGAUGGCACCCACUUUGCCUGGAACUACCACGCAACAGGCGCCAAUGGCGCUGAUAUGAGCGCCCAA |
| | GCUCCUGCCACAAACAACGUGGCUCCAUCUGCCGUGCAGGCCAACCAGGCCAACCAGGUGCAGAGCCA |
| | GGAGGUGGAAGCCCCUCAGAACGCCCAGACCCAGCAGCCCCAGGCCAGCACCAGCAACAACAGCCAGG |
| | UCACAGCCACCCCUACAGAGAGCAAGGCCAGCGAAGGCAGCAGCGUCAACGUGAACGCCCAUCUGAAG |
| | CAGAUCGCCCAAAGAGAAUCCGGCGGAAAUAUCCACGCCGUGAACCCCACCAGCGGCGCCGCCGGAAA |
| | GUACCAGUUCCUGCAGUCUACCUGGGACAGCGUGGCCCCCGCCAAGUACAAGGGCGUGUCCCCUGCUA |
| | AUGCCCCUGAGAGCGUGCAGGACGCCGCUGCUGUUAAGCUGUACAACACCGGAGGCGCCGGCCACUGG |
| | GUGACAGCCUGA (SEQ ID NO: 111) |
| SceD, | APSAVQANQANQVQSQEVEAPQNAQTQQPQASTSNNSQVTATPTESKASEGSSVNVNAHLKQIAQRES |
| Ant1, | GGNIHAVNPTSGAAGKYQFLQSTWDSVAPAKYKGVSPANAPES (SEQ ID NO: 112) |
| PP | |
| SceD, | GCTCCATCTGCCGTGCAGGCCAACCAGGCCAACCAGGTGCAGAGCCAGGAGGTGGAAGCCCCTCAGAA |
| Ant1, | CGCCCAGACCCAGCAGCCCCAGGCCAGCACCAGCAACAACAGCCAGGTCACAGCCACCCCTACAGAGA |
| DNA | GCAAGGCCAGCGAAGGCAGCAGCGTCAACGTGAACGCCCATCTGAAGCAGATCGCCCAAAGAGAATCC |
| | GGCGGAAATATCCACGCCGTGAACCCCACCAGCGGCGCCGCCGGAAAGTACCAGTTCCTGCAGTCTAC |
| | CTGGGACAGCGTGGCCCCCGCCAAGTACAAGGGCGTGTCCCCTGCTAATGCCCCTGAGAGC (SEQ |
| | ID NO: 113) |
| SceD, | GCUCCUGCCACAAACAACGUGGCUCCAUCUGCCGUGCAGGCCAACCAGGCCAACCAGGUGCAGAGCCA |
| Ant1, | GGAGGUGGAAGCCCCUCAGAACGCCCAGACCCAGCAGCCCCAGGCCAGCACCAGCAACAACAGCCAGG |
| RNA | UCACAGCCACCCCUACAGAGAGCAAGGCCAGCGAAGGCAGCAGCGUCAACGUGAACGCCCAUCUGAAG |
| | CAGAUCGCCCAAAGAGAAUCCGGCGGAAAUAUCCACGCCGUGAACCCCACCAGCGGCGCCGCCGGAAA |
| | GUACCAGUUCCUGCAGUCUACCUGGGACAGCGUGGCCCCCGCCAAGUACAAGGGCGUGUCCCCUGCUA |
| | AUGCCCCUGAGAGC (SEQ ID NO: 114) |

TABLE 2
Exemplary wild-type S. aureus antigen coding sequences

| | Sequence |
|---|---|
| At1, | ATGTTAGGAGTAATAAATAGAATGGCGAAAAAATTCAATTACAAACTACCATCAATGGTTGCATTAACGCTTGT |
| FL, | AGGTTCAGCAGTCACTGCACATCAAGTTCAAGCAGCTGAGACGACACAAGATCAAACTACTAATAAAAATGTTT |
| WT, | TAGATAGTAATAAAGTTAAAGCAACTACTGAACAAGCAAAAGCTGAGGTAAAAAATCCAACGCAAAACATTTCT |
| DNA | GGCACTCAAGTATATCAAGACCCTGCTATTGTCCAACCAAAAACAGCAAATAACAAAACAGGCAATGCTCAAGT |
| | AAGTCAAAAAGTTGATACTGCACAAGTAAATGGTGACACTCGTGCTAATCAATCAGCGACTACAAATAATACGC |
| | AGCCTGTTGCAAAGTCAACAAGCACTACAGCACCTAAAACTAACACTAATGTTACAAATGCTGGTTATAGTTTA |
| | GTTGATGATGAAGATGATAATTCAGAACATCAAATTAATCCAGAATTAATTAAATCAGCTGCTAAACCTGCAGC |
| | TCTTGAAACGCAATATAAAGCCGCAGCACCTAAAGCTAAAACTGAAGCGACACCTAAAGTAACTACTTTTAGCG |
| | CTTCAGCACAACCAAGATCAGTTGCTGCAACACCAAAAACGAGTTTGCCAAAATATAAACCACAAGTAAACTCT |
| | TCAATTAACGATTACATTCGTAAAAATAACTTAAAAGCACCTAAAATTGAAGAAGATTATACATCTTACTTCCC |
| | TAAATACGCATACCGTAACGGCGTAGGTCGTCCTGAAGGTATCGTAGTTCATGATACAGCTAATGATCGTTCGA |
| | CGATAAATGGTGAAATTAGTTATATGAAAAATAACTATCAAAACGCATTCGTACATGCATTTGTTGATGGGGAT |
| | CGTATAATCGAAACAGCACCAACGGATTACTTATCTTGGGGTGTCGGTGCAGTCGGTAACCCTAGATTCATCAA |
| | TGTTGAAATCGTACACACACACGACTATGCTTCATTTGCACGTTCAATGAATAACTATGCTGACTATGCAGCTA |
| | CACAATTACAATATTATGGTTTAAAACCAGACAGTGCTGAGTATGATGGAAATGGTACAGTATGGACTCACTAC |
| | GCTGTAAGTAAATATTTAGGTGGTACGGACCATGCCGATCCACATGGATATTTAAGAAGTCATAATTATAGTTA |
| | TGATCAATTATATGACTTAATTAATGAAAAATATTTAATAAAAATGGGTAAAGTGGCGCCATGGGGTACGCAAT |
| | TTACAACTACCCCTACTACACCATCAAAACCAACAACACCGTCGAAACCATCAACTGGTAAATTAACAGTTGCA |
| | GCAAACAATGGTGTCGCACAAATCAAACCAACAAATAGTGGTTTATATACTACTGTTTACGACAAAACTGGTAA |
| | AGCAACTAATGAAGTTCAAAAAACATTTGCTGTATCTAAAACAGCTACATTAGGTAATCAAAAATTCTATCTTG |
| | TTCAAGATTACAATTCTGGTAATAAATTTGGTTGGGTTAAAGAAGGCGATGTGGTTTACAACACAGCTAAATCA |
| | CCTGTAAATGTAAATCAATCATATTCAATCAAATCTGGTACGAAACTTTATACAGTACCTTGGGGTACATCTAA |
| | ACAAGTTGCTGGTAGCGTGTCTGGCTCTGGAAACCAAACATTTAAGGCTTCAAAGCAACAACAAATTGATAAAT |
| | CAATTTATTTATATGGCTCTGTGAATGGTAAATCTGGTTGGGTAAGTAAAGCATATTTAGTTGATACTGCTAAA |
| | CCTACGCCTACACCAATACCTAAGCCATCAACACCTACAACAAATAATAAATTAACAGTTTCATCATTAAACGG |
| | TGTTGCTCAAATTAATGCTAAAAACAATGGCTTATTCACTACAGTTTATGACAAAACTGGTAAGCCAACGAAAG |
| | AAGTTCAAAAAACATTTGCTGTAACAAAAGAAGCAAGTTTAGGTGGAAACAAATTCTACTTAGTTAAAGATTAC |
| | AATAGTCCAACTTTAATTGGTTGGGTTAAACAAGGTGACGTTATTTATAACAATGCAAAATCACCTGTAAATGT |
| | AATGCAAACATATACAGTAAAACCAGGCACTAAATTATATTCAGTACCTTGGGGCACTTATAAACAAGAAGCTG |
| | GTGCAGTTTCTGGTACAGGTAACCAAACTTTTAAAGCGACTAAGCAACAACAAATTGATAAATCTATCTATTTA |
| | TTTGGAACTGTAAATGGTAAATCTGGTTGGGTAAGTAAAGCATATTTAGCTGTACCTGCTGCACCTAAAAAAGC |
| | AGTAGCACAACCAAAAACAGCTGTAAAAGCTTATACTGTTACTAAACCACAAACGACTCAAACAGTTAGCAAGA |
| | TTGCTCAAGTTAAACCAAACAACACTGGTATTCGTGCTTCTGTTTATGAAAAAACAGCGAAAAACGGTGCGAAA |
| | TATGCAGACCGTACGTTCTATGTAACAAAAGAGCGTGCTCATGGTAATGAAACGTATGTATTATTAAACAATAC |
| | AAGCCATAACATCCCATTAGGTTGGTTCAATGTAAAAGACTTAAATGTTCAAAACCTAGGCAAAGAAGTTAAAA |
| | CGACTCAAAAATATACTGTTAATAAATCAAATAACGGCTTATCAATGGTTCCTTGGGGTACTAAAAACCAAGTC |
| | ATTTTAACAGGCAATAACATTGCTCAAGGTACATTTAATGCAACGAAACAAGTATCTGTAGGCAAAGATGTTTA |
| | TTTATACGGTACTATTAATAACCGCACTGGTTGGGTAAATGCAAAAGATTTAACTGCACCAACTGCTGTGAAAC |
| | CAACTACATCAGCTGCCAAAGATTATAACTACACTTATGTAATTAAAAATGGTAATGGTTATTACTATGTAACA |
| | CCAAATTCTGATACAGCTAAATACTCATTAAAAGCATTTAATGAACAACCATTCGCAGTTGTTAAAGAACAAGT |
| | CATTAATGGACAAACTTGGTACTATGGTAAATTATCTAACGGTAAATTAGCATGGATTAAATCAACTGATTTAG |
| | CTAAAGAATTAATTAAGTATAATCAAACAGGTATGACATTAAACCAAGTTGCTCAAATACAAGCTGGTTTACAA |
| | TATAAACCACAAGTACAACGTGTACCAGGTAAGTGGACAGATGCTAACTTTAATGATGTTAAGCATGCAATGGA |
| | TACGAAGCGTTTAGCTCAAGATCCAGCATTAAAATATCAATTCTTACGCTTAGACCAACCACAAAATATTTCTA |
| | TTGATAAAATTAATCAATTCTTAAAAGGTAAAGGTGTATTAGAAAACCAAGGTGCTGCATTTAACAAAGCTGCT |
| | CAAATGTATGGCATTAATGAAGTTTATCTTATCTCACATGCCCTATTAGAAACAGGTAACGGTACTTCTCAATT |
| | AGCGAAAGGTGCAGATGTAGTGAACAACAAAGTTGTAACTAACTCAAACACGAAATACCATAACGTATTTGGTA |
| | TTGCTGCATATGATAACGATCCTTTACGTGAAGGTATTAAATATGCTAAACAAGCTGGTTGGGACACAGTATCA |
| | AAAGCAATCGTTGGTGGTGCTAAATTCATCGGCAACTCATATGTAAAAGCTGGTCAAAATACACTTTACAAAAT |
| | GAGATGGAATCCTGCACATCCAGGAACACACCAATATGCTACAGATGTAGATTGGGCTAACATCAATGCTAAAA |
| | TCATCAAAGGCTACTATGATAAAATTGGCGAAGTCGGCAAATACTTCGACATCCCACAATATAAATAA |
| | (SEQ ID NO: 76) |
| Atl, | AUGUUAGGAGUAAUAAAUAGAAUGGCGAAAAAAUUCAAUUACAAACUACCAUCAAUGGUUGCAUUAACGCUUGU |
| FL, | AGGUUCAGCAGUCACUGCACAUCAAGUUCAAGCAGCUGAGACGACACAAGAUCAAACUACUAAUAAAAAUGUUU |
| WT, | UAGAUAGUAAUAAAGUUAAAGCAACUACUGAACAAGCAAAAGCUGAGGUAAAAAAUCCAACGCAAAACAUUUCU |
| RNA | GGCACUCAAGUAUAUCAAGACCCUGCUAUUGUCCAACCAAAAACAGCAAAUAACAAAACAGGCAAUGCUCAAGU |
| | AAGUCAAAAAGUUGAUACUGCACAAGUAAAUGGUGACACUCGUGCUAAUCAAUCAGCGACUACAAAUAAUACGC |
| | AGCCUGUUGCAAAGUCAACAAGCACUACAGCACCUAAAACUAACACUAAUGUUACAAAUGCUGGUUAUAGUUUA |
| | GUUGAUGAUGAAGAUGAUAAUUCAGAACAUCAAAUUAAUCCAGAAUUAAUUAAAUCAGCUGCUAAACCUGCAGC |
| | UCUUGAAACGCAAUAUAAAGCCGCAGCACCUAAAGCUAAAACUGAAGCGACACCUAAAGUAACUACUUUUAGCG |
| | CUUCAGCACAACCAAGAUCAGUUGCUGCAACACCAAAAACGAGUUUGCCAAAAUAUAAACCACAAGUAAACUCU |
| | UCAAUUAACGAUUACAUUCGUAAAAAUAACUUAAAAGCACCUAAAAUUGAAGAAGAUUAUACAUCUUACUUCCC |
| | UAAAUACGCAUACCGUAACGGCGUAGGUCGUCCUGAAGGUAUCGUAGUUCAUGAUACAGCUAAUGAUCGUUCGA |
| | CGAUAAAUGGUGAAAUUAGUUAUAUGAAAAAUAACUAUCAAAACGCAUUCGUACAUGCAUUUGUUGAUGGGGAU |
| | CGUAUAAUCGAAACAGCACCAACGGAUUACUUAUCUUGGGGUGUCGGUGCAGUCGGUAACCCUAGAUUCAUCAA |
| | UGUUGAAAUCGUACACACACACGACUAUGCUUCAUUUGCACGUUCAAUGAAUAACUAUGCUGACUAUGCAGCUA |
| | CACAAUUACAAUAUUAUGGUUUAAAACCAGACAGUGCUGAGUAUGAUGGAAAUGGUACAGUAUGGACUCACUAC |
| | GCUGUAAGUAAAUAUUUAGGUGGUACGGACCAUGCCGAUCCACAUGGAUAUUUAAGAAGUCAUAAUUAUAGUUA |
| | UGAUCAAUUAUAUGACUUAAUUAAUGAAAAAUAUUUAAUAAAAAUGGGUAAAGUGGCGCCAUGGGGUACGCAAU |
| | UUACAACUACCCCUACUACACCAUCAAAACCAACAACACCGUCGAAACCAUCAACUGGUAAAUUAACAGUUGCA |
| | GCAAACAAUGGUGUCGCACAAAUCAAACCAACAAAUAGUGGUUUAUAUACUACUGUUUACGACAAAACUGGUAA |
| | AGCAACUAAUGAAGUUCAAAAAACAUUUGCUGUAUCUAAAACAGCUACAUUAGGUAAUCAAAAAUUCUAUCUUG |
| | UUCAAGAUUACAAUUCUGGUAAUAAAUUUGGUUGGGUUAAAGAAGGCGAUGUGGUUUACAACACAGCUAAAUCA |
| | CCUGUAAAUGUAAAUCAAUCAUAUUCAAUCAAAUCUGGUACGAAACUUUAUACAGUACCUUGGGGUACAUCUAA |
| | ACAAGUUGCUGGUAGCGUGUCUGGCUCUGGAAACCAAACAUUUAAGGCUUCAAAGCAACAACAAAUUGAUAAAU |
| | CAAUUUAUUUAUAUGGCUCUGUGAAUGGUAAAUCUGGUUGGGUAAGUAAAGCAUAUUUAGUUGAUACUGCUAAA |
| | CCUACGCCUACACCAAUACCUAAGCCAUCAACACCUACAACAAAUAAUAAAUUAACAGUUUCAUCAUUAAACGG |
| | UGUUGCUCAAAUUAAUGCUAAAAACAAUGGCUUAUUCACUACAGUUUAUGACAAAACUGGUAAGCCAACGAAAG |
| | AAGUUCAAAAAACAUUUGCUGUAACAAAAGAAGCAAGUUUAGGUGGAAACAAAUUCUACUUAGUUAAAGAUUAC |
| | AAUAGUCCAACUUUAAUUGGUUGGGUUAAACAAGGUGACGUUAUUUAUAACAAUGCAAAAUCACCUGUAAAUGU |
| | AAUGCAAACAUAUACAGUAAAACCAGGCACUAAAUUAUAUUCAGUACCUUGGGGCACUUAUAAACAAGAAGCUG |
| | GUGCAGUUUCUGGUACAGGUAACCAAACUUUUAAAGCGACUAAGCAACAACAAAUUGAUAAAUCUAUCUAUUUA |
| | UUUGGAACUGUAAAUGGUAAAUCUGGUUGGGUAAGUAAAGCAUAUUUAGCUGUACCUGCUGCACCUAAAAAAGC |
| | AGUAGCACAACCAAAAACAGCUGUAAAAGCUUAUACUGUUACUAAACCACAAACGACUCAAACAGUUAGCAAGA |
| | UUGCUCAAGUUAAACCAAACAACACUGGUAUUCGUGCUUCUGUUUAUGAAAAAACAGCGAAAAACGGUGCGAAA |
| | UAUGCAGACCGUACGUUCUAUGUAACAAAAGAGCGUGCUCAUGGUAAUGAAACGUAUGUAUUAUUAAACAAUAC |
| | AAGCCAUAACAUCCCAUUAGGUUGGUUCAAUGUAAAAGACUUAAAUGUUCAAAACCUAGGCAAAGAAGUUAAAA |
| | CGACUCAAAAAUAUACUGUUAAUAAAUCAAAUAACGGCUUAUCAAUGGUUCCUUGGGGUACUAAAAACCAAGUC |
| | AUUUUAACAGGCAAUAACAUUGCUCAAGGUACAUUUAAUGCAACGAAACAAGUAUCUGUAGGCAAAGAUGUUUA |
| | UUUAUACGGUACUAUUAAUAACCGCACUGGUUGGGUAAAUGCAAAAGAUUUAACUGCACCAACUGCUGUGAAAC |
| | CAACUACAUCAGCUGCCAAAGAUUAUAACUACACUUAUGUAAUUAAAAAUGGUAAUGGUUAUUACUAUGUAACA |
| | CCAAAUUCUGAUACAGCUAAAUACUCAUUAAAAGCAUUUAAUGAACAACCAUUCGCAGUUGUUAAAGAACAAGU |
| | CAUUAAUGGACAAACUUGGUACUAUGGUAAAUUAUCUAACGGUAAAUUAGCAUGGAUUAAAUCAACUGAUUUAG |
| | CUAAAGAAUUAAUUAAGUAUAAUCAAACAGGUAUGACAUUAAACCAAGUUGCUCAAAUACAAGCUGGUUUACAA |
| | UAUAAACCACAAGUACAACGUGUACCAGGUAAGUGGACAGAUGCUAACUUUAAUGAUGUUAAGCAUGCAAUGGA |
| | UACGAAGCGUUUAGCUCAAGAUCCAGCAUUAAAAUAUCAAUUCUUACGCUUAGACCAACCACAAAAUAUUUCUA |
| | UUGAUAAAAUUAAUCAAUUCUUAAAAGGUAAAGGUGUAUUAGAAAACCAAGGUGCUGCAUUUAACAAAGCUGCU |
| | CAAAUGUAUGGCAUUAAUGAAGUUUAUCUUAUCUCACAUGCCCUAUUAGAAACAGGUAACGGUACUUCUCAAUU |
| | AGCGAAAGGUGCAGAUGUAGUGAACAACAAAGUUGUAACUAACUCAAACACGAAAUACCAUAACGUAUUUGGUA |
| | UUGCUGCAUAUGAUAACGAUCCUUUACGUGAAGGUAUUAAAUAUGCUAAACAAGCUGGUUGGGACACAGUAUCA |
| | AAAGCAAUCGUUGGUGGUGCUAAAUUCAUCGGCAACUCAUAUGUAAAAGCUGGUCAAAAUACACUUUACAAAAU |
| | GAGAUGGAAUCCUGCACAUCCAGGAACACACCAAUAUGCUACAGAUGUAGAUUGGGCUAACAUCAAUGCUAAAA |
| | UCAUCAAAGGCUACUAUGAUAAAAUUGGCGAAGUCGGCAAAUACUUCGACAUCCCACAAUAUAAAUAA |
| | (SEQ ID NO: 77) |
| CHIPS, | ATGAAAAAGAAATTAGCAACAACAGTTTTAGCATTAAGTTTTTTAACGGCAGGAATCAGTACACACCATCATTC |
| FL, | AGCGAAAGCTTTTACTTTTGAACCGTTTCCTACAAATGAAGAAATAGAATCAAATAAGAAAATGTTAGAGAAAG |
| WT, | AAAAAGCTTATAAAGAATCATTTAAAAATAGTGGTCTTCCTACAACGCTAGGAAAATTAGATGAACGTTTGAGA |
| DNA | AATTATTTAAAGAAAGGCACAAAAAATTCTGCTCAATTTGAAAAAATGGTTATTTTAACTGAAAATAAAGGTTA |
| | CTATACAGTATATCTGAATACACCACTTGCTGAAGATAGAAAAAATGTTGAGTTACTAGGTAAAATGTATAAAA |
| | CATACTTCTTTAAAAAAGGAGAGTCTAAATCATCTTATGTAATTAATGGTCCTGGAAAAACTAATGAATATGCA |
| | TACTAA (SEQ ID NO: 78) |
| CHIPS, | AUGAAAAAGAAAUUAGCAACAACAGUUUUAGCAUUAAGUUUUUUAACGGCAGGAAUCAGUACACACCAUCAUUC |
| FL, | AGCGAAAGCUUUUACUUUUGAACCGUUUCCUACAAAUGAAGAAAUAGAAUCAAAUAAGAAAAUGUUAGAGAAAG |
| WT, | AAAAAGCUUAUAAAGAAUCAUUUAAAAAUAGUGGUCUUCCUACAACGCUAGGAAAAUUAGAUGAACGUUUGAGA |
| RNA | AAUUAUUUAAAGAAAGGCACAAAAAAUUCUGCUCAAUUUGAAAAAAUGGUUAUUUUAACUGAAAAUAAAGGUUA |
| | CUAUACAGUAUAUCUGAAUACACCACUUGCUGAAGAUAGAAAAAAUGUUGAGUUACUAGGUAAAAUGUAUAAAA |
| | CAUACUUCUUUAAAAAAGGAGAGUCUAAAUCAUCUUAUGUAAUUAAUGGUCCUGGAAAAACUAAUGAAUAUGCA |
| | UACUAA (SEQ ID NO: 79) |
| SCIN, | ATATTTACTTTTTAGTGCTTCGTCAATTTCATTATAAATCTTTTGAAGTTGATATTTTGCTTCTGACATTTTCT |
| FL, | TAAAGTCTTTTGACTTAAGAGCATACATTGCTTTTTGACCTGAAATTTTTATAGTTCGCTTATAATAAGTGTTT |
| WT, | AAACTTCCAGTAGCTAATTCATTAACATTTAGTTCATCTAATAACGATTTTAATTCATTAGCTAACTTTTCGTT |
| DNA | TTGATATTCATTCGATGTTGGCAAGCTTGTGCTAGCTTGTGCCTCATTTTTATCTAGATTAGTTACTAGTGGTG |
| | ATGCTAAAACGATTGCTAAAGTTCCCGCAAGTATAGATTTTCTAATTTTCAT SEQ ID NO: 80) |
| SCIN, | AUAUUUACUUUUUAGUGCUUCGUCAAUUUCAUUAUAAAUCUUUUGAAGUUGAUAUUUUGCUUCUGACAUUUUCU |
| FL, | UAAAGUCUUUUGACUUAAGAGCAUACAUUGCUUUUUGACCUGAAAUUUUUAUAGUUCGCUUAUAAUAAGUGUUU |
| WT, | AAACUUCCAGUAGCUAAUUCAUUAACAUUUAGUUCAUCUAAUAACGAUUUUAAUUCAUUAGCUAACUUUUCGUU |
| RNA | UUGAUAUUCAUUCGAUGUUGGCAAGCUUGUGCUAGCUUGUGCCUCAUUUUUAUCUAGAUUAGUUACUAGUGGUG |
| | AUGCUAAAACGAUUGCUAAAGUUCCCGCAAGUAUAGAUUUUCUAAUUUUCAU (SEQ ID NO: 81) |
| Hla, | ATGAAAACACGTATAGTCAGCTCAGTAACAACAACACTATTGCTAGGTTCCATATTAATGAATCCTGTCGCTAA |
| FL, | TGCCGCAGATTCTGATATTAATATTAAAACCGGTACTACAGATATTGGAAGCAATACTACAGTAAAAACAGGTG |
| WT, | ATTTAGTCACTTATGATAAAGAAAATGGCATGCACAAAAAAGTATTTTATAGTTTTATCGATGATAAAAATCAT |
| DNA | AATAAAAAACTGCTAGTTATTAGAACGAAAGGTACCATTGCTGGTCAATATAGAGTTTATAGCGAAGAAGGTGC |
| | TAACAAAAGTGGTTTAGCCTGGCCTTCAGCCTTTAAGGTACAGTTGCAACTACCTGATAATGAAGTAGCTCAAA |
| | TATCTGATTACTATCCAAGAAATTCGATTGATACAAAAGAGTATATGAGTACTTTAACTTATGGATTCAACGGT |
| | AATGTTACTGGTGATGATACAGGAAAAATTGGCGGCCTTATTGGTGCAAATGTTTCGATTGGTCATACACTGAA |
| | ATATGTTCAACCTGATTTCAAAACAATTTTAGAGAGCCCAACTGATAAAAAAGTAGGCTGGAAAGTGATATTTA |
| | ACAATATGGTGAATCAAAATTGGGGACCATATGATAGAGATTCTTGGAACCCGGTATATGGCAATCAACTTTTC |
| | ATGAAAACTAGAAATGGCTCTATGAAAGCAGCAGATAACTTCCTTGATCCTAACAAAGCAAGTTCTCTATTATC |
| | TTCAGGGTTTTCACCAGACTTCGCTACAGTTATTACTATGGATAGAAAAGCATCCAAACAACAAACAAATATAG |
| | ATGTAATATACGAACGAGTTCGTGATGACTACCAATTGCACTGGACTTCAACAAATTGGAAAGGTACCAATACT |
| | AAAGATAAATGGATAGATCGTTCTTCAGAAAGATATAAAATCGATTGGGAAAAAGAAGAAATGACAAAT |
| | (SEQ ID NO: 82) |
| Hla, | AUGAAAACACGUAUAGUCAGCUCAGUAACAACAACACUAUUGCUAGGUUCCAUAUUAAUGAAUCCUGUCGCUAA |
| FL, | UGCCGCAGAUUCUGAUAUUAAUAUUAAAACCGGUACUACAGAUAUUGGAAGCAAUACUACAGUAAAAACAGGUG |
| WT, | AUUUAGUCACUUAUGAUAAAGAAAAUGGCAUGCACAAAAAAGUAUUUUAUAGUUUUAUCGAUGAUAAAAAUCAU |
| RNA | AAUAAAAAACUGCUAGUUAUUAGAACGAAAGGUACCAUUGCUGGUCAAUAUAGAGUUUAUAGCGAAGAAGGUGC |
| | UAACAAAAGUGGUUUAGCCUGGCCUUCAGCCUUUAAGGUACAGUUGCAACUACCUGAUAAUGAAGUAGCUCAAA |
| | UAUCUGAUUACUAUCCAAGAAAUUCGAUUGAUACAAAAGAGUAUAUGAGUACUUUAACUUAUGGAUUCAACGGU |
| | AAUGUUACUGGUGAUGAUACAGGAAAAAUUGGCGGCCUUAUUGGUGCAAAUGUUUCGAUUGGUCAUACACUGAA |
| | AUAUGUUCAACCUGAUUUCAAAACAAUUUUAGAGAGCCCAACUGAUAAAAAAGUAGGCUGGAAAGUGAUAUUUA |
| | ACAAUAUGGUGAAUCAAAAUUGGGGACCAUAUGAUAGAGAUUCUUGGAACCCGGUAUAUGGCAAUCAACUUUUC |
| | AUGAAAACUAGAAAUGGCUCUAUGAAAGCAGCAGAUAACUUCCUUGAUCCUAACAAAGCAAGUUCUCUAUUAUC |
| | UUCAGGGUUUUCACCAGACUUCGCUACAGUUAUUACUAUGGAUAGAAAAGCAUCCAAACAACAAACAAAUAUAG |
| | AUGUAAUAUACGAACGAGUUCGUGAUGACUACCAAUUGCACUGGACUUCAACAAAUUGGAAAGGUACCAAUACU |
| | AAAGAUAAAUGGAUAGAUCGUUCUUCAGAAAGAUAUAAAAUCGAUUGGGAAAAAGAAGAAAUGACAAAU |
| | (SEQ ID NO: 83) |
| CgoX, | TCGTATCGTAAAGATGGTTATATGATTGAACTAGGGCCTGAATCTTATTTAGGTAGAAAAACGATTATGACAGA |
| FL, | ATTAGCGAAAGATATTGGATTAGAACAAGATATTGTTACAAATACGACTGGACAATCATATATTTTTGCGAAAA |
| WT, | ATAAATTATATCCGATTCCAGGTGGTTCAATTATGGGTATTCCAACAGATATTAAACCATTTGTTACTACAAAA |
| DNA | TTAATATCGCCACTTGGTAAATTAAGAGCAGGGTTTGATTTATTAAAAAAACCTACTCAAATGCAGGATGGTGA |
| | CATTTCTGTTGGTGCATTTTTCAGAGCAAGATTAGGTAATGAGGTACTTGAGAATTTAATAGAGCCTTTAATGG |
| | GTGGTATTTATGGTACCGATATTGATAAATTAAGTTTGATGAGTACGTTTCCTAATTTTAAAGAAAAAGAAGAG |
| | GCATTCGGAAGTCTGATAAAAGGTATGAAGGATGAGAAAAATAAGCGTCTGAAACAAAGACAATTATATCCTGG |
| | CGCACCGAAAGGACAATTCAAACAATTTAAGCATGGTTTAAGCTCATTTATTGAAGCATTAGAACAAGATGTTA |
| | AAAATAAAGGTGTGACAATACGCTACAATACGTCAGTGGATGATATAATTACATCTCAAAAACAATATAAAATT |
| | GTTTACAATGATCAACTAGAAGAAGTGTATGATGGCGTATTAGTAACGACACCACATCAAGTGTTTTTAAATTG |
| | GTTCGGACAAGATCCAGCATTTGATTACTTTAAAACGATGGATAGTACGACTGTTGCAACTGTTGTATTGGCAT |
| | TTGATGAAAAAGACATTGAAAATACCCATGATGGTACTGGCTTCGTAATTGCGAGAACGAGTGATACAGACATT |
| | ACCGCATGTACTTGGACATCGAAAAAATGGCCATTTACTACACCGGAAGGTAAGGTTTTGATTCGTGCGTATGT |
| | AGGTAAACCAGGTGATACTGTGGTTGATGATCATACAGATAATGAATTAGTATCGATTGTACGTAGAGATTTAA |
| | GTCAAATGATGACATTTAAAGGTGATCCTGAATTTACAATTGTCAATCGTTTGCCGAAAAGTATGCCACAGTAC |
| | CATGTCGGTCATATTCAACAAATTAGACAGATTCAAGCACATATTAAACAAACATATCCACGACTTAGAGTAAC |
| | TGGTGCATCTTTTGAAGCGGTTGGACTACCTGATTGTATTACGCAAGGTAAAGTTGCTGCTGAAGAAGTAATCG |
| | CAGAGTTGTAA (SEQ ID NO: 84) |
| CgoX, | UCGUAUCGUAAAGAUGGUUAUAUGAUUGAACUAGGGCCUGAAUCUUAUUUAGGUAGAAAAACGAUUAUGACAGA |
| FL, | AUUAGCGAAAGAUAUUGGAUUAGAACAAGAUAUUGUUACAAAUACGACUGGACAAUCAUAUAUUUUUGCGAAAA |
| WT, | AUAAAUUAUAUCCGAUUCCAGGUGGUUCAAUUAUGGGUAUUCCAACAGAUAUUAAACCAUUUGUUACUACAAAA |
| RNA | UUAAUAUCGCCACUUGGUAAAUUAAGAGCAGGGUUUGAUUUAUUAAAAAAACCUACUCAAAUGCAGGAUGGUGA |
| | CAUUUCUGUUGGUGCAUUUUUCAGAGCAAGAUUAGGUAAUGAGGUACUUGAGAAUUUAAUAGAGCCUUUAAUGG |
| | GUGGUAUUUAUGGUACCGAUAUUGAUAAAUUAAGUUUGAUGAGUACGUUUCCUAAUUUUAAAGAAAAAGAAGAG |
| | GCAUUCGGAAGUCUGAUAAAAGGUAUGAAGGAUGAGAAAAAUAAGCGUCUGAAACAAAGACAAUUAUAUCCUGG |
| | CGCACCGAAAGGACAAUUCAAACAAUUUAAGCAUGGUUUAAGCUCAUUUAUUGAAGCAUUAGAACAAGAUGUUA |
| | AAAAUAAAGGUGUGACAAUACGCUACAAUACGUCAGUGGAUGAUAUAAUUACAUCUCAAAAACAAUAUAAAAUU |
| | GUUUACAAUGAUCAACUAGAAGAAGUGUAUGAUGGCGUAUUAGUAACGACACCACAUCAAGUGUUUUUAAAUUG |
| | GUUCGGACAAGAUCCAGCAUUUGAUUACUUUAAAACGAUGGAUAGUACGACUGUUGCAACUGUUGUAUUGGCAU |
| | UUGAUGAAAAAGACAUUGAAAAUACCCAUGAUGGUACUGGCUUCGUAAUUGCGAGAACGAGUGAUACAGACAUU |
| | ACCGCAUGUACUUGGACAUCGAAAAAAUGGCCAUUUACUACACCGGAAGGUAAGGUUUUGAUUCGUGCGUAUGU |
| | AGGUAAACCAGGUGAUACUGUGGUUGAUGAUCAUACAGAUAAUGAAUUAGUAUCGAUUGUACGUAGAGAUUUAA |
| | GUCAAAUGAUGACAUUUAAAGGUGAUCCUGAAUUUACAAUUGUCAAUCGUUUGCCGAAAAGUAUGCCACAGUAC |
| | CAUGUCGGUCAUAUUCAACAAAUUAGACAGAUUCAAGCACAUAUUAAACAAACAUAUCCACGACUUAGAGUAAC |
| | UGGUGCAUCUUUUGAAGCGGUUGGACUACCUGAUUGUAUUACGCAAGGUAAAGUUGCUGCUGAAGAAGUAAUCG |
| | CAGAGUUGUAA (SEQ ID NO: 85) |
| IsaA, | ATGAAAAAGACAATTATGGCATCATCATTAGCAGTGGCATTAGGTGTAACAGGTTACGCAGCAGGTACAGGACA |
| FL, | TCAAGCACACGCTGCTGAAGTAAACGTTGATCAAGCACACTTAGTTGACTTAGCGCATAATCACCAAGATCAAT |
| WT, | TAAATGCAGCTCCAATCAAAGATGGTGCATATGACATCCACTTTGTAAAAGATGGTTTCCAATATAACTTCACT |
| DNA | TCAAATGGTACTACATGGTCATGGAGCTATGAAGCAGCTAATGGTCAAACTGCTGGTTTCTCAAACGTTGCAGG |
| | TGCAGACTACACTACTTCATACAACCAAGGTTCAAATGTACAATCAGTAAGCTACAATGCACAATCAAGTAACT |
| | CAAACGTTGAAGCTGTTTCAGCTCCAACTTACCATAACTACAGCACTTCAACTACTTCAAGTTCAGTGAGATTA |
| | AGCAATGGTAATACTGCAGGTGCTACTGGTTCATCAGCAGCTCAAATCATGGCTCAACGTACTGGTGTTTCAGC |
| | TTCTACATGGGCTGCAATCATCGCTCGTGAATCAAATGGTCAAGTAAATGCTTACAACCCATCAGGTGCTTCAG |
| | GTTTATTCCAAACTATGCCAGGTTGGGGTCCAACAAACACTGTTGACCAACAAATCAACGCAGCTGTTAAAGCA |
| | TACAAAGCACAAGGTTTAGGTGCTTGGGGATTCTAA (SEQ ID NO: 115) |
| IsaA, | AUGAAAAAGACAAUUAUGGCAUCAUCAUUAGCAGUGGCAUUAGGUGUAACAGGUUACGCAGCAGGUACAGGACA |
| FL, | UCAAGCACACGCUGCUGAAGUAAACGUUGAUCAAGCACACUUAGUUGACUUAGCGCAUAAUCACCAAGAUCAAU |
| WT, | UAAAUGCAGCUCCAAUCAAAGAUGGUGCAUAUGACAUCCACUUUGUAAAAGAUGGUUUCCAAUAUAACUUCACU |
| RNA | UCAAAUGGUACUACAUGGUCAUGGAGCUAUGAAGCAGCUAAUGGUCAAACUGCUGGUUUCUCAAACGUUGCAGG |
| | UGCAGACUACACUACUUCAUACAACCAAGGUUCAAAUGUACAAUCAGUAAGCUACAAUGCACAAUCAAGUAACU |
| | CAAACGUUGAAGCUGUUUCAGCUCCAACUUACCAUAACUACAGCACUUCAACUACUUCAAGUUCAGUGAGAUUA |
| | AGCAAUGGUAAUACUGCAGGUGCUACUGGUUCAUCAGCAGCUCAAAUCAUGGCUCAACGUACUGGUGUUUCAGC |
| | UUCUACAUGGGCUGCAAUCAUCGCUCGUGAAUCAAAUGGUCAAGUAAAUGCUUACAACCCAUCAGGUGCUUCAG |
| | GUUUAUUCCAAACUAUGCCAGGUUGGGGUCCAACAAACACUGUUGACCAACAAAUCAACGCAGCUGUUAAAGCA |
| | UACAAAGCACAAGGUUUAGGUGCUUGGGGAUUCUAA (SEQ ID NO: 116) |
| SceD, | ATGAAGAAAACATTACTCGCATCATCATTAGCAGTAGGTTTAGGAATCGTAGCAGGAAATGCAGGTCACGAAGC |
| FL, | CCATGCAAGTGAAGCGGACTTAAATAAAGCATCTTTAGCGCAAATGGCGCAATCAAATGATCAAACATTAAATC |
| WT, | AAAAACCAATTGAAGCTGGGGCTTATAATTATACATTTGACTATGAAGGGTTTACTTATCACTTTGAATCAGAT |
| DNA | GGTACACACTTTGCTTGGAATTACCATGCAACAGGTACTAATGGAGCAGACATGAGTGCACAAGCACCTGCAAC |
| | TAATAATGTTGCACCATCAGCTGTTCAAGCTAATCAAGTACAATCACAAGAAGTTGAAGCACCACAAAATGCTC |
| | AAACTCAACAACCACAAGCATCAACATCAAACAATTCACAAGTTACTGCAACACCAACTGAATCAAAATCATCA |
| | GAAGGTTCATCAGTAAATGTGAATGCTCATCTAAAACAAATTGCTCAACGTGAATCAGGTGGCAATATTCATGC |
| | TGTAAATCCAACATCAGGTGCAGCTGGTAAGTATCAATTCTTACAATCAACTTGGGATTCAGTAGCACCTGCTA |
| | AATATAAAGGTGTATCACCAGCAAATGCTCCTGAAAGTGTTCAAGATGCCGCAGCAGTAAAATTATATAACACT |
| | GGTGGCGCTGGACATTGGGTTACTGCATAA (SEQ ID NO: 117) |
| SceD, | AUGAAGAAAACAUUACUCGCAUCAUCAUUAGCAGUAGGUUUAGGAAUCGUAGCAGGAAAUGCAGGUCACGAAGC |
| FL, | CCAUGCAAGUGAAGCGGACUUAAAUAAAGCAUCUUUAGCGCAAAUGGCGCAAUCAAAUGAUCAAACAUUAAAUC |
| WT | AAAAACCAAUUGAAGCUGGGGCUUAUAAUUAUACAUUUGACUAUGAAGGGUUUACUUAUCACUUUGAAUCAGAU |
| RNA | GGUACACACUUUGCUUGGAAUUACCAUGCAACAGGUACUAAUGGAGCAGACAUGAGUGCACAAGCACCUGCAAC |
| | UAAUAAUGUUGCACCAUCAGCUGUUCAAGCUAAUCAAGUACAAUCACAAGAAGUUGAAGCACCACAAAAUGCUC |
| | AAACUCAACAACCACAAGCAUCAACAUCAAACAAUUCACAAGUUACUGCAACACCAACUGAAUCAAAAUCAUCA |
| | GAAGGUUCAUCAGUAAAUGUGAAUGCUCAUCUAAAACAAAUUGCUCAACGUGAAUCAGGUGGCAAUAUUCAUGC |
| | UGUAAAUCCAACAUCAGGUGCAGCUGGUAAGUAUCAAUUCUUACAAUCAACUUGGGAUUCAGUAGCACCUGCUA |
| | AAUAUAAAGGUGUAUCACCAGCAAAUGCUCCUGAAAGUGUUCAAGAUGCCGCAGCAGUAAAAUUAUAUAACACU |
| | GGUGGCGCUGGACAUUGGGUUACUGCAUAA (SEQ ID NO: 118) |

The sequence associated with each accession number listed in Tables 3-7 below corresponds to the sequences (nucleic acid and protein) corresponding to the accession number as of Jun. 1, 2021.

TABLE 3
Exemplary ATL (and Amd and Gmd) Sequences

| ATL Acc. # | QHK82268.1 | ARH71669.1 | AVU06521.1 | ADL65054.1 | QIT40948.1 |
|---|---|---|---|---|---|
| AIA27545.1 | AUG73417.1 | QBP95831.1 | SQE80535.1 | AZL91048.1 | BBG07794.1 |
| VDZ26364.1 | SQE66125.1 | QHK67001.1 | AEZ37049.1 | SQF44913.1 | AXJ32877.1 |
| QHL22074.1 | QHL19437.1 | AKA99053.1 | AUU50652.1 | AWE66323.1 | QDX09302.1 |
| QHK80000.1 | AGU54772.1 | AUU56998.1 | QBS23517.1 | QNU56098.1 | AUU61377.1 |
| QCW91169.1 | QAZ56892.1 | QBX61579.1 | ATC71080.1 | AXJ67355.1 | ATN57397.1 |
| SQI84544.1 | QCW88502.1 | AMO16628.1 | QHG56624.1 | VDZ24317.1 | QBB19530.1 |
| BBN30061.1 | AJC28318.1 | SQE99548.1 | SQE33349.1 | VDY47909.1 | ABD30118.1 |
| QCA26276.1 | ALS85314.1 | ANI71313.1 | AVS04626.1 | ATW79747.1 | AWR18876.1 |
| AJC36855.1 | AUU42880.1 | VDZ92305.1 | VDZ34849.1 | QHK76644.1 | AOO98268.1 |
| ALY17727.1 | AUU67039.1 | AZB46278.1 | AJC42542.1 | AJC34008.1 | QNU59028.1 |
| AXP52621.1 | QAZ58994.1 | AXJ55891.1 | AYV01683.1 | VEG25618.1 | QIT37207.1 |
| AXG01202.1 | AMV79555.1 | ARH69052.1 | AWR05733.1 | AUJ56535.1 | QBS04279.1 |
| QHK39598.1 | APD03885.1 | QBY47984.1 | AWW93107.1 | AIW26676.1 | AXG03171.1 |
| VEB64990.1 | ALY20567.1 | BAB94801.1 | ALQ99246.1 | QBQ51056.1 | AVU13370.1 |
| BAR08491.1 | AIU85259.1 | AFR73070.1 | VDZ19682.1 | SQE87799.1 | BBJ12315.1 |
| AGW33435.1 | QNU49285.1 | AXJ22705.1 | AXJ48290.1 | QHK95765.1 | AVG57298.1 |
| AZH09543.1 | AUU57602.1 | VEE67863.1 | AJE64455.1 | AXJ45725.1 | AUJ54815.1 |
| BAR11215.1 | QNU51625.1 | SQE83302.1 | QHL03451.1 | AXJ60858.1 | AND02216.1 |
| AYC77770.1 | CRL34056.1 | QCT54225.1 | QHL60610.1 | AUU63559.1 | AUU79604.1 |
| QHK52288.1 | ADC37218.1 | ARH74474.1 | QDX02790.1 | SQE76355.1 | AJC31165.1 |
| AZG95347.1 | QLL20295.1 | QHL76527.1 | QBC23571.1 | QHL40570.1 | QHL45883.1 |
| VDZ16961.1 | VED79543.1 | QCV70548.1 | AXR96132.1 | SQF73254.1 | QLI00317.1 |
| QHK31481.1 | ATN49398.1 | QJR41389.1 | QBX59079.1 | QHL08420.1 | QKV59586.1 |
| AID39491.1 | AXN69218.1 | QAZ54108.1 | QCV76124.1 | QHK47421.1 | QBS01776.1 |
| ARG45532.1 | ADI97512.1 | QHL24191.1 | ARH66348.1 | SQE35891.1 | AHW66597.1 |
| CAG40030.1 | QJR07161.1 | AVS39866.1 | AXJ40698.1 | AVG52384.1 | VDZ14418.1 |
| AXJ36348.1 | AXS24625.1 | ARH57627.1 | AUS75090.1 | AII55508.1 | QHL65975.1 |
| AXJ43207.1 | ARH77311.1 | BAF67194.1 | ATV03764.1 | AEB88134.1 | QHK87893.1 |
| QHK93181.1 | AXJ63523.1 | AHZ98841.1 | AWQ31838.1 | AMQ78466.1 | AUW97674.1 |
| QHK60057.1 | ANI73938.1 | APW75432.1 | AWW96194.1 | AXJ50849.1 | BBK64506.1 |
| QHK49659.1 | ASC50554.1 | AUU48045.1 | VED73018.1 | ARI73288.1 | QBS26543.1 |
| AXJ39012.1 | AYU99258.1 | QHK85316.1 | CEH26150.1 | ATW76945.1 | AJC39695.1 |
| QHL37994.1 | QHL57873.1 | ALO31063.1 | AUU73468.1 | ASC53246.1 | AVG61550.1 |
| SQE68782.1 | QLL17621.1 | ASF31912.1 | SQE40470.1 | QBS06770.1 | QBZ85184.1 |
| AMO52144.1 | QHK26185.1 | CCG15599.1 | QNU47487.1 | APC76919.1 | QEQ41074.1 |
| QHL79454.1 | AXJ58449.1 | AXJ53370.1 | AWQ88832.1 | ATH57064.1 | AGW35968.1 |
| VDZ37637.1 | SQE60986.1 | QCW85812.1 | QBC20808.1 | QBB15878.1 | CAI80607.1 |
| SQE63617.1 | AXI05783.1 | VDZ11822.1 | QNU54079.1 | ATC67235.1 | ATF38539.1 |
| AUU52521.1 | ATN60129.1 | BAF77928.1 | QHK98312.1 | AXU08131.1 | |

TABLE 4
Exemplary HLA Sequences

| HLA Acc. # | SQE66230.1 | QHK67112.1 | AEZ37155.1 | SQF45105.1 | AXJ32979.1 |
|---|---|---|---|---|---|
| AIA27648.1 | QHL20577.1 | AKA99160.1 | AUU50763.1 | AWE65815.1 | QDX09134.1 |
| VDZ27659.1 | AGU54876.1 | AUU56887.1 | QBS23634.1 | QNU57195.1 | AUU62623.1 |
| QHL21908.1 | QAZ56778.1 | QBX61684.1 | ATC71258.1 | AXJ67181.1 | ATN58869.1 |
| QHK80108.1 | QCW88617.1 | AMO16733.1 | QHG56734.1 | VDZ24583.1 | QBB17650.1 |
| QCW91348.1 | AJC28433.1 | SQE99653.1 | SQE33453.1 | VDY48013.1 | ABD30233.1 |
| SQI85144.1 | ALS85426.1 | ANI71187.1 | AVS04738.1 | ATW79862.1 | AWR18986.1 |
| BBN29957.1 | AUU42992.1 | VDZ92411.1 | VDZ34970.1 | QHK78583.1 | AOO98445.1 |
| QCA26382.1 | AUU66924.1 | AZB46394.1 | AJC42657.1 | AJC34123.1 | QNU59124.1 |
| AJC36970.1 | QAZ60579.1 | AXJ57236.1 | AYV01787.1 | VEG25812.1 | QIT37097.1 |
| ALY17878.1 | AMV79665.1 | ARH69166.1 | AWR05710.1 | AUJ58387.1 | QBS04383.1 |
| AXP52506.1 | APD03992.1 | QBY48148.1 | AWW93223.1 | AIW26779.1 | AXG03284.1 |
| AXG00591.1 | ALY20729.1 | BAB94909.1 | ALQ99354.1 | QBQ52484.1 | AVU14833.1 |
| QHK40738.1 | AIU85108.1 | AFR73182.1 | VDZ19787.1 | SQE88700.1 | BBJ12426.1 |
| VEB65171.1 | QNU49392.1 | AXJ22810.1 | AXJ48397.1 | QHK95656.1 | AVG57477.1 |
| BAR09922.1 | AUU60105.1 | VEE67966.1 | AJE64560.1 | AXJ45832.1 | AUJ55523.1 |
| AGW33544.1 | QNU51722.1 | SQE83406.1 | QHL04586.1 | AXJ61031.1 | AND02328.1 |
| AZH09717.1 | CRL34380.1 | QCT54338.1 | QHL60502.1 | AUU63390.1 | AUU79493.1 |
| BAR12646.1 | ADC37330.1 | ARH74590.1 | QDX02958.1 | SQE76461.1 | AJC31280.1 |
| AYC77872.1 | QLL21754.1 | QHL76650.1 | QBC23686.1 | QHL40463.1 | QHL45719.1 |
| QHK53664.1 | VED79887.1 | QCV70664.1 | AXR96305.1 | SQF73358.1 | QLI00424.1 |
| AZG95461.1 | ATN49564.1 | QJR41499.1 | QBX59184.1 | QHL09813.1 | QKV60930.1 |
| VDZ17069.1 | AXN69337.1 | QAZ53995.1 | QCV76240.1 | QHK47531.1 | QBS01881.1 |
| QHK31370.1 | ADI97674.1 | QHL25333.1 | ARH66463.1 | SQE35996.1 | AHW67255.1 |
| AID39599.1 | QJR07270.1 | AVS39979.1 | AXJ42026.1 | AVG52269.1 | VDZ14523.1 |
| ARG45647.1 | AXS24512.1 | ARH57741.1 | AUS74977.1 | AII55617.1 | QHL65868.1 |
| CAG41491.1 | ARH77426.1 | BAF67345.1 | ATV03870.1 | AEB88242.1 | QHK87767.1 |
| AXJ36174.1 | AXJ63696.1 | AHZ98946.1 | AWQ31724.1 | AMQ79365.1 | AUW97786.1 |
| AXJ44537.1 | ANI74052.1 | APW75546.1 | AWW96309.1 | AXJ52194.1 | BBK64614.1 |
| QHK94299.1 | ASC50666.1 | AUU48160.1 | VED73365.1 | ARI73403.1 | QBS26659.1 |
| QHK59933.1 | AYU99360.1 | QHK85210.1 | CEH25978.1 | ATW77121.1 | AJC39810.1 |
| QHK49770.1 | QHL57763.1 | ALO31169.1 | AUU73583.1 | ASC53358.1 | AVG62493.1 |
| AXJ38838.1 | QLL19077.1 | ASF32021.1 | SQE40576.1 | QBS06874.1 | QBZ85296.1 |
| QHL37871.1 | QHK26074.1 | CCG15709.1 | QNU46905.1 | APC77024.1 | QEQ41243.1 |
| SQE68886.1 | AXJ58551.1 | AXJ54715.1 | AWQ89143.1 | ATH57173.1 | AGW36077.1 |
| AMO51969.1 | SQE61092.1 | QCW85926.1 | QBC20925.1 | QBB15983.1 | QBP95930.1 |
| QHL80774.1 | AXI05959.1 | VDZ11933.1 | QNU55663.1 | ATC67413.1 | SQE80642.1 |
| VDZ37740.1 | ATN60238.1 | BAF78036.1 | QHK98420.1 | AXU08234.1 | AZL91161.1 |
| SQE63722.1 | AUU52637.1 | AVU06631.1 | ADL65161.1 | CAI80715.1 | BBG07902.1 |
| AUG73537.1 | QHK83680.1 | ARH71784.1 | QIT41057.1 | ATF38654.1 | |

TABLE 5
Exemplary CHIPS Sequences

| CHIPS Acc. # | SQE66224.1 | QHK67758.1 | AEZ37149.1 | SQF45093.1 | AXJ32973.1 |
|---|---|---|---|---|---|
| AIA28459.1 | QHL18409.1 | AKB00045.1 | AUU50757.1 | AWE66399.1 | QDX09141.1 |
| VDZ27671.1 | AGU54870.1 | AUU56894.1 | QBS23627.1 | QNU57604.1 | AUU60457.1 |
| QHL21167.1 | QAZ56785.1 | QBX61678.1 | ATC72126.1 | AXJ66415.1 | ATN58387.1 |
| QHK80846.1 | QCW89354.1 | AMO17502.1 | QHG56727.1 | VDZ24569.1 | QBB19362.1 |
| QCW91340.1 | AJC29328.1 | SQE99647.1 | SQE33447.1 | VDY48781.1 | ABD31214.1 |
| SQI89279.1 | ALS85419.1 | ANI70332.1 | AVS04732.1 | ATW80682.1 | AWR18979.1 |
| BBN29963.1 | AUU41156.1 | VDZ92405.1 | VDZ34964.1 | QHK76374.1 | AOO98437.1 |
| QCA26376.1 | AUU66931.1 | AZB47297.1 | AJC43551.1 | AJC35017.1 | QNU59594.1 |
| AJC37867.1 | QAZ59103.1 | AXJ57030.1 | AYV01781.1 | VEG28168.1 | QIT38710.1 |
| ALY18727.1 | AMV80467.1 | ARH69938.1 | AWR04270.1 | AUJ57909.1 | QBS04377.1 |
| AXP52513.1 | APD03985.1 | QBY48142.1 | AWW93217.1 | AIW27897.1 | AXG04088.1 |
| AXG01378.1 | ALY21521.1 | BAB94903.1 | ALR00130.1 | QBQ52018.1 | AVU14347.1 |
| QHK41220.1 | AIU84325.1 | AFR73176.1 | VDZ19781.1 | SQE88885.1 | BBJ13297.1 |
| VEB66594.1 | QNU50825.1 | AXJ22804.1 | AXJ48391.1 | QHK94888.1 | AVG57470.1 |
| BAR09439.1 | AUU59298.1 | VEE67960.1 | AJE64554.1 | AXJ45826.1 | AUJ55671.1 |
| AGW34247.1 | QNU51717.1 | SQE83400.1 | QHL02572.1 | AXJ61798.1 | AND03197.1 |
| AZH09711.1 | CRL34360.1 | QCT54331.1 | QHL59686.1 | AUU63396.1 | AUU78698.1 |
| BAR12163.1 | ADC38113.1 | ARH75435.1 | QDX02952.1 | SQE76455.1 | AJC32174.1 |
| AYC77216.1 | QLL21270.1 | QHL77410.1 | QBC23679.1 | QHL40469.1 | QHL44965.1 |
| QHK53234.1 | VED79875.1 | QCV70657.1 | AXR96298.1 | SQF74254.1 | QLI00417.1 |
| AZG96447.1 | ATN50208.1 | QJR42339.1 | QBX59179.1 | QHL09356.1 | QKV59688.1 |
| VDZ17954.1 | AXN69328.1 | QAZ54002.1 | QCV76233.1 | QHK47525.1 | QBS01875.1 |
| QHK31376.1 | ADI98513.1 | QHL26270.1 | ARH67238.1 | SQE35990.1 | AHW66166.1 |
| AID39592.1 | QJR07962.1 | AVS39972.1 | AXJ41576.1 | AVG52276.1 | VDZ14517.1 |
| ARG46515.1 | AXS24519.1 | ARH58610.1 | AUS74983.1 | AII56324.1 | QHL65061.1 |
| CAG41022.1 | ARH77419.1 | BAF68149.1 | ATV03864.1 | AEB88236.1 | QHK87010.1 |
| AXJ35408.1 | AXJ64462.1 | AHZ99788.1 | AWQ31731.1 | AMQ80350.1 | AUX00166.1 |
| AXJ44087.1 | ANI74044.1 | APW75539.1 | AWW97124.1 | AXJ51988.1 | BBK65405.1 |
| QHK92234.1 | ASC50658.1 | AUU49005.1 | VED76227.1 | ARI73396.1 | QBS26276.1 |
| QHK61566.1 | AYU99354.1 | QHK84406.1 | CEH25983.1 | ATW77115.1 | AJC40705.1 |
| QHK50534.1 | QHL57004.1 | ALO31162.1 | AUU71756.1 | ASC53351.1 | AVG59861.1 |
| AXJ38072.1 | QLL18593.1 | ASF32014.1 | SQE40569.1 | QBS06868.1 | QBZ86272.1 |
| QHL37877.1 | QHK25316.1 | CCG16570.1 | QNU46900.1 | APC77018.1 | QEQ41160.1 |
| SQE69678.1 | AXJ58545.1 | AXJ54509.1 | AWQ89201.1 | ATH57884.1 | AGW36071.1 |
| AMO53831.1 | SQE61086.1 | QCW85918.1 | QBC21720.1 | QBB15976.1 | QBP95923.1 |
| QHL81094.1 | AXI05951.1 | VDZ11927.1 | QNU54917.1 | ATC68280.1 | SQE80636.1 |
| VDZ38536.1 | ATN60233.1 | BAF78029.1 | QHK99173.1 | AXU08228.1 | AZL92123.1 |
| SQE63716.1 | AUU52630.1 | AVU07408.1 | ADL65155.1 | CAI80707.1 | BBG07895.1 |
| AUG72858.1 | QHK82432.1 | ARH72650.1 | QIT41818.1 | ATF39457.1 | |

TABLE 6
Exemplary SCIN Sequences

| SCIN Acc. No. | AUG72858.1 | QBP96681.1 | SQE81531.1 | AZL91156.1 | BBG08678.1 |
|---|---|---|---|---|---|
| AIA28458.1 | SQE67009.1 | QHK67757.1 | AEZ37152.1 | SQF46692.1 | AXJ32976.1 |
| VDZ28307.1 | QHL18410.1 | AKB00044.1 | AUU51645.1 | AWE65797.1 | QDX08358.1 |
| QHL21168.1 | AGU54458.1 | AUU56095.1 | QBS24494.1 | QNU57605.1 | AUU60458.1 |
| QHK80845.1 | QAZ55969.1 | QBX62518.1 | ATC72125.1 | AXJ66416.1 | ATN58386.1 |
| QCW91343.1 | QCW89353.1 | AMO17501.1 | QHG57475.1 | VDZ26736.1 | QBB19363.1 |
| SQI89276.1 | AJC29327.1 | SQF00435.1 | SQE33450.1 | VDY48780.1 | ABD31213.1 |
| BBN29217.1 | ALS85422.1 | ANI70333.1 | AVS04735.1 | ATW80681.1 | AWR19766.1 |
| QCA25713.1 | AUU41155.1 | VDZ93198.1 | VDZ34967.1 | QHK76375.1 | AOO98440.1 |
| AJC37866.1 | AUU66077.1 | AZB47296.1 | AJC43550.1 | AJC35016.1 | QNU58736.1 |
| ALY18726.1 | QAZ59984.1 | AXJ55989.1 | AYV02537.1 | VEG27622.1 | QIT38711.1 |
| AXP51635.1 | AMV80466.1 | ARH69937.1 | AWR05817.1 | AUJ57912.1 | QBS05012.1 |
| AXG00588.1 | APD04624.1 | QBY48997.1 | AWW94041.1 | AIW27638.1 | AXG03281.1 |
| QHK38654.1 | ALY21520.1 | BAB95749.1 | ALR00129.1 | QBQ52017.1 | AVU14346.1 |
| VEB66593.1 | AIU84326.1 | AFR73179.1 | VDZ20570.1 | SQE88884.1 | BBJ13296.1 |
| BAR09437.1 | QNU50100.1 | AXJ23629.1 | AXJ49202.1 | QHK94889.1 | AVG57473.1 |
| AGW34246.1 | AUU59299.1 | VEE67963.1 | AJE65418.1 | AXJ46639.1 | AUJ55674.1 |
| AZH09714.1 | QNU52321.1 | SQE82627.1 | QHL02573.1 | AXJ61797.1 | AND03196.1 |
| BAR12161.1 | CRL32976.1 | QCT55115.1 | QHL59687.1 | AUU65373.1 | AUU78699.1 |
| AYC77216.1 | ADC38112.1 | ARH75434.1 | QDX02955.1 | SQE76458.1 | AJC32173.1 |
| QHK53233.1 | QLL21269.1 | QHL77409.1 | QBC22715.1 | QHL39664.1 | QHL44966.1 |
| AZG95456.1 | VED79881.1 | QCV71528.1 | AXR96301.1 | SQF74253.1 | QLI01212.1 |
| VDZ17953.1 | ATN50207.1 | QJR42338.1 | QBX59182.1 | QHL09355.1 | QKV60498.1 |
| QHK30583.1 | AXN67420.1 | QAZ54663.1 | QCV77103.1 | QHK47528.1 | QBS02717.1 |
| AID40486.1 | ADI98513.1 | QHL24086.1 | ARH67237.1 | SQE36887.1 | AHW67595.1 |
| ARG46514.1 | QJR07961.1 | AVS39975.1 | AXJ41575.1 | AVG51422.1 | VDZ15309.1 |
| CAG41021.1 | AXS25249.1 | ARH58609.1 | AUS74097.1 | AII56323.1 | QHL65062.1 |
| AXJ35409.1 | ARH77422.1 | BAF68148.1 | ATV03867.1 | AEB89035.1 | QHK87011.1 |
| AXJ44086.1 | AXJ64461.1 | AHZ99787.1 | AWQ31728.1 | AMQ80549.1 | AUW97782.1 |
| QHK92235.1 | ANI74856.1 | APW75542.1 | AWW97123.1 | AXJ50947.1 | BBK65404.1 |
| QHK61567.1 | ASC50661.1 | AUU49004.1 | VED76224.1 | ARI74271.1 | QBS26277.1 |
| QHK50533.1 | AYV00088.1 | QHK84407.1 | CEH25980.1 | ATW78003.1 | AJC40704.1 |
| AXJ38073.1 | QHL57005.1 | ALO32037.1 | AUU71755.1 | ASC53354.1 | AVG59860.1 |
| QHL37013.1 | QLL18592.1 | ASF32860.1 | SQE41312.1 | QBS06201.1 | QBZ86271.1 |
| SQE69677.1 | QHK25317.1 | CCG16569.1 | QNU47627.1 | APC77021.1 | QEQ41161.1 |
| AMO53832.1 | AXJ58548.1 | AXJ53468.1 | AWQ87959.1 | ATH57883.1 | AGW36711.1 |
| QHL79558.1 | SQE61950.1 | QCW86118.1 | QBC21719.1 | QBB16785.1 | ARH72649.1 |
| VDZ38535.1 | AXI05954.1 | VDZ11930.1 | QNU54916.1 | ATC68279.1 | QIT41817.1 |
| SQE64518.1 | ATN60235.1 | BAF78810.1 | QHK99172.1 | AXU09076.1 | ATF39456.1 |
| QHK82433.1 | AUU52633.1 | AVU07407.1 | ADL65984.1 | CAI80710.1 | |

TABLE 7
Exemplary Lytic Transglycosylases Sequences

| Acc. No. | AUG74852.1 | QHK83809.1 | AVU08026.1 | QHK99721.1 | ATC68908.1 |
|---|---|---|---|---|---|
| WP085056953.1 | SQE67616.1 | QBP97229.1 | ARH73270.1 | ADL66605.1 | AXU09612.1 |
| AIA29042.1 | QHL20447.1 | QHK68394.1 | SQE82139.1 | QIT39968.1 | CAI82131.1 |
| VDZ28840.1 | AGU56164.1 | AKB00635.1 | AEZ38490.1 | AZL92471.1 | ATF40081.1 |
| QHL23053.1 | QAZ55358.1 | AUU55078.1 | AUU49588.1 | SQF47563.1 | BBG09282.1 |
| QHK81364.1 | QCW89946.1 | QBX63049.1 | QBS25109.1 | AWE65593.1 | AXJ34329.1 |
| QCW92588.1 | AJC29976.1 | AMO18088.1 | ATC72753.1 | QNU57058.1 | QDX07811.1 |
| SQI91272.1 | ALS86882.1 | SQF01045.1 | QHG58036.1 | AXJ67977.1 | AUU62490.1 |
| BBN31134.1 | AUU41776.1 | ANI72414.1 | SQE34785.1 | VDZ27961.1 | ATN59009.1 |
| QCA27690.1 | AUU68133.1 | VDZ93788.1 | AVS03580.1 | VDY49376.1 | QBB18762.1 |
| AJC38513.1 | QAZ60728.1 | AZB47890.1 | VDZ36516.1 | ATW81292.1 | ABD31883.1 |
| ALY16524.1 | AMV81024.1 | AXJ57367.1 | AJC44198.1 | QHK78455.1 | AWR20367.1 |
| AXP53344.1 | APD05343.1 | ARH70547.1 | AYV03103.1 | AJC35664.1 | AOO99871.1 |
| AXG01987.1 | ALY19493.1 | QBY49526.1 | AWR05819.1 | VEG28863.1 | QNU60294.1 |
| QHK40608.1 | AIU86486.1 | BAB96355.1 | AWW94802.1 | AUJ57123.1 | QIT38187.1 |
| VEB68472.1 | QNU50647.1 | AFR74567.1 | ALR00683.1 | AIW28238.1 | QBS05662.1 |
| BAR10083.1 | AUU58667.1 | AXJ24218.1 | VDZ21181.1 | QBQ52614.1 | AXG04701.1 |
| AGW34897.1 | QNU52988.1 | VEE69360.1 | AXJ49801.1 | SQE89491.1 | AVU14965.1 |
| AZH11092.1 | CRL39530.1 | SQE84723.1 | AJE65945.1 | QHK96768.1 | BBJ13929.1 |
| BAR12807.1 | ADC38717.1 | QCT55639.1 | QHL04458.1 | AXJ47238.1 | AVG58910.1 |
| AYC79185.1 | QLL21889.1 | ARH76048.1 | QHL61614.1 | AXJ62649.1 | AUJ53206.1 |
| QHK53792.1 | VED82772.1 | QHL77997.1 | QDX04312.1 | AUU64735.1 | AND03826.1 |
| AZG96794.1 | ATN50881.1 | QCV72136.1 | QBC25023.1 | SQE78573.1 | AUU78054.1 |
| VDZ18566.1 | AXN68062.1 | QJR42951.1 | AXR97652.1 | QHL41590.1 | AJC32821.1 |
| QHK32559.1 | ADI99053.1 | QAZ52566.1 | QBX60518.1 | SQF74972.1 | QHL46908.1 |
| AID41260.1 | QJR08682.1 | QHL25203.1 | QCV77702.1 | QHL09940.1 | QLI01771.1 |
| ARG47142.1 | AXS23161.1 | AVS41325.1 | ARH67847.1 | QHK48798.1 | QKV61069.1 |
| CAG41627.1 | ARH78744.1 | ARH59240.1 | AXJ42163.1 | SQE37528.1 | QBS03248.1 |
| AXJ36970.1 | AXJ65314.1 | BAF68741.1 | AUS76539.1 | AVG53477.1 | AHW67115.1 |
| AXJ44674.1 | ANI75455.1 | AIA00385.1 | ATV05173.1 | AII57052.1 | VDZ15906.1 |
| QHK94169.1 | ASC52104.1 | APW76923.1 | AWQ32688.1 | AEB89661.1 | QHL66999.1 |
| QHK61045.1 | AYV00641.1 | AUU46968.1 | AWW97752.1 | AMQ79858.1 | QHK88884.1 |
| QHK51097.1 | QHL58995.1 | QHK86314.1 | VED78438.1 | AXJ52325.1 | AUW99658.1 |
| AXJ39633.1 | QLL19212.1 | ALO32639.1 | CEH27385.1 | ARI75045.1 | BBK65996.1 |
| QHL38959.1 | QHK27301.1 | ASF33458.1 | AUU72380.1 | ATW78630.1 | QBS28062.1 |
| SQE70278.1 | AXJ59987.1 | CCG17180.1 | SQE41905.1 | ASC54811.1 | AJC41353.1 |
| AMO53207.1 | SQE62570.1 | AXJ54846.1 | QNU48150.1 | QBS08230.1 | AVG60490.1 |
| QHL80902.1 | AXI07345.1 | QCW87316.1 | AWQ88011.1 | APC78293.1 | QBZ86871.1 |
| VDZ39159.1 | ATN61540.1 | VDZ13294.1 | QBC22330.1 | ATH58551.1 | QEQ42497.1 |
| SQE65115.1 | AUU54007.1 | BAF79436.1 | QNU55465.1 | QBB14808.1 | AGW37363.1 |
| A6QIT9.1 | SQE67165.1 | QBP96797.1 | AVU07553.1 | ADL66154.1 | AXU09155.1 |
| AIA28591.1 | QHL20890.1 | QHK67939.1 | ARH72781.1 | QIT41945.1 | CAI81669.1 |
| VDZ28385.1 | AGU55700.1 | AKB00177.1 | SQE81672.1 | AZL92011.1 | ATF39601.1 |
| QHL23491.1 | QAZ55823.1 | AUU57339.1 | AEZ38024.1 | SQF46953.1 | BBG08823.1 |
| QHK80926.1 | QCW89482.1 | QBX62597.1 | AUU51817.1 | AWE66115.1 | AXJ33872.1 |
| QCW92130.1 | AJC29474.1 | AMO17631.1 | QBS24632.1 | QNU57480.1 | QDX08265.1 |
| SQI90030.1 | ALS86399.1 | SQF00596.1 | ATC72270.1 | AXJ67524.1 | AUU60298.1 |
| BBN31594.1 | AUU41295.1 | ANI70254.1 | QHG57602.1 | VDZ27044.1 | ATN58543.1 |
| QCA27238.1 | AUU68608.1 | VDZ93337.1 | SQE34336.1 | VDY48918.1 | QBB19232.1 |
| AJC38012.1 | QAZ60257.1 | AZB47436.1 | AVS03112.1 | ATW80819.1 | ABD31367.1 |
| ALY18859.1 | AMV80544.1 | AXJ56922.1 | VDZ36022.1 | QHK78891.1 | AWR19891.1 |
| AXP51473.1 | APD04868.1 | ARH70075.1 | AJC43696.1 | AJC35162.1 | AOO99402.1 |
| AXG01513.1 | ALY19032.1 | QBY49075.1 | AYV02665.1 | VEG27874.1 | QNU59893.1 |
| QHK41050.1 | AIU86033.1 | BAB95885.1 | AWR04008.1 | AUJ57949.1 | QIT38636.1 |
| VEB66778.1 | QNU50221.1 | AFR74058.1 | AWW94318.1 | AIW27786.1 | QBS05193.1 |
| BAR09583.1 | AUU59155.1 | AXJ23764.1 | ALR00199.1 | QBQ52174.1 | AXG04224.1 |
| AGW34432.1 | QNU52590.1 | VEE68899.1 | VDZ20731.1 | SQE89024.1 | AVU14507.1 |
| AZH10641.1 | CRL37855.1 | SQE84261.1 | AXJ49341.1 | QHK97220.1 | BBJ13457.1 |
| BAR12307.1 | ADC38247.1 | QCT55185.1 | AJE65490.1 | AXJ46778.1 | AVG58422.1 |
| AYC78745.1 | QLL21431.1 | ARH75575.1 | QHL04892.1 | AXJ60689.1 | AUJ55209.1 |
| QHK53360.1 | VED81833.1 | QHL77537.1 | QHL62052.1 | AUU65222.1 | AND03352.1 |
| AZG96334.1 | ATN50402.1 | QCV71665.1 | QDX03844.1 | SQE77640.1 | AUU78540.1 |
| VDZ18099.1 | AXN67580.1 | QJR42472.1 | QBC24557.1 | QHL42031.1 | AJC32319.1 |
| QHK33019.1 | ADI98592.1 | QAZ53033.1 | AXR97171.1 | SQF74394.1 | QHL47363.1 |
| AID40782.1 | QJR08214.1 | QHL25643.1 | QBX60063.1 | QHL09505.1 | QLI01335.1 |
| ARG46658.1 | AXS23631.1 | AVS40847.1 | QCV77237.1 | QHK48350.1 | QKV60628.1 |
| CAG41165.1 | ARH78261.1 | ARH58753.1 | ARH67373.1 | SQE37056.1 | QBS02797.1 |
| AXJ36517.1 | AXJ63354.1 | BAF68271.1 | AXJ41711.1 | AVG53953.1 | AHW66104.1 |
| AXJ44222.1 | ANI74989.1 | AHZ99926.1 | AUS76054.1 | AII56574.1 | VDZ15443.1 |
| QHK94607.1 | ASC51617.1 | APW76466.1 | ATV04730.1 | AEB89194.1 | QHL67438.1 |
| QHK61493.1 | AYV00214.1 | AUU49102.1 | AWQ30792.1 | AMQ79869.1 | QHK89336.1 |
| QHK50659.1 | QHL59454.1 | QHK86751.1 | AWW97269.1 | AXJ51880.1 | AUW99997.1 |
| AXJ39181.1 | QLL18754.1 | ALO32176.1 | VED76805.1 | ARI74563.1 | BBK65545.1 |
| QHL39419.1 | QHK27759.1 | ASF33000.1 | CEH26882.1 | ATW78144.1 | QBS27587.1 |
| SQE69818.1 | AXJ59519.1 | CCG16715.1 | AUU71898.1 | ASC54329.1 | AJC40851.1 |
| AMO53695.1 | SQE62098.1 | AXJ54401.1 | SQE41445.1 | QBS07771.1 | AVG60003.1 |
| QHL80467.1 | AXI06885.1 | QCW86851.1 | QNU47742.1 | APC77852.1 | QBZ86400.1 |
| VDZ38697.1 | ATN61083.1 | VDZ12815.1 | AWQ88536.1 | ATH58077.1 | QEQ42047.1 |
| SQE64662.1 | AUU53520.1 | BAF78963.1 | QBC21855.1 | QBB16854.1 | AGW36897.1 |
| AUG74405.1 | QHK83371.1 | AVU07553.1 | QNU55041.1 | ATC68425.1 | QHK99277.1 |

1. A composition for inducing an immune response against Staphylococcus aureus (S. aureus) in a subject, the composition comprising one or more nucleic acid molecules encoding one or more S. aureus antigenic polypeptide, immunogenic variant or fragment thereof.

2. The composition of claim 1, wherein the one or more S. aureus antigenic polypeptide, immunogenic variant or fragment corresponds to an S. aureus protein selected from: autolysin (Atl), N-acetylmuramyl-L-alanine amidase (Amd), endo-β-N-acetylglucosaminidase (Gmd), alpha-hemolysin (Hla), chemotaxis inhibiting protein of S. aureus (CHIPS), Staphylococcal complement inhibitor (SCIN), coproporphyrinogen III oxidase (CgoX), immunodominant staphylococcal antigen A (IsaA), and lytic transglycosylase SACOL2088 (SceD).

3. The composition according to claim 1, wherein:
(a) the one or more nucleic acid molecule comprises at least one of:
(i) a polynucleotide sequence that does not occur in nature;
(ii) a polynucleotide sequence codon optimized for expression in cells of a mammalian subject; and
(iii) a modified nucleoside;
(b) the one or more nucleic acid molecules is encapsulated by a lipid nanoparticle; and/or
(c) the one or more nucleic acid molecules is contained within one or more vector (e.g., an RNA viral vector, a DNA viral vector, and a plasmid).

4. The composition according to any of claim 1, wherein the one or more nucleic acid molecules comprises one or more selected from:
(a) a polynucleotide sequence encoding an Atl polypeptide comprising the amino acid sequence of SEQ ID NO: 1 (e.g., the nucleic acid sequence of SEQ ID NO:2 or the RNA sequence of SEQ ID NO: 3) or SEQ ID NO:72 (e.g., the nucleic acid sequence of SEQ ID NO:73 or the RNA sequence of SEQ ID NO: 74), or an immunogenic variant or fragment thereof;
(b) a polynucleotide sequence encoding an Amd polypeptide comprising the amino acid sequence of SEQ ID NO: 4 (e.g., the nucleic acid sequence of SEQ ID NO:5 or the RNA sequence of SEQ ID NO: 6), or an immunogenic variant or fragment thereof;
(c) a polynucleotide sequence encoding an Amd polypeptide fragment comprising:
(i) the amino acid sequence of SEQ ID NO: 7 (e.g., the nucleic acid sequence of SEQ ID NO:8 or the RNA sequence of SEQ ID NO:9), or an immunogenic variant or fragment thereof;
(ii) the amino acid sequence of SEQ ID NO: 10 (e.g., the nucleic acid sequence of SEQ ID NO:11 or the RNA sequence of SEQ ID NO:12), or an immunogenic variant or fragment thereof;
(iii) the amino acid sequence of SEQ ID NO: 13 (e.g., the nucleic acid sequence of SEQ ID NO:14 or the RNA sequence of SEQ ID NO:15), or an immunogenic variant or fragment thereof; and/or
(iv) the amino acid sequence of SEQ ID NO: 16 (e.g., the nucleic acid sequence of SEQ ID NO:17 or the RNA sequence of SEQ ID NO:18), or an immunogenic variant or fragment thereof;
(d) a polynucleotide sequence encoding an Gmd polypeptide comprising the amino acid sequence of SEQ ID NO: 19 (e.g., the nucleic acid sequence of SEQ ID NO:20 or the RNA sequence of SEQ ID NO: 21), or an immunogenic variant or fragment thereof; and
(e) a polynucleotide sequence encoding an Gmd polypeptide fragment comprising:
(i) the amino acid sequence of SEQ ID NO: 22 (e.g., the nucleic acid sequence of SEQ ID NO:23 or the RNA sequence of SEQ ID NO:24), or an immunogenic variant or fragment thereof;
(ii) the amino acid sequence of SEQ ID NO: 25 (e.g., the nucleic acid sequence of SEQ ID NO:26 or the RNA sequence of SEQ ID NO:27), or an immunogenic variant or fragment thereof; and/or
(iii) the amino acid sequence of SEQ ID NO: 28 (e.g., the nucleic acid sequence of SEQ ID NO:29 or the RNA sequence of SEQ ID NO:30), or an immunogenic variant or fragment thereof.

5. The composition according to claim 1, wherein the one or more nucleic acid molecules comprises one or more selected from:
(a) a polynucleotide sequence encoding a CHIPs polypeptide comprising the amino acid sequence of SEQ ID NO: 31 (e.g., the nucleic acid sequence of SEQ ID NO:32 or the RNA sequence of SEQ ID NO: 33), or SEQ ID NO: 34 (e.g., the nucleic acid sequence of SEQ ID NO:35 or the RNA sequence of SEQ ID NO: 36), or an immunogenic variant or fragment thereof;
(b) a polynucleotide sequence encoding an CHIPs polypeptide fragment comprising the amino acid sequence of SEQ ID NO: 37 (e.g., the nucleic acid sequence of SEQ ID NO:38 or the RNA sequence of SEQ ID NO:39), or an immunogenic variant or fragment thereof;
(c) a polynucleotide sequence encoding a CHIPs polypeptide comprising the amino acid sequence of SEQ ID NO: 86 (e.g., the nucleic acid sequence of SEQ ID NO:87 or the RNA sequence of SEQ ID NO: 88 or an immunogenic variant or fragment thereof;
(d) a polynucleotide sequence encoding an CHIPs polypeptide fragment comprising the amino acid sequence of SEQ ID NO: 89 (e.g., the nucleic acid sequence of SEQ ID NO:90 or the RNA sequence of SEQ ID NO:119), or an immunogenic variant or fragment thereof;
(e) a polynucleotide sequence encoding an SCIN polypeptide comprising the amino acid sequence of SEQ ID NO: 40 (e.g., the nucleic acid sequence of SEQ ID NO:41 or the RNA sequence of SEQ ID NO: 42), or SEQ ID NO: 43 (e.g., the nucleic acid sequence of SEQ ID NO:75 or the RNA sequence of SEQ ID NO: 44), or an immunogenic variant or fragment thereof;
(f) a polynucleotide sequence encoding an SCIN polypeptide fragment comprising:
(i) the amino acid sequence of SEQ ID NO: 45 (e.g., the nucleic acid sequence of SEQ ID NO:46 or the RNA sequence of SEQ ID NO:47), or an immunogenic variant or fragment thereof; and/or
(ii) the amino acid sequence of SEQ ID NO: 48 (e.g., the nucleic acid sequence of SEQ ID NO:49 or the RNA sequence of SEQ ID NO:50, or an immunogenic variant or fragment thereof;
(g) a polynucleotide sequence encoding a Hla polypeptide comprising the amino acid sequence of SEQ ID NO: 51 (e.g., the nucleic acid sequence of SEQ ID NO:52 or the RNA sequence of SEQ ID NO: 53), or SEQ ID NO: 54 (e.g., the nucleic acid sequence of SEQ ID NO:55 or the RNA sequence of SEQ ID NO: 56), or an immunogenic variant or fragment thereof;
(h) a polynucleotide sequence encoding an Hla polypeptide fragment comprising the amino acid sequence of SEQ ID NO: 57 (e.g., the nucleic acid sequence of SEQ ID NO:58 or the RNA sequence of SEQ ID NO:59), or an immunogenic variant or fragment thereof;
(i) a polynucleotide sequence encoding an CgoX polypeptide comprising the amino acid sequence of SEQ ID NO: 60 (e.g., the nucleic acid sequence of SEQ ID NO:61 or the RNA sequence of SEQ ID NO: 62), or an immunogenic variant or fragment thereof; and/or
(j) a polynucleotide sequence encoding an CgoX polypeptide fragment comprising:
(i) the amino acid sequence of SEQ ID NO: 63 (e.g., the nucleic acid sequence of SEQ ID NO:64 or the RNA sequence of SEQ ID NO:65) or an immunogenic variant or fragment thereof;
(ii) the amino acid sequence of SEQ ID NO: 66 (e.g., the nucleic acid sequence of SEQ ID NO:67 or the RNA sequence of SEQ ID NO:68,) or an immunogenic variant or fragment thereof; and/or
(iii) the amino acid sequence of SEQ ID NO: 69 (e.g., the nucleic acid sequence of SEQ ID NO:70 or the RNA sequence of SEQ ID NO:71), or an immunogenic variant or fragment thereof; and/or
(k) a polynucleotide sequence encoding an IsaA polypeptide comprising the amino acid sequence of SEQ ID NO: 91 (e.g., the nucleic acid sequence of SEQ ID NO:92 or the RNA sequence of SEQ ID NO: 93), or SEQ ID NO: 94 (e.g., the nucleic acid sequence of SEQ ID NO:95 or the RNA sequence of SEQ ID NO: 96), or an immunogenic variant or fragment thereof;
(l) a polynucleotide sequence encoding an IsaA polypeptide fragment comprising:
(i) the amino acid sequence of SEQ ID NO: 97 (e.g., the nucleic acid sequence of SEQ ID NO:98 or the RNA sequence of SEQ ID NO:99), or an immunogenic variant or fragment thereof; and/or
(ii) the amino acid sequence of SEQ ID NO: 100 (e.g., the nucleic acid sequence of SEQ ID NO:101 or the RNA sequence of SEQ ID NO:102), or an immunogenic variant or fragment thereof;
(iii) the amino acid sequence of SEQ ID NO: 103 (e.g., the nucleic acid sequence of SEQ ID NO:104 or the RNA sequence of SEQ ID NO:105), or an immunogenic variant or fragment thereof; and/or
(m) a polynucleotide sequence encoding an SceD polypeptide comprising the amino acid sequence of SEQ ID NO: 106 (e.g., the nucleic acid sequence of SEQ ID NO:107 or the RNA sequence of SEQ ID NO: 108), or SEQ ID NO: 109 (e.g., the nucleic acid sequence of SEQ ID NO:110 or the RNA sequence of SEQ ID NO: 111), or an immunogenic variant or fragment thereof; and/or
(n) a polynucleotide sequence encoding an SceD polypeptide fragment comprising the amino acid sequence of SEQ ID NO: 112 (e.g., the nucleic acid sequence of SEQ ID NO:113 or the RNA sequence of SEQ ID NO:114), or an immunogenic variant or fragment thereof.

6. The composition of claim 1, wherein one or more nucleic acid molecules is an mRNA.

7. The composition of claim 6, wherein the mRNA comprises a 5′ untranslated (UTR) region, an open reading frame, and a 3′ UTR and a polyA tail.

8. The composition of claim 1, wherein the one or more nucleic acid molecules is encapsulated by a lipid nanoparticle.

9. The composition of claim 1, wherein the one or more nucleic acid molecules is contained within one or more vector (e.g., a RNA viral vector, a DNA viral vector, and a plasmid).

10. The composition of claim 1, wherein the composition is a vaccine and optionally further comprises an adjuvant.

11. A method for inducing an antigen specific immune response in a subject, comprising administering to the subject composition according to claim 1 in an amount effective to produce an antigen specific immune response.

12. The method of claim 11, wherein the one or more S. aureus antigenic polypeptide, immunogenic variant or fragment corresponds to an S. aureus protein selected from: autolysin (Atl), N-acetylmuramyl-L-alanine amidase (Amd), endo-β-N-acetylglucosaminidase (Gmd), alpha-hemolysin (Hla), chemotaxis inhibiting protein of S. aureus (CHIPS), Staphylococcal complement inhibitor (SCIN), and coproporphyrinogen III oxidase (CgoX), immunodominant staphylococcal antigen A (IsaA), and lytic transglycosylase SACOL2088 (SceD).

13. The method according to claim 11, wherein:
(a) the one or more nucleic acid molecule comprises at least one of:
(i) a polynucleotide sequence that does not occur in nature;
(ii) a polynucleotide sequence codon optimized for expression in cells of a mammalian subject; and
(iii) a substituted polynucleotide;
(b) the one or more nucleic acid molecules is encapsulated by a lipid nanoparticle; and/or
(c) the one or more nucleic acid molecules is contained within one or more vector (e.g., an RNA viral vector, a DNA viral vector, and a plasmid).

14. The method according to claim 11, wherein the one or more nucleic acid molecules comprises one or more selected from:
(a) a polynucleotide sequence encoding an Atl polypeptide comprising the amino acid sequence of SEQ ID NO: 1 (e.g., the nucleic acid sequence of SEQ ID NO:2 or the RNA sequence of SEQ ID NO: 3) or SEQ ID NO:72 (e.g., the nucleic acid sequence of SEQ ID NO:73 or the RNA sequence of SEQ ID NO:74), or an immunogenic variant or fragment thereof;
(b) a polynucleotide sequence encoding an Amd polypeptide comprising the amino acid sequence of SEQ ID NO: 4 (e.g., the nucleic acid sequence of SEQ ID NO:5 or the RNA sequence of SEQ ID NO: 6), or an immunogenic variant or fragment thereof;
(c) a polynucleotide sequence encoding an Amd polypeptide fragment comprising:
(i) the amino acid sequence of SEQ ID NO: 7 (e.g., the nucleic acid sequence of SEQ ID NO:8 or the RNA sequence of SEQ ID NO:9), or an immunogenic variant or fragment thereof;
(ii) the amino acid sequence of SEQ ID NO: 10 (e.g., the nucleic acid sequence of SEQ ID NO:11 or the RNA sequence of SEQ ID NO:12), or an immunogenic variant or fragment thereof;
(iii) the amino acid sequence of SEQ ID NO: 13 (e.g., the nucleic acid sequence of SEQ ID NO:14 or the RNA sequence of SEQ ID NO:15), or an immunogenic variant or fragment thereof; and/or
(iv) the amino acid sequence of SEQ ID NO: 16 (e.g., the nucleic acid sequence of SEQ ID NO:17 or the RNA sequence of SEQ ID NO:18), or an immunogenic variant or fragment thereof;
(d) a polynucleotide sequence encoding an Gmd polypeptide comprising the amino acid sequence of SEQ ID NO: 19 (e.g., the nucleic acid sequence of SEQ ID NO:20 or the RNA sequence of SEQ ID NO: 21), or an immunogenic variant or fragment thereof; and
(e) a polynucleotide sequence encoding an Gmd polypeptide fragment comprising:
(i) the amino acid sequence of SEQ ID NO: 22 (e.g., the nucleic acid sequence of SEQ ID NO:23 or the RNA sequence of SEQ ID NO:24), or an immunogenic variant or fragment thereof;
(ii) the amino acid sequence of SEQ ID NO: 25 (e.g., the nucleic acid sequence of SEQ ID NO:26 or the RNA sequence of SEQ ID NO:27), or an immunogenic variant or fragment thereof; and/or
(iii) the amino acid sequence of SEQ ID NO: 28 (e.g., the nucleic acid sequence of SEQ ID NO:29 or the RNA sequence of SEQ ID NO:30), or an immunogenic variant or fragment thereof.

15. The method according to claim 11, wherein the one or more nucleic acid molecules comprises one or more selected from:
(a) a polynucleotide sequence encoding a CHIPs polypeptide comprising the amino acid sequence of SEQ ID NO: 31 (e.g., the nucleic acid sequence of SEQ ID NO:32 or the RNA sequence of SEQ ID NO: 33), or SEQ ID NO: 34 (e.g., the nucleic acid sequence of SEQ ID NO:35 or the RNA sequence of SEQ ID NO: 36), or an immunogenic variant or fragment thereof;
(b) a polynucleotide sequence encoding an CHIPs polypeptide fragment comprising the amino acid sequence of SEQ ID NO: 37 (e.g., the nucleic acid sequence of SEQ ID NO:38 or the RNA sequence of SEQ ID NO:39), or an immunogenic variant or fragment thereof;
(c) a polynucleotide sequence encoding a CHIPs polypeptide comprising the amino acid sequence of SEQ ID NO: 86 (e.g., the nucleic acid sequence of SEQ ID NO:87 or the RNA sequence of SEQ ID NO: 88 or an immunogenic variant or fragment thereof;
(d) a polynucleotide sequence encoding an CHIPs polypeptide fragment comprising the amino acid sequence of SEQ ID NO: 89 (e.g., the nucleic acid sequence of SEQ ID NO:90 or the RNA sequence of SEQ ID NO:119), or an immunogenic variant or fragment thereof;
(e) a polynucleotide sequence encoding an SCIN polypeptide comprising the amino acid sequence of SEQ ID NO: 40 (e.g., the nucleic acid sequence of SEQ ID NO:41 or the RNA sequence of SEQ ID NO: 42), or SEQ ID NO: 43 (e.g., the nucleic acid sequence of SEQ ID NO:75 or the RNA sequence of SEQ ID NO: 44), or an immunogenic variant or fragment thereof;
(f) a polynucleotide sequence encoding an SCIN polypeptide fragment comprising:
(i) the amino acid sequence of SEQ ID NO: 45 (e.g., the nucleic acid sequence of SEQ ID NO:46 or the RNA sequence of SEQ ID NO:47), or an immunogenic variant or fragment thereof; and/or
(ii) the amino acid sequence of SEQ ID NO: 48 (e.g., the nucleic acid sequence of SEQ ID NO:49 or the RNA sequence of SEQ ID NO:50,), or an immunogenic variant or fragment thereof;
(g) a polynucleotide sequence encoding a Hla polypeptide comprising the amino acid sequence of SEQ ID NO: 51 (e.g., the nucleic acid sequence of SEQ ID NO:52 or the RNA sequence of SEQ ID NO: 53), or SEQ ID NO: 54 (e.g., the nucleic acid sequence of SEQ ID NO:55 or the RNA sequence of SEQ ID NO: 56), or an immunogenic variant or fragment thereof;
(h) a polynucleotide sequence encoding an Hla polypeptide fragment comprising the amino acid sequence of SEQ ID NO: 57 (e.g., the nucleic acid sequence of SEQ ID NO:58 or the RNA sequence of SEQ ID NO:59), or an immunogenic variant or fragment thereof;
(i) a polynucleotide sequence encoding an CgoX polypeptide comprising the amino acid sequence of SEQ ID NO: 60 (e.g., the nucleic acid sequence of SEQ ID NO:61 or the RNA sequence of SEQ ID NO: 62), or an immunogenic variant or fragment thereof; and/or
(j) a polynucleotide sequence encoding an CgoX polypeptide fragment comprising:
(i) the amino acid sequence of SEQ ID NO: 63 (e.g., the nucleic acid sequence of SEQ ID NO:64 or the RNA sequence of SEQ ID NO:65) or an immunogenic variant or fragment thereof;
(ii) the amino acid sequence of SEQ ID NO: 66 (e.g., the nucleic acid sequence of SEQ ID NO:67 or the RNA sequence of SEQ ID NO:68,) or an immunogenic variant or fragment thereof; and/or
(iii) the amino acid sequence of SEQ ID NO: 69 (e.g., the nucleic acid sequence of SEQ ID NO:70 or the RNA sequence of SEQ ID NO:71), or an immunogenic variant or fragment thereof; and/or
(k) a polynucleotide sequence encoding an IsaA polypeptide comprising the amino acid sequence of SEQ ID NO: 91 (e.g., the nucleic acid sequence of SEQ ID NO:92 or the RNA sequence of SEQ ID NO: 93), or SEQ ID NO: 94 (e.g., the nucleic acid sequence of SEQ ID NO:95 or the RNA sequence of SEQ ID NO: 96), or an immunogenic variant or fragment thereof;
(l) a polynucleotide sequence encoding an IsaA polypeptide fragment comprising:
(i) the amino acid sequence of SEQ ID NO: 97 (e.g., the nucleic acid sequence of SEQ ID NO:98 or the RNA sequence of SEQ ID NO:99), or an immunogenic variant or fragment thereof; and/or
(ii) the amino acid sequence of SEQ ID NO: 100 (e.g., the nucleic acid sequence of SEQ ID NO:101 or the RNA sequence of SEQ ID NO:102), or an immunogenic variant or fragment thereof; and/or
(iii) the amino acid sequence of SEQ ID NO: 103 (e.g., the nucleic acid sequence of SEQ ID NO:104 or the RNA sequence of SEQ ID NO:105), or an immunogenic variant or fragment thereof; and/or
(m) a polynucleotide sequence encoding an SceD polypeptide comprising the amino acid sequence of SEQ ID NO: 106 (e.g., the nucleic acid sequence of SEQ ID NO:107 or the RNA sequence of SEQ ID NO: 108), or SEQ ID NO: 109 (e.g., the nucleic acid sequence of SEQ ID NO:110 or the RNA sequence of SEQ ID NO: 111), or an immunogenic variant or fragment thereof; and/or
(n) a polynucleotide sequence encoding an SceD polypeptide fragment comprising the amino acid sequence of SEQ ID NO: 112 (e.g., the nucleic acid sequence of SEQ ID NO:113 or the RNA sequence of SEQ ID NO:114), or an immunogenic variant or fragment thereof.

16. The method of claim 11, wherein one or more nucleic acid molecules is an mRNA.

17. The method of claim 16, wherein the mRNA comprises at 5′ UTR, an open reading frame, a 3′ UTR and a polyA tail.

18. The method of claim 11, wherein the one or more nucleic acid molecules is encapsulated by a lipid nanoparticle.

19. The method of claim 11, wherein the antigen specific antibody titre in the blood or serum of the subject against the administered antigenic polypeptide, immunogenic variant or fragment reaches >0.2 μg/ml by 30 days following administration.

20. The method of claim 11, wherein the method treats or prevents S. aureus infection.

21. The method of claim 11, wherein the method treats or prevents one or more disease or disorder associated with S. aureus infection.

22. The method of claim 21, wherein the disease or disorder is osteomyelitis.


SEQUENCE LISTING (125 sequences)

SEQ ID NO: 1 - protein, 1268 aa, Artificial Sequence
  Chemically synthesized sequence
MAKKFNYKLPSMVALTLVGSAVTAHQVQAAETTQDQTTNKNVLDSNKVKATTEQAKAEVKNPTQNISGTQVYQDPAIVQPKTANNKTGNAQVSQKVDTAQVNGDTRANQSATTNNTQPVAKSTSTTAPKTNTNVTNAGYSLVDDEDDNSANTENQINPELIKSAAKPAALETQYKAAAPKAATTSAPKAKTEATPKVTTFSASAQPRSVAATPKTSLPKYKPQVNSSINDYIRKNNLKAPKIEEDYTSYFPKYAYRNGVGRPEGIVVHDTANDRSTINGEISYMKNNYQNAFVHAFVDGDRIIETAPTDYLSWGVGAVGNPRFINVEIVHTHDYASFARSMNNYADYAATQLQYYGLKPDSAEYDGNGTVWTHYAVSKYLGGTDHADPHGYLRSHNYSYDQLYDLINEKYLIKMGKVAPWGTQSTTTPTTPSKPTTPSTPSKPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKPGTKLYTVPWGTSKQVAGSVSGSGNQTFKASKQQQIDKSIYLYGSVNGKSGWVSKAYLVDTAKPTPTPTPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLFGTVNGKSGWVSKAYLAVPAAPKKAVAQPKTAVKAYAVTKPQTTQTTQTVSKIAQVKPNNTGIRASVYEKTAKNGAKYADRTFYVTKERAHGNETYVLLNNTSHNIPLGWFNVKDLNVQNLGKEVKTTQKYTVNKSNNGLSMVPWGTKNQVILTGNNIAQGTFNATKQVSVGKDVYLYGTINNRTGWVNAKDLTAPTAVKPTTSAAKDYNYTYVIKNGNGYYYVTPNSDTAKYSLKAFNEQPFAVVKEQVINGQTWYYGKLSNGKLAWIKSTDLAKELIKYNQTGMTLNQVAQIQAGLQYKPQVQRVPGKWTDANFNDVKHAMDTKRLAQDPALKYQFLRLDQPQNISIDKINQFLKGKGVLENQGAAFNKAAQMYGINEVYLISHALLETGNGTSQLAKGADVVNNKVVTNSNTKYHNVFGIAAYDNDPLREGIKYAKQAGWDTVSKAIVGGAKFIGNSYVKAGQNTLYKMRWNPAHPGTHQYATDVDWANINAKIIKGYYDKIGEVGKYFDIPQYK

SEQ ID NO: 2 - DNA, 3804 bp, Artificial Sequence
  Chemically synthesized sequence
atggccaagaaattcaattataagctgcctagcatggtggccctgacactggtgggaagcgccgtgacagcccaccaggtccaggccgctgaaaccacccaggaccagacgacgaacaagaacgtgctggacagcaataaagtcaaggccaccaccgagcaggccaaagctgaggtgaagaatcccacccaaaacatctctggcacccaggtgtaccaggaccctgccatcgtgcagcctaagaccgccaacaacaagactggcaacgcccaggtgtcccagaaggtggataccgcacaagtgaacggcgacaccagagccaatcagagcgccacaaccaacaacacccagcctgtggccaagagcacctccacaactgcccccaagacaaatactaatgtgacaaacgccggttattctctggtcgatgacgaggatgataactctgccaataccgagaaccagatcaacccagagctgatcaaaagcgccgccaaacctgccgccctggaaacacagtataaggccgctgctccaaaggccgccaccaccagcgccccaaaggccaaaaccgaagccactcctaaggtgacaacattttctgccagcgcccagccaagaagcgtagccgccacccctaagacgagcctgcccaaatacaagccccaggtgaatagcagcattaacgactacatcagaaagaataatctgaaggccccaaagatcgaggaagattacaccagctacttccccaagtacgcctatagaaacggcgtgggcagacctgagggcatcgtggttcacgacacagctaatgaccggagcaccatcaacggcgaaatcagctacatgaagaacaactaccagaatgccttcgtgcacgccttcgtggatggcgaccggatcatcgagaccgctcctaccgactacctgagctggggcgtgggcgccgtgggcaaccccagattcatcaacgtggaaatcgtgcatacacacgactacgccagcttcgctagaagcatgaacaactacgccgattacgccgctacacaactgcaatactacggcctgaagcctgatagcgccgagtatgacggcaacggcacagtttggacccactacgccgtgagcaagtacctcggcggcacagaccacgccgatcctcacggctacctgcggtcccacaactactcttacgatcagctgtacgacctgatcaacgaaaagtatcttatcaaaatgggcaaggtggcaccttggggcacccagagcaccaccacacctacaacaccttccaagcctacaaccccttctaccccttctaagccctctaagcccagcaccgggaaactgacagtggccgctaacaacggagtagcccagatcaaacctaccaacagcggcctgtacaccaccgtctacgacaagacaggcaaggctaccaacgaggtgcagaagacctttgccgtgtccaaaacagcaacgctgggcaaccagaagttctacctggtgcaggactacaacagtggcaacaagttcggctgggtgaaggaaggagatgtggtgtacaacaccgccaagagccctgtgaatgtgaaccagtcttatagcatcaagcccggcacaaagctgtacaccgttccttggggaacctccaaacaggtggccggcagtgtgagtggctccggcaatcagaccttcaaggccagcaagcagcagcagatcgacaaatctatctacctgtatggaagtgtgaacgggaagtccggctgggtatccaaggcatacctagttgataccgccaagcccacccctaccccgacacctaaacctagcacacccacaaccaacaacaagctgaccgtgtcttctctgaacggagtggcccagatcaacgccaagaacaatggcctgttcaccacagtgtacgacaagacaggcaagcctaccaaggaggtgcagaagaccttcgccgtgaccaaggaagcaagcctgggaggcaacaagttctacctcgtgaaggactacaatagccctaccctgattggctgggtgaagcagggagatgtaatctacaacaacgctaagagccccgtgaacgtgatgcagacctacaccgtgaagcctggcacaaagctgtacagcgtgccctggggcacatacaagcaggaggccggcgccgtgagcggaaccggcaaccagacctttaaggccaccaagcagcaacagatcgacaagagtatatacctgttcggcactgtaaacggtaagtccggctgggtgagcaaagcgtacctggccgttcctgctgcccctaaaaaggccgtggctcaaccgaaaaccgccgtgaaagcctacgccgtcaccaaacctcagacaacacagacaactcaaaccgtaagcaagatcgcccaggtgaagcccaacaacaccggaattcgggccagcgtgtacgagaagaccgccaagaacggcgccaagtacgctgatagaacattctacgtgaccaaggaacgggcccacggtaacgagacatacgtgctgctgaacaacacctcccacaacatccccctgggatggttcaacgtgaaagatctgaacgtgcagaacctgggcaaagaggtgaagaccacacagaaatacaccgtgaacaagagtaacaacggcctcagcatggtgccttggggcaccaagaaccaggtgatcctgaccggcaacaatatcgcccaaggcaccttcaacgccaccaagcaggtgagcgtgggcaaggacgtgtacctgtacggcacaatcaacaacagaaccggatgggtcaacgccaaggacctgaccgcccctactgccgtgaaaccaacaaccagcgccgccaaggactacaattacacctacgtgattaagaatggaaatggctactactacgtgacaccaaatagtgataccgccaaatatagcctgaaggccttcaacgagcagccattcgcggtggtgaaggagcaggtgatcaacggacagacctggtactacggcaagctgagcaacggcaaactggcctggatcaagagcactgacctggccaaagagctgattaaatacaaccagacgggcatgacccttaaccaagtggcccagatccaggccggactgcagtacaagcctcaggtgcagagagtgccagggaagtggaccgacgccaacttcaacgacgtgaagcacgctatggacacaaagcgcctggcccaggatcctgctctgaagtaccagtttctgagactggaccagcctcagaacatctccatcgacaagatcaaccagttcctgaaaggcaagggcgttctggagaaccaaggcgccgccttcaacaaagctgctcagatgtacggcatcaatgaggtgtacctgatcagccacgccctgctggaaaccggcaacggcaccagccagctggctaagggcgccgacgtggtgaacaacaaggtggtgaccaactctaataccaagtatcacaacgtctttggaatcgccgcctatgacaacgaccccctgagggaaggcatcaaatacgccaagcaggccggctgggatacagtgagcaaggctatcgtggggggcgctaagttcatcggaaattcttacgtgaaggctggccagaacaccctgtacaagatgcggtggaaccctgctcatcctggcacccaccaatacgccacggacgtggactgggccaacatcaacgccaagatcatcaagggttactacgacaagattggagaggtgggcaagtacttcgacatccctcagtacaag

SEQ ID NO: 3 - RNA, 3804 nt, Artificial Sequence
  Chemically synthesized sequence
auggccaagaaauucaauuauaagcugccuagcaugguggcccugacacuggugggaagcgccgugacagcccaccagguccaggccgcugaaaccacccaggaccagacgacgaacaagaacgugcuggacagcaauaaagucaaggccaccaccgagcaggccaaagcugaggugaagaaucccacccaaaacaucucuggcacccagguguaccaggacccugccaucgugcagccuaagaccgccaacaacaagacuggcaacgcccaggugucccagaagguggauaccgcacaagugaacggcgacaccagagccaaucagagcgccacaaccaacaacacccagccuguggccaagagcaccuccacaacugcccccaagacaaauacuaaugugacaaacgccgguuauucucuggucgaugacgaggaugauaacucugccaauaccgagaaccagaucaacccagagcugaucaaaagcgccgccaaaccugccgcccuggaaacacaguauaaggccgcugcuccaaaggccgccaccaccagcgccccaaaggccaaaaccgaagccacuccuaaggugacaacauuuucugccagcgcccagccaagaagcguagccgccaccccuaagacgagccugcccaaauacaagccccaggugaauagcagcauuaacgacuacaucagaaagaauaaucugaaggccccaaagaucgaggaagauuacaccagcuacuuccccaaguacgccuauagaaacggcgugggcagaccugagggcaucgugguucacgacacagcuaaugaccggagcaccaucaacggcgaaaucagcuacaugaagaacaacuaccagaaugccuucgugcacgccuucguggauggcgaccggaucaucgagaccgcuccuaccgacuaccugagcuggggcgugggcgccgugggcaaccccagauucaucaacguggaaaucgugcauacacacgacuacgccagcuucgcuagaagcaugaacaacuacgccgauuacgccgcuacacaacugcaauacuacggccugaagccugauagcgccgaguaugacggcaacggcacaguuuggacccacuacgccgugagcaaguaccucggcggcacagaccacgccgauccucacggcuaccugcggucccacaacuacucuuacgaucagcuguacgaccugaucaacgaaaaguaucuuaucaaaaugggcaagguggcaccuuggggcacccagagcaccaccacaccuacaacaccuuccaagccuacaaccccuucuaccccuucuaagcccucuaagcccagcaccgggaaacugacaguggccgcuaacaacggaguagcccagaucaaaccuaccaacagcggccuguacaccaccgucuacgacaagacaggcaaggcuaccaacgaggugcagaagaccuuugccguguccaaaacagcaacgcugggcaaccagaaguucuaccuggugcaggacuacaacaguggcaacaaguucggcugggugaaggaaggagaugugguguacaacaccgccaagagcccugugaaugugaaccagucuuauagcaucaagcccggcacaaagcuguacaccguuccuuggggaaccuccaaacagguggccggcagugugaguggcuccggcaaucagaccuucaaggccagcaagcagcagcagaucgacaaaucuaucuaccuguauggaagugugaacgggaaguccggcuggguauccaaggcauaccuaguugauaccgccaagcccaccccuaccccgacaccuaaaccuagcacacccacaaccaacaacaagcugaccgugucuucucugaacggaguggcccagaucaacgccaagaacaauggccuguucaccacaguguacgacaagacaggcaagccuaccaaggaggugcagaagaccuucgccgugaccaaggaagcaagccugggaggcaacaaguucuaccucgugaaggacuacaauagcccuacccugauuggcugggugaagcagggagauguaaucuacaacaacgcuaagagccccgugaacgugaugcagaccuacaccgugaagccuggcacaaagcuguacagcgugcccuggggcacauacaagcaggaggccggcgccgugagcggaaccggcaaccagaccuuuaaggccaccaagcagcaacagaucgacaagaguauauaccuguucggcacuguaaacgguaaguccggcugggugagcaaagcguaccuggccguuccugcugccccuaaaaaggccguggcucaaccgaaaaccgccgugaaagccuacgccgucaccaaaccucagacaacacagacaacucaaaccguaagcaagaucgcccaggugaagcccaacaacaccggaauucgggccagcguguacgagaagaccgccaagaacggcgccaaguacgcugauagaacauucuacgugaccaaggaacgggcccacgguaacgagacauacgugcugcugaacaacaccucccacaacaucccccugggaugguucaacgugaaagaucugaacgugcagaaccugggcaaagaggugaagaccacacagaaauacaccgugaacaagaguaacaacggccucagcauggugccuuggggcaccaagaaccaggugauccugaccggcaacaauaucgcccaaggcaccuucaacgccaccaagcaggugagcgugggcaaggacguguaccuguacggcacaaucaacaacagaaccggaugggucaacgccaaggaccugaccgccccuacugccgugaaaccaacaaccagcgccgccaaggacuacaauuacaccuacgugauuaagaauggaaauggcuacuacuacgugacaccaaauagugauaccgccaaauauagccugaaggccuucaacgagcagccauucgcgguggugaaggagcaggugaucaacggacagaccugguacuacggcaagcugagcaacggcaaacuggccuggaucaagagcacugaccuggccaaagagcugauuaaauacaaccagacgggcaugacccuuaaccaaguggcccagauccaggccggacugcaguacaagccucaggugcagagagugccagggaaguggaccgacgccaacuucaacgacgugaagcacgcuauggacacaaagcgccuggcccaggauccugcucugaaguaccaguuucugagacuggaccagccucagaacaucuccaucgacaagaucaaccaguuccugaaaggcaagggcguucuggagaaccaaggcgccgccuucaacaaagcugcucagauguacggcaucaaugagguguaccugaucagccacgcccugcuggaaaccggcaacggcaccagccagcuggcuaagggcgccgacguggugaacaacaagguggugaccaacucuaauaccaaguaucacaacgucuuuggaaucgccgccuaugacaacgacccccugagggaaggcaucaaauacgccaagcaggccggcugggauacagugagcaaggcuaucguggggggcgcuaaguucaucggaaauucuuacgugaaggcuggccagaacacccuguacaagaugcgguggaacccugcucauccuggcacccaccaauacgccacggacguggacugggccaacaucaacgccaagaucaucaaggguuacuacgacaagauuggagaggugggcaaguacuucgacaucccucaguacaag

SEQ ID NO: 4 - protein, 591 aa, Artificial Sequence
  Chemically synthesized sequence
MSASAQPRSVAATPKTSLPKYKPQVNSSINDYIRKNNLKAPKIEEDYTSYFPKYAYRNGVGRPEGIVVHDTANDRSTINGEISYMKNNYQNAFVHAFVDGDRIIETAPTDYLSWGVGAVGNPRFINVEIVHTHDYASFARSMNNYADYAATQLQYYGLKPDSAEYDGNGTVWTHYAVSKYLGGTDHADPHGYLRSHNYSYDQLYDLINEKYLIKMGKVAPWGTQSTTTPTTPSKPTTPSTPSKPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKPGTKLYTVPWGTSKQVAGSVSGSGNQTFKASKQQQIDKSIYLYGSVNGKSGWVSKAYLVDTAKPTPTPTPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLFGTVNGKSGWVSKAYLAVPAAPKKAVAQPKTAVKAYAVTK

SEQ ID NO: 5 - DNA, 1776 bp, Artificial Sequence
  Chemically synthesized sequence
atgtctgccagcgcccagccaagaagcgtagccgccacccctaagacgagcctgcccaaatacaagccccaggtgaatagcagcattaacgactacatcagaaagaataatctgaaggccccaaagatcgaggaagattacaccagctacttccccaagtacgcctatagaaacggcgtgggcagacctgagggcatcgtggttcacgacacagctaatgaccggagcaccatcaacggcgaaatcagctacatgaagaacaactaccagaatgccttcgtgcacgccttcgtggatggcgaccggatcatcgagaccgctcctaccgactacctgagctggggcgtgggcgccgtgggcaaccccagattcatcaacgtggaaatcgtgcatacacacgactacgccagcttcgctagaagcatgaacaactacgccgattacgccgctacacaactgcaatactacggcctgaagcctgatagcgccgagtatgacggcaacggcacagtttggacccactacgccgtgagcaagtacctcggcggcacagaccacgccgatcctcacggctacctgcggtcccacaactactcttacgatcagctgtacgacctgatcaacgaaaagtatcttatcaaaatgggcaaggtggcaccttggggcacccagagcaccaccacacctacaacaccttccaagcctacaaccccttctaccccttctaagccctctaagcccagcaccgggaaactgacagtggccgctaacaacggagtagcccagatcaaacctaccaacagcggcctgtacaccaccgtctacgacaagacaggcaaggctaccaacgaggtgcagaagacctttgccgtgtccaaaacagcaacgctgggcaaccagaagttctacctggtgcaggactacaacagtggcaacaagttcggctgggtgaaggaaggagatgtggtgtacaacaccgccaagagccctgtgaatgtgaaccagtcttatagcatcaagcccggcacaaagctgtacaccgttccttggggaacctccaaacaggtggccggcagtgtgagtggctccggcaatcagaccttcaaggccagcaagcagcagcagatcgacaaatctatctacctgtatggaagtgtgaacgggaagtccggctgggtatccaaggcatacctagttgataccgccaagcccacccctaccccgacacctaaacctagcacacccacaaccaacaacaagctgaccgtgtcttctctgaacggagtggcccagatcaacgccaagaacaatggcctgttcaccacagtgtacgacaagacaggcaagcctaccaaggaggtgcagaagaccttcgccgtgaccaaggaagcaagcctgggaggcaacaagttctacctcgtgaaggactacaatagccctaccctgattggctgggtgaagcagggagatgtaatctacaacaacgctaagagccccgtgaacgtgatgcagacctacaccgtgaagcctggcacaaagctgtacagcgtgccctggggcacatacaagcaggaggccggcgccgtgagcggaaccggcaaccagacctttaaggccaccaagcagcaacagatcgacaagagtatatacctgttcggcactgtaaacggtaagtccggctgggtgagcaaagcgtacctggccgttcctgctgcccctaaaaaggccgtggctcaaccgaaaaccgccgtgaaagcctacgccgtcaccaaatga

SEQ ID NO: 6 - RNA, 1776 nt, Artificial Sequence
  Chemically synthesized sequence
augucugccagcgcccagccaagaagcguagccgccaccccuaagacgagccugcccaaauacaagccccaggugaauagcagcauuaacgacuacaucagaaagaauaaucugaaggccccaaagaucgaggaagauuacaccagcuacuuccccaaguacgccuauagaaacggcgugggcagaccugagggcaucgugguucacgacacagcuaaugaccggagcaccaucaacggcgaaaucagcuacaugaagaacaacuaccagaaugccuucgugcacgccuucguggauggcgaccggaucaucgagaccgcuccuaccgacuaccugagcuggggcgugggcgccgugggcaaccccagauucaucaacguggaaaucgugcauacacacgacuacgccagcuucgcuagaagcaugaacaacuacgccgauuacgccgcuacacaacugcaauacuacggccugaagccugauagcgccgaguaugacggcaacggcacaguuuggacccacuacgccgugagcaaguaccucggcggcacagaccacgccgauccucacggcuaccugcggucccacaacuacucuuacgaucagcuguacgaccugaucaacgaaaaguaucuuaucaaaaugggcaagguggcaccuuggggcacccagagcaccaccacaccuacaacaccuuccaagccuacaaccccuucuaccccuucuaagcccucuaagcccagcaccgggaaacugacaguggccgcuaacaacggaguagcccagaucaaaccuaccaacagcggccuguacaccaccgucuacgacaagacaggcaaggcuaccaacgaggugcagaagaccuuugccguguccaaaacagcaacgcugggcaaccagaaguucuaccuggugcaggacuacaacaguggcaacaaguucggcugggugaaggaaggagaugugguguacaacaccgccaagagcccugugaaugugaaccagucuuauagcaucaagcccggcacaaagcuguacaccguuccuuggggaaccuccaaacagguggccggcagugugaguggcuccggcaaucagaccuucaaggccagcaagcagcagcagaucgacaaaucuaucuaccuguauggaagugugaacgggaaguccggcuggguauccaaggcauaccuaguugauaccgccaagcccaccccuaccccgacaccuaaaccuagcacacccacaaccaacaacaagcugaccgugucuucucugaacggaguggcccagaucaacgccaagaacaauggccuguucaccacaguguacgacaagacaggcaagccuaccaaggaggugcagaagaccuucgccgugaccaaggaagcaagccugggaggcaacaaguucuaccucgugaaggacuacaauagcccuacccugauuggcugggugaagcagggagauguaaucuacaacaacgcuaagagccccgugaacgugaugcagaccuacaccgugaagccuggcacaaagcuguacagcgugcccuggggcacauacaagcaggaggccggcgccgugagcggaaccggcaaccagaccuuuaaggccaccaagcagcaacagaucgacaagaguauauaccuguucggcacuguaaacgguaaguccggcugggugagcaaagcguaccuggccguuccugcugccccuaaaaaggccguggcucaaccgaaaaccgccgugaaagccuacgccgucaccaaauga

SEQ ID NO: 7 - protein, 175 aa, Artificial Sequence
  Chemically synthesized sequence
TTTPTTPSKPTTPSTPSKPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKPGTKLYTVPWGTSKQVAGSVSGSGNQTFKASKQQQIDKSIYLYGSVNGKSGWVSKAYLVD

SEQ ID NO: 8 - DNA, 525 bp, Artificial Sequence
  Chemically synthesized sequence
accaccacacctacaacaccttccaagcctacaaccccttctaccccttctaagccctctaagcccagcaccgggaaactgacagtggccgctaacaacggagtagcccagatcaaacctaccaacagcggcctgtacaccaccgtctacgacaagacaggcaaggctaccaacgaggtgcagaagacctttgccgtgtccaaaacagcaacgctgggcaaccagaagttctacctggtgcaggactacaacagtggcaacaagttcggctgggtgaaggaaggagatgtggtgtacaacaccgccaagagccctgtgaatgtgaaccagtcttatagcatcaagcccggcacaaagctgtacaccgttccttggggaacctccaaacaggtggccggcagtgtgagtggctccggcaatcagaccttcaaggccagcaagcagcagcagatcgacaaatctatctacctgtatggaagtgtgaacgggaagtccggctgggtatccaaggcatacctagttgat

SEQ ID NO: 9 - RNA, 525 nt, Artificial Sequence
  Chemically synthesized sequence
accaccacaccuacaacaccuuccaagccuacaaccccuucuaccccuucuaagcccucuaagcccagcaccgggaaacugacaguggccgcuaacaacggaguagcccagaucaaaccuaccaacagcggccuguacaccaccgucuacgacaagacaggcaaggcuaccaacgaggugcagaagaccuuugccguguccaaaacagcaacgcugggcaaccagaaguucuaccuggugcaggacuacaacaguggcaacaaguucggcugggugaaggaaggagaugugguguacaacaccgccaagagcccugugaaugugaaccagucuuauagcaucaagcccggcacaaagcuguacaccguuccuuggggaaccuccaaacagguggccggcagugugaguggcuccggcaaucagaccuucaaggccagcaagcagcagcagaucgacaaaucuaucuaccuguauggaagugugaacgggaaguccggcuggguauccaaggcauaccuaguugau

SEQ ID NO: 10 - protein, 194 aa, Artificial Sequence
  Chemically synthesized sequence
TAKPTPTPTPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLFGTVNGKSGWVSKAYLAVPAAPKKAVAQPKTAVKAYAVTKQTT

SEQ ID NO: 11 - DNA, 513 bp, Artificial Sequence
  Chemically synthesized sequence
accgccaagcccacccctaccccgacacctaaacctagcacacccacaaccaacaacaagctgaccgtgtcttctctgaacggagtggcccagatcaacgccaagaacaatggcctgttcaccacagtgtacgacaagacaggcaagcctaccaaggaggtgcagaagaccttcgccgtgaccaaggaagcaagcctgggaggcaacaagttctacctcgtgaaggactacaatagccctaccctgattggctgggtgaagcagggagatgtaatctacaacaacgctaagagccccgtgaacgtgatgcagacctacaccgtgaagcctggcacaaagctgtacagcgtgccctggggcacatacaagcaggaggccggcgccgtgagcggaaccggcaaccagacctttaaggccaccaagcagcaacagatcgacaagagtatatacctgttcggcactgtaaacggtaagtccggctgggtgagcaaagcgtacctggccgttcctgct

SEQ ID NO: 12 - RNA, 513 nt, Artificial Sequence
  Chemically synthesized sequence
accgccaagcccaccccuaccccgacaccuaaaccuagcacacccacaaccaacaacaagcugaccgugucuucucugaacggaguggcccagaucaacgccaagaacaauggccuguucaccacaguguacgacaagacaggcaagccuaccaaggaggugcagaagaccuucgccgugaccaaggaagcaagccugggaggcaacaaguucuaccucgugaaggacuacaauagcccuacccugauuggcugggugaagcagggagauguaaucuacaacaacgcuaagagccccgugaacgugaugcagaccuacaccgugaagccuggcacaaagcuguacagcgugcccuggggcacauacaagcaggaggccggcgccgugagcggaaccggcaaccagaccuuuaaggccaccaagcagcaacagaucgacaagaguauauaccuguucggcacuguaaacgguaaguccggcugggugagcaaagcguaccuggccguuccugcu

SEQ ID NO: 13 - protein, 365 aa, Artificial Sequence
  Chemically synthesized sequence
TTPTTPSKPTTPSTPSKPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKPGTKLYTVPWGTSKQVAGSVSGSGNQTFKASKQQQIDKSIYLYGSVNGKSGWVSKAYLVDTAKPTPTPTPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLFGTVNGKSGWVSKAYLAVPAAPKKAVAQPKTAVKAYAVTK

SEQ ID NO: 14 - DNA, 1038 bp, Artificial Sequence
  Chemically synthesized sequence
accaccacacctacaacaccttccaagcctacaaccccttctaccccttctaagccctctaagcccagcaccgggaaactgacagtggccgctaacaacggagtagcccagatcaaacctaccaacagcggcctgtacaccaccgtctacgacaagacaggcaaggctaccaacgaggtgcagaagacctttgccgtgtccaaaacagcaacgctgggcaaccagaagttctacctggtgcaggactacaacagtggcaacaagttcggctgggtgaaggaaggagatgtggtgtacaacaccgccaagagccctgtgaatgtgaaccagtcttatagcatcaagcccggcacaaagctgtacaccgttccttggggaacctccaaacaggtggccggcagtgtgagtggctccggcaatcagaccttcaaggccagcaagcagcagcagatcgacaaatctatctacctgtatggaagtgtgaacgggaagtccggctgggtatccaaggcatacctagttgataccgccaagcccacccctaccccgacacctaaacctagcacacccacaaccaacaacaagctgaccgtgtcttctctgaacggagtggcccagatcaacgccaagaacaatggcctgttcaccacagtgtacgacaagacaggcaagcctaccaaggaggtgcagaagaccttcgccgtgaccaaggaagcaagcctgggaggcaacaagttctacctcgtgaaggactacaatagccctaccctgattggctgggtgaagcagggagatgtaatctacaacaacgctaagagccccgtgaacgtgatgcagacctacaccgtgaagcctggcacaaagctgtacagcgtgccctggggcacatacaagcaggaggccggcgccgtgagcggaaccggcaaccagacctttaaggccaccaagcagcaacagatcgacaagagtatatacctgttcggcactgtaaacggtaagtccggctgggtgagcaaagcgtacctggccgttcctgct

SEQ ID NO: 15 - RNA, 1038 nt, Artificial Sequence
  Chemically synthesized sequence
accaccacaccuacaacaccuuccaagccuacaaccccuucuaccccuucuaagcccucuaagcccagcaccgggaaacugacaguggccgcuaacaacggaguagcccagaucaaaccuaccaacagcggccuguacaccaccgucuacgacaagacaggcaaggcuaccaacgaggugcagaagaccuuugccguguccaaaacagcaacgcugggcaaccagaaguucuaccuggugcaggacuacaacaguggcaacaaguucggcugggugaaggaaggagaugugguguacaacaccgccaagagcccugugaaugugaaccagucuuauagcaucaagcccggcacaaagcuguacaccguuccuuggggaaccuccaaacagguggccggcagugugaguggcuccggcaaucagaccuucaaggccagcaagcagcagcagaucgacaaaucuaucuaccuguauggaagugugaacgggaaguccggcuggguauccaaggcauaccuaguugauaccgccaagcccaccccuaccccgacaccuaaaccuagcacacccacaaccaacaacaagcugaccgugucuucucugaacggaguggcccagaucaacgccaagaacaauggccuguucaccacaguguacgacaagacaggcaagccuaccaaggaggugcagaagaccuucgccgugaccaaggaagcaagccugggaggcaacaaguucuaccucgugaaggacuacaauagcccuacccugauuggcugggugaagcagggagauguaaucuacaacaacgcuaagagccccgugaacgugaugcagaccuacaccgugaagccuggcacaaagcuguacagcgugcccuggggcacauacaagcaggaggccggcgccgugagcggaaccggcaaccagaccuuuaaggccaccaagcagcaacagaucgacaagaguauauaccuguucggcacuguaaacgguaaguccggcugggugagcaaagcguaccuggccguuccugcu

SEQ ID NO: 16 - protein, 224 aa, Artificial Sequence
  Chemically synthesized sequence
MSASAQPRSVAATPKTSLPKYKPQVNSSINDYIRKNNLKAPKIEEDYTSYFPKYAYRNGVGRPEGIVVHDTANDRSTINGEISYMKNNYQNAFVHAFVDGDRIIETAPTDYLSWGVGAVGNPRFINVEIVHTHDYASFARSMNNYADYAATQLQYYGLKPDSAEYDGNGTVWTHYAVSKYLGGTDHADPHGYLRSHNYSYDQLYDLINEKYLIKMGKVAPWGTQ

SEQ ID NO: 17 - DNA, 673 bp, Artificial Sequence
  Chemically synthesized sequence
atgtctgccagcgcccagccaagaagcgtagccgccacccctaagacgagcctgcccaaatacaagccccaggtgaatagcagcattaacgactacatcagaaagaataatctgaaggccccaaagatcgaggaagattacaccagctacttccccaagtacgcctatagaaacggcgtgggcagacctgagggcatcgtggttcacgacacagctaatgaccggagcaccatcaacggcgaaatcagctacatgaagaacaactaccagaatgccttcgtgcacgccttcgtggatggcgaccggatcatcgagaccgctcctaccgactacctgagctggggcgtgggcgccgtgggcaaccccagattcatcaacgtggaaatcgtgcatacacacgactacgccagcttcgctagaagcatgaacaactacgccgattacgccgctacacaactgcaatactacggcctgaagcctgatagcgccgagtatgacggcaacggcacagtttggacccactacgccgtgagcaagtacctcggcggcacagaccacgccgatcctcacggctacctgcggtcccacaactactcttacgatcagctgtacgacctgatcaacgaaaagtatcttatcaaaatgggcaaggtggcaccttggggcacccaga

SEQ ID NO: 18 - RNA, 673 nt, Artificial Sequence
  Chemically synthesized sequence
augucugccagcgcccagccaagaagcguagccgccaccccuaagacgagccugcccaaauacaagccccaggugaauagcagcauuaacgacuacaucagaaagaauaaucugaaggccccaaagaucgaggaagauuacaccagcuacuuccccaaguacgccuauagaaacggcgugggcagaccugagggcaucgugguucacgacacagcuaaugaccggagcaccaucaacggcgaaaucagcuacaugaagaacaacuaccagaaugccuucgugcacgccuucguggauggcgaccggaucaucgagaccgcuccuaccgacuaccugagcuggggcgugggcgccgugggcaaccccagauucaucaacguggaaaucgugcauacacacgacuacgccagcuucgcuagaagcaugaacaacuacgccgauuacgccgcuacacaacugcaauacuacggccugaagccugauagcgccgaguaugacggcaacggcacaguuuggacccacuacgccgugagcaaguaccucggcggcacagaccacgccgauccucacggcuaccugcggucccacaacuacucuuacgaucagcuguacgaccugaucaacgaaaaguaucuuaucaaaaugggcaagguggcaccuuggggcacccaga

SEQ ID NO: 19 - protein, 483 aa, Artificial Sequence
  Chemically synthesized sequence
MAVTKPQTTQTTQTVSKIAQVKPNNTGIRASVYEKTAKNGAKYADRTFYVTKERAHGNETYVLLNNTSHNIPLGWFNVKDLNVQNLGKEVKTTQKYTVNKSNNGLSMVPWGTKNQVILTGNNIAQGTFNATKQVSVGKDVYLYGTINNRTGWVNAKDLTAPTAVKPTTSAAKDYNYTYVIKNGNGYYYVTPNSDTAKYSLKAFNEQPFAVVKEQVINGQTWYYGKLSNGKLAWIKSTDLAKELIKYNQTGMTLNQVAQIQAGLQYKPQVQRVPGKWTDANFNDVKHAMDTKRLAQDPALKYQFLRLDQPQNISIDKINQFLKGKGVLENQGAAFNKAAQMYGINEVYLISHALLETGNGTSQLAKGADVVNNKVVTNSNTKYHNVFGIAAYDNDPLREGIKYAKQAGWDTVSKAIVGGAKFIGNSYVKAGQNTLYKMRWNPAHPGTHQYATDVDWANINAKIIKGYYDKIGEVGKYFDIPQYK

SEQ ID NO: 20 - DNA, 1452 bp, Artificial Sequence
  Chemically synthesized sequence
atggccgtcaccaaacctcagacaacacagacaactcaaaccgtaagcaagatcgcccaggtgaagcccaacaacaccggaattcgggccagcgtgtacgagaagaccgccaagaacggcgccaagtacgctgatagaacattctacgtgaccaaggaacgggcccacggtaacgagacatacgtgctgctgaacaacacctcccacaacatccccctgggatggttcaacgtgaaagatctgaacgtgcagaacctgggcaaagaggtgaagaccacacagaaatacaccgtgaacaagagtaacaacggcctcagcatggtgccttggggcaccaagaaccaggtgatcctgaccggcaacaatatcgcccaaggcaccttcaacgccaccaagcaggtgagcgtgggcaaggacgtgtacctgtacggcacaatcaacaacagaaccggatgggtcaacgccaaggacctgaccgcccctactgccgtgaaaccaacaaccagcgccgccaaggactacaattacacctacgtgattaagaatggaaatggctactactacgtgacaccaaatagtgataccgccaaatatagcctgaaggccttcaacgagcagccattcgcggtggtgaaggagcaggtgatcaacggacagacctggtactacggcaagctgagcaacggcaaactggcctggatcaagagcactgacctggccaaagagctgattaaatacaaccagacgggcatgacccttaaccaagtggcccagatccaggccggactgcagtacaagcctcaggtgcagagagtgccagggaagtggaccgacgccaacttcaacgacgtgaagcacgctatggacacaaagcgcctggcccaggatcctgctctgaagtaccagtttctgagactggaccagcctcagaacatctccatcgacaagatcaaccagttcctgaaaggcaagggcgttctggagaaccaaggcgccgccttcaacaaagctgctcagatgtacggcatcaatgaggtgtacctgatcagccacgccctgctggaaaccggcaacggcaccagccagctggctaagggcgccgacgtggtgaacaacaaggtggtgaccaactctaataccaagtatcacaacgtctttggaatcgccgcctatgacaacgaccccctgagggaaggcatcaaatacgccaagcaggccggctgggatacagtgagcaaggctatcgtggggggcgctaagttcatcggaaattcttacgtgaaggctggccagaacaccctgtacaagatgcggtggaaccctgctcatcctggcacccaccaatacgccacggacgtggactgggccaacatcaacgccaagatcatcaagggttactacgacaagattggagaggtgggcaagtacttcgacatccctcagtacaagtga

SEQ ID NO: 21 - RNA, 1452 nt, Artificial Sequence
  Chemically synthesized sequence
auggccgucaccaaaccucagacaacacagacaacucaaaccguaagcaagaucgcccaggugaagcccaacaacaccggaauucgggccagcguguacgagaagaccgccaagaacggcgccaaguacgcugauagaacauucuacgugaccaaggaacgggcccacgguaacgagacauacgugcugcugaacaacaccucccacaacaucccccugggaugguucaacgugaaagaucugaacgugcagaaccugggcaaagaggugaagaccacacagaaauacaccgugaacaagaguaacaacggccucagcauggugccuuggggcaccaagaaccaggugauccugaccggcaacaauaucgcccaaggcaccuucaacgccaccaagcaggugagcgugggcaaggacguguaccuguacggcacaaucaacaacagaaccggaugggucaacgccaaggaccugaccgccccuacugccgugaaaccaacaaccagcgccgccaaggacuacaauuacaccuacgugauuaagaauggaaauggcuacuacuacgugacaccaaauagugauaccgccaaauauagccugaaggccuucaacgagcagccauucgcgguggugaaggagcaggugaucaacggacagaccugguacuacggcaagcugagcaacggcaaacuggccuggaucaagagcacugaccuggccaaagagcugauuaaauacaaccagacgggcaugacccuuaaccaaguggcccagauccaggccggacugcaguacaagccucaggugcagagagugccagggaaguggaccgacgccaacuucaacgacgugaagcacgcuauggacacaaagcgccuggcccaggauccugcucugaaguaccaguuucugagacuggaccagccucagaacaucuccaucgacaagaucaaccaguuccugaaaggcaagggcguucuggagaaccaaggcgccgccuucaacaaagcugcucagauguacggcaucaaugagguguaccugaucagccacgcccugcuggaaaccggcaacggcaccagccagcuggcuaagggcgccgacguggugaacaacaagguggugaccaacucuaauaccaaguaucacaacgucuuuggaaucgccgccuaugacaacgacccccugagggaaggcaucaaauacgccaagcaggccggcugggauacagugagcaaggcuaucguggggggcgcuaaguucaucggaaauucuuacgugaaggcuggccagaacacccuguacaagaugcgguggaacccugcucauccuggcacccaccaauacgccacggacguggacugggccaacaucaacgccaagaucaucaaggguuacuacgacaagauuggagaggugggcaaguacuucgacaucccucaguacaaguga

SEQ ID NO: 22 - protein, 137 aa, Artificial Sequence
  Chemically synthesized sequence
QTTQTVSKIAQVKPNNTGIRASVYEKTAKNGAKYADRTFYVTKERAHGNETYVLLNNTSHNIPLGWFNVKDLNVQNLGKEVKTTQKYTVNKSNNGLSMVPWGTKNQVILTGNNIAQGTFNATKQVSVGKDVYLYGTI

SEQ ID NO: 23 - DNA, 420 bp, Artificial Sequence
  Chemically synthesized sequence
cagacaacacagacaactcaaaccgtaagcaagatcgcccaggtgaagcccaacaacaccggaattcgggccagcgtgtacgagaagaccgccaagaacggcgccaagtacgctgatagaacattctacgtgaccaaggaacgggcccacggtaacgagacatacgtgctgctgaacaacacctcccacaacatccccctgggatggttcaacgtgaaagatctgaacgtgcagaacctgggcaaagaggtgaagaccacacagaaatacaccgtgaacaagagtaacaacggcctcagcatggtgccttggggcaccaagaaccaggtgatcctgaccggcaacaatatcgcccaaggcaccttcaacgccaccaagcaggtgagcgtgggcaaggacgtgtacctgtacggcacaatc

SEQ ID NO: 24 - RNA, 420 nt, Artificial Sequence
  Chemically synthesized sequence
cagacaacacagacaacucaaaccguaagcaagaucgcccaggugaagcccaacaacaccggaauucgggccagcguguacgagaagaccgccaagaacggcgccaaguacgcugauagaacauucuacgugaccaaggaacgggcccacgguaacgagacauacgugcugcugaacaacaccucccacaacaucccccugggaugguucaacgugaaagaucugaacgugcagaaccugggcaaagaggugaagaccacacagaaauacaccgugaacaagaguaacaacggccucagcauggugccuuggggcaccaagaaccaggugauccugaccggcaacaauaucgcccaaggcaccuucaacgccaccaagcaggugagcgugggcaaggacguguaccuguacggcacaauc

SEQ ID NO: 25 - protein, 337 aa, Artificial Sequence
  Chemically synthesized sequence
NNRTGWVNAKDLTAPTAVKPTTSAAKDYNYTYVIKNGNGYYYVTPNSDTAKYSLKAFNEQPFAVVKEQVINGQTWYYGKLSNGKLAWIKSTDLAKELIKYNQTGMTLNQVAQIQAGLQYKPQVQRVPGKWTDANFNDVKHAMDTKRLAQDPALKYQFLRLDQPQNISIDKINQFLKGKGVLENQGAAFNKAAQMYGINEVYLISHALLETGNGTSQLAKGADVVNNKVVTNSNTKYHNVFGIAAYDNDPLREGIKYAKQAGWDTVSKAIVGGAKFIGNSYVKAGQNTLYKMRWNPAHPGTHQYATDVDWANINAKIIKGYYDKIGEVGKYFDIPQYK

SEQ ID NO: 26 - DNA, 1011 bp, Artificial Sequence
  Chemically synthesized sequence
aacaacagaaccggatgggtcaacgccaaggacctgaccgcccctactgccgtgaaaccaacaaccagcgccgccaaggactacaattacacctacgtgattaagaatggaaatggctactactacgtgacaccaaatagtgataccgccaaatatagcctgaaggccttcaacgagcagccattcgcggtggtgaaggagcaggtgatcaacggacagacctggtactacggcaagctgagcaacggcaaactggcctggatcaagagcactgacctggccaaagagctgattaaatacaaccagacgggcatgacccttaaccaagtggcccagatccaggccggactgcagtacaagcctcaggtgcagagagtgccagggaagtggaccgacgccaacttcaacgacgtgaagcacgctatggacacaaagcgcctggcccaggatcctgctctgaagtaccagtttctgagactggaccagcctcagaacatctccatcgacaagatcaaccagttcctgaaaggcaagggcgttctggagaaccaaggcgccgccttcaacaaagctgctcagatgtacggcatcaatgaggtgtacctgatcagccacgccctgctggaaaccggcaacggcaccagccagctggctaagggcgccgacgtggtgaacaacaaggtggtgaccaactctaataccaagtatcacaacgtctttggaatcgccgcctatgacaacgaccccctgagggaaggcatcaaatacgccaagcaggccggctgggatacagtgagcaaggctatcgtggggggcgctaagttcatcggaaattcttacgtgaaggctggccagaacaccctgtacaagatgcggtggaaccctgctcatcctggcacccaccaatacgccacggacgtggactgggccaacatcaacgccaagatcatcaagggttactacgacaagattggagaggtgggcaagtacttcgacatccctcagtacaag

SEQ ID NO: 27 - RNA, 1011 nt, Artificial Sequence
  Chemically synthesized sequence
aacaacagaaccggaugggucaacgccaaggaccugaccgccccuacugccgugaaaccaacaaccagcgccgccaaggacuacaauuacaccuacgugauuaagaauggaaauggcuacuacuacgugacaccaaauagugauaccgccaaauauagccugaaggccuucaacgagcagccauucgcgguggugaaggagcaggugaucaacggacagaccugguacuacggcaagcugagcaacggcaaacuggccuggaucaagagcacugaccuggccaaagagcugauuaaauacaaccagacgggcaugacccuuaaccaaguggcccagauccaggccggacugcaguacaagccucaggugcagagagugccagggaaguggaccgacgccaacuucaacgacgugaagcacgcuauggacacaaagcgccuggcccaggauccugcucugaaguaccaguuucugagacuggaccagccucagaacaucuccaucgacaagaucaaccaguuccugaaaggcaagggcguucuggagaaccaaggcgccgccuucaacaaagcugcucagauguacggcaucaaugagguguaccugaucagccacgcccugcuggaaaccggcaacggcaccagccagcuggcuaagggcgccgacguggugaacaacaagguggugaccaacucuaauaccaaguaucacaacgucuuuggaaucgccgccuaugacaacgacccccugagggaaggcaucaaauacgccaagcaggccggcugggauacagugagcaaggcuaucguggggggcgcuaaguucaucggaaauucuuacgugaaggcuggccagaacacccuguacaagaugcgguggaacccugcucauccuggcacccaccaauacgccacggacguggacugggccaacaucaacgccaagaucaucaaggguuacuacgacaagauuggagaggugggcaaguacuucgacaucccucaguacaag

SEQ ID NO: 28 - protein, 31 aa, Artificial Sequence
  Chemically synthesized sequence
NIPLGWFNVKDLNVQNLGKEVKTTQKYTVNK

SEQ ID NO: 29 - DNA, 93 bp, Artificial Sequence
  Chemically synthesized sequence
aacatccccctgggatggttcaacgtgaaagatctgaacgtgcagaacctgggcaaagaggtgaagaccacacagaaatacaccgtgaacaag

SEQ ID NO: 30 - RNA, 93 nt, Artificial Sequence
  Chemically synthesized sequence
aacaucccccugggaugguucaacgugaaagaucugaacgugcagaaccugggcaaagaggugaagaccacacagaaauacaccgugaacaag

SEQ ID NO: 31 - protein, 168 aa, Artificial Sequence
  Chemically synthesized sequence
MLPSSEMKKKLATTDPVLALSFLTAGLSTHHPHSAKAFFTFEPFPTNEEIESNKKLLEKEKKDDERIDKAYKESFKNSGLPTTLGKLDERLRNYLKKGTKGSAKQAEKMVILTENKGYYTFYLNTPLAEDRKNIELLGKMYKTYFFKKGESKSSYVINGPGKTNEYAY

SEQ ID NO: 32 - DNA, 507 bp, Artificial Sequence
  Chemically synthesized sequence
atgctccccagcagcgagatgaagaaaaaactggctacaaccgatcctgtgctggccctgagcttcctgaccgccggcctgtctacacaccacccccacagcgccaaggccttcttcacctttgagcctttcccaacaaatgaggaaatcgagagcaacaagaagctgctggaaaaagaaaagaaggacgacgagcggatcgacaaggcttataaggaatcttttaagaacagcggcctgcctaccaccctgggaaaactggacgaaagactgagaaactacctgaagaagggaacaaagggctccgccaagcaggccgagaagatggtgatcctgacagagaacaagggctactacaccttctacctgaacacccctctggccgaagatagaaagaatatcgagctgctgggcaagatgtacaagacctacttcttcaagaaaggcgagagcaagtccagctacgtgatcaacggccctggcaaaaccaacgagtacgcctactga

SEQ ID NO: 33 - RNA, 507 nt, Artificial Sequence
  Chemically synthesized sequence
augcuccccagcagcgagaugaagaaaaaacuggcuacaaccgauccugugcuggcccugagcuuccugaccgccggccugucuacacaccacccccacagcgccaaggccuucuucaccuuugagccuuucccaacaaaugaggaaaucgagagcaacaagaagcugcuggaaaaagaaaagaaggacgacgagcggaucgacaaggcuuauaaggaaucuuuuaagaacagcggccugccuaccacccugggaaaacuggacgaaagacugagaaacuaccugaagaagggaacaaagggcuccgccaagcaggccgagaagauggugauccugacagagaacaagggcuacuacaccuucuaccugaacaccccucuggccgaagauagaaagaauaucgagcugcugggcaagauguacaagaccuacuucuucaagaaaggcgagagcaaguccagcuacgugaucaacggcccuggcaaaaccaacgaguacgccuacuga

SEQ ID NO: 34 - protein, 130 aa, Artificial Sequence
  Chemically synthesized sequence
FTFEPFPTNEEIESNKKLLEKEKKDDERIDKAYKESFKNSGLPTTLGKLDERLRNYLKKGTKGSAKQAEKMVILTENKGYYTFYLNTPLAEDRKNIELLGKMYKTYFFKKGESKSSYVINGPGKTNEYAY

SEQ ID NO: 35 - DNA, 393 bp, Artificial Sequence
  Chemically synthesized sequence
ttcacctttgagcctttcccaacaaatgaggaaatcgagagcaacaagaagctgctggaaaaagaaaagaaggacgacgagcggatcgacaaggcttataaggaatcttttaagaacagcggcctgcctaccaccctgggaaaactggacgaaagactgagaaactacctgaagaagggaacaaagggctccgccaagcaggccgagaagatggtgatcctgacagagaacaagggctactacaccttctacctgaacacccctctggccgaagatagaaagaatatcgagctgctgggcaagatgtacaagacctacttcttcaagaaaggcgagagcaagtccagctacgtgatcaacggccctggcaaaaccaacgagtacgcctactga

SEQ ID NO: 36 - RNA, 393 nt, Artificial Sequence
  Chemically synthesized sequence
uucaccuuugagccuuucccaacaaaugaggaaaucgagagcaacaagaagcugcuggaaaaagaaaagaaggacgacgagcggaucgacaaggcuuauaaggaaucuuuuaagaacagcggccugccuaccacccugggaaaacuggacgaaagacugagaaacuaccugaagaagggaacaaagggcuccgccaagcaggccgagaagauggugauccugacagagaacaagggcuacuacaccuucuaccugaacaccccucuggccgaagauagaaagaauaucgagcugcugggcaagauguacaagaccuacuucuucaagaaaggcgagagcaaguccagcuacgugaucaacggcccuggcaaaaccaacgaguacgccuacuga

SEQ ID NO: 37 - protein, 62 aa, Artificial Sequence
  Chemically synthesized sequence
LRNYLKKGTKGSAKQAEKMVILTENKGYYTFYLNTPLAEDRKNIELLGKMYKTYFFKKGESK

SEQ ID NO: 38 - RNA, 186 nt, Artificial Sequence
  Chemically synthesized sequence
cugagaaacuaccugaagaagggaacaaagggcuccgccaagcaggccgagaagauggugauccugacagagaacaagggcuacuacaccuucuaccugaacaccccucuggccgaagauagaaagaauaucgagcugcugggcaagauguacaagaccuacuucuucaagaaaggcgagagcaag

SEQ ID NO: 39 - DNA, 186 bp, Artificial Sequence
  Chemically synthesized sequence
ctgagaaactacctgaagaagggaacaaagggctccgccaagcaggccgagaagatggtgatcctgacagagaacaagggctactacaccttctacctgaacacccctctggccgaagatagaaagaatatcgagctgctgggcaagatgtacaagacctacttcttcaagaaaggcgagagcaag

SEQ ID NO: 40 - protein, 118 aa, Artificial Sequence
  Chemically synthesized sequence
MKIRKSILAGTLAIVLASPLVTNLDKNEAQASASTSLPTSNEYQNEKLANELKSLLDELNVNELATGSLNTYYKRTIKISGQKAMYALKSKDFKKMSEAKYQLQKIYNEIDEALKSKY

SEQ ID NO: 41 - DNA, 357 bp, Artificial Sequence
  Chemically synthesized sequence
atgaaaattagaaagagcatcctggccggaaccctggccatcgtgctggccagccccctggtgaccaacctggataagaacgaggcccaggccagcgcctccaccagcctgcctacaagcaacgagtaccagaacgagaagctggctaatgaactgaagtctctgctggacgagctgaacgtgaacgaactcgctacaggcagcctgaatacctactacaagcggaccatcaagatcagcggccagaaggccatgtacgccctgaagtccaaggacttcaagaaaatgagcgaggctaagtaccaactgcagaaaatctacaacgaaatcgacgaggccctgaaatctaagtattga

SEQ ID NO: 42 - RNA, 357 nt, Artificial Sequence
  Chemically synthesized sequence
augaaaauuagaaagagcauccuggccggaacccuggccaucgugcuggccagcccccuggugaccaaccuggauaagaacgaggcccaggccagcgccuccaccagccugccuacaagcaacgaguaccagaacgagaagcuggcuaaugaacugaagucucugcuggacgagcugaacgugaacgaacucgcuacaggcagccugaauaccuacuacaagcggaccaucaagaucagcggccagaaggccauguacgcccugaaguccaaggacuucaagaaaaugagcgaggcuaaguaccaacugcagaaaaucuacaacgaaaucgacgaggcccugaaaucuaaguauuga

SEQ ID NO: 43 - protein, 85 aa, Artificial Sequence
  Chemically synthesized sequence
STSLPTSNEYQNEKLANELKSLLDELNVNELATGSLNTYYKRTIKISGQKAMYALKSKDFKKMSEAKYQLQKIYNEIDEALKSKY

SEQ ID NO: 44 - RNA, 258 nt, Artificial Sequence
  Chemically synthesized sequence
uccaccagccugccuacaagcaacgaguaccagaacgagaagcuggcuaaugaacugaagucucugcuggacgagcugaacgugaacgaacucgcuacaggcagccugaauaccuacuacaagcggaccaucaagaucagcggccagaaggccauguacgcccugaaguccaaggacuucaagaaaaugagcgaggcuaaguaccaacugcagaaaaucuacaacgaaaucgacgaggcccugaaaucuaaguauuga

SEQ ID NO: 45 - protein, 50 aa, Artificial Sequence
  Chemically synthesized sequence
STSLPTSNEYQNEKLANELKSLLDELNVNELATGSLNTYYKRTIKISGQK

SEQ ID NO: 46 - DNA, 150 bp, Artificial Sequence
  Chemically synthesized sequence
tccaccagcctgcctacaagcaacgagtaccagaacgagaagctggctaatgaactgaagtctctgctggacgagctgaacgtgaacgaactcgctacaggcagcctgaatacctactacaagcggaccatcaagatcagcggccagaag

SEQ ID NO: 47 - RNA, 150 nt, Artificial Sequence
  Chemically synthesized sequence
uccaccagccugccuacaagcaacgaguaccagaacgagaagcuggcuaaugaacugaagucucugcuggacgagcugaacgugaacgaacucgcuacaggcagccugaauaccuacuacaagcggaccaucaagaucagcggccagaag

SEQ ID NO: 48 - protein, 18 aa, Artificial Sequence
  Chemically synthesized sequence
LATGSLNTYYKRTIKISG

SEQ ID NO: 49 - DNA, 54 bp, Artificial Sequence
  Chemically synthesized sequence
ctcgctacaggcagcctgaatacctactacaagcggaccatcaagatcagcggc

SEQ ID NO: 50 - RNA, 54 nt, Artificial Sequence
  Chemically synthesized sequence
cucgcuacaggcagccugaauaccuacuacaagcggaccaucaagaucagcggc

SEQ ID NO: 51 - protein, 321 aa, Artificial Sequence
  Chemically synthesized sequence
MKTRIVSSVTTTLLLGSILMNPVANAADSDINIKTGTTDKIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 52 - DNA, 966 bp, Artificial Sequence
  Chemically synthesized sequence
atgaaaacaagaatcgtgtccagcgtgacgaccacactgctgctcggcagcatcctgatgaaccccgtggccaatgccgctgatagcgatatcaacatcaagaccggcaccacagataagatcggctctaataccaccgtgaagaccggcgacctggtgacctacgacaaggagaacggcatgcacaagaaggtgttctacagcttcatcgacgataagaaccacaacaagaaactgctggtcatcagaacaaaaggcaccatcgccggccagtacagagtgtattctgaagagggcgccaacaagagcggactggcctggccttccgcctttaaggtgcagctgcaactgcctgataacgaggtggcccagattagcgactactaccccagaaacagcatcgacaccaaggaatacatgagcaccctgacctacggcttcaacggaaatgtgacaggagatgatacaggcaagattggcggcctgatcggcgctaacgtgtccatcggacacacactgaagtatgtgcaacctgacttcaagaccatcctggaatctcccaccacagacaagaaggtcggatggaaggtgatcttcaacaacatggtgaaccagaactggggcccttacgacagggacagctggaaccctgtgtacggcaatcagctgttcatgaagacccgcaacggtagcatgaaggccgccgataacttcctggacccaaacaaagccagcagcctgctgagctccggcttcagccctgattttgccacagttatcaccatggacagaaaggcttctaaacagcagaccaacatcgacgtgatctacgagagagtgcgggacgactaccagctgcattggacaagcaccaattggaaaggcacaaacaccaaggacaaatggaccgaccggagctctgagcggtacaagatcgactgggagaaggaggaaatgaccaattga

SEQ ID NO: 53 - RNA, 966 nt, Artificial Sequence
  Chemically synthesized sequence
augaaaacaagaaucguguccagcgugacgaccacacugcugcucggcagcauccugaugaaccccguggccaaugccgcugauagcgauaucaacaucaagaccggcaccacagauaagaucggcucuaauaccaccgugaagaccggcgaccuggugaccuacgacaaggagaacggcaugcacaagaagguguucuacagcuucaucgacgauaagaaccacaacaagaaacugcuggucaucagaacaaaaggcaccaucgccggccaguacagaguguauucugaagagggcgccaacaagagcggacuggccuggccuuccgccuuuaaggugcagcugcaacugccugauaacgagguggcccagauuagcgacuacuaccccagaaacagcaucgacaccaaggaauacaugagcacccugaccuacggcuucaacggaaaugugacaggagaugauacaggcaagauuggcggccugaucggcgcuaacguguccaucggacacacacugaaguaugugcaaccugacuucaagaccauccuggaaucucccaccacagacaagaaggucggauggaaggugaucuucaacaacauggugaaccagaacuggggcccuuacgacagggacagcuggaacccuguguacggcaaucagcuguucaugaagacccgcaacgguagcaugaaggccgccgauaacuuccuggacccaaacaaagccagcagccugcugagcuccggcuucagcccugauuuugccacaguuaucaccauggacagaaaggcuucuaaacagcagaccaacaucgacgugaucuacgagagagugcgggacgacuaccagcugcauuggacaagcaccaauuggaaaggcacaaacaccaaggacaaauggaccgaccggagcucugagcgguacaagaucgacugggagaaggaggaaaugaccaauuga

SEQ ID NO: 54 - protein, 295 aa, Artificial Sequence
  Chemically synthesized sequence
ADSDINIKTGTTDKIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 55 - DNA, 888 bp, Artificial Sequence
  Chemically synthesized sequence
gctgatagcgatatcaacatcaagaccggcaccacagataagatcggctctaataccaccgtgaagaccggcgacctggtgacctacgacaaggagaacggcatgcacaagaaggtgttctacagcttcatcgacgataagaaccacaacaagaaactgctggtcatcagaacaaaaggcaccatcgccggccagtacagagtgtattctgaagagggcgccaacaagagcggactggcctggccttccgcctttaaggtgcagctgcaactgcctgataacgaggtggcccagattagcgactactaccccagaaacagcatcgacaccaaggaatacatgagcaccctgacctacggcttcaacggaaatgtgacaggagatgatacaggcaagattggcggcctgatcggcgctaacgtgtccatcggacacacactgaagtatgtgcaacctgacttcaagaccatcctggaatctcccaccacagacaagaaggtcggatggaaggtgatcttcaacaacatggtgaaccagaactggggcccttacgacagggacagctggaaccctgtgtacggcaatcagctgttcatgaagacccgcaacggtagcatgaaggccgccgataacttcctggacccaaacaaagccagcagcctgctgagctccggcttcagccctgattttgccacagttatcaccatggacagaaaggcttctaaacagcagaccaacatcgacgtgatctacgagagagtgcgggacgactaccagctgcattggacaagcaccaattggaaaggcacaaacaccaaggacaaatggaccgaccggagctctgagcggtacaagatcgactgggagaaggaggaaatgaccaattga

SEQ ID NO: 56 - RNA, 888 nt, Artificial Sequence
  Chemically synthesized sequence
gcugauagcgauaucaacaucaagaccggcaccacagauaagaucggcucuaauaccaccgugaagaccggcgaccuggugaccuacgacaaggagaacggcaugcacaagaagguguucuacagcuucaucgacgauaagaaccacaacaagaaacugcuggucaucagaacaaaaggcaccaucgccggccaguacagaguguauucugaagagggcgccaacaagagcggacuggccuggccuuccgccuuuaaggugcagcugcaacugccugauaacgagguggcccagauuagcgacuacuaccccagaaacagcaucgacaccaaggaauacaugagcacccugaccuacggcuucaacggaaaugugacaggagaugauacaggcaagauuggcggccugaucggcgcuaacguguccaucggacacacacugaaguaugugcaaccugacuucaagaccauccuggaaucucccaccacagacaagaaggucggauggaaggugaucuucaacaacauggugaaccagaacuggggcccuuacgacagggacagcuggaacccuguguacggcaaucagcuguucaugaagacccgcaacgguagcaugaaggccgccgauaacuuccuggacccaaacaaagccagcagccugcugagcuccggcuucagcccugauuuugccacaguuaucaccauggacagaaaggcuucuaaacagcagaccaacaucgacgugaucuacgagagagugcgggacgacuaccagcugcauuggacaagcaccaauuggaaaggcacaaacaccaaggacaaauggaccgaccggagcucugagcgguacaagaucgacugggagaaggaggaaaugaccaauuga

SEQ ID NO: 57 - protein, 65 aa, Artificial Sequence
  Chemically synthesized sequence
ADSDINIKTGTTDIGSNTTVKTGDLVTYDKENGMHKKVFYSFIDDKNHNKKLLVIRTKGTIAGQY

SEQ ID NO: 58 - DNA, 198 bp, Artificial Sequence
  Chemically synthesized sequence
gctgatagcgatatcaacatcaagaccggcaccacagataagatcggctctaataccaccgtgaagaccggcgacctggtgacctacgacaaggagaacggcatgcacaagaaggtgttctacagcttcatcgacgataagaaccacaacaagaaactgctggtcatcagaacaaaaggcaccatcgccggccagtac

SEQ ID NO: 59 - RNA, 198 nt, Artificial Sequence
  Chemically synthesized sequence
gcugauagcgauaucaacaucaagaccggcaccacagauaagaucggcucuaauaccaccgugaagaccggcgaccuggugaccuacgacaaggagaacggcaugcacaagaagguguucuacagcuucaucgacgauaagaaccacaacaagaaacugcuggucaucagaacaaaaggcaccaucgccggccaguac

SEQ ID NO: 60 - protein, 466 aa, Artificial Sequence
  Chemically synthesized sequence
MTKSVAIIGAGITGLSSAYFLKQQDPNIDVTIFEASNRPGGKIQSYRKDGYMIELGPESYLGRKTIMTELAKDIGLEQDIVTNTTGQSYIFAKNKLYPIPGGSIMGIPTDIKPFVTTKLISPLGKLRAGFDLLKKPTQMQDGDISVGAFFRARLGNEVLENLIEPLMGGIYGTDIDKLSLMSTFPNFKEKEEAFGSLIKGMKDEKNKRLKQRQLYPGAPKGQFKQFKHGLSSFIEALEQDVKNKGVTIRYNTSVDDIITSQKQYKIVYNDQLEEVYDGVLVTTPHQVFLNWFGQDPAFDYFKTMDSTTVATVVLAFDEKDIENTHDGTGFVIARTSDTDITACTWTSKKWPFTTPEGKVLIRAYVGKPGDTVVDDHTDNELVSIVRRDLSQMMTFKGDPEFTIVNRLPKSMPQYHVGHIQQIRQIQAHIKQTYPRLRVTGASFEAVGLPDCITQGKVAAEEVIAEL

SEQ ID NO: 61 - DNA, 1398 bp, Artificial Sequence
  Chemically synthesized sequence
atgacaaagagcgtagcaatcattggtgcaggcattaccggcctgagctccgcttacttcctgaaacaacaagaccctaatatcgatgtaaccatcttcgaagcctccaatcgccctgggggcaagatccagtcttatagaaaagacggttacatgatagagctggggcccgaaagctatctcggtcgaaaaacgatcatgaccgagcttgccaaagacattggcctggagcaggacattgtgacaaacaccacgggacaaagctatatattcgctaagaataagctctatccaatcccgggcgggtcaattatgggcattcccacggatatcaaaccattcgtaaccacaaaattgatctctcccttggggaagctgagagctgggtttgacctgctgaagaaaccaacccagatgcaagacggggacatctctgtgggggccttctttagagctaggctgggaaacgaagtgctggagaacctgatcgagcccctcatgggcggcatctatgggactgatattgacaagcttagcctgatgtctactttcccaaactttaaggagaaggaagaagcattcggctccctgattaaggggatgaaggacgaaaagaacaagcggctgaagcagagacaactgtatcctggggcccccaagggacagttcaagcaattcaagcatgggctgagttccttcattgaggctctggagcaggacgtgaagaacaagggcgtgaccatcagatataatacatctgtggatgacattattacctcacagaaacagtataagatcgtgtacaatgaccagttggaggaagtgtacgacggggtccttgtgacaaccccacaccaggttttcctcaattggtttgggcaggatcccgcgtttgactactttaagaccatggattcaaccacagtggccaccgtcgtattggcattcgacgagaaggacattgaaaatactcacgatggcacgggatttgtgatcgctcggacgtcagacacagacatcaccgcgtgcacttggacatctaagaaatggcccttcactacaccagagggaaaagtattgattagggcctacgtcgggaagcctggggacacggttgtggacgatcacacagacaacgaactggtgtcaattgtgagacgagatctctctcagatgatgactttcaaaggggacccagaattcactatcgtgaatcggcttcccaagagcatgccccagtaccacgtggggcacatccagcagatccggcagatccaagcccatataaagcaaacatatccacgccttcgggttactggcgcctcttttgaggctgtagggctgcctgattgtataacacagggcaaggtggcagctgaggaggtgattgccgaactg

SEQ ID NO: 62 - RNA, 1398 nt, Artificial Sequence
  Chemically synthesized sequence
augacaaagagcguagcaaucauuggugcaggcauuaccggccugagcuccgcuuacuuccugaaacaacaagacccuaauaucgauguaaccaucuucgaagccuccaaucgcccugggggcaagauccagucuuauagaaaagacgguuacaugauagagcuggggcccgaaagcuaucucggucgaaaaacgaucaugaccgagcuugccaaagacauuggccuggagcaggacauugugacaaacaccacgggacaaagcuauauauucgcuaagaauaagcucuauccaaucccgggcgggucaauuaugggcauucccacggauaucaaaccauucguaaccacaaaauugaucucucccuuggggaagcugagagcuggguuugaccugcugaagaaaccaacccagaugcaagacggggacaucucugugggggccuucuuuagagcuaggcugggaaacgaagugcuggagaaccugaucgagccccucaugggcggcaucuaugggacugauauugacaagcuuagccugaugucuacuuucccaaacuuuaaggagaaggaagaagcauucggcucccugauuaaggggaugaaggacgaaaagaacaagcggcugaagcagagacaacuguauccuggggcccccaagggacaguucaagcaauucaagcaugggcugaguuccuucauugaggcucuggagcaggacgugaagaacaagggcgugaccaucagauauaauacaucuguggaugacauuauuaccucacagaaacaguauaagaucguguacaaugaccaguuggaggaaguguacgacgggguccuugugacaaccccacaccagguuuuccucaauugguuugggcaggaucccgcguuugacuacuuuaagaccauggauucaaccacaguggccaccgucguauuggcauucgacgagaaggacauugaaaauacucacgauggcacgggauuugugaucgcucggacgucagacacagacaucaccgcgugcacuuggacaucuaagaaauggcccuucacuacaccagagggaaaaguauugauuagggccuacgucgggaagccuggggacacgguuguggacgaucacacagacaacgaacuggugucaauugugagacgagaucucucucagaugaugacuuucaaaggggacccagaauucacuaucgugaaucggcuucccaagagcaugccccaguaccacguggggcacauccagcagauccggcagauccaagcccauauaaagcaaacauauccacgccuucggguuacuggcgccucuuuugaggcuguagggcugccugauuguauaacacagggcaagguggcagcugaggaggugauugccgaacug

SEQ ID NO: 63 - protein, 12 aa, Artificial Sequence
  Chemically synthesized sequence
TDNELVSIVRRD

SEQ ID NO: 64 - DNA, 36 bp, Artificial Sequence
  Chemically synthesized sequence
accgacaatgagctggtgagtatcgtgaggagggac

SEQ ID NO: 65 - RNA, 36 nt, Artificial Sequence
  Chemically synthesized sequence
accgacaaugagcuggugaguaucgugaggagggac

SEQ ID NO: 66 - protein, 12 aa, Artificial Sequence
  Chemically synthesized sequence
TDDKLVSIVRRD

SEQ ID NO: 67 - DNA, 36 bp, Artificial Sequence
  Chemically synthesized sequence
accgacgataagctggtctccatcgtccggcgcgac

SEQ ID NO: 68 - RNA, 36 nt, Artificial Sequence
  Chemically synthesized sequence
accgacgauaagcuggucuccaucguccggcgcgac

SEQ ID NO: 69 - protein, 12 aa, Artificial Sequence
  Chemically synthesized sequence
TDDELVSIVRRD

SEQ ID NO: 70 - DNA, 36 bp, Artificial Sequence
  Chemically synthesized sequence
actgacgatgaactggtcagcatcgtgcggcgggac

SEQ ID NO: 71 - RNA, 36 nt, Artificial Sequence
  Chemically synthesized sequence
acugacgaugaacuggucagcaucgugcggcgggac

SEQ ID NO: 72 - protein, 1068 aa, Artificial Sequence
  Chemically synthesized sequence
SASAQPRSVAATPKTSLPKYKPQVNSSINDYIRKNNLKAPKIEEDYTSYFPKYAYRNGVGRPEGIVVHDTANDRSTINGEISYMKNNYQNAFVHAFVDGDRIIETAPTDYLSWGVGAVGNPRFINVEIVHTHDYASFARSMNNYADYAATQLQYYGLKPDSAEYDGNGTVWTHYAVSKYLGGTDHADPHGYLRSHNYSYDQLYDLINEKYLIKMGKVAPWGTQSTTTPTTPSKPTTPSTPSKPSKPSTGKLTVAANNGVAQIKPTNSGLYTTVYDKTGKATNEVQKTFAVSKTATLGNQKFYLVQDYNSGNKFGWVKEGDVVYNTAKSPVNVNQSYSIKPGTKLYTVPWGTSKQVAGSVSGSGNQTFKASKQQQIDKSIYLYGSVNGKSGWVSKAYLVDTAKPTPTPTPKPSTPTTNNKLTVSSLNGVAQINAKNNGLFTTVYDKTGKPTKEVQKTFAVTKEASLGGNKFYLVKDYNSPTLIGWVKQGDVIYNNAKSPVNVMQTYTVKPGTKLYSVPWGTYKQEAGAVSGTGNQTFKATKQQQIDKSIYLFGTVNGKSGWVSKAYLAVPAAPKKAVAQPKTAVKAYAVTKPQTTQTTQTVSKIAQVKPNNTGIRASVYEKTAKNGAKYADRTFYVTKERAHGNETYVLLNNTSHNIPLGWFNVKDLNVQNLGKEVKTTQKYTVNKSNNGLSMVPWGTKNQVILTGNNIAQGTFNATKQVSVGKDVYLYGTINNRTGWVNAKDLTAPTAVKPTTSAAKDYNYTYVIKNGNGYYYVTPNSDTAKYSLKAFNEQPFAVVKEQVINGQTWYYGKLSNGKLAWIKSTDLAKELIKYNQTGMTLNQVAQIQAGLQYKPQVQRVPGKWTDANFNDVKHAMDTKRLAQDPALKYQFLRLDQPQNISIDKINQFLKGKGVLENQGAAFNKAAQMYGINEVYLISHALLETGNGTSQLAKGADVVNNKVVTNSNTKYHNVFGIAAYDNDPLREGIKYAKQAGWDTVSKAIVGGAKFIGNSYVKAGQNTLYKMRWNPAHPGTHQYATDVDWANINAKIIKGYYDKIGEVGKYFDIPQYK

SEQ ID NO: 73 - DNA, 3205 bp, Artificial Sequence
  Chemically synthesized sequence
ttctgccagcgcccagccaagaagcgtagccgccacccctaagacgagcctgcccaaatacaagccccaggtgaatagcagcattaacgactacatcagaaagaataatctgaaggccccaaagatcgaggaagattacaccagctacttccccaagtacgcctatagaaacggcgtgggcagacctgagggcatcgtggttcacgacacagctaatgaccggagcaccatcaacggcgaaatcagctacatgaagaacaactaccagaatgccttcgtgcacgccttcgtggatggcgaccggatcatcgagaccgctcctaccgactacctgagctggggcgtgggcgccgtgggcaaccccagattcatcaacgtggaaatcgtgcatacacacgactacgccagcttcgctagaagcatgaacaactacgccgattacgccgctacacaactgcaatactacggcctgaagcctgatagcgccgagtatgacggcaacggcacagtttggacccactacgccgtgagcaagtacctcggcggcacagaccacgccgatcctcacggctacctgcggtcccacaactactcttacgatcagctgtacgacctgatcaacgaaaagtatcttatcaaaatgggcaaggtggcaccttggggcacccagagcaccaccacacctacaacaccttccaagcctacaaccccttctaccccttctaagccctctaagcccagcaccgggaaactgacagtggccgctaacaacggagtagcccagatcaaacctaccaacagcggcctgtacaccaccgtctacgacaagacaggcaaggctaccaacgaggtgcagaagacctttgccgtgtccaaaacagcaacgctgggcaaccagaagttctacctggtgcaggactacaacagtggcaacaagttcggctgggtgaaggaaggagatgtggtgtacaacaccgccaagagccctgtgaatgtgaaccagtcttatagcatcaagcccggcacaaagctgtacaccgttccttggggaacctccaaacaggtggccggcagtgtgagtggctccggcaatcagaccttcaaggccagcaagcagcagcagatcgacaaatctatctacctgtatggaagtgtgaacgggaagtccggctgggtatccaaggcatacctagttgataccgccaagcccacccctaccccgacacctaaacctagcacacccacaaccaacaacaagctgaccgtgtcttctctgaacggagtggcccagatcaacgccaagaacaatggcctgttcaccacagtgtacgacaagacaggcaagcctaccaaggaggtgcagaagaccttcgccgtgaccaaggaagcaagcctgggaggcaacaagttctacctcgtgaaggactacaatagccctaccctgattggctgggtgaagcagggagatgtaatctacaacaacgctaagagccccgtgaacgtgatgcagacctacaccgtgaagcctggcacaaagctgtacagcgtgccctggggcacatacaagcaggaggccggcgccgtgagcggaaccggcaaccagacctttaaggccaccaagcagcaacagatcgacaagagtatatacctgttcggcactgtaaacggtaagtccggctgggtgagcaaagcgtacctggccgttcctgctgcccctaaaaaggccgtggctcaaccgaaaaccgccgtgaaagcctacgccgtcaccaaacctcagacaacacagacaactcaaaccgtaagcaagatcgcccaggtgaagcccaacaacaccggaattcgggccagcgtgtacgagaagaccgccaagaacggcgccaagtacgctgatagaacattctacgtgaccaaggaacgggcccacggtaacgagacatacgtgctgctgaacaacacctcccacaacatccccctgggatggttcaacgtgaaagatctgaacgtgcagaacctgggcaaagaggtgaagaccacacagaaatacaccgtgaacaagagtaacaacggcctcagcatggtgccttggggcaccaagaaccaggtgatcctgaccggcaacaatatcgcccaaggcaccttcaacgccaccaagcaggtgagcgtgggcaaggacgtgtacctgtacggcacaatcaacaacagaaccggatgggtcaacgccaaggacctgaccgcccctactgccgtgaaaccaacaaccagcgccgccaaggactacaattacacctacgtgattaagaatggaaatggctactactacgtgacaccaaatagtgataccgccaaatatagcctgaaggccttcaacgagcagccattcgcggtggtgaaggagcaggtgatcaacggacagacctggtactacggcaagctgagcaacggcaaactggcctggatcaagagcactgacctggccaaagagctgattaaatacaaccagacgggcatgacccttaaccaagtggcccagatccaggccggactgcagtacaagcctcaggtgcagagagtgccagggaagtggaccgacgccaacttcaacgacgtgaagcacgctatggacacaaagcgcctggcccaggatcctgctctgaagtaccagtttctgagactggaccagcctcagaacatctccatcgacaagatcaaccagttcctgaaaggcaagggcgttctggagaaccaaggcgccgccttcaacaaagctgctcagatgtacggcatcaatgaggtgtacctgatcagccacgccctgctggaaaccggcaacggcaccagccagctggctaagggcgccgacgtggtgaacaacaaggtggtgaccaactctaataccaagtatcacaacgtctttggaatcgccgcctatgacaacgaccccctgagggaaggcatcaaatacgccaagcaggccggctgggatacagtgagcaaggctatcgtggggggcgctaagttcatcggaaattcttacgtgaaggctggccagaacaccctgtacaagatgcggtggaaccctgctcatcctggcacccaccaatacgccacggacgtggactgggccaacatcaacgccaagatcatcaagggttactacgacaagattggagaggtgggcaagtacttcgacatccctcagtacaag

SEQ ID NO: 74 - RNA, 3205 nt, Artificial Sequence
  Chemically synthesized sequence
uucugccagcgcccagccaagaagcguagccgccaccccuaagacgagccugcccaaauacaagccccaggugaauagcagcauuaacgacuacaucagaaagaauaaucugaaggccccaaagaucgaggaagauuacaccagcuacuuccccaaguacgccuauagaaacggcgugggcagaccugagggcaucgugguucacgacacagcuaaugaccggagcaccaucaacggcgaaaucagcuacaugaagaacaacuaccagaaugccuucgugcacgccuucguggauggcgaccggaucaucgagaccgcuccuaccgacuaccugagcuggggcgugggcgccgugggcaaccccagauucaucaacguggaaaucgugcauacacacgacuacgccagcuucgcuagaagcaugaacaacuacgccgauuacgccgcuacacaacugcaauacuacggccugaagccugauagcgccgaguaugacggcaacggcacaguuuggacccacuacgccgugagcaaguaccucggcggcacagaccacgccgauccucacggcuaccugcggucccacaacuacucuuacgaucagcuguacgaccugaucaacgaaaaguaucuuaucaaaaugggcaagguggcaccuuggggcacccagagcaccaccacaccuacaacaccuuccaagccuacaaccccuucuaccccuucuaagcccucuaagcccagcaccgggaaacugacaguggccgcuaacaacggaguagcccagaucaaaccuaccaacagcggccuguacaccaccgucuacgacaagacaggcaaggcuaccaacgaggugcagaagaccuuugccguguccaaaacagcaacgcugggcaaccagaaguucuaccuggugcaggacuacaacaguggcaacaaguucggcugggugaaggaaggagaugugguguacaacaccgccaagagcccugugaaugugaaccagucuuauagcaucaagcccggcacaaagcuguacaccguuccuuggggaaccuccaaacagguggccggcagugugaguggcuccggcaaucagaccuucaaggccagcaagcagcagcagaucgacaaaucuaucuaccuguauggaagugugaacgggaaguccggcuggguauccaaggcauaccuaguugauaccgccaagcccaccccuaccccgacaccuaaaccuagcacacccacaaccaacaacaagcugaccgugucuucucugaacggaguggcccagaucaacgccaagaacaauggccuguucaccacaguguacgacaagacaggcaagccuaccaaggaggugcagaagaccuucgccgugaccaaggaagcaagccugggaggcaacaaguucuaccucgugaaggacuacaauagcccuacccugauuggcugggugaagcagggagauguaaucuacaacaacgcuaagagccccgugaacgugaugcagaccuacaccgugaagccuggcacaaagcuguacagcgugcccuggggcacauacaagcaggaggccggcgccgugagcggaaccggcaaccagaccuuuaaggccaccaagcagcaacagaucgacaagaguauauaccuguucggcacuguaaacgguaaguccggcugggugagcaaagcguaccuggccguuccugcugccccuaaaaaggccguggcucaaccgaaaaccgccgugaaagccuacgccgucaccaaaccucagacaacacagacaacucaaaccguaagcaagaucgcccaggugaagcccaacaacaccggaauucgggccagcguguacgagaagaccgccaagaacggcgccaaguacgcugauagaacauucuacgugaccaaggaacgggcccacgguaacgagacauacgugcugcugaacaacaccucccacaacaucccccugggaugguucaacgugaaagaucugaacgugcagaaccugggcaaagaggugaagaccacacagaaauacaccgugaacaagaguaacaacggccucagcauggugccuuggggcaccaagaaccaggugauccugaccggcaacaauaucgcccaaggcaccuucaacgccaccaagcaggugagcgugggcaaggacguguaccuguacggcacaaucaacaacagaaccggaugggucaacgccaaggaccugaccgccccuacugccgugaaaccaacaaccagcgccgccaaggacuacaauuacaccuacgugauuaagaauggaaauggcuacuacuacgugacaccaaauagugauaccgccaaauauagccugaaggccuucaacgagcagccauucgcgguggugaaggagcaggugaucaacggacagaccugguacuacggcaagcugagcaacggcaaacuggccuggaucaagagcacugaccuggccaaagagcugauuaaauacaaccagacgggcaugacccuuaaccaaguggcccagauccaggccggacugcaguacaagccucaggugcagagagugccagggaaguggaccgacgccaacuucaacgacgugaagcacgcuauggacacaaagcgccuggcccaggauccugcucugaaguaccaguuucugagacuggaccagccucagaacaucuccaucgacaagaucaaccaguuccugaaaggcaagggcguucuggagaaccaaggcgccgccuucaacaaagcugcucagauguacggcaucaaugagguguaccugaucagccacgcccugcuggaaaccggcaacggcaccagccagcuggcuaagggcgccgacguggugaacaacaagguggugaccaacucuaauaccaaguaucacaacgucuuuggaaucgccgccuaugacaacgacccccugagggaaggcaucaaauacgccaagcaggccggcugggauacagugagcaaggcuaucguggggggcgcuaaguucaucggaaauucuuacgugaaggcuggccagaacacccuguacaagaugcgguggaacccugcucauccuggcacccaccaauacgccacggacguggacugggccaacaucaacgccaagaucaucaaggguuacuacgacaagauuggagaggugggcaaguacuucgacaucccucaguacaag

SEQ ID NO: 75 - DNA, 258 bp, Artificial Sequence
  Chemically synthesized sequence
tccaccagcctgcctacaagcaacgagtaccagaacgagaagctggctaatgaactgaagtctctgctggacgagctgaacgtgaacgaactcgctacaggcagcctgaatacctactacaagcggaccatcaagatcagcggccagaaggccatgtacgccctgaagtccaaggacttcaagaaaatgagcgaggctaagtaccaactgcagaaaatctacaacgaaatcgacgaggccctgaaatctaagtattga

SEQ ID NO: 76 - DNA, 3768 bp, Artificial Sequence
  Chemically synthesized sequence
atgttaggagtaataaatagaatggcgaaaaaattcaattacaaactaccatcaatggttgcattaacgcttgtaggttcagcagtcactgcacatcaagttcaagcagctgagacgacacaagatcaaactactaataaaaatgttttagatagtaataaagttaaagcaactactgaacaagcaaaagctgaggtaaaaaatccaacgcaaaacatttctggcactcaagtatatcaagaccctgctattgtccaaccaaaaacagcaaataacaaaacaggcaatgctcaagtaagtcaaaaagttgatactgcacaagtaaatggtgacactcgtgctaatcaatcagcgactacaaataatacgcagcctgttgcaaagtcaacaagcactacagcacctaaaactaacactaatgttacaaatgctggttatagtttagttgatgatgaagatgataattcagaacatcaaattaatccagaattaattaaatcagctgctaaacctgcagctcttgaaacgcaatataaagccgcagcacctaaagctaaaactgaagcgacacctaaagtaactacttttagcgcttcagcacaaccaagatcagttgctgcaacaccaaaaacgagtttgccaaaatataaaccacaagtaaactcttcaattaacgattacattcgtaaaaataacttaaaagcacctaaaattgaagaagattatacatcttacttccctaaatacgcataccgtaacggcgtaggtcgtcctgaaggtatcgtagttcatgatacagctaatgatcgttcgacgataaatggtgaaattagttatatgaaaaataactatcaaaacgcattcgtacatgcatttgttgatggggatcgtataatcgaaacagcaccaacggattacttatcttggggtgtcggtgcagtcggtaaccctagattcatcaatgttgaaatcgtacacacacacgactatgcttcatttgcacgttcaatgaataactatgctgactatgcagctacacaattacaatattatggtttaaaaccagacagtgctgagtatgatggaaatggtacagtatggactcactacgctgtaagtaaatatttaggtggtacggaccatgccgatccacatggatatttaagaagtcataattatagttatgatcaattatatgacttaattaatgaaaaatatttaataaaaatgggtaaagtggcgccatggggtacgcaatttacaactacccctactacaccatcaaaaccaacaacaccgtcgaaaccatcaactggtaaattaacagttgcagcaaacaatggtgtcgcacaaatcaaaccaacaaatagtggtttatatactactgtttacgacaaaactggtaaagcaactaatgaagttcaaaaaacatttgctgtatctaaaacagctacattaggtaatcaaaaattctatcttgttcaagattacaattctggtaataaatttggttgggttaaagaaggcgatgtggtttacaacacagctaaatcacctgtaaatgtaaatcaatcatattcaatcaaatctggtacgaaactttatacagtaccttggggtacatctaaacaagttgctggtagcgtgtctggctctggaaaccaaacatttaaggcttcaaagcaacaacaaattgataaatcaatttatttatatggctctgtgaatggtaaatctggttgggtaagtaaagcatatttagttgatactgctaaacctacgcctacaccaatacctaagccatcaacacctacaacaaataataaattaacagtttcatcattaaacggtgttgctcaaattaatgctaaaaacaatggcttattcactacagtttatgacaaaactggtaagccaacgaaagaagttcaaaaaacatttgctgtaacaaaagaagcaagtttaggtggaaacaaattctacttagttaaagattacaatagtccaactttaattggttgggttaaacaaggtgacgttatttataacaatgcaaaatcacctgtaaatgtaatgcaaacatatacagtaaaaccaggcactaaattatattcagtaccttggggcacttataaacaagaagctggtgcagtttctggtacaggtaaccaaacttttaaagcgactaagcaacaacaaattgataaatctatctatttatttggaactgtaaatggtaaatctggttgggtaagtaaagcatatttagctgtacctgctgcacctaaaaaagcagtagcacaaccaaaaacagctgtaaaagcttatactgttactaaaccacaaacgactcaaacagttagcaagattgctcaagttaaaccaaacaacactggtattcgtgcttctgtttatgaaaaaacagcgaaaaacggtgcgaaatatgcagaccgtacgttctatgtaacaaaagagcgtgctcatggtaatgaaacgtatgtattattaaacaatacaagccataacatcccattaggttggttcaatgtaaaagacttaaatgttcaaaacctaggcaaagaagttaaaacgactcaaaaatatactgttaataaatcaaataacggcttatcaatggttccttggggtactaaaaaccaagtcattttaacaggcaataacattgctcaaggtacatttaatgcaacgaaacaagtatctgtaggcaaagatgtttatttatacggtactattaataaccgcactggttgggtaaatgcaaaagatttaactgcaccaactgctgtgaaaccaactacatcagctgccaaagattataactacacttatgtaattaaaaatggtaatggttattactatgtaacaccaaattctgatacagctaaatactcattaaaagcatttaatgaacaaccattcgcagttgttaaagaacaagtcattaatggacaaacttggtactatggtaaattatctaacggtaaattagcatggattaaatcaactgatttagctaaagaattaattaagtataatcaaacaggtatgacattaaaccaagttgctcaaatacaagctggtttacaatataaaccacaagtacaacgtgtaccaggtaagtggacagatgctaactttaatgatgttaagcatgcaatggatacgaagcgtttagctcaagatccagcattaaaatatcaattcttacgcttagaccaaccacaaaatatttctattgataaaattaatcaattcttaaaaggtaaaggtgtattagaaaaccaaggtgctgcatttaacaaagctgctcaaatgtatggcattaatgaagtttatcttatctcacatgccctattagaaacaggtaacggtacttctcaattagcgaaaggtgcagatgtagtgaacaacaaagttgtaactaactcaaacacgaaataccataacgtatttggtattgctgcatatgataacgatcctttacgtgaaggtattaaatatgctaaacaagctggttgggacacagtatcaaaagcaatcgttggtggtgctaaattcatcggcaactcatatgtaaaagctggtcaaaatacactttacaaaatgagatggaatcctgcacatccaggaacacaccaatatgctacagatgtagattgggctaacatcaatgctaaaatcatcaaaggctactatgataaaattggcgaagtcggcaaatacttcgacatcccacaatataaataa

SEQ ID NO: 77 - RNA, 3768 nt, Artificial Sequence
  Chemically synthesized sequence
auguuaggaguaauaaauagaauggcgaaaaaauucaauuacaaacuaccaucaaugguugcauuaacgcuuguagguucagcagucacugcacaucaaguucaagcagcugagacgacacaagaucaaacuacuaauaaaaauguuuuagauaguaauaaaguuaaagcaacuacugaacaagcaaaagcugagguaaaaaauccaacgcaaaacauuucuggcacucaaguauaucaagacccugcuauuguccaaccaaaaacagcaaauaacaaaacaggcaaugcucaaguaagucaaaaaguugauacugcacaaguaaauggugacacucgugcuaaucaaucagcgacuacaaauaauacgcagccuguugcaaagucaacaagcacuacagcaccuaaaacuaacacuaauguuacaaaugcugguuauaguuuaguugaugaugaagaugauaauucagaacaucaaauuaauccagaauuaauuaaaucagcugcuaaaccugcagcucuugaaacgcaauauaaagccgcagcaccuaaagcuaaaacugaagcgacaccuaaaguaacuacuuuuagcgcuucagcacaaccaagaucaguugcugcaacaccaaaaacgaguuugccaaaauauaaaccacaaguaaacucuucaauuaacgauuacauucguaaaaauaacuuaaaagcaccuaaaauugaagaagauuauacaucuuacuucccuaaauacgcauaccguaacggcguaggucguccugaagguaucguaguucaugauacagcuaaugaucguucgacgauaaauggugaaauuaguuauaugaaaaauaacuaucaaaacgcauucguacaugcauuuguugauggggaucguauaaucgaaacagcaccaacggauuacuuaucuuggggugucggugcagucgguaacccuagauucaucaauguugaaaucguacacacacacgacuaugcuucauuugcacguucaaugaauaacuaugcugacuaugcagcuacacaauuacaauauuaugguuuaaaaccagacagugcugaguaugauggaaaugguacaguauggacucacuacgcuguaaguaaauauuuaggugguacggaccaugccgauccacauggauauuuaagaagucauaauuauaguuaugaucaauuauaugacuuaauuaaugaaaaauauuuaauaaaaauggguaaaguggcgccaugggguacgcaauuuacaacuaccccuacuacaccaucaaaaccaacaacaccgucgaaaccaucaacugguaaauuaacaguugcagcaaacaauggugucgcacaaaucaaaccaacaaauagugguuuauauacuacuguuuacgacaaaacugguaaagcaacuaaugaaguucaaaaaacauuugcuguaucuaaaacagcuacauuagguaaucaaaaauucuaucuuguucaagauuacaauucugguaauaaauuugguuggguuaaagaaggcgaugugguuuacaacacagcuaaaucaccuguaaauguaaaucaaucauauucaaucaaaucugguacgaaacuuuauacaguaccuugggguacaucuaaacaaguugcugguagcgugucuggcucuggaaaccaaacauuuaaggcuucaaagcaacaacaaauugauaaaucaauuuauuuauauggcucugugaaugguaaaucugguuggguaaguaaagcauauuuaguugauacugcuaaaccuacgccuacaccaauaccuaagccaucaacaccuacaacaaauaauaaauuaacaguuucaucauuaaacgguguugcucaaauuaaugcuaaaaacaauggcuuauucacuacaguuuaugacaaaacugguaagccaacgaaagaaguucaaaaaacauuugcuguaacaaaagaagcaaguuuagguggaaacaaauucuacuuaguuaaagauuacaauaguccaacuuuaauugguuggguuaaacaaggugacguuauuuauaacaaugcaaaaucaccuguaaauguaaugcaaacauauacaguaaaaccaggcacuaaauuauauucaguaccuuggggcacuuauaaacaagaagcuggugcaguuucugguacagguaaccaaacuuuuaaagcgacuaagcaacaacaaauugauaaaucuaucuauuuauuuggaacuguaaaugguaaaucugguuggguaaguaaagcauauuuagcuguaccugcugcaccuaaaaaagcaguagcacaaccaaaaacagcuguaaaagcuuauacuguuacuaaaccacaaacgacucaaacaguuagcaagauugcucaaguuaaaccaaacaacacugguauucgugcuucuguuuaugaaaaaacagcgaaaaacggugcgaaauaugcagaccguacguucuauguaacaaaagagcgugcucaugguaaugaaacguauguauuauuaaacaauacaagccauaacaucccauuagguugguucaauguaaaagacuuaaauguucaaaaccuaggcaaagaaguuaaaacgacucaaaaauauacuguuaauaaaucaaauaacggcuuaucaaugguuccuugggguacuaaaaaccaagucauuuuaacaggcaauaacauugcucaagguacauuuaaugcaacgaaacaaguaucuguaggcaaagauguuuauuuauacgguacuauuaauaaccgcacugguuggguaaaugcaaaagauuuaacugcaccaacugcugugaaaccaacuacaucagcugccaaagauuauaacuacacuuauguaauuaaaaaugguaaugguuauuacuauguaacaccaaauucugauacagcuaaauacucauuaaaagcauuuaaugaacaaccauucgcaguuguuaaagaacaagucauuaauggacaaacuugguacuaugguaaauuaucuaacgguaaauuagcauggauuaaaucaacugauuuagcuaaagaauuaauuaaguauaaucaaacagguaugacauuaaaccaaguugcucaaauacaagcugguuuacaauauaaaccacaaguacaacguguaccagguaaguggacagaugcuaacuuuaaugauguuaagcaugcaauggauacgaagcguuuagcucaagauccagcauuaaaauaucaauucuuacgcuuagaccaaccacaaaauauuucuauugauaaaauuaaucaauucuuaaaagguaaagguguauuagaaaaccaaggugcugcauuuaacaaagcugcucaaauguauggcauuaaugaaguuuaucuuaucucacaugcccuauuagaaacagguaacgguacuucucaauuagcgaaaggugcagauguagugaacaacaaaguuguaacuaacucaaacacgaaauaccauaacguauuugguauugcugcauaugauaacgauccuuuacgugaagguauuaaauaugcuaaacaagcugguugggacacaguaucaaaagcaaucguugguggugcuaaauucaucggcaacucauauguaaaagcuggucaaaauacacuuuacaaaaugagauggaauccugcacauccaggaacacaccaauaugcuacagauguagauugggcuaacaucaaugcuaaaaucaucaaaggcuacuaugauaaaauuggcgaagucggcaaauacuucgacaucccacaauauaaauaa

SEQ ID NO: 78 - DNA, 450 bp, Artificial Sequence
  Chemically synthesized sequence
atgaaaaagaaattagcaacaacagttttagcattaagttttttaacggcaggaatcagtacacaccatcattcagcgaaagcttttacttttgaaccgtttcctacaaatgaagaaatagaatcaaataagaaaatgttagagaaagaaaaagcttataaagaatcatttaaaaatagtggtcttcctacaacgctaggaaaattagatgaacgtttgagaaattatttaaagaaaggcacaaaaaattctgctcaatttgaaaaaatggttattttaactgaaaataaaggttactatacagtatatctgaatacaccacttgctgaagatagaaaaaatgttgagttactaggtaaaatgtataaaacatacttctttaaaaaaggagagtctaaatcatcttatgtaattaatggtcctggaaaaactaatgaatatgcatactaa

SEQ ID NO: 79 - RNA, 450 nt, Artificial Sequence
  Chemically synthesized sequence
augaaaaagaaauuagcaacaacaguuuuagcauuaaguuuuuuaacggcaggaaucaguacacaccaucauucagcgaaagcuuuuacuuuugaaccguuuccuacaaaugaagaaauagaaucaaauaagaaaauguuagagaaagaaaaagcuuauaaagaaucauuuaaaaauaguggucuuccuacaacgcuaggaaaauuagaugaacguuugagaaauuauuuaaagaaaggcacaaaaaauucugcucaauuugaaaaaaugguuauuuuaacugaaaauaaagguuacuauacaguauaucugaauacaccacuugcugaagauagaaaaaauguugaguuacuagguaaaauguauaaaacauacuucuuuaaaaaaggagagucuaaaucaucuuauguaauuaaugguccuggaaaaacuaaugaauaugcauacuaa

SEQ ID NO: 80 - DNA, 348 bp, Artificial Sequence
  Chemically synthesized sequence
atatttactttttagtgcttcgtcaatttcattataaatcttttgaagttgatattttgcttctgacattttcttaaagtcttttgacttaagagcatacattgctttttgacctgaaatttttatagttcgcttataataagtgtttaaacttccagtagctaattcattaacatttagttcatctaataacgattttaattcattagctaacttttcgttttgatattcattcgatgttggcaagcttgtgctagcttgtgcctcatttttatctagattagttactagtggtgatgctaaaacgattgctaaagttcccgcaagtatagattttctaattttcat

SEQ ID NO: 81 - RNA, 348 nt, Artificial Sequence
  Chemically synthesized sequence
auauuuacuuuuuagugcuucgucaauuucauuauaaaucuuuugaaguugauauuuugcuucugacauuuucuuaaagucuuuugacuuaagagcauacauugcuuuuugaccugaaauuuuuauaguucgcuuauaauaaguguuuaaacuuccaguagcuaauucauuaacauuuaguucaucuaauaacgauuuuaauucauuagcuaacuuuucguuuugauauucauucgauguuggcaagcuugugcuagcuugugccucauuuuuaucuagauuaguuacuaguggugaugcuaaaacgauugcuaaaguucccgcaaguauagauuuucuaauuuucau

SEQ ID NO: 82 - DNA, 957 bp, Artificial Sequence
  Chemically synthesized sequence
atgaaaacacgtatagtcagctcagtaacaacaacactattgctaggttccatattaatgaatcctgtcgctaatgccgcagattctgatattaatattaaaaccggtactacagatattggaagcaatactacagtaaaaacaggtgatttagtcacttatgataaagaaaatggcatgcacaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaat

SEQ ID NO: 83 - RNA, 957 nt, Artificial Sequence
  Chemically synthesized sequence
augaaaacacguauagucagcucaguaacaacaacacuauugcuagguuccauauuaaugaauccugucgcuaaugccgcagauucugauauuaauauuaaaaccgguacuacagauauuggaagcaauacuacaguaaaaacaggugauuuagucacuuaugauaaagaaaauggcaugcacaaaaaaguauuuuauaguuuuaucgaugauaaaaaucauaauaaaaaacugcuaguuauuagaacgaaagguaccauugcuggucaauauagaguuuauagcgaagaaggugcuaacaaaagugguuuagccuggccuucagccuuuaagguacaguugcaacuaccugauaaugaaguagcucaaauaucugauuacuauccaagaaauucgauugauacaaaagaguauaugaguacuuuaacuuauggauucaacgguaauguuacuggugaugauacaggaaaaauuggcggccuuauuggugcaaauguuucgauuggucauacacugaaauauguucaaccugauuucaaaacaauuuuagagagcccaacugauaaaaaaguaggcuggaaagugauauuuaacaauauggugaaucaaaauuggggaccauaugauagagauucuuggaacccgguauauggcaaucaacuuuucaugaaaacuagaaauggcucuaugaaagcagcagauaacuuccuugauccuaacaaagcaaguucucuauuaucuucaggguuuucaccagacuucgcuacaguuauuacuauggauagaaaagcauccaaacaacaaacaaauauagauguaauauacgaacgaguucgugaugacuaccaauugcacuggacuucaacaaauuggaaagguaccaauacuaaagauaaauggauagaucguucuucagaaagauauaaaaucgauugggaaaaagaagaaaugacaaau

SEQ ID NO: 84 - DNA, 1269 bp, Artificial Sequence
  Chemically synthesized sequence
tcgtatcgtaaagatggttatatgattgaactagggcctgaatcttatttaggtagaaaaacgattatgacagaattagcgaaagatattggattagaacaagatattgttacaaatacgactggacaatcatatatttttgcgaaaaataaattatatccgattccaggtggttcaattatgggtattccaacagatattaaaccatttgttactacaaaattaatatcgccacttggtaaattaagagcagggtttgatttattaaaaaaacctactcaaatgcaggatggtgacatttctgttggtgcatttttcagagcaagattaggtaatgaggtacttgagaatttaatagagcctttaatgggtggtatttatggtaccgatattgataaattaagtttgatgagtacgtttcctaattttaaagaaaaagaagaggcattcggaagtctgataaaaggtatgaaggatgagaaaaataagcgtctgaaacaaagacaattatatcctggcgcaccgaaaggacaattcaaacaatttaagcatggtttaagctcatttattgaagcattagaacaagatgttaaaaataaaggtgtgacaatacgctacaatacgtcagtggatgatataattacatctcaaaaacaatataaaattgtttacaatgatcaactagaagaagtgtatgatggcgtattagtaacgacaccacatcaagtgtttttaaattggttcggacaagatccagcatttgattactttaaaacgatggatagtacgactgttgcaactgttgtattggcatttgatgaaaaagacattgaaaatacccatgatggtactggcttcgtaattgcgagaacgagtgatacagacattaccgcatgtacttggacatcgaaaaaatggccatttactacaccggaaggtaaggttttgattcgtgcgtatgtaggtaaaccaggtgatactgtggttgatgatcatacagataatgaattagtatcgattgtacgtagagatttaagtcaaatgatgacatttaaaggtgatcctgaatttacaattgtcaatcgtttgccgaaaagtatgccacagtaccatgtcggtcatattcaacaaattagacagattcaagcacatattaaacaaacatatccacgacttagagtaactggtgcatcttttgaagcggttggactacctgattgtattacgcaaggtaaagttgctgctgaagaagtaatcgcagagttgtaa

SEQ ID NO: 85 - RNA, 1269 nt, Artificial Sequence
  Chemically synthesized sequence
ucguaucguaaagaugguuauaugauugaacuagggccugaaucuuauuuagguagaaaaacgauuaugacagaauuagcgaaagauauuggauuagaacaagauauuguuacaaauacgacuggacaaucauauauuuuugcgaaaaauaaauuauauccgauuccaggugguucaauuauggguauuccaacagauauuaaaccauuuguuacuacaaaauuaauaucgccacuugguaaauuaagagcaggguuugauuuauuaaaaaaaccuacucaaaugcaggauggugacauuucuguuggugcauuuuucagagcaagauuagguaaugagguacuugagaauuuaauagagccuuuaaugggugguauuuaugguaccgauauugauaaauuaaguuugaugaguacguuuccuaauuuuaaagaaaaagaagaggcauucggaagucugauaaaagguaugaaggaugagaaaaauaagcgucugaaacaaagacaauuauauccuggcgcaccgaaaggacaauucaaacaauuuaagcaugguuuaagcucauuuauugaagcauuagaacaagauguuaaaaauaaaggugugacaauacgcuacaauacgucaguggaugauauaauuacaucucaaaaacaauauaaaauuguuuacaaugaucaacuagaagaaguguaugauggcguauuaguaacgacaccacaucaaguguuuuuaaauugguucggacaagauccagcauuugauuacuuuaaaacgauggauaguacgacuguugcaacuguuguauuggcauuugaugaaaaagacauugaaaauacccaugaugguacuggcuucguaauugcgagaacgagugauacagacauuaccgcauguacuuggacaucgaaaaaauggccauuuacuacaccggaagguaagguuuugauucgugcguauguagguaaaccaggugauacugugguugaugaucauacagauaaugaauuaguaucgauuguacguagagauuuaagucaaaugaugacauuuaaaggugauccugaauuuacaauugucaaucguuugccgaaaaguaugccacaguaccaugucggucauauucaacaaauuagacagauucaagcacauauuaaacaaacauauccacgacuuagaguaacuggugcaucuuuugaagcgguuggacuaccugauuguauuacgcaagguaaaguugcugcugaagaaguaaucgcagaguuguaa

SEQ ID NO: 86 - protein, 121 aa, Artificial Sequence
  Chemically synthesized sequence
FTFEPFPTNEEIESNKKMLEKEKAYKESFKNSGLPTTLGKLDERLRNYLKKGTKNSAQFEKMVILTENKGYYTVYLNTPLAEDRKNVELLGKMYKTYFFKKGESKSSYVINGPGKTNEYAY

SEQ ID NO: 87 - DNA, 366 bp, Artificial Sequence
  Chemically synthesized sequence
ttcacctttgagcctttccccaccaatgaggaaatcgagagcaacaaaaagatgctggaaaaagagaaggcctataaggaatcttttaagaatagcggcctgcctacaacactgggcaagctggacgagcggctgagaaactacctcaagaaaggaaccaagaacagcgcccagttcgagaagatggtcatcctgaccgagaacaagggctactacaccgtgtacctgaacacccctctggctgaagatagaaagaacgtggaactgctgggaaaaatgtacaagacctacttcttcaagaagggcgagagcaagtccagctacgtgatcaacggcccaggcaagacaaacgagtacgcctactga

SEQ ID NO: 88 - RNA, 366 nt, Artificial Sequence
  Chemically synthesized sequence
uucaccuuugagccuuuccccaccaaugaggaaaucgagagcaacaaaaagaugcuggaaaaagagaaggccuauaaggaaucuuuuaagaauagcggccugccuacaacacugggcaagcuggacgagcggcugagaaacuaccucaagaaaggaaccaagaacagcgcccaguucgagaagauggucauccugaccgagaacaagggcuacuacaccguguaccugaacaccccucuggcugaagauagaaagaacguggaacugcugggaaaaauguacaagaccuacuucuucaagaagggcgagagcaaguccagcuacgugaucaacggcccaggcaagacaaacgaguacgccuacuga

SEQ ID NO: 89 - protein, 61 aa, Artificial Sequence
  Chemically synthesized sequence
LRNYLKKGTKNSAQFEKMVILTENKGYYTVYLNTPLAEDRKNVELLGKMYKTYFFKKGESK

SEQ ID NO: 90 - DNA, 183 bp, Artificial Sequence
  Chemically synthesized sequence
ctgagaaactacctcaagaaaggaaccaagaacagcgcccagttcgagaagatggtcatcctgaccgagaacaagggctactacaccgtgtacctgaacacccctctggctgaagatagaaagaacgtggaactgctgggaaaaatgtacaagacctacttcttcaagaagggcgagagcaag

SEQ ID NO: 91 - protein, 233 aa, Artificial Sequence
  Chemically synthesized sequence
MKKTIMASSLAVALGVTGYAAGTGHQAHAAEVNVDQAHLVDLAHNHQDQLNAAPIKDGAYDIHFVKDGFQYNFTSNGTTWSWSYEAANGQTAGFSNVAGADYTTSYNQGSNVQSVSYNAQSSNSNVEAVSAPTYHNYSTSTTSSSVRLSNGNTAGATGSSAAQIMAQRTGVSASTWAAIIARESNGQVNAYNPSGASGLFQTMPGWGPTNTVDQQINAAVKAYKAQGLGAWGF

SEQ ID NO: 92 - DNA, 702 bp, Artificial Sequence
  Chemically synthesized sequence
atgaaaaagaccatcatggcctctagcctggccgtcgccctgggcgtgacaggctacgccgctggtacaggccaccaggcccacgccgccgaagtgaacgtggatcaagcccatctggtggacctggcccacaaccaccaggaccagctgaacgccgctccaatcaaggatggcgcctacgacatccacttcgtgaaggacggcttccaatacaacttcacctctaatggcaccacatggtcctggagctacgaggccgccaacggccagacagccggatttagcaatgtcgccggcgcagattacaccaccagctacaaccagggcagcaacgtgcagtccgtgtcttacaatgcccagagcagcaacagcaacgtggaagccgtgtccgctcctacctaccacaactacagcaccagcaccacatctagcagcgtgcggctgagcaacggcaacaccgccggcgccaccggctcttctgctgcccagatcatggcccaaagaaccggcgtgtccgccagcacatgggccgctatcatcgccagagagagcaatggacaggtgaacgcctacaaccccagcggcgctagcggcctgttccagaccatgcctggctggggccctacaaacaccgtggaccagcagattaacgccgctgttaaggcctataaggcccagggactcggagcttggggattctga

SEQ ID NO: 93 - RNA, 702 nt, Artificial Sequence
  Chemically synthesized sequence
augaaaaagaccaucauggccucuagccuggccgucgcccugggcgugacaggcuacgccgcugguacaggccaccaggcccacgccgccgaagugaacguggaucaagcccaucugguggaccuggcccacaaccaccaggaccagcugaacgccgcuccaaucaaggauggcgccuacgacauccacuucgugaaggacggcuuccaauacaacuucaccucuaauggcaccacaugguccuggagcuacgaggccgccaacggccagacagccggauuuagcaaugucgccggcgcagauuacaccaccagcuacaaccagggcagcaacgugcaguccgugucuuacaaugcccagagcagcaacagcaacguggaagccguguccgcuccuaccuaccacaacuacagcaccagcaccacaucuagcagcgugcggcugagcaacggcaacaccgccggcgccaccggcucuucugcugcccagaucauggcccaaagaaccggcguguccgccagcacaugggccgcuaucaucgccagagagagcaauggacaggugaacgccuacaaccccagcggcgcuagcggccuguuccagaccaugccuggcuggggcccuacaaacaccguggaccagcagauuaacgccgcuguuaaggccuauaaggcccagggacucggagcuuggggauucuga

SEQ ID NO: 94 - protein, 204 aa, Artificial Sequence
  Chemically synthesized sequence
AEVNVDQAHLVDLAHNHQDQLNAAPIKDGAYDIHFVKDGFQYNFTSNGTTWSWSYEAANGQTAGFSNVAGADYTTSYNQGSNVQSVSYNAQSSNSNVEAVSAPTYHNYSTSTTSSSVRLSNGNTAGATGSSAAQIMAQRTGVSASTWAAIIARESNGQVNAYNPSGASGLFQTMPGWGPTNTVDQQINAAVKAYKAQGLGAWGF

SEQ ID NO: 95 - DNA, 615 bp, Artificial Sequence
  Chemically synthesized sequence
gccgaagtgaacgtggatcaagcccatctggtggacctggcccacaaccaccaggaccagctgaacgccgctccaatcaaggatggcgcctacgacatccacttcgtgaaggacggcttccaatacaacttcacctctaatggcaccacatggtcctggagctacgaggccgccaacggccagacagccggatttagcaatgtcgccggcgcagattacaccaccagctacaaccagggcagcaacgtgcagtccgtgtcttacaatgcccagagcagcaacagcaacgtggaagccgtgtccgctcctacctaccacaactacagcaccagcaccacatctagcagcgtgcggctgagcaacggcaacaccgccggcgccaccggctcttctgctgcccagatcatggcccaaagaaccggcgtgtccgccagcacatgggccgctatcatcgccagagagagcaatggacaggtgaacgcctacaaccccagcggcgctagcggcctgttccagaccatgcctggctggggccctacaaacaccgtggaccagcagattaacgccgctgttaaggcctataaggcccagggactcggagcttggggattctga

SEQ ID NO: 96 - RNA, 615 nt, Artificial Sequence
  Chemically synthesized sequence
gccgaagugaacguggaucaagcccaucugguggaccuggcccacaaccaccaggaccagcugaacgccgcuccaaucaaggauggcgccuacgacauccacuucgugaaggacggcuuccaauacaacuucaccucuaauggcaccacaugguccuggagcuacgaggccgccaacggccagacagccggauuuagcaaugucgccggcgcagauuacaccaccagcuacaaccagggcagcaacgugcaguccgugucuuacaaugcccagagcagcaacagcaacguggaagccguguccgcuccuaccuaccacaacuacagcaccagcaccacaucuagcagcgugcggcugagcaacggcaacaccgccggcgccaccggcucuucugcugcccagaucauggcccaaagaaccggcguguccgccagcacaugggccgcuaucaucgccagagagagcaauggacaggugaacgccuacaaccccagcggcgcuagcggccuguuccagaccaugccuggcuggggcccuacaaacaccguggaccagcagauuaacgccgcuguuaaggccuauaaggcccagggacucggagcuuggggauucuga

SEQ ID NO: 97 - protein, 55 aa, Artificial Sequence
  Chemically synthesized sequence
AEVNVDQAHLVDLAHNHQDQLNAAPIKDGAYDIHFVKDGFQYNFTSNGTTWSWSY

SEQ ID NO: 98 - DNA, 165 bp, Artificial Sequence
  Chemically synthesized sequence
gccgaagtgaacgtggatcaagcccatctggtggacctggcccacaaccaccaggaccagctgaacgccgctccaatcaaggatggcgcctacgacatccacttcgtgaaggacggcttccaatacaacttcacctctaatggcaccacatggtcctggagctac

SEQ ID NO: 99 - RNA, 165 nt, Artificial Sequence
  Chemically synthesized sequence
gccgaagugaacguggaucaagcccaucugguggaccuggcccacaaccaccaggaccagcugaacgccgcuccaaucaaggauggcgccuacgacauccacuucgugaaggacggcuuccaauacaacuucaccucuaauggcaccacaugguccuggagcuac

SEQ ID NO: 100 - protein, 16 aa, Artificial Sequence
  Chemically synthesized sequence
SNVAGADYTTSYNQGS

SEQ ID NO: 101 - DNA, 60 bp, Artificial Sequence
  Chemically synthesized sequence
agcaatgtcgccggcgcagattacaccaccagctacaaccagggcagcaacgtgcagtcc

SEQ ID NO: 102 - RNA, 60 nt, Artificial Sequence
  Chemically synthesized sequence
agcaaugucgccggcgcagauuacaccaccagcuacaaccagggcagcaacgugcagucc

SEQ ID NO: 103 - protein, 38 aa, Artificial Sequence
  Chemically synthesized sequence
SGASGLFQTMPGWGPTNTVDQQINAAVKAYKAQGLGAW

SEQ ID NO: 104 - DNA, 111 bp, Artificial Sequence
  Chemically synthesized sequence
agcggcgctagcggcctgttccagaccatgcctggctggggccctacaaacaccgtggaccagcagattaacgccgctgttaaggcctataaggcccagggactcggagct

SEQ ID NO: 105 - RNA, 111 nt, Artificial Sequence
  Chemically synthesized sequence
agcggcgcuagcggccuguuccagaccaugccuggcuggggcccuacaaacaccguggaccagcagauuaacgccgcuguuaaggccuauaaggcccagggacucggagcu

SEQ ID NO: 106 - protein, 234 aa, Artificial Sequence
  Chemically synthesized sequence
MKKTLLASSLAVGLGIVAGNAGHEAHASEADLNKASLAQMAQSNDQTLNQKPIEAGAYNYTFDYEGFTYHFESDGTHFAWNYHATGANGADMSAQAPATNNVAPSAVQANQANQVQSQEVEAPQNAQTQQPQASTSNNSQVTATPTESKASEGSSVNVNAHLKQIAQRESGGNIHAVNPTSGAAGKYQFLQSTWDSVAPAKYKGVSPANAPESVQDAAAVKLYNTGGAGHWVTA

SEQ ID NO: 107 - DNA, 705 bp, Artificial Sequence
  Chemically synthesized sequence
atgaaaaagaccctgctggcctccagcctggctgtgggcctgggcatcgtggccggcaacgccggccacgaggctcacgcctctgaagccgacctgaacaaggccagcctggcccagatggcccaatctaacgaccagacactcaaccagaaacctatcgaggccggagcttataattacaccttcgactacgagggcttcacctaccacttcgagtctgatggcacccactttgcctggaactaccacgcaacaggcgccaatggcgctgatatgagcgcccaagctcctgccacaaacaacgtggctccatctgccgtgcaggccaaccaggccaaccaggtgcagagccaggaggtggaagcccctcagaacgcccagacccagcagccccaggccagcaccagcaacaacagccaggtcacagccacccctacagagagcaaggccagcgaaggcagcagcgtcaacgtgaacgcccatctgaagcagatcgcccaaagagaatccggcggaaatatccacgccgtgaaccccaccagcggcgccgccggaaagtaccagttcctgcagtctacctgggacagcgtggcccccgccaagtacaagggcgtgtcccctgctaatgcccctgagagcgtgcaggacgccgctgctgttaagctgtacaacaccggaggcgccggccactgggtgacagcctga

SEQ ID NO: 108 - RNA, 705 nt, Artificial Sequence
  Chemically synthesized sequence
augaaaaagacccugcuggccuccagccuggcugugggccugggcaucguggccggcaacgccggccacgaggcucacgccucugaagccgaccugaacaaggccagccuggcccagauggcccaaucuaacgaccagacacucaaccagaaaccuaucgaggccggagcuuauaauuacaccuucgacuacgagggcuucaccuaccacuucgagucugauggcacccacuuugccuggaacuaccacgcaacaggcgccaauggcgcugauaugagcgcccaagcuccugccacaaacaacguggcuccaucugccgugcaggccaaccaggccaaccaggugcagagccaggagguggaagccccucagaacgcccagacccagcagccccaggccagcaccagcaacaacagccaggucacagccaccccuacagagagcaaggccagcgaaggcagcagcgucaacgugaacgcccaucugaagcagaucgcccaaagagaauccggcggaaauauccacgccgugaaccccaccagcggcgccgccggaaaguaccaguuccugcagucuaccugggacagcguggcccccgccaaguacaagggcguguccccugcuaaugccccugagagcgugcaggacgccgcugcuguuaagcuguacaacaccggaggcgccggccacugggugacagccuga

SEQ ID NO: 109 - protein, 207 aa, Artificial Sequence
  Chemically synthesized sequence
SEADLNKASLAQMAQSNDQTLNQKPIEAGAYNYTFDYEGFTYHFESDGTHFAWNYHATGANGADMSAQAPATNNVAPSAVQANQANQVQSQEVEAPQNAQTQQPQASTSNNSQVTATPTESKASEGSSVNVNAHLKQIAQRESGGNIHAVNPTSGAAGKYQFLQSTWDSVAPAKYKGVSPANAPESVQDAAAVKLYNTGGAGHWVTA

SEQ ID NO: 110 - DNA, 624 bp, Artificial Sequence
  Chemically synthesized sequence
tctgaagccgacctgaacaaggccagcctggcccagatggcccaatctaacgaccagacactcaaccagaaacctatcgaggccggagcttataattacaccttcgactacgagggcttcacctaccacttcgagtctgatggcacccactttgcctggaactaccacgcaacaggcgccaatggcgctgatatgagcgcccaagctcctgccacaaacaacgtggctccatctgccgtgcaggccaaccaggccaaccaggtgcagagccaggaggtggaagcccctcagaacgcccagacccagcagccccaggccagcaccagcaacaacagccaggtcacagccacccctacagagagcaaggccagcgaaggcagcagcgtcaacgtgaacgcccatctgaagcagatcgcccaaagagaatccggcggaaatatccacgccgtgaaccccaccagcggcgccgccggaaagtaccagttcctgcagtctacctgggacagcgtggcccccgccaagtacaagggcgtgtcccctgctaatgcccctgagagcgtgcaggacgccgctgctgttaagctgtacaacaccggaggcgccggccactgggtgacagcctga

SEQ ID NO: 111 - RNA, 624 nt, Artificial Sequence
  Chemically synthesized sequence
ucugaagccgaccugaacaaggccagccuggcccagauggcccaaucuaacgaccagacacucaaccagaaaccuaucgaggccggagcuuauaauuacaccuucgacuacgagggcuucaccuaccacuucgagucugauggcacccacuuugccuggaacuaccacgcaacaggcgccaauggcgcugauaugagcgcccaagcuccugccacaaacaacguggcuccaucugccgugcaggccaaccaggccaaccaggugcagagccaggagguggaagccccucagaacgcccagacccagcagccccaggccagcaccagcaacaacagccaggucacagccaccccuacagagagcaaggccagcgaaggcagcagcgucaacgugaacgcccaucugaagcagaucgcccaaagagaauccggcggaaauauccacgccgugaaccccaccagcggcgccgccggaaaguaccaguuccugcagucuaccugggacagcguggcccccgccaaguacaagggcguguccccugcuaaugccccugagagcgugcaggacgccgcugcuguuaagcuguacaacaccggaggcgccggccacugggugacagccuga

SEQ ID NO: 112 - protein, 111 aa, Artificial Sequence
  Chemically synthesized sequence
APSAVQANQANQVQSQEVEAPQNAQTQQPQASTSNNSQVTATPTESKASEGSSVNVNAHLKQIAQRESGGNIHAVNPTSGAAGKYQFLQSTWDSVAPAKYKGVSPANAPES

SEQ ID NO: 113 - DNA, 333 bp, Artificial Sequence
  Chemically synthesized sequence
gctccatctgccgtgcaggccaaccaggccaaccaggtgcagagccaggaggtggaagcccctcagaacgcccagacccagcagccccaggccagcaccagcaacaacagccaggtcacagccacccctacagagagcaaggccagcgaaggcagcagcgtcaacgtgaacgcccatctgaagcagatcgcccaaagagaatccggcggaaatatccacgccgtgaaccccaccagcggcgccgccggaaagtaccagttcctgcagtctacctgggacagcgtggcccccgccaagtacaagggcgtgtcccctgctaatgcccctgagagc

SEQ ID NO: 114 - RNA, 354 nt, Artificial Sequence
  Chemically synthesized sequence
gcuccugccacaaacaacguggcuccaucugccgugcaggccaaccaggccaaccaggugcagagccaggagguggaagccccucagaacgcccagacccagcagccccaggccagcaccagcaacaacagccaggucacagccaccccuacagagagcaaggccagcgaaggcagcagcgucaacgugaacgcccaucugaagcagaucgcccaaagagaauccggcggaaauauccacgccgugaaccccaccagcggcgccgccggaaaguaccaguuccugcagucuaccugggacagcguggcccccgccaaguacaagggcguguccccugcuaaugccccugagagc

SEQ ID NO: 115 - DNA, 702 bp, Artificial Sequence
  Chemically synthesized sequence
atgaaaaagacaattatggcatcatcattagcagtggcattaggtgtaacaggttacgcagcaggtacaggacatcaagcacacgctgctgaagtaaacgttgatcaagcacacttagttgacttagcgcataatcaccaagatcaattaaatgcagctccaatcaaagatggtgcatatgacatccactttgtaaaagatggtttccaatataacttcacttcaaatggtactacatggtcatggagctatgaagcagctaatggtcaaactgctggtttctcaaacgttgcaggtgcagactacactacttcatacaaccaaggttcaaatgtacaatcagtaagctacaatgcacaatcaagtaactcaaacgttgaagctgtttcagctccaacttaccataactacagcacttcaactacttcaagttcagtgagattaagcaatggtaatactgcaggtgctactggttcatcagcagctcaaatcatggctcaacgtactggtgtttcagcttctacatgggctgcaatcatcgctcgtgaatcaaatggtcaagtaaatgcttacaacccatcaggtgcttcaggtttattccaaactatgccaggttggggtccaacaaacactgttgaccaacaaatcaacgcagctgttaaagcatacaaagcacaaggtttaggtgcttggggattctaa

SEQ ID NO: 116 - RNA, 702 nt, Artificial Sequence
  Chemically synthesized sequence
augaaaaagacaauuauggcaucaucauuagcaguggcauuagguguaacagguuacgcagcagguacaggacaucaagcacacgcugcugaaguaaacguugaucaagcacacuuaguugacuuagcgcauaaucaccaagaucaauuaaaugcagcuccaaucaaagauggugcauaugacauccacuuuguaaaagaugguuuccaauauaacuucacuucaaaugguacuacauggucauggagcuaugaagcagcuaauggucaaacugcugguuucucaaacguugcaggugcagacuacacuacuucauacaaccaagguucaaauguacaaucaguaagcuacaaugcacaaucaaguaacucaaacguugaagcuguuucagcuccaacuuaccauaacuacagcacuucaacuacuucaaguucagugagauuaagcaaugguaauacugcaggugcuacugguucaucagcagcucaaaucauggcucaacguacugguguuucagcuucuacaugggcugcaaucaucgcucgugaaucaaauggucaaguaaaugcuuacaacccaucaggugcuucagguuuauuccaaacuaugccagguugggguccaacaaacacuguugaccaacaaaucaacgcagcuguuaaagcauacaaagcacaagguuuaggugcuuggggauucuaa

SEQ ID NO: 117 - DNA, 696 bp, Artificial Sequence
  Chemically synthesized sequence
atgaagaaaacattactcgcatcatcattagcagtaggtttaggaatcgtagcaggaaatgcaggtcacgaagcccatgcaagtgaagcggacttaaataaagcatctttagcgcaaatggcgcaatcaaatgatcaaacattaaatcaaaaaccaattgaagctggggcttataattatacatttgactatgaagggtttacttatcactttgaatcagatggtacacactttgcttggaattaccatgcaacaggtactaatggagcagacatgagtgcacaagcacctgcaactaataatgttgcaccatcagctgttcaagctaatcaagtacaatcacaagaagttgaagcaccacaaaatgctcaaactcaacaaccacaagcatcaacatcaaacaattcacaagttactgcaacaccaactgaatcaaaatcatcagaaggttcatcagtaaatgtgaatgctcatctaaaacaaattgctcaacgtgaatcaggtggcaatattcatgctgtaaatccaacatcaggtgcagctggtaagtatcaattcttacaatcaacttgggattcagtagcacctgctaaatataaaggtgtatcaccagcaaatgctcctgaaagtgttcaagatgccgcagcagtaaaattatataacactggtggcgctggacattgggttactgcataa

SEQ ID NO: 118 - RNA, 696 nt, Artificial Sequence
  Chemically synthesized sequence
augaagaaaacauuacucgcaucaucauuagcaguagguuuaggaaucguagcaggaaaugcaggucacgaagcccaugcaagugaagcggacuuaaauaaagcaucuuuagcgcaaauggcgcaaucaaaugaucaaacauuaaaucaaaaaccaauugaagcuggggcuuauaauuauacauuugacuaugaaggguuuacuuaucacuuugaaucagaugguacacacuuugcuuggaauuaccaugcaacagguacuaauggagcagacaugagugcacaagcaccugcaacuaauaauguugcaccaucagcuguucaagcuaaucaaguacaaucacaagaaguugaagcaccacaaaaugcucaaacucaacaaccacaagcaucaacaucaaacaauucacaaguuacugcaacaccaacugaaucaaaaucaucagaagguucaucaguaaaugugaaugcucaucuaaaacaaauugcucaacgugaaucagguggcaauauucaugcuguaaauccaacaucaggugcagcugguaaguaucaauucuuacaaucaacuugggauucaguagcaccugcuaaauauaaagguguaucaccagcaaaugcuccugaaaguguucaagaugccgcagcaguaaaauuauauaacacugguggcgcuggacauuggguuacugcauaa

SEQ ID NO: 119 - RNA, 183 nt, Artificial Sequence
  Chemically synthesized sequence
cugagaaacuaccucaagaaaggaaccaagaacagcgcccaguucgagaagauggucauccugaccgagaacaagggcuacuacaccguguaccugaacaccccucuggcugaagauagaaagaacguggaacugcugggaaaaauguacaagaccuacuucuucaagaagggcgagagcaag

SEQ ID NO: 120 - protein, 86 aa, Artificial Sequence
  Chemically synthesized sequence
MSTSLPTSNEYQNEKLANELKSLLDELNVNELATGSLNTYYKRTIKISGQKAMYALKSKDFKKMSEAKYQLQKIYNEIDEALKSKY

SEQ ID NO: 121 - DNA, 261 bp, Artificial Sequence
  Chemically synthesized sequence
atgtctacaagcctgcctaccagcaacgagtaccagaacgagaagctggctaatgaactcaagtccctgctggatgagctgaacgtgaacgaactggccacaggcagcctgaatacctactacaagagaaccatcaagatcagcggccagaaggccatgtacgccctgaagagcaaggacttcaagaaaatgagcgaggccaagtaccaactgcagaaaatctacaacgaaatcgacgaggccctgaaatctaagtattga

SEQ ID NO: 122 - RNA, 261 nt, Artificial Sequence
  Chemically synthesized sequence
augucuacaagccugccuaccagcaacgaguaccagaacgagaagcuggcuaaugaacucaagucccugcuggaugagcugaacgugaacgaacuggccacaggcagccugaauaccuacuacaagagaaccaucaagaucagcggccagaaggccauguacgcccugaagagcaaggacuucaagaaaaugagcgaggccaaguaccaacugcagaaaaucuacaacgaaaucgacgaggcccugaaaucuaaguauuga

SEQ ID NO: 123 - protein, 205 aa, Artificial Sequence
  Chemically synthesized sequence
MAEVNVDQAHLVDLAHNHQDQLNAAPIKDGAYDIHFVKDGFQYNFTSNGTTWSWSYEAANGQTAGFSNVAGADYTTSYNQGSNVQSVSYNAQSSNSNVEAVSAPTYHNYSTSTTSSSVRLSNGNTAGATGSSAAQIMAQRTGVPASTWAAIIARESNGQVNAYNPSGASGLFQTMPGWGPTNTVDQQINAAVKAYKAQGLGAWGF

SEQ ID NO: 124 - DNA, 618 bp, Artificial Sequence
  Chemically synthesized sequence
atggctgaagtaaacgttgatcaagcacacttagttgacttagcgcataatcatcaagatcaattaaatgcagctccaatcaaagatggtgcatatgacatccactttgtaaaagatggtttccaatataactttacttcaaatggtactacatggtcatggagctatgaagcagctaatggtcaaactgctggtttctcaaacgttgcaggtgcagactacactacttcatacaaccaaggttcaaatgtacaatcagtaagctacaatgcacaatcaagtaactcaaacgttgaagctgtttcagctccaacttaccataactacagcacttcaactacttcaagttcagtgagattaagcaatggtaatactgcaggtgctactggttcatcagcagctcaaatcatggctcaacgtactggtgttccagcttctacatgggctgcaatcatcgctcgtgaatcaaatggtcaagtaaatgcttacaacccatcaggtgcttcaggtttattccaaactatgccaggttggggcccaacaaacactgttgaccaacaaatcaacgcagctgttaaagcatacaaagcacaaggtttaggtgcttggggattctaa

SEQ ID NO: 125 - RNA, 618 nt, Artificial Sequence
  Chemically synthesized sequence
auggcugaaguaaacguugaucaagcacacuuaguugacuuagcgcauaaucaucaagaucaauuaaaugcagcuccaaucaaagauggugcauaugacauccacuuuguaaaagaugguuuccaauauaacuuuacuucaaaugguacuacauggucauggagcuaugaagcagcuaauggucaaacugcugguuucucaaacguugcaggugcagacuacacuacuucauacaaccaagguucaaauguacaaucaguaagcuacaaugcacaaucaaguaacucaaacguugaagcuguuucagcuccaacuuaccauaacuacagcacuucaacuacuucaaguucagugagauuaagcaaugguaauacugcaggugcuacugguucaucagcagcucaaaucauggcucaacguacugguguuccagcuucuacaugggcugcaaucaucgcucgugaaucaaauggucaaguaaaugcuuacaacccaucaggugcuucagguuuauuccaaacuaugccagguuggggcccaacaaacacuguugaccaacaaaucaacgcagcuguuaaagcauacaaagcacaagguuuaggugcuuggggauucuaa